(19) United States
(12) Patent Application Publication
(10) Pub. No.: US 2022/0348698 A1
(43) Pub. Date: Nov. 3, 2022

(54) COMPOUND, POLYMER, COMPOSITION, COMPOSITION FOR FILM FORMATION, PATTERN FORMATION METHOD, INSULATING FILM FORMATION METHOD, AND METHOD FOR PRODUCING COMPOUND, AS WELL AS METHOD FOR PRODUCING IODINE-CONTAINING VINYL POLYMER AND ACETYLATED DERIVATIVE THEREOF

(71) Applicant: Mitsubishi Gas Chemical Company, Inc., Tokyo (JP)

(72) Inventors: Tadashi OMATSU, Hiratsuka-shi, Kanagawa (JP); Kentaro KATAOKA, Niigata-shi, Niigata (JP); Masahiro MATSUMOTO, Niigata-shi, Niigata (JP); Yushi NIIMI, Niigata-shi, Niigata (JP); Takashi MAKINOSHIMA, Hiratsuka-shi, Kanagawa (JP); Masatoshi ECHIGO, Chiyoda-ku,Tokyo (JP)

(21) Appl. No.: 17/633,856

(22) PCT Filed: Aug. 7, 2020
(86) PCT No.: PCT/JP2020/030501

§ 371 Date: Feb. 8, 2022

(30) Foreign Application Priority Data
Aug. 9, 2019 (JP) 2019-147847
Dec. 23, 2019 (JP) 2019-232130
May 8, 2020 (JP) 2020-082764

(51) Int. Cl. C08F 12/16; G03F 7/039; G03F 7/20; G03F 7/32

(52) U.S. Cl. CPC C08F 12/16; G03F 7/0397; G03F 7/20; G03F 7/32

(57) ABSTRACT

Provided is a compound having one or more halogens and an unsaturated double bond. Provided is a method for producing an iodine-containing vinyl monomer comprising:
a) a step of providing an iodine-containing alcohol substrate having a general structure represented by the formula (1-1):

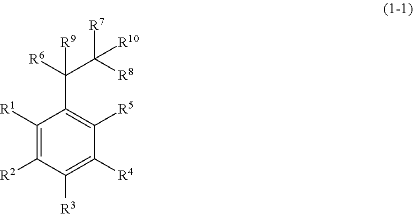

(the definitions of the variables in the formula (1-1) are as described in the specification); and
b) a step of dehydrating the iodine-containing alcohol substrate to obtain the iodine-containing vinyl monomer having a general structure represented by the formula (1):

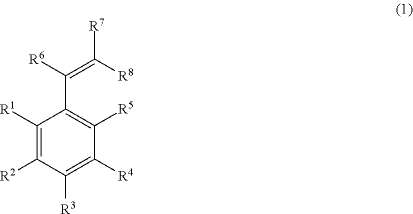

(the definitions of the variables in the formula (1) are as described in the specification).

### TECHNICAL FIELD

The present invention relates to a compound, a polymer, a composition, a composition for film formation, a pattern formation method, an insulating film formation method, and a method for producing a compound. The present invention also relates to a method for producing an iodine-containing vinyl polymer and acetylated derivative thereof.

### BACKGROUND ART

In recent years, in the production of semiconductor elements and liquid crystal display elements, semiconductors (patterns) and pixels have been rapidly miniaturized due to the advance in lithography technology. For pixel miniaturization, the exposure light source has been shifted to have a shorter wavelength, in general. Specifically, ultraviolet rays typified by g-ray and i-ray have been used conventionally, but nowadays, an exposure method for using far ultraviolet such as KrF excimer laser (248 nm) and ArF excimer laser (193 nm) is being the center of mass production. Furthermore, the introduction of extreme ultraviolet (EUV) lithography (13.5 nm) is progressing. In addition, electron beam (EB) is also used for forming a fine pattern.

Up to now, typical resist materials are polymer based resist materials capable of forming an amorphous film. Examples include polymer based resist compositions such as polymethyl methacrylate, polyhydroxy styrene with an acid dissociation group, and polyalkyl methacrylate (see, for example, Non Patent Literature 1). Conventionally, a line pattern of about 10 to 100 nm is formed by irradiating a resist thin film made by coating a substrate with a solution of these resist compositions with ultraviolet, far ultraviolet, electron beam, extreme ultraviolet or the like.

In addition, lithography using electron beam or extreme ultraviolet has a reaction mechanism different from that of normal photolithography (see Non Patent Literature 2, Non Patent Literature 3). Furthermore, lithography with electron beam or extreme ultraviolet aims at forming fine patterns of several nm to ten-odd nm. Accordingly, there is a demand for a resist composition having higher sensitivity to an exposure light source when the dimension of the resist pattern is reduced. In particular, lithography with extreme ultraviolet is required to further increase sensitivity in terms of throughput.

As a resist material that solves the problems as mentioned above, a resist composition having a metallic complex such as titanium, tin, hafnium and zirconium has been proposed (see, for example, Patent Literature 1).

There is a demand for a resist composition having higher sensitivity to an exposure light source when the dimension of the resist pattern is reduced, and 4-hydroxystyrene containing iodine is proposed as a raw material monomer of the resist composition, (see, for example, Patent Literatures 2 to 3), but the method for synthesizing an iodine-containing hydroxystyrene and acetylated derivative thereof is not disclosed.

On the other hand, many methods for synthesizing hydroxystyrene containing no iodine and acetylated derivative thereof are known (for example, Patent Literatures 4 to 6). However, these methods require an expensive reagent and stringent conditions, and gives a low yield, in general. When these synthesis methods are applied to iodine-containing hydroxystyrene and acetylated derivative thereof, the yield is further reduced, in general.

### CITATION LIST

### Patent Literature

Patent Literature 1: Japanese Patent Laid-Open No. 2015-108781
Patent Literature 2: US2019/0,187,342
Patent Literature 3: International Publication No. WO 2019/187881
Patent Literature 4: U.S. Pat. No. 4,316,995
Patent Literature 5: U.S. Pat. No. 5,274,060
Patent Literature 6: International Publication No. WO 2005/097719

### Non Patent Literature

Non Patent Literature 1: Shinji Okazaki et al., and eight others “Development of Lithography Technology in These 40 Years”, S&T Publishing Inc., Dec. 9, 2016
Non Patent Literature 2: H. Yamamoto, et al., Jpn. J. Appl. Phys. 46, L142 (2007)
Non Patent Literature 3: H. Yamamoto, et al., J. Vac. Sci. Technol. b 23, 2728 (2005)

### SUMMARY OF INVENTION

### Technical Problem

However, conventionally developed compositions for film formation have a problem of insufficient sensitivity to an exposure light source in the formation of a further thinned pattern.

To solve these problems, an object of the present invention is to provide a compound, a polymer, a composition, a resist composition, a pattern formation method, an insulating film formation method, and a method for producing a compound, by which a resist having excellent exposure sensitivity can be obtained.

As mentioned above, the method for producing iodine-containing hydroxystyrene and acetylated derivative thereof is not known, and in general, there are problems of requiring an expensive reagent and stringent conditions as well as low yield.

To solve these problems, an object of the present invention is to provide a method for producing an iodine-containing vinyl polymer (iodine-containing hydroxystyrene) and acetylated derivative thereof, without an expensive reagent and stringent conditions and with high yield.

### Solution to Problem

The inventors have, as a result of devoted examinations to solve the aforementioned problems, found out that a compound having a specific structure, or a polymer including the compound as a structural unit can increase the exposure sensitivity of a resist composition, and reached the present invention.

More specifically, the present invention is as follows.
[1]

A compound having one or more halogens and an unsaturated double bond.
[2]

The compound according to the above [1] having one or more hydrophilic groups or one decomposable group.
[3]

A compound according to the above [1] or [2] represented by the following formula (1):

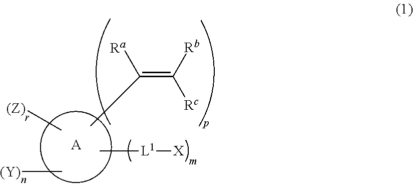

wherein

each X is independently I, F, Cl, Br, or an organic group having 1 to 30 carbon atoms and having 1 or more and 5 or less substituents selected from the group consisting of I, F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;

each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, I, F, Cl, Br, or an organic group having 1 to 60 carbon atoms and optionally having a substituent;

A is an organic group having 1 to 30 carbon atoms;

each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and

p is an integer of 1 or more, m is an integer of 1 or more, n is an integer of 0 or more, and r is an integer of 0 or more.
[4]

The compound according to the above [3] represented by the following formula (1a):

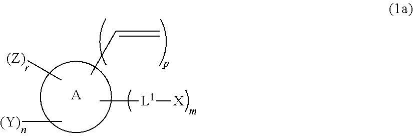

wherein

X, L¹, Y, A, Z, p, m, n, and r are as defined in the formula (1).
[5]

The compound according to the above [3] represented by the following formula (1b):

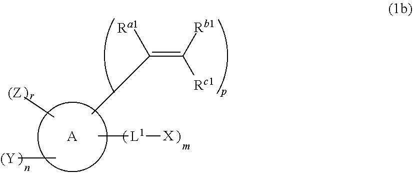

wherein

X, L¹, Y, A, Z, p, m, n, and r are as defined in the formula (1);

each of R^{a1}, R^{b1}, and R^{c1} is independently H, I, F, Cl, Br, or an organic group having 1 to 60 carbon atoms and optionally having a substituent; and

at least one of R^{a1}, R^{b1}, and R^{c1} is I, F, Cl, Br, or an organic group having 1 to 60 carbon atoms and optionally having a substituent.
[6]

The compound according to any one of the above [3] to [5], wherein n+r is an integer of 1 or more.
[7]

The compound according to any one of the above [3] to [6], wherein each Y is independently a group represented by the following formula (Y-1):

-L²-R² (Y-1)

wherein

L² is a group which is cleaved by an action of an acid or a base; and

R² is a linear, branched, or cyclic aliphatic group having 1 to 30 carbon atoms, an aromatic group having 1 to 30 carbon atoms, a linear, branched, or cyclic aliphatic group containing a heteroatom and having 1 to 30 carbon atoms, or an aromatic group containing a heteroatom and having 1 to 30 carbon atoms, and the aliphatic group, the aromatic group, the aliphatic group containing a heteroatom, and the aromatic group containing a heteroatom of R² optionally further have a substituent.
[8]

The compound according to any one of the above [3] to [7], wherein A is an aromatic ring.
[9]

The compound according to any one of the above [3] to [7], wherein A is an alicyclic structure.
[10]

The compound according to any one of the above [3] to [9], wherein A is a heterocyclic structure.
[11]

The compound according to any one of the above [3] to [10], wherein n is 2 or more.
[12]

The compound according to any one of the above [1] to [11], comprising a functional group for improving solubility in an alkaline developing solution by the action of an acid or a base.
[13]

The compound according to any one of the above [3] to [12], wherein X is I, and L¹ is a single bond.
[14]

The compound according to any one of the above [3] to [12], wherein X is an aromatic group into which one or more F, Cl, Br, or I are introduced.
[15]

The compound according to any one of the above [3] to [12], wherein X is an alicyclic group into which one or more F, Cl, Br, or I are introduced.
[16]

A composition comprising a compound represented by the formula (1C) in an amount of 1 ppm by mass or more and 10% by mass or less, based on the total compound according to any one of the [1] to [15]:

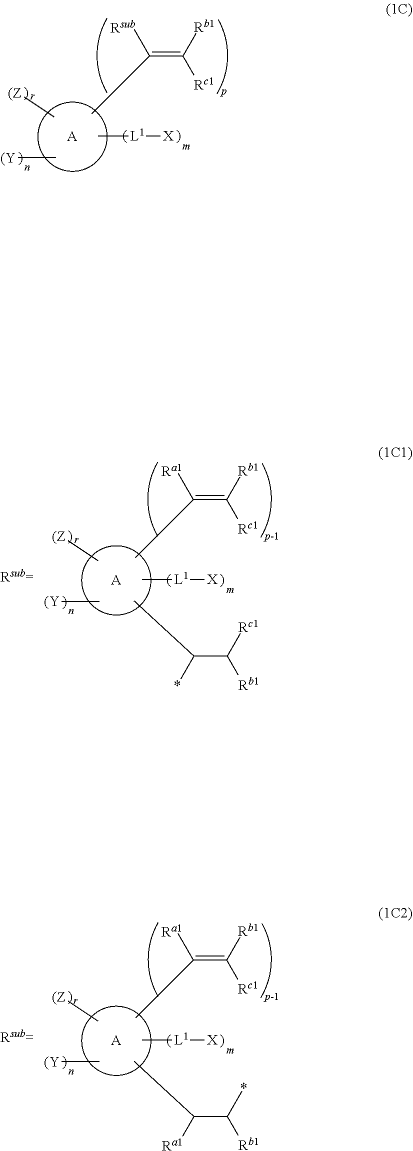

wherein

X, L¹, Y, A, Z, p, m, n, and r are as defined in the formula (1);

R^{sub} represents the formula (1C1) or the formula (1C2);

each of R^{a1}, R^{b1}, and R^{c1} is independently H, I, F, Cl, Br, or an organic group having 1 to 60 carbon atoms and optionally having a substituent;

at least one of R^{a1}, R^{b1}, and R^{c1} is I, F, Cl, Br, or an organic group having 1 to 60 carbon atoms and optionally having a substituent;

p-1 is an integer of 0 or more; and

* is a site for binding with an adjacent constitutional unit.
[17]

A composition comprising the compound according to the above [1] to [15] and a compound represented by the formula (1D) in an amount of 1 ppm by mass or more and 10% by mass or less, based on the compound according to the above [1] to [15]:

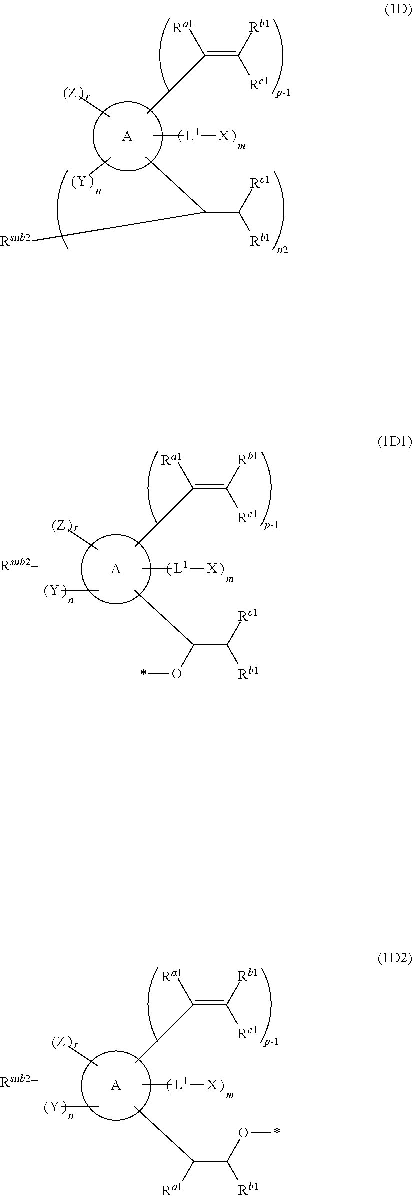

wherein

X, L¹, Y, A, Z, p, m, n, and r are as defined in the formula (1);

R^{sub2} represents the formula (1D1) or the formula (1D2);

each of R^{a1}, R^{b1}, and R^{c1} is independently H, I, F, Cl, Br, or an organic group having 1 to 60 carbon atoms and optionally having a substituent;

at least one of R^{a1}, R^{b1}, and R^{c1} is I, F, Cl, Br, or an organic group having 1 to 60 carbon atoms and optionally having a substituent;

n2 represents an integer of 0 or more and 4 or less;

p-1 is an integer of 0 or more; and

* is a site for binding with an adjacent constitutional unit.
[18]

A composition comprising a compound represented by the formula (1E) in an amount of 1 ppm by mass or more and 10% by mass or less, based on the compound according to any one of the above [3] to [15]:

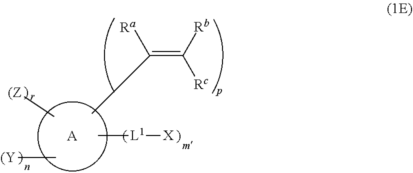

wherein
each X is independently F, Cl, Br, or an organic group having 1 to 30 carbon atoms and having 1 or more and 5 or less substituents selected from the group consisting of F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, F, Cl, Br, or an organic group having 1 to 60 carbon atoms and optionally having a substituent;

A is an organic group having 1 to 30 carbon atoms;

each Z is independently an alkoxy group, an ester group, an acetal group, or a carbonate ester group;

provided that none of X, L¹, Y, R^{a}, R^{b}, R^{c}, A, and Z contains I; and

p is an integer of 1 or more, m′ is an integer of 0 or more, n is an integer of 0 or more, and r is an integer of 0 or more.
[19]

A composition comprising the compound according to any one of the above [1] to [15],

wherein impurities containing K are 1 ppm by mass or less, in terms of element, based on the compound.
[20]

The composition according to the above [19], wherein peroxide is 10 ppm by mass or less, based on the compound.
[21]

The composition according to the above [19] or [20], wherein impurities containing one or more elements selected from the group consisting of Mn, Al, Si, and Li are 1 ppm by mass or less, in terms of element, based on the compound.
[22]

The composition according to any one of the above [19] to [21], wherein a phosphorus-containing compound is 10 ppm by mass or less, based on the compound.
[23]

The composition according to any one of the above [19] to [22], wherein maleic acid is 10 ppm by mass or less, based on the compound.
[24]

A polymer comprising a constitutional unit derived from the compound according to any one of the above [1] to [15].
[25]

The polymer according to the above [24], further comprising a constitutional unit represented by the following formula (C6):

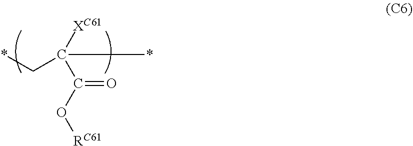

wherein

X^{C61} is a hydroxyl group or a halogen group;

each R^{C61} is independently an alkyl group having 1 to 20 carbon atoms; and

* is a site for binding with an adjacent constitutional unit.
[26]

A composition for film formation comprising the compound according to any one of the above [1] to [15] or the polymer according to the above [24] or [25].
[27]

The composition for film formation according to the above [26], further comprising an acid generating agent, a base generating agent, or a base compound.
[28]

A resist pattern formation method comprising:

a step of forming a resist film on a substrate by using a composition for film formation containing the compound according to any one of the above [1] to [15] or the polymer according to the above [24] or [25];

a step of exposing a pattern on the resist film; and

a step of subjecting the resist film to a development treatment after exposure.
[29]

An insulating film formation method comprising the method according to the above [28].
[30]

A method for producing a compound represented by the following formula (0), comprising a double bond introduction step of introducing an unsaturated double bond into a substituent Q of a compound represented by the following formula (S1):

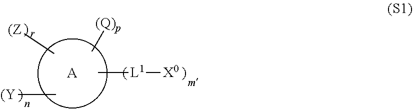

wherein

X⁰ is an organic group having 1 to 30 carbon atoms;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

A is an organic group having 1 to 30 carbon atoms;

each Z is independently an alkoxy group, an ester group, an acetal group, or a carbonate ester group;

Q is an organic group having 1 to 30 carbon atoms and having a hydroxyl group, an aldehyde group, a carboxyl group, or a ketone group; and

p is an integer of 1 or more, m′ is an integer of 0 or more, n is an integer of 0 or more, and r is an integer of 0 or more;

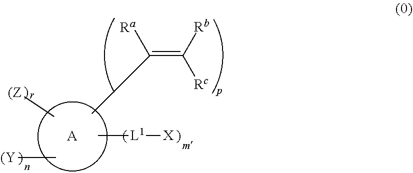

wherein

each X is independently I, F, Cl, Br, or an organic group having 1 to 30 carbon atoms and having 1 or more and 5 or less substituents selected from the group consisting of I, F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, I, F, Cl, Br, or an organic group having 1 to 60 carbon atoms and optionally having a substituent;

A is an organic group having 1 to 30 carbon atoms;

each Z is independently an alkoxy group, an ester group, an acetal group, or a carbonate ester group; and

p is an integer of 1 or more, m′ is an integer of 0 or more, n is an integer of 0 or more, and r is an integer of 0 or more.
[31]

The method for producing a compound according to the above [30], wherein the compound represented by the formula (S1) is a compound represented by the following formula (SA1), and

the method comprises a step designated by the following A1 and a step designated by the following A2: A1) a step of obtaining a compound represented by the following formula (SA2) by using the compound represented by the formula (SA1), and a compound represented by the following formula (RM1) or malononitrile; and A2) a step of obtaining the compound represented by the formula (0) by using a compound represented by the formula (SA2) and a fluoride source,

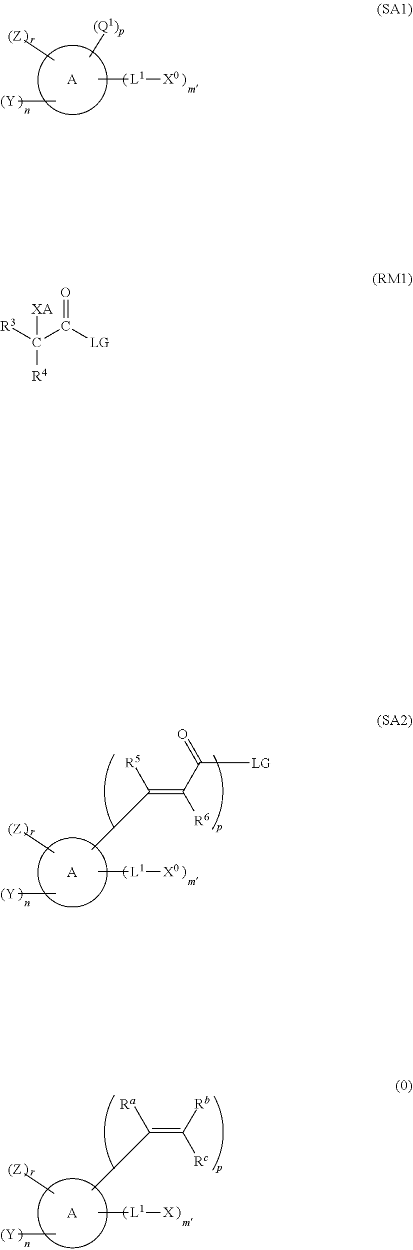

wherein

X⁰, L¹, Y, A, Z, p, m′, n, and r are as defined in the formulas (S1) and (0);

Q¹ is aldehyde or ketone;

LG is a group selected from a hydroxy groups, an alkoxy group, a carbonate ester group, an acetal group, and a carboxy group, and the alkoxy group, the carbonate ester group, the acetal group, and the carboxy group contain an aliphatic group or an aromatic group optionally having a substituent having 1 to 60 carbon atoms;

R³ is a hydrogen group, or a carboxy group or ester group optionally having a substituent having 1 to 60 carbon atoms;

R⁴ is a hydrogen group;

each of R⁵ and R⁶ is independently H, F, Cl, Br, or an organic group having 1 to 60 carbon atoms and optionally having a substituent; and

XA is a group selected from a hydrogen group and a halogen group.
[32]

The method for producing a compound according to the above [31], wherein in the step designated by A2, the compound represented by the formula (SA2) is subjected to a decarboxylation reaction at 100° C. or less by using the fluoride source.
[33]

The method for producing a compound according to the above [31] or [32], wherein in the step designated by A1, the compound represented by the formula (SA2) is obtained by further using a reducing agent.
[34]

The method for producing a compound according to any one of the above [30] to [33], wherein in the above formula (S1), A is benzene, toluene, or a heteroaromatic ring.
[35]

A method for producing the compound represented by the following formula (1), comprising: a step of forming a compound represented by the following formula (SB1) by at least one of compounds represented by the following formula (SB2A) and the following formula (SB3A) obtained through a step designated by the following B1A and at least one of steps designated by the following B2A and B3A; and a double bond introduction step of introducing an unsaturated double bond into a substituent Qb of the compound represented by the formula (SB1):
B1A) a step of providing the following substrate SB1A comprising one or more amino groups, and a core B having an aldehyde group or a ketone group;
B2A) a step of obtaining the compound represented by the following formula (SB2A) in which iodine is introduced into the core B; and
B3A) a step of obtaining the compound represented by the formula (SB3A) in which an amino group is substituted with a halogen group by a Sandmeyer reaction,

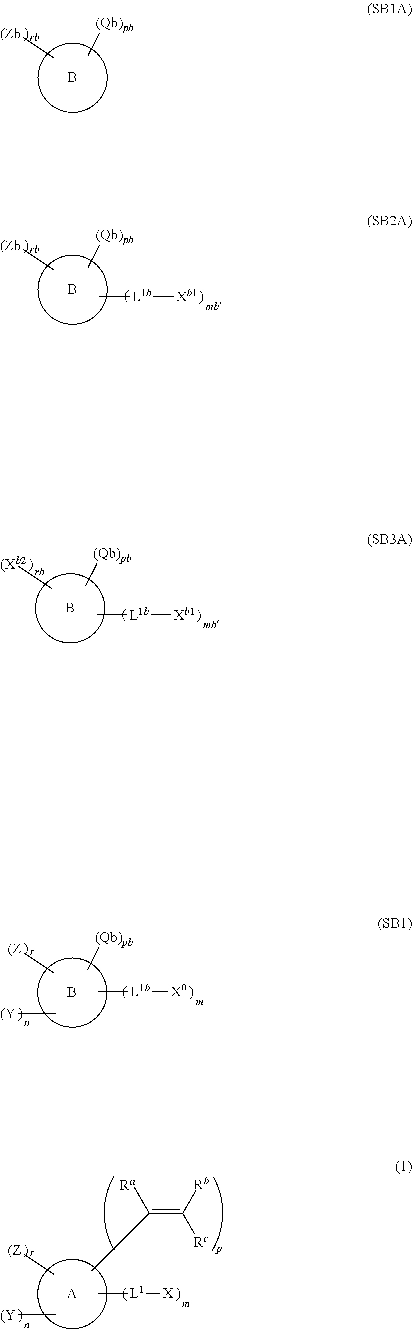

wherein, in the formula (1),

each X is independently I, F, Cl, Br, or an organic group having 1 to 30 carbon atoms and having 1 or more and 5 or less substituents selected from the group consisting of I, F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;

each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, I, F, Cl, Br, or an organic group having 1 to 60 carbon atoms and optionally having a substituent;

A is an organic group having 1 to 30 carbon atoms;

each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and

p is an integer of 1 or more, m is an integer of 1 or more, n is an integer of 0 or more, and r is an integer of 0 or more;

wherein, in the formulas (SB1A), (SB2A), (SB3A), and (SB1),

Zb represents an amino group optionally having a substituent consisting of a hydrogen group or a hydrocarbon group having 1 to 30 carbon atoms and optionally having a substituent, rb represents an integer of 1 or more, Qb, L^{1b}, X^{b1}, B, pb, and mb′ have the same meanings as Q, L, X, A, p, and m in the formula (1), respectively; and X^{b2} represents I, F, Cl, Br, or an organic group having 1 to 30 carbon atoms and having 1 or more and 5 or less substituents selected from the group consisting of I, F, Cl, and Br.
[36]

The production method according to the above [35], wherein the step of introducing a double bond comprises using an organic phosphorus compound and a base.
[37]

The method for producing a compound according to the above [30], comprising a halogen introduction step of introducing a halogen atom into the compound represented by the above formula (S1) by reaction with a halogenating agent.
[38]

The method for producing a compound according to any one of the above the [30] to the above [34], wherein the compound represented by the formula (SA1) is at least one of the compounds represented by the following formula (SB2A) and the following formula (SB3A) obtained through a step designated by the following B1A and at least one of steps designated by the following B2A and B3A:
B1A) a step of providing the following substrate SB1A comprising one or more amino groups, and a core B having an aldehyde group or a ketone group;
B2A) a step of obtaining the compound represented by the following formula (SB2A) in which iodine is introduced into the core B; and
B3A) a step of obtaining the compound represented by the formula (SB3A) in which an amino group is substituted with a halogen group by a Sandmeyer reaction,

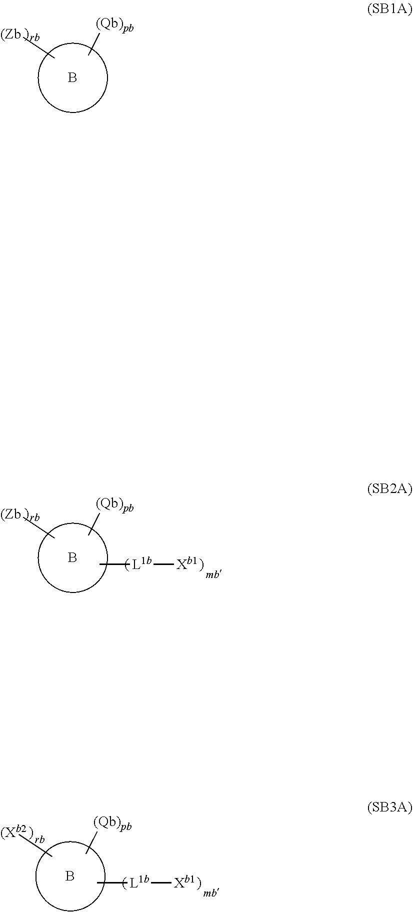

wherein

Zb represents an amino group optionally having a substituent consisting of a hydrogen group or a hydrocarbon group having 1 to 30 carbon atoms and optionally having a substituent, rb represents an integer of 1 or more, Qb, L^{1b}, X^{b1}, B, pb, and mb′ have the same meanings as Q, L, X, A, p, and m in the formula (1), respectively; and X^{b2} represents I, F, Cl, Br, or an organic group having 1 to 30 carbon atoms and having 1 or more and 5 or less substituents selected from the group consisting of I, F, Cl, and Br.
[39]

The method for producing a compound according to the above [36], wherein in the step designated by B2A, iodine is introduced into the core B at least using an iodine source and an oxidizing agent.
[40]

The method for producing a compound according to the above [30], wherein the compound represented by the formula (SA1) is a compound produced by a step designated by the following B1B, and at least either one of steps designated by the following B2B and B3B:
B1B) a step of providing the following substrate SB1B comprising one or more amino groups, and a core B having an aldehyde group or a ketone group;
B2B) a step of obtaining a compound represented by the formula (SB2B) in which iodine is introduced into the core B; and
B3B) a step of obtaining a compound represented by the formula (SB3B) in which an amino group is substituted with a halogen group,

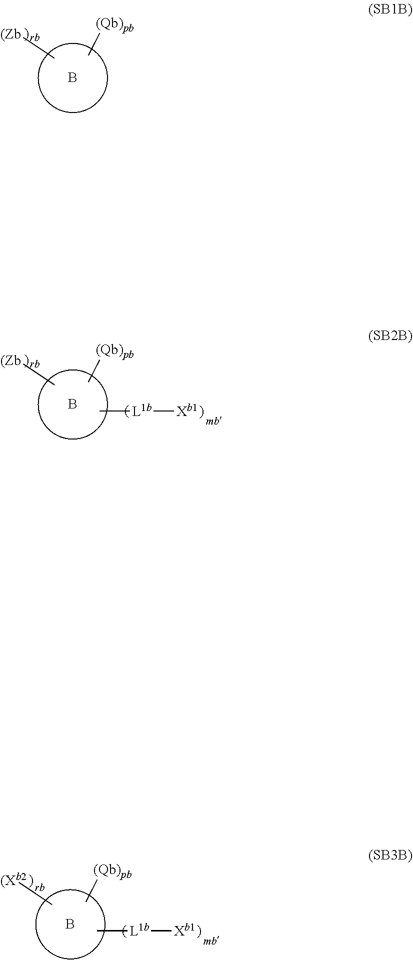

wherein

Zb represents an amino group optionally having a substituent consisting of a hydrogen group or a hydrocarbon group having 1 to 30 carbon atoms and optionally having a substituent, rb represents an integer of 1 or more, Qb, L^{1b}, X^{b1}, B, pb, and mb′ have the same meanings as Q, L, X, A, p, and m in the formula (1), respectively; and

X^{b2} represents I, F, Cl, Br, or an organic group having 1 to 30 carbon atoms and having 1 or more and 5 or less substituents selected from the group consisting of I, F, Cl, and Br.
[41]

The method for producing a compound according to the above [40], further comprising a step designated by the following B4a:

### B4a) Wittig Step

[42]

The method for producing a compound according to the above [38] or the above [41], wherein in the step designated by B2B, iodine is introduced into the core B by at least using an iodine source and an oxidizing agent.
[43]

The method for producing a compound according to any one of the above [40] to [42], wherein the core B has an aromatic ring structure optionally having a heteroatom.
[44]

A method for producing a compound represented by the following formula (1), comprising:

a halogen introduction step of introducing a halogen atom into a compound represented by the following formula (S1) by reaction with a halogenating agent; and

a double bond introduction step of introducing an unsaturated double bond into a substituent Q;

wherein the double bond introduction step comprises using an organic phosphorus compound and a base:

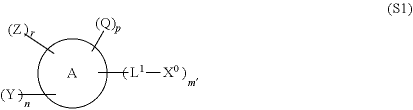

wherein

X⁰ is an organic group having 1 to 30 carbon atoms;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

A is an organic group having 1 to 30 carbon atoms;

each Z is independently an alkoxy group, an ester group, an acetal group, or a carbonate ester group;

Q is an organic group having 1 to 30 carbon atoms and having a hydroxyl group, an aldehyde group, a carboxyl group, or a ketone group; and

p is an integer of 1 or more, m′ is an integer of 0 or more, n is an integer of 0 or more, and r is an integer of 0 or more;

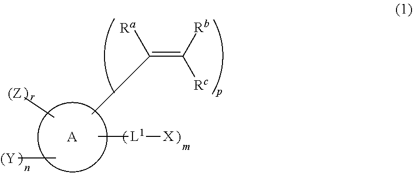

wherein

each X is independently I, F, Cl, Br, or an organic group having 1 to 30 carbon atoms and having 1 or more and 5 or less substituents selected from the group consisting of I, F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, I, F, Cl, Br, or an organic group having 1 to 60 carbon atoms and optionally having a substituent;

A is an organic group having 1 to 30 carbon atoms;

each Z is independently an alkoxy group, an ester group, an acetal group, or a carbonate ester group; and

p is an integer of 1 or more, m is an integer of 1 or more, n is an integer of 0 or more, and r is an integer of 0 or more.

The inventors have, as a result of devoted examinations to solve the aforementioned problems, found out that a method for producing an iodine-containing vinyl polymer and acetylated derivative thereof, without expensive reagent and stringent conditions and with high yield can be provided through specific steps, and reached the present invention.

More specifically, the present invention is as follows.
[45]

A method for producing an iodine-containing vinyl monomer comprising:

a) a step of providing an iodine-containing alcohol substrate having a general structure represented by the formula (1-1):

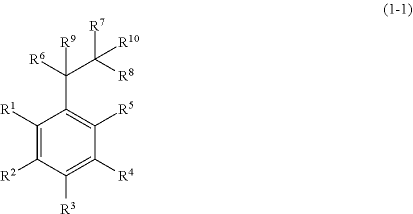

wherein

each of R¹ to R⁵ is independently H, OH, OCH₃, a halogen, or a linear or branched alkyl,

each of R⁶ to R¹⁰ is independently H, OH, OCH₃, a halogen, or a cyano group,

provided that at least one of R¹ to R⁵ is OH, at least one of R¹ to R⁵ is iodine, and at least one of R⁶ to R¹⁰ is OH or OCH₃; and
b) a step of dehydrating the iodine-containing alcohol substrate to obtain the iodine-containing vinyl monomer having a general structure represented by the formula (1):

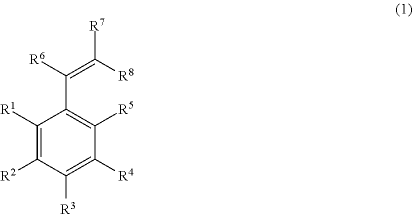

wherein

each of R¹ to R⁵ is independently H, OH, OCH₃, a halogen, or a linear or branched alkyl,

each of R⁶ to R⁸ is independently H, OH, OCH₃, a halogen, or a cyano group,

provided that at least one of R¹ to R⁵ is OH, and at least one of R¹ to R⁵ is iodine.
[46]

The method for producing an iodine-containing vinyl monomer according to the above [45], wherein the step of providing an iodine-containing alcohol substrate having a general structure represented by the formula (1-1) comprises:
c) a step of providing an iodine-containing ketone substrate having a general structure represented by the formula (1-2):

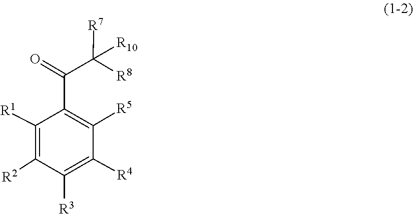

wherein

each of R¹ to R⁵ is independently H, OH, OCH₃, a halogen, or a linear or branched alkyl,

each of R⁷, R⁸, and R¹⁰ is independently H, OH, OCH₃, a halogen, or a cyano group,

provided that at least one of R¹ to R⁵ is OH and at least one of R¹ to R⁵ is iodine; and
d) a step of reducing the iodine-containing ketone substrate to obtain the iodine-containing alcohol substrate having the general structure represented by the formula (1-1). [47]

The method for producing an iodine-containing vinyl monomer according to the above [45], wherein the step of providing an iodine-containing alcohol substrate having a general structure represented by the formula (1-1) comprises:
e) a step of providing an alcohol substrate having a general structure represented by the formula (1-3):

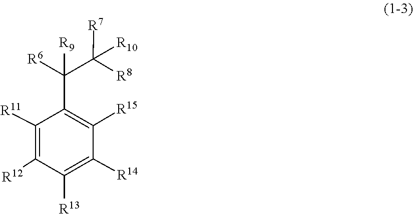

wherein

each of R¹¹ to R¹⁵ is independently H, OH, OCH₃, or a linear or branched alkyl,

each of R⁶ to R¹⁰ is independently H, OH, OCH₃, a halogen, or a cyano group,

provided that at least one of R¹¹ to R¹⁵ is OH and at least one of R⁶ to R¹⁰ is OH or OCH₃; and
f) a step of introducing iodine into the alcohol substrate to obtain the iodine-containing alcohol substrate having the general structure represented by the formula (1-1).
[48]

The method for producing an iodine-containing vinyl monomer according to the above [46], wherein the step of providing an iodine-containing ketone substrate having a general structure represented by the formula (1-2) comprises:
g) a step of providing a ketone substrate having a general structure represented by the formula (1-4):

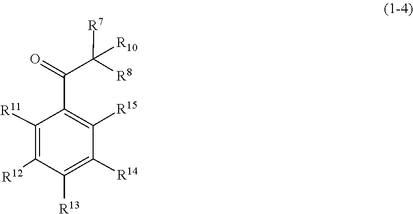

wherein

each of R¹¹ to R¹⁵ is independently H, OH, OCH₃, or a linear or branched alkyl,

each of R⁷, R⁸, and R¹⁰ is independently H, OH, OCH₃, a halogen, or a cyano group,

provided that at least one of R¹¹ to R¹⁵ is OH; and
h) a step of introducing iodine into the ketone substrate to obtain the iodine-containing ketone substrate having the general structure represented by the formula (1-2).
[49]

The method for producing an iodine-containing vinyl monomer according to the above [47], wherein the step of providing an alcohol substrate having a general structure represented by the formula (1-3) comprises:
i) a step of providing a ketone substrate having a general structure represented by the formula (1-4):

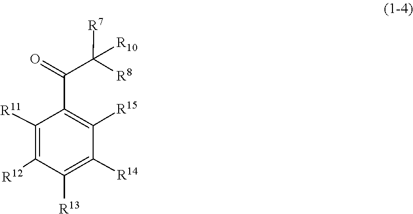

wherein

each of R¹¹ to R¹⁵ is independently H, OH, OCH₃, or a linear or branched alkyl,

each of R⁷, R⁸, and R¹⁰ is independently H, OH, OCH₃, a halogen, or a cyano group,

provided that at least one of R¹¹ to R¹⁵ is OH; and
j) a step of reducing the ketone substrate to obtain the alcohol substrate having the general structure represented by the formula (1-3).
[50]

A method for producing an iodine-containing acetylated vinyl monomer comprising:

k) a step of providing an iodine-containing vinyl monomer having a general structure represented by the formula (1):

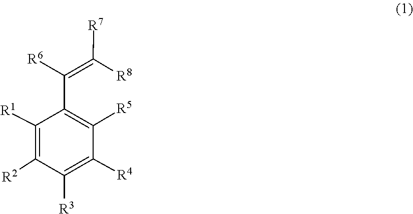

wherein

each of R¹ to R⁵ is independently H, OH, OCH₃, a halogen, or a linear or branched alkyl,

each of R⁶ to R⁸ is independently H, OH, OCH₃, a halogen, or a cyano group,

provided that at least one of R¹ to R⁵ is OH and at least one of R¹ to R⁵ is iodine; and
l) a step of acetylating the iodine-containing vinyl monomer to obtain the iodine-containing acetylated vinyl monomer having a general structure represented by the formula (2):

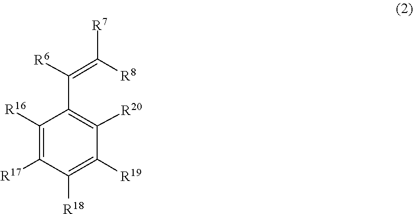

wherein

each of R¹⁶ to R²⁰ is independently H, OH, OCH₃, OAc, a halogen, or a linear or branched alkyl,

each of R⁶ to R⁸ is independently H, OH, OCH₃, a halogen, or a cyano group,

provided that at least one of R¹⁶ to R²⁰ is OAc and at least one of R¹⁶ to R²⁰ is iodine.
[51]

A method for producing an iodine-containing alcohol substrate comprising:

c) a step of providing an iodine-containing ketone substrate having a general structure represented by the formula (1-2):

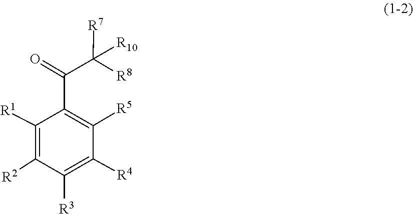

wherein

each of R¹ to R⁵ is independently H, OH, OCH₃, a halogen, or a linear or branched alkyl,

each of R⁷, R⁸, and R¹⁰ is independently H, OH, OCH₃, a halogen, or a cyano group,

provided that at least one of R¹ to R⁵ is OH and at least one of R¹ to R⁵ is iodine; and
d) a step of reducing the iodine-containing ketone substrate to obtain the iodine-containing alcohol substrate having a general structure represented by the formula (1-1):

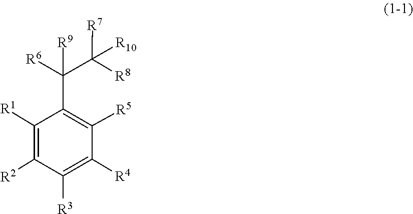

wherein

each of R¹ to R⁵ is independently H, OH, OCH₃, a halogen, or a linear or branched alkyl,

each of R⁶ to R¹⁰ is independently H, OH, OCH₃, a halogen, or a cyano group,

provided that at least one of R¹ to R⁵ is OH, at least one of R¹ to R⁵ is iodine, and at least one of R⁶ to R¹⁰ is OH or OCH₃.
[52]

A method for producing an iodine-containing alcohol substrate comprising:

e) a step of providing an alcohol substrate having a general structure represented by the formula (1-3):

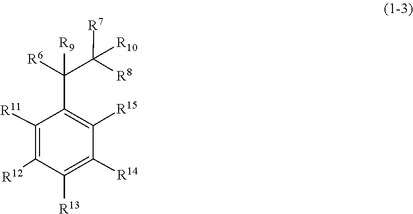

wherein

each of R¹¹ to R¹⁵ is independently H, OH, OCH₃, or a linear or branched alkyl,

each of R⁶ to R¹⁰ is independently H, OH, OCH₃, a halogen, or a cyano group,

provided that at least one of R¹¹ to R¹⁵ is OH and at least one of R⁶ to R¹⁰ is OH or OCH₃; and
f) a step of introducing iodine into the alcohol substrate to obtain the iodine-containing alcohol substrate having a general structure represented by the formula (1-1):

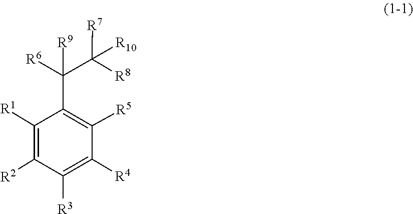

wherein

each of R¹ to R⁵ is independently H, OH, OCH₃, a halogen, or a linear or branched alkyl,

each of R⁶ to R¹⁰ is independently H, OH, OCH₃, a halogen, or a cyano group,

provided that at least one of R¹ to R⁵ is OH, at least one of R¹ to R⁵ is iodine, and at least one of R⁶ to R¹⁰ is OH or OCH₃.
[53]

A method for producing an iodine-containing ketone substrate comprising:

g) a step of providing a ketone substrate having a general structure represented by the formula (1-4):

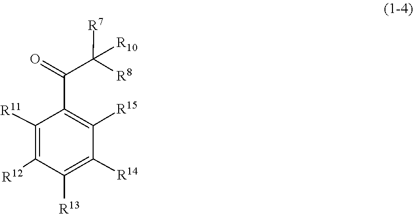

wherein

each of R¹¹ to R¹⁵ is independently H, OH, OCH₃, or a linear or branched alkyl,

each of R⁷, R⁸, and R¹⁰ is independently H, OH, OCH₃, a halogen, or a cyano group,

provided that at least one of R¹¹ to R¹⁵ is OH; and
h) a step of introducing iodine into the ketone substrate to obtain the iodine-containing ketone substrate having a general structure represented by the formula (1-2):

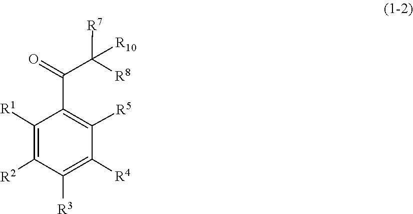

wherein

each of R¹ to R⁵ is independently H, OH, OCH₃, a halogen, or a linear or branched alkyl,

each of R⁷, R⁸, and R¹⁰ is independently H, OH, OCH₃, a halogen, or a cyano group,

provided that at least one of R¹ to R⁵ is OH, and at least one of R¹ to R⁵ is iodine.
[54]

A method for producing an alcohol substrate comprising:

i) a step of providing a ketone substrate having a general structure represented by the formula (1-4):

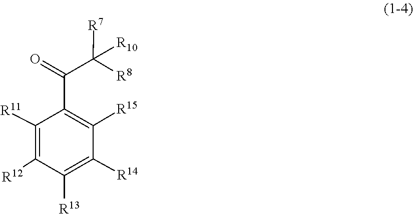

wherein

each of R¹¹ to R¹⁵ is independently H, OH, OCH₃, or a linear or branched alkyl,

each of R⁷, R⁸, and R¹⁰ is independently H, OH, OCH₃, a halogen, or a cyano group,

provided that at least one of R¹¹ to R¹⁵ is OH; and
j) a step of reducing the ketone substrate to obtain the alcohol substrate having a general structure represented by the formula (1-3):

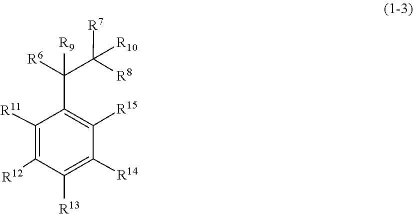

wherein

each of R¹¹ to R¹⁵ is independently H, OH, OCH₃, or a linear or branched alkyl,

each of R⁶ to R¹⁰ is independently H, OH, OCH₃, a halogen, or a cyano group,

provided that at least one of R¹¹ to R¹⁵ is OH and at least one of R⁶ to R¹⁰ is OH or OCH₃.

### Advantageous Effects of Invention

The present invention can provide a compound, a polymer, a composition, a resist composition, a pattern formation method, an insulating film formation method, and a method for producing a compound, by which a resist having excellent exposure sensitivity can be obtained.

Also, the present invention can provide a method for producing an iodine-containing vinyl polymer and acetylated derivative thereof, without an expensive reagent and stringent conditions and with high yield.

### DESCRIPTION OF EMBODIMENT

### First Embodiment

Hereinafter, the first embodiment of the present invention will be described (hereinafter, may be referred to as the “present embodiment”). The present embodiment is given in order to illustrate the present invention. The present invention is not limited to only the present embodiment.

As used herein, the meaning of each term is as follows.

The term “(meth)acrylate” means at least one selected from acrylate, haloacrylate, and methacrylate. The term haloacrylate means an acrylate in which the position of the methyl group in methacrylate is substituted with a halogen. Other terms having the expression (meth) should be similarly interpreted as (meth)acrylate.

The term “(co)polymer” means at least one selected from a homopolymer and a copolymer.

### [Compound (A)]

The compound according to the present embodiment (hereinafter, also referred to as the “compound (A)”) has one or more halogens and an unsaturated double bond. In addition, the compound (A) optionally further has one or more hydrophilic groups or one decomposable group. From the viewpoint of the roughness of a pattern, the compound (A) preferably has one or more hydrophilic groups or one decomposable group. That is, the compound according to the present embodiment has one or more halogens, one or more hydrophilic groups or one decomposable group, and an unsaturated double bond. In addition, the compound (A) optionally further has one or more hydrophilic groups or one decomposable group.

Examples of halogen include I, F, Cl, and Br. Among them, I, F, or Br is preferable, I or F is more preferable, and I is further preferable, from the viewpoint of the sensitizing effect of EUV and reducing the roughness of a pattern. The number of halogen is preferably an integer of 1 or more and 5 or less, more preferably an integer of 2 or more and 4 or less, and further preferably 2 or 3.

The term “hydrophilic group” means a group that binds to an organic compound, thereby improving the affinity between the organic compound and water. Examples of the hydrophilic group include a hydroxyl group, a nitro group, an amino group, a carboxyl group, a thiol group, a phosphine group, a phosphone group, a phosphate group, an ether group, a thioether group, a urethane group, a urea group, an amide group, and an imide group. Among them, a hydroxyl group or a carboxyl group is preferable, and a hydroxyl group is more preferable, from the viewpoint of the sensitizing effect of EUV and reducing the roughness of a pattern. The number of hydrophilic group is preferably an integer of 1 or more and 5 or less, more preferably an integer of 1 or more and 3 or less, further preferably 1 or 2, and particularly preferably 2.

The term “decomposable group” means a group that is decomposed in the presence of an acid or a base, or by an action of irradiation from a light source such as radiation, electron beam, extreme ultraviolet (EUV), ArF, or KrF. The decomposable group is not particularly limited, and for example, an acid dissociable functional group described in International Publication No. WO 2013/024778 can be used. Among the decomposable groups, a hydrolyzable group is preferable. The term “hydrolyzable group” means a group that is hydrolyzed in the presence of an acid or a base. Examples of the hydrolyzable group include an alkoxy group, an ester group, an acetal group, and a carbonate ester group. The number of decomposable group is preferably an integer of 1 or more and 5 or less, more preferably an integer of 1 or more and 3 or less, further preferably 1 or 2, and particularly preferably 2.

The unsaturated double bond is preferably a polymerizable unsaturated double bond. Examples of the group having an unsaturated double bond include, but are not particularly limited to, a vinyl group, an isopropenyl group, a (meth)acryloyl group, and a haloacryloyl group. Examples of the haloacryloyl group include an α-fluoroacryloyl group, an α-chloroacryloyl group, an α-bromoacryloyl group, an α-iodoacryloyl group, an α,β-dichloroacryloyl group, and an α,β-diiodoacryloyl group. Among these unsaturated double bonds, an isopropenyl group or a vinyl group is preferable. The number of unsaturated double bond is preferably an integer of 1 or more and 3 or less, more preferably an integer of 1 or more and 2 or less, and further preferably 1.

The compound according to the present embodiment (A) is preferably represented by the following formula (1). The compound (A) preferably comprises a functional group for improving solubility in an alkaline developing solution by the action of an acid or a base. The functional group for improving solubility in an alkaline developing solution by the action of an acid or a base is preferably contained in any of Z, Y, and X described below.

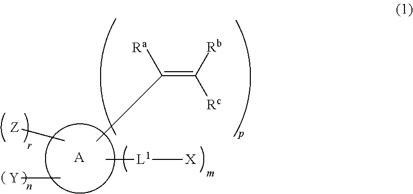

In the formula (1),

each X is independently I, F, Cl, Br, or an organic group having 1 to 30 carbon atoms and having 1 or more and 5 or less substituents selected from the group consisting of I, F, Cl, and Br. Among them, each X is preferably independently, I, F, Cl, or Br, more preferably independently, I, F, or Br, more preferably independently, I or F, and further preferably independently I.

In the present embodiment, unless otherwise defined, the term “substituted” means that one or more hydrogen atoms in a functional group are substituted with a substituent. Examples of the “substituent” include, but are not particularly limited to, a halogen atom, a hydroxyl group, a carboxyl group, a cyano group, a nitro group, a thiol group, a heterocyclic ring group, an alkyl group having 1 to 30 carbon atoms, an aryl group having 6 to 30 carbon atoms, an alkoxyl group having 1 to 30 carbon atoms, an alkenyl group having 2 to 30 carbon atoms, an alkynyl group having 2 to 30 carbon atoms, an acyl group having 1 to 30 carbon atoms, and an amino group having 0 to 30 carbon atoms.

The alkyl group may be any of a linear aliphatic hydrocarbon group, a branched aliphatic hydrocarbon group, and a cyclic aliphatic hydrocarbon group.

Examples of the alkyl group having 1 to 30 carbon atoms include, but are not limited to, a methyl group, an ethyl group, an n-propyl group, an i-propyl group, an n-butyl group, an i-butyl group, a t-butyl group, an n-pentyl group, an n-hexyl group, an n-dodecyl group, and a valeryl group.

Examples of the aryl group having 6 to 30 carbon atoms include, but are not limited to, a phenyl group, a naphthalene group, a biphenyl group, an anthracyl group, a pyrenyl group, and a perylene group.

Examples of the alkenyl group having 2 to 30 carbon atoms include, but are not limited to, an ethynyl group, a propenyl group, a butynyl group, and a pentynyl group.

Examples of the alkynyl group having 2 to 30 carbon atoms include, but are not limited to, an acetylene group and an ethynyl group.

Examples of an alkoxy group having 1 to 30 carbon atoms include, but are not limited to, a methoxy group, an ethoxy group, a propoxy group, a butoxy group, and a pentoxy group.

Examples of “the organic group having 1 to 30 carbon atoms and having 1 or more and 5 or less substituents selected from the group consisting of I, F, Cl, and Br” include, but are not particularly limited to, a monoiodophenyl group, a diiodophenyl group, a triiodophenyl group, a tetraiodophenyl group, a pentaiodophenyl group, a monoiodohydroxyphenyl group, a diiodohydroxyphenyl group, a triiodohydroxyphenyl group, a monoiodoacetoxyphenyl group, a diiodoacetoxyphenyl group, a triiodoacetoxyphenyl group, a monoiodo-t-butoxycarbonylphenyl group, a diiodo-t-butoxycarbonylphenyl group, a triiodo-t-butoxycarbonylphenyl group, a monoiododihydroxyphenyl group, a diiododihydroxyphenyl group, a triiododihydroxyphenyl group, a monoiododiacetoxyphenyl group, a diiododiacetoxyphenyl group, a triiododiacetoxyphenyl group, a monoiodo-di-t-butoxycarbonylphenyl group, a diiodo-di-t-butoxycarbonylphenyl group, a triiodo-di-t-butoxycarbonylphenyl group, a monoiodotrihydroxyphenyl group, a diiodotrihydroxyphenyl group, a monoiodotriacetoxyphenyl group, a diiodotriacetoxyphenyl group, a monoiodo-tri-t-butoxycarbonylphenyl group, a diiodo-tri-t-butoxycarbonylphenyl group, a monoiodonaphthyl group, a diiodonaphthyl group, a triiodonaphthyl group, a tetraiodonaphthyl group, a pentaiodonaphthyl group, a monoiodohydroxynaphthyl group, a diiodohydroxynaphthyl group, a triiodohydroxynaphthyl group, a monoiodoacetoxynaphthyl group, a diiodoacetoxynaphthyl group, a triiodoacetoxynaphthyl group, a monoiodo-t-butoxycarbonylnaphthyl group, a diiodo-t-butoxycarbonylnaphthyl group, a triiodo-t-butoxycarbonylnaphthyl group, a monoiododihydroxynaphthyl group, a diiododihydroxynaphthyl group, a triiododihydroxynaphthyl group, a monoiododiacetoxynaphthyl group, a diiododiacetoxynaphthyl group, a triiododiacetoxynaphthyl group, a monoiodo-di-t-butoxycarbonylnaphthyl group, a diiodo-di-t-butoxycarbonylnaphthyl group, a triiodo-di-t-butoxycarbonylnaphthyl group,

a monoiodotrihydroxynaphthyl group, a diiodotrihydroxynaphthyl group, a monoiodotriacetoxynaphthyl group, a diiodotriacetoxynaphthyl group, a monoiodo-tri-t-butoxycarbonylnaphthyl group, a diiodo-tri-t-butoxycarbonylnaphthyl group, a monoiodoadamantyl group, a diiodoadamantyl group, a triiodoadamantyl group, a monoiodohydroxyadamantyl group, a diiodohydroxynaphthyl group, a monoiodoacetoxynaphthyl group, a diiodoacetoxyadamantyl group, a monoiodo-t-butoxycarbonyladamantyl group, a diiodo-t-butoxycarbonyladamantyl group, a triiodo-t-butoxycarbonyladamantyl group, a monoiododihydroxyadamantyl group, a monoiododiacetoxyadamantyl group, a monoiodo-di-t-butoxycarbonyladamantyl group, a monoiodocyclohexyl group, a diiodocyclohexyl group, a triiodocyclohexyl group, a monoiodohydroxycyclohexyl group, a diiodohydroxynaphthyl group, a monoiodoacetoxynaphthyl group, a diiodoacetoxycyclohexyl group, a monoiodo-t-butoxycarbonylcyclohexyl group, a diiodo-t-butoxycarbonylcyclohexyl group, a triiodo-t-butoxycarbonylcyclohexyl group, a monoiododihydroxycyclohexyl group, a monoiododiacetoxycyclohexyl group, a monoiodo-di-t-butoxycarbonylcyclohexyl group,

a monobromophenyl group, a dibromophenyl group, a tribromophenyl group, a tetrabromophenyl group, a pentabromophenyl group, a monobromohydroxyphenyl group, a dibromohydroxyphenyl group, a tribromohydroxyphenyl group, a monobromoacetoxyphenyl group, a dibromoacetoxyphenyl group, a tribromoacetoxyphenyl group, a monobromo t-butoxycarbonylphenyl group, a dibromo t-butoxycarbonylphenyl group, a tribromo t-butoxycarbonylphenyl group, a monobromodihydroxyphenyl group, a dibromodihydroxyphenyl group, a tribromodihydroxyphenyl group, a monobromodiacetoxyphenyl group, a dibromodiacetoxyphenyl group, a tribromodiacetoxyphenyl group, a monobromo di-t-butoxycarbonylphenyl group, a dibromo di-t-butoxycarbonylphenyl group, a tribromo di-t-butoxycarbonylphenyl group,

a monobromotrihydroxyphenyl group, a dibromotrihydroxyphenyl group, a monobromotriacetoxyphenyl group, a dibromotriacetoxyphenyl group, a monobromotri-t-butoxycarbonylphenyl group, a dibromotri-t-butoxycarbonylphenyl group, a monobromoadamantyl group, a dibromoadamantyl group, a tribromoadamantyl group, a monobromohydroxyadamantyl group, a dibromohydroxynaphthyl group, a monobromoacetoxynaphthyl group, a dibromoacetoxyadamantyl group, a monobromo t-butoxycarbonyladamantyl group, a dibromo t-butoxycarbonyladamantyl group, a tribromo t-butoxycarbonyladamantyl group, a monobromodihydroxyadamantyl group, a monobromodiacetoxyadamantyl group, a monobromo-di-t-butoxycarbonyladamantyl group,

a monofluorophenyl group, a difluorophenyl group, a trifluorophenyl group, a tetrafluorophenyl group, a pentafluorophenyl group, a monofluorohydroxyphenyl group, a difluorohydroxyphenyl group, a trifluorohydroxyphenyl group, a monofluoroacetoxyphenyl group, a difluoroacetoxyphenyl group, a trifluoroacetoxyphenyl group, a monofluoro t-butoxycarbonylphenyl group, a difluoro t-butoxycarbonylphenyl group, a trifluoro t-butoxycarbonylphenyl group, a monofluorodihydroxyphenyl group, a difluorodihydroxyphenyl group, a trifluorodihydroxyphenyl group, a monofluorodiacetoxyphenyl group, a difluorodiacetoxyphenyl group, a trifluorodiacetoxyphenyl group, a monofluorodi-t-butoxycarbonylphenyl group, a difluorodi-t-butoxycarbonylphenyl group, a trifluorodi-t-butoxycarbonylphenyl group, a monofluorotrihydroxyphenyl group, a difluorotrihydroxyphenyl group, a monofluorotriacetoxyphenyl group, a difluorotriacetoxyphenyl group, a monofluorotri-t-butoxycarbonylphenyl group, a difluorotri-t-butoxycarbonylphenyl group, a monofluoroadamantyl group, a difluoroadamantyl group, a trifluoroadamantyl group, a monofluorohydroxyadamantyl group, a difluorohydroxynaphthyl group, a monofluoroacetoxynaphthyl group, a difluoroacetoxyadamantyl group, a monofluoro t-butoxycarbonyladamantyl group, a difluoro t-butoxycarbonyladamantyl group, a trifluoro t-butoxycarbonyladamantyl group, a monofluorodihydroxyadamantyl group, a monofluorodiacetoxyadamantyl group, a monofluoro-di-t-butoxycarbonyladamantyl group,

a monochlorophenyl group, a dichlorophenyl group, a trichlorophenyl group, a tetrachlorophenyl group, a pentachlorophenyl group, a monochlorohydroxyphenyl group, a dichlorohydroxyphenyl group, a trichlorohydroxyphenyl group, a monochloroacetoxyphenyl group, a dichloroacetoxyphenyl group, a trichloroacetoxyphenyl group, a monochioro t-butoxycarbonylphenyl group, a dichloro t-butoxycarbonylphenyl group, a trichloro t-butoxycarbonylphenyl group, a monochlorodihydroxyphenyl group, a dichlorodihydroxyphenyl group, a trichlorodihydroxyphenyl group, a monochlorodiacetoxyphenyl group, a dichlorodiacetoxyphenyl group, a trichlorodiacetoxyphenyl group, a monochlorodi-t-butoxycarbonylphenyl group, a dichlorodi-t-butoxycarbonylphenyl group, a trichlorodi-t-butoxycarbonylphenyl group,

a monochlorotrihydroxyphenyl group, a dichlorotrihydroxyphenyl group, a monochlorotriacetoxyphenyl group, a dichlorotriacetoxyphenyl group, a monochlorotri-t-butoxycarbonylphenyl group, a dichlorotri-t-butoxycarbonylphenyl group, a monochloroadamantyl group, a dichloroadamantyl group, a trichloroadamantyl group, a monochlorohydroxyadamantyl group, a dichlorohydroxynaphthyl group, a monochloroacetoxynaphthyl group, a dichloroacetoxyadamantyl group, a monochloro t-butoxycarbonyladamantyl group, a dichloro t-butoxycarbonyladamantyl group, a trichloro t-butoxycarbonyladamantyl group, a monochlorodihydroxyadamantyl group, a monochlorodiacetoxyadamantyl group, and a monochlorodi-t-butoxycarbonyladamantyl group.

For example, X may be an aromatic group into which one or more F, Cl, Br, or I are introduced. Examples of such an aromatic group include a group having a benzene ring such as a phenyl group and having 1 to 5 halogens, and a group having a heteroaromatic ring such as furan, thiophene, and pyridine and having 1 to 5 halogens. Examples thereof include a phenyl group having 1 to 5 I, a phenyl group having 1 to 5 F, a phenyl group having 1 to 5 Cl, a phenyl group having 1 to 5 Br, a naphthyl group having 1 to 5 F, a naphthyl group having 1 to 5 Cl, a naphthyl group having 1 to 5 Br, a naphthyl group having 1 to 5 I, a phenol group having 1 to 4 F, a phenol group having 1 to 4 Cl, a phenol group having 1 to 4 Br, a phenol group having 1 to 4 I, a furan group having 1 to 3 F, a furan group having 1 to 3 Cl, a furan group having 1 to 3 Br, a furan group having 1 to 3 I, a thiophene group having 1 to 3 F, a thiophene group having 1 to 3 Cl, a thiophene group having 1 to 3 Br, a thiophene group having 1 to 3 I, a pyridine group having 1 to 4 F, a pyridine group having 1 to 4 Cl, a pyridine group having 1 to 4 Br, a pyridine group having 1 to 4 I, a benzodiazole group having 1 to 5 F, a benzodiazole group having 1 to 5 Cl, a benzodiazole group having 1 to 5 Br, a benzodiazole group having 1 to 5 I, a benzimidazole group having 1 to 4 F, a benzimidazole group having 1 to 4 Cl, a benzimidazole group having 1 to 4 Br, a benzimidazole group having 1 to 4 I, a benzoxazole group having 1 to 4 F, a benzoxazole group having 1 to 4 Cl, a benzoxazole group having 1 to 4 Br, a benzoxazole group having 1 to 4 I, a benzothiophene group having 1 to 4 F, a benzothiophene group having 1 to 4 Cl, a benzothiophene group having 1 to 4 Br, and a benzothiophene group having 1 to 4 I. X may be an alicyclic group into which one or more F, Cl, Br, or I are introduced. Examples of such an alicyclic group include an adamantyl group having 1 to 3 halogens, an adamantyl group having 1 to 3 F, an adamantyl group having 1 to 3 Cl, an adamantyl group having 1 to 3 Br, an adamantyl group having 1 to 3 I, a cyclopentyl group having 1 to 3 F, a cyclopentyl group having 1 to 3 Cl, a cyclopentyl group having 1 to 3 Br, a cyclopentyl group having 1 to 3 I, a bicycloundecyl group having 1 to 3 F, a bicycloundecyl group having 1 to 3 Cl, a bicycloundecyl group having 1 to 3 Br, a bicycloundecyl group having 1 to 3 I, a norbornyl group having 1 to 3 F, a norbornyl group having 1 to 3 Cl, a norbornyl group having 1 to 3 Br, and a norbornyl group having 1 to 3 I.

L¹ is a single bond, an ether group, an ester group, a thioether group, an amino group, a thioester group, an acetal group, a phosphine group, a phosphone group, a urethane group, a urea group, an amide group, an imide group, or a phosphate group. Among them, L¹ is preferably a single bond. The ether group, the ester group, the thioether group, the amino group, the thioester group, the acetal group, the phosphine group, the phosphone group, the urethane group, the urea group, the amide group, the imide group, or the phosphate group of L¹ optionally has a substituent. Examples of such a substituent are as described above.

m is an integer of 1 or more, preferably an integer of 1 or more and 5 or less, more preferably an integer of 2 or more and 4 or less, and further preferably 2 or 3.

Each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent.

Examples of Y include at least one group selected from the group consisting of an alkoxy group [*³—O—R²], an ester group [*³—O—(C═O)—R2 or *³—(C═O)—O—R²], an acetal group [*³—O—(C(R²¹)₂)—O—R² (wherein each R²¹ is independently H or a hydrocarbon group having 1 to 10 carbon atoms)], a carboxyalkoxy group [*³—O—R²²—(C═O)—O—R² (wherein R²² is a divalent hydrocarbon group having 1 to 10 carbon atoms)], and a carbonate ester group [*³—O—(C═O)—O—R²]. The ester group is preferably a tertiary ester group, from the viewpoint of achieving high sensitivity. In the formula, *³ is a site for binding with A.

Among them, Y is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

Each Y is preferably independently a group represented by the following formula (Y-1).

-L²-R² (Y-1)

In the formula (Y-1),

L² is a group which is cleaved by the action of an acid or a base. Examples of the group which is cleaved by the action of an acid or a base include at least one divalent linking group selected from the group consisting of an ester group [*¹—O—(C═O)—*² or *¹—(C═O)—O—*²], an acetal group [*¹—O—(R²¹)₂)—O—*² (each R²¹ is independently H or a hydrocarbon group having 1 to 10 carbon atoms)], a carboxyalkoxy group [*¹—O—R²²—(C═O)—O—*² (R²² is a divalent hydrocarbon group having 1 to 10 carbon atoms)], and a carbonate ester group [*¹—O—(C═O)—O—*²]. The ester group is preferably a tertiary ester group, from the viewpoint of achieving high sensitivity. In the formula, *¹ is a site for binding with A, and *² is a site for binding with R². Among them, L² is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

As another effect, Y is preferably a group represented by the formula (Y-1) to control the polymerization properties of resin and the degree of polymerization in a desired range, when the compound (A) of the present embodiment is used as a polymerization unit of a copolymer. Since the compound A has a large influence on activity species in the polymer formation reaction due to having an X group and thus the desired control is difficult, variation of copolymer formation derived from the hydrophilic group and polymerization inhibition can be suppressed by having a group represented by the formula (Y-1) in the hydrophilic group in the compound A as a protecting group.

R² is a linear, branched, or cyclic aliphatic group having 1 to 30 carbon atoms, an aromatic group having 6 to 30 carbon atoms, a linear, branched, or cyclic aliphatic group containing a heteroatom and having 1 to 30 carbon atoms, or a linear, branched, or cyclic aromatic group containing a heteroatom and having 1 to 30 carbon atoms, and the aliphatic group, the aromatic group, the aliphatic group containing a heteroatom, and the aromatic group containing a heteroatom of R² optionally further have a substituent. As the substituent here, the aforementioned substituents are used, but a linear, branched, or cyclic aliphatic group having 1 to 20 carbon atoms or an aromatic group having 6 to 20 carbon atoms are preferable. Among them, R² is preferably an aliphatic group. The aliphatic group in R² is preferably a branched or cyclic aliphatic group. The number of carbon atoms of the aliphatic group is preferably 1 or more and 20 or less, more preferably 3 or more and 10 or less, and further preferably 4 or more and 8 or less. Examples of the aliphatic group include, but are not particularly limited to, a methyl group, an isopropyl group, a sec-butyl group, a tert-butyl group, an isobutyl group, a cyclohexyl group, a methylcyclohexyl group, and an adamantyl group. Among them, a tert-butyl group, a cyclohexyl group, or an adamantyl group is preferable.

L² is preferably *¹—(C═O)—O—*² or a carboxyalkoxy group, because, when L² is cleaved by the action of an acid or a base, a carboxylic acid group is formed and the difference in the solubility and the difference in the dissolution rate between a cleaved portion and an uncleaved portion are increased in the development treatment, so that the resolution is improved and, in particular, residues at the pattern bottom in thin line patterns are suppressed.

Specific examples of Y include the followings. Each of them is independently a group represented by any of the following formulas (Y-1-1) to (Y-1-7).

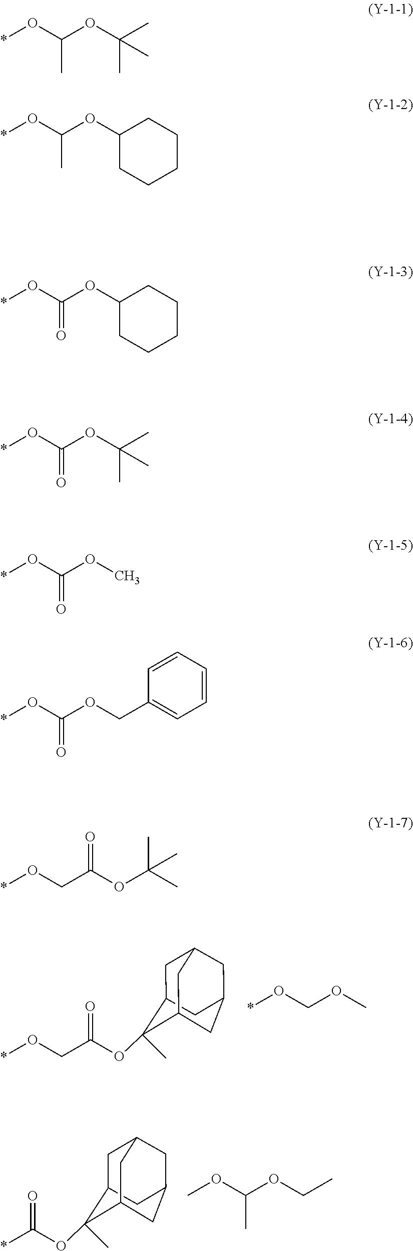

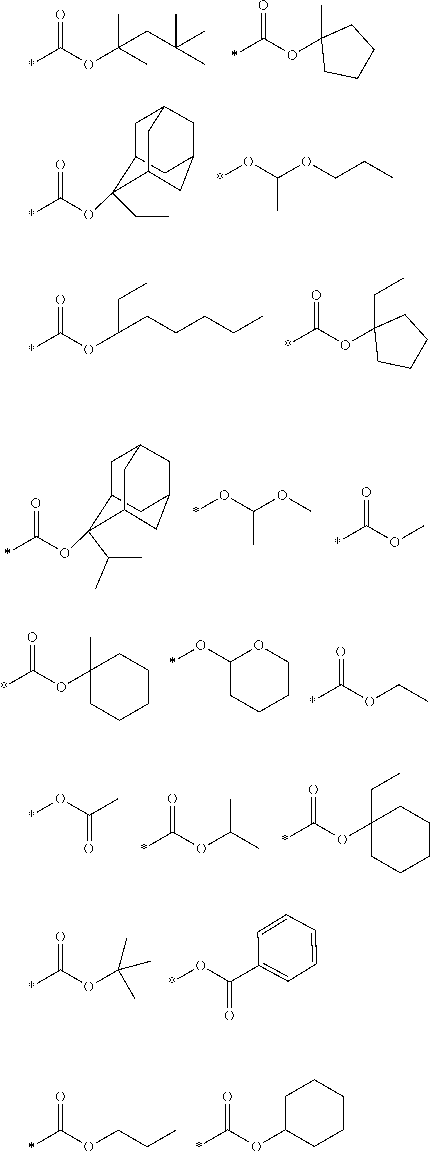

Examples of the alkoxy group that can be used as Y include an alkoxy group having 1 or more carbon atoms, and from the viewpoint of the solubility of a resin after the compound is combined with other monomers to form the resin, an alkoxy group having 2 or more carbon atoms is preferable, and an alkoxy group having 3 or more carbon atoms or having a cyclic structure is preferable.

Specific examples of the alkoxy group that can be used as Y include, but are not limited to, the followings.

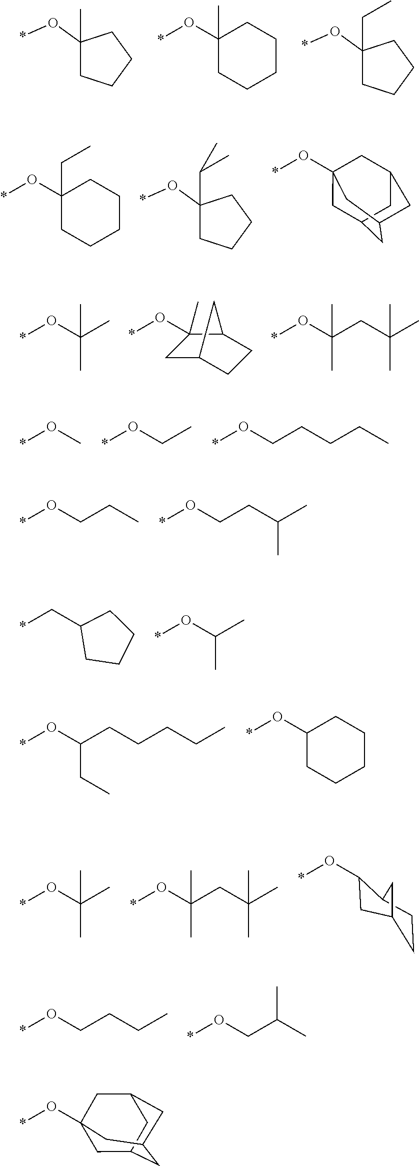

As the amino group and amide group that can be used as Y, a primary amino group, a secondary amino group, a tertiary amino group, a group having a quaternary ammonium salt structure, an amide having a substituent, or the like can be arbitrarily used. Specific examples of the amino group or amide group that can be used include, but are not limited to, the followings.

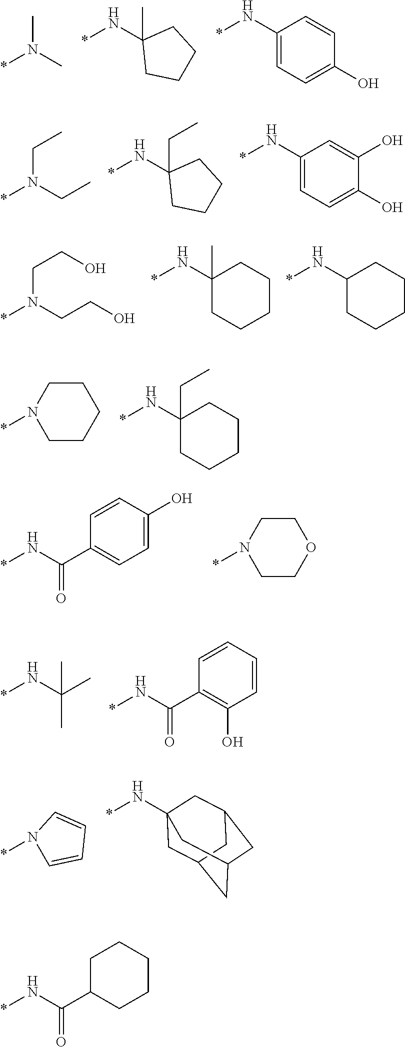

n is an integer of 0 or more, preferably an integer of 1 or more, more preferably an integer of 1 or more and 5 or less, further preferably an integer of 1 or more and 3 or less, still more preferably 1 or 2, and particularly preferably 2.

Each of R^{a}, R^{b}, and R^{c} is independently H, I, F, Cl, Br, or an organic group having 1 to 60 carbon atoms and optionally having a substituent. Examples of the substituent of the organic group having 1 to 60 carbon atoms include, but are not particularly limited to, I, F, Cl, Br, or other substituents. Examples of other substituents include, but are not particularly limited to, a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, and a phosphate group. Among them, the alkoxy group, the ester group, the carbonate ester group, the amino group, the ether group, the thioether group, the phosphine group, the phosphone group, the urethane group, the urea group, the amide group, the imide group, and the phosphate group optionally further have a substituent. Examples of the substituent here include a linear, branched, or cyclic aliphatic group having 1 to 20 carbon atoms and an aromatic group having 6 to 20 carbon atoms.

The number of carbon atoms of the organic group optionally having a substituent in R^{a}, R^{b}, and R^{c} is preferably 1 to 30.

Examples of the organic group having 1 to 60 carbon atoms and optionally having a substituent include, but are not particularly limited to, a linear or branched aliphatic hydrocarbon group having 1 to 60 carbon atoms, a cycloaliphatic hydrocarbon group having 4 to 60 carbon atoms, and an aromatic group having 6 to 60 carbon atoms and optionally having a heteroatom.

Examples of the linear or branched aliphatic hydrocarbon group having 1 to 60 carbon atoms include, but are not particularly limited to, a methyl group, an ethyl group, an n-propyl group, an isopropyl group, an n-butyl group, an isobutyl group, a tert-butyl group, an n-pentyl group, an n-hexyl group, an n-dodecyl group, a valeryl group, and a 2-ethylhexyl group.

Examples of the cycloaliphatic hydrocarbon group include, but are not particularly limited to, a cyclohexyl group, a cyclododecyl group, a dicyclopentyl group, a tricyclodecyl group, and an adamantyl group. Further, an aromatic group optionally having a heteroatom such as a benzodiazole group, a benzotriazole group, and a benzothiadiazole group can be arbitrarily selected. A combination of these organic groups can also be selected.

Examples of the aromatic group having 6 to 60 carbon atoms and optionally having a heteroatom include, but are not particularly limited to, a phenyl group, a naphthalene group, a biphenyl group, an anthracyl group, a pyrenyl group, a benzodiazole group, a benzotriazole group, and a benzothiadiazole group.

Among the organic group having 1 to 60 carbon atoms and optionally having a substituent, a methyl group is preferable, from the viewpoint of producing a polymer having a stable quality.

When R^{a} is an organic group having 1 or more and 8 or less carbon atoms, or a group selected from F, Cl, and I, n and r are preferably 0 or more.

A is an organic group having 1 to 30 carbon atoms. A may be a monocyclic organic group or a polycyclic organic group, or optionally has a substituent. A is preferably an aromatic ring optionally having a substituent. The number of carbon atoms of A is preferably 6 to 14, and more preferably 6 to 10.

A is preferably a group represented by any of the following formulas (A-1) to (A-4), more preferably a group represented by the following formulas (A-1) to (A-2), and further preferably a group represented by the following formula (A-1).

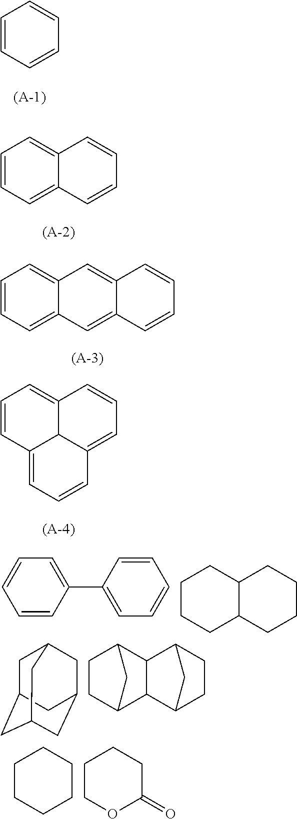

A may be an alicyclic structure optionally having a substituent. Here, the “alicyclic structure” refers to a saturated or unsaturated carbocycle having no aromatic properties. Examples of the alicyclic structure include a saturated or unsaturated carbocycle having 3 to 30 carbon atoms, and a saturated or unsaturated carbocycle having 3 to 20 carbon atoms is preferable. Examples of the alicyclic structure include a group having cyclopropyl, cyclobutyl, cyclopentyl, cyclohexyl, cycloheptyl, cyclooctyl, cyclononyl, cyclodecyl, cycloicosyl, cyclopropenyl, cyclobutenyl, cyclopentenyl, cyclohexenyl, cycloheptenyl, cyclooctenyl, cyclopentadienyl, cyclooctadienyl, adamantyl, bicycloundecyl, decahydronaphthyl, norbornyl, norbornadienyl, cubane, basketane, or housane.

A may also be a heterocyclic structure optionally having a substituent. Examples of the heterocyclic structure include, but are not particularly limited to, a nitrogen-containing cyclic structure such as pyridine, piperidine, piperidone, benzodiazole, and benzotriazole; a cyclic ether such as triazine, a cyclic urethane structure, cyclic urea, cyclic amide, cyclic imide, furan, pyran, and dioxolane; an alicyclic group having a lactone structure such as caprolactone, butyrolactone, nonalactone, decalactone, undecalactone, bicycloundecalactone, and phthalide.

p is an integer of 1 or more, preferably an integer of 1 or more and 3 or less, more preferably an integer of 1 or more and 2 or less, and further preferably 1.

Each Z is independently an alkoxy group, an ester group, an acetal group, a carboxyalkoxy group, or a carbonate ester group. These groups optionally have a substituent, and examples of the substituent include a hydrocarbon group having 1 to 60 carbon atoms and optionally further having a substituent. r is an integer of 0 or more, preferably an integer of 0 or more and 2 or less, more preferably an integer of 0 or more and 1 or less, and further preferably 0.

Examples of Z include at least one group selected from the group consisting of an alkoxy group [*³—O—R²], an ester group [*³—O—(C═O)—R2 or *³—(C═O)—O—R²], an acetal group [*3—O—(C(R²¹)²)—O—R² (wherein each R²¹ is independently H or a hydrocarbon group having 1 to 10 carbon atoms)], a carboxyalkoxy group [*³—O—R²²—(C═O)—O—R² (wherein R²² is a divalent hydrocarbon group having 1 to 10 carbon atoms)], and a carbonate ester group [*³—O—(C═O)—O—R²]. The ester group is preferably a tertiary ester group, from the viewpoint of achieving high sensitivity. In the formula, *³ is a site for binding with A.

Among them, Z is preferably a tertiary ester group, an acetal group, a carbonate ester group, or a carboxyalkoxy group, more preferably an acetal group, a carbonate ester group, or a carboxyalkoxy group, and further preferably an acetal group or a carboxyalkoxy group, from the viewpoint of high sensitivity. From the viewpoint of producing a polymer having a stable quality by radical polymerization, an ester group, a carboxyalkoxy group, and a carbonate ester group are preferable.

As described above, n is an integer of 0 or more, r is an integer of 0 or more, but at least one of n or r may be an integer of 1 or more. That is, n+r may be an integer of 1 or more.

Among the compounds (A) described above, a compound represented by the following formula (1a) is preferable.

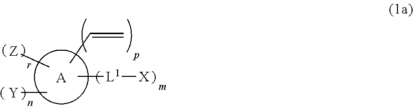

(In the formula (1a),

X, L¹, Y, A, Z, p, m, n, and r are as defined in the formula (1).)

Examples of the compound (A) according to the present embodiment (above all, the compound represented by the formula (1a)) include compounds having the structures given below.

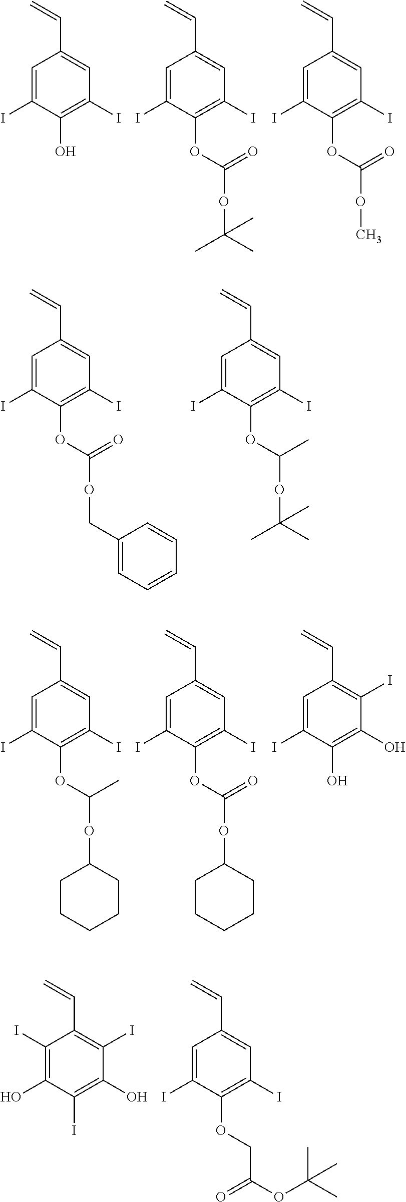

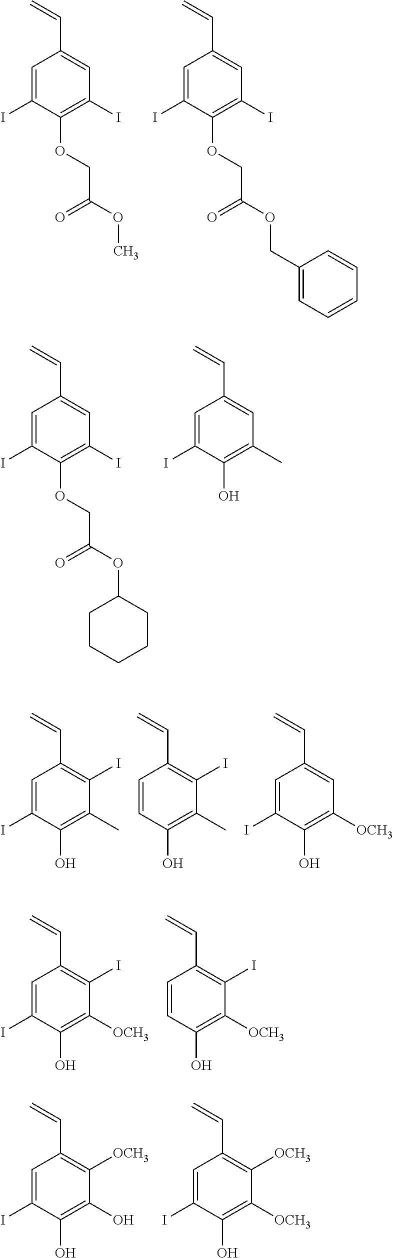

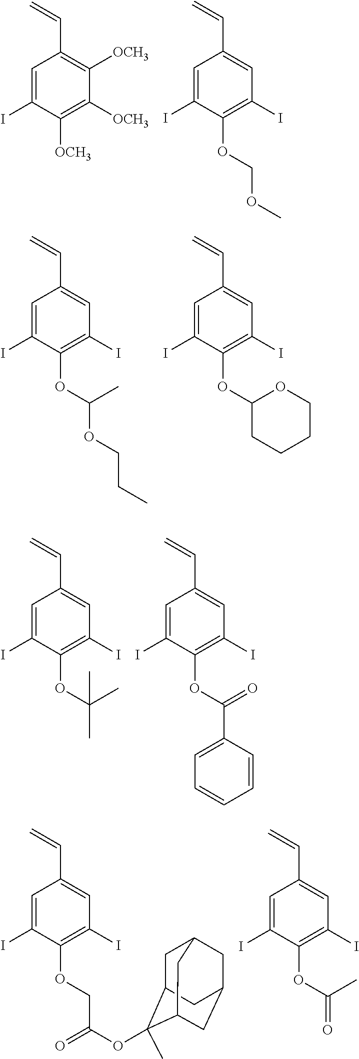

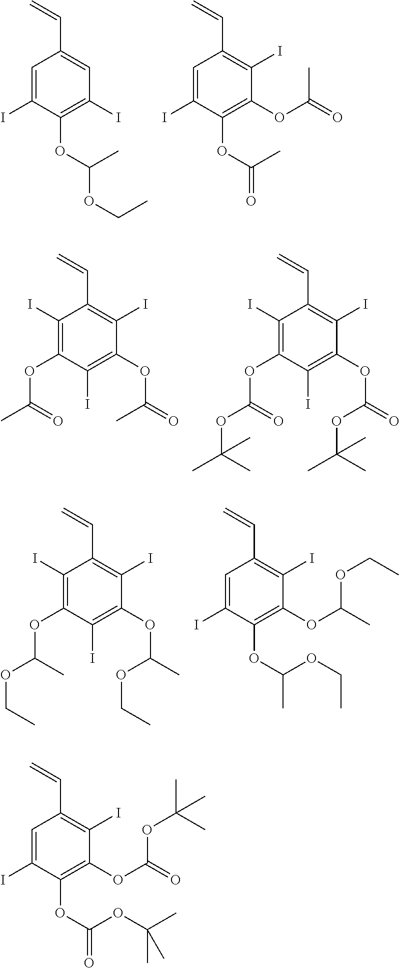

Among the compounds (A) described above, a compound represented by the following formula (1b) is preferable, from the viewpoint of improving the sensitivity.

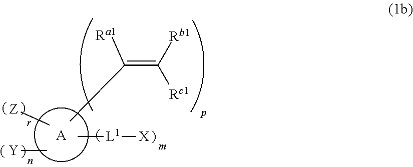

(In the formula (1b),

X, L¹, Y, A, Z, p, m, n, and r are as defined in the formula (1);

each of R^{a1}, R^{b1}, and R^{c1} is independently H, I, F, Cl, Br, or an organic group having 1 to 60 carbon atoms and optionally having a substituent; and

at least one of R^{a1}, R^{b1}, and R^{c1} is I, F, Cl, Br, or an organic group having 1 to 60 carbon atoms and optionally having a substituent.)

The organic group having 1 to 60 carbon atoms and optionally having a substituent in R^{a1}, R^{b1}, and R^{c1} has the same meaning as the aforementioned organic group having 1 to 60 carbon atoms and optionally having a substituent in R^{a}, R^{b}, and R^{c}. R^{a1} is preferably an organic group having 1 to 60 carbon atoms and optionally having a substituent, and more preferably a methyl group. R^{b1} and R^{c1} are preferably H.

Examples of the compound (A) according to the present embodiment (above all, the compound represented by the formula (1b)) include compounds having the structures given below.

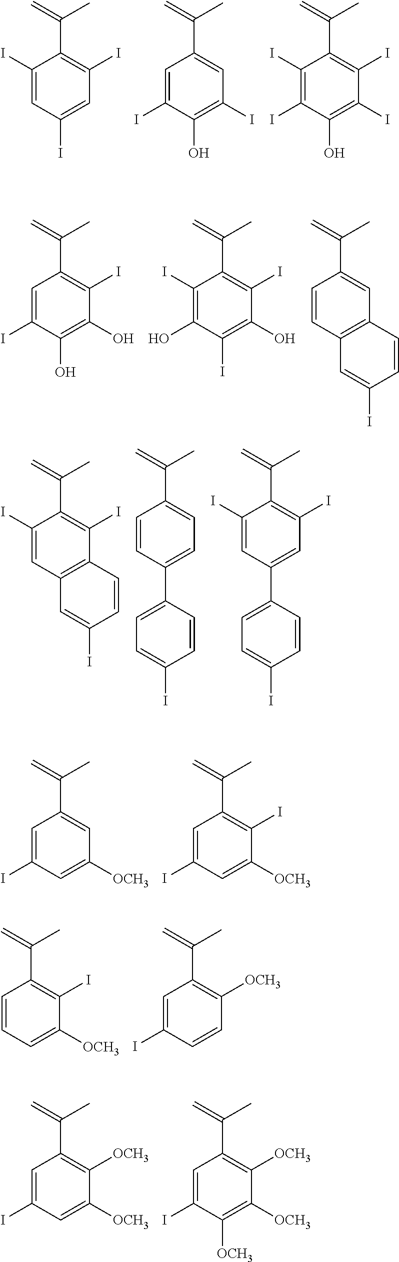

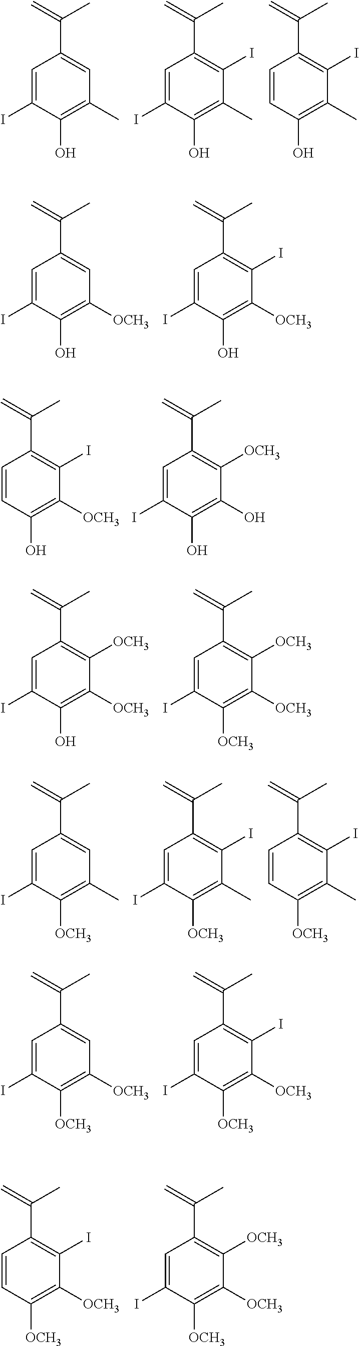

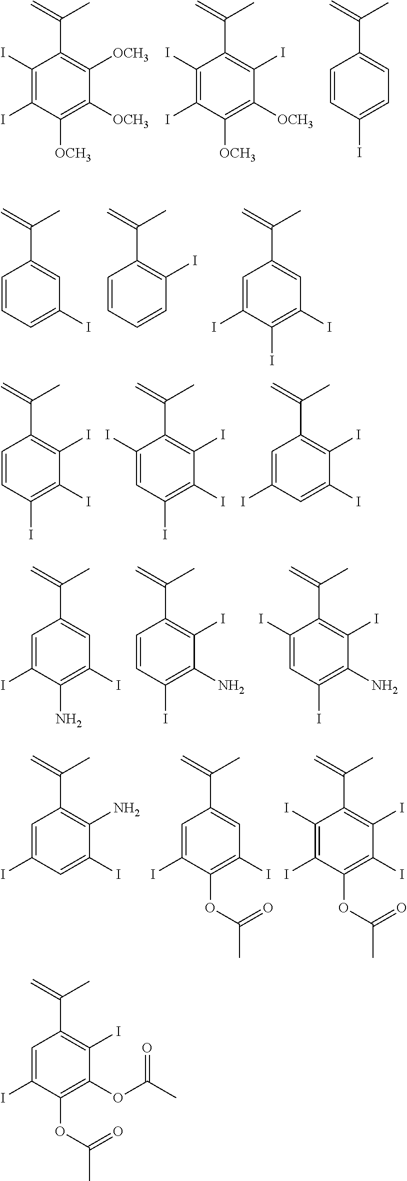

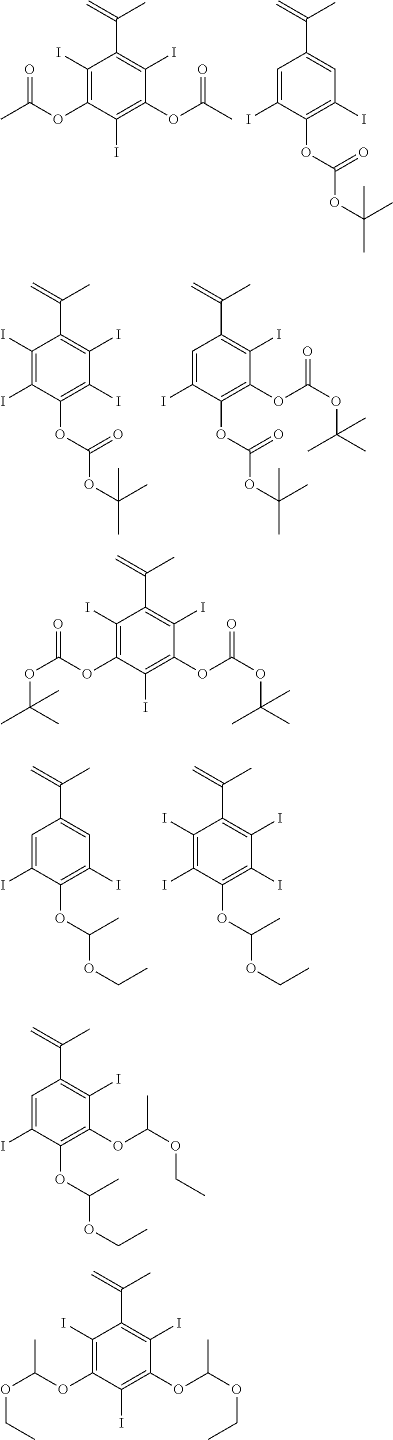

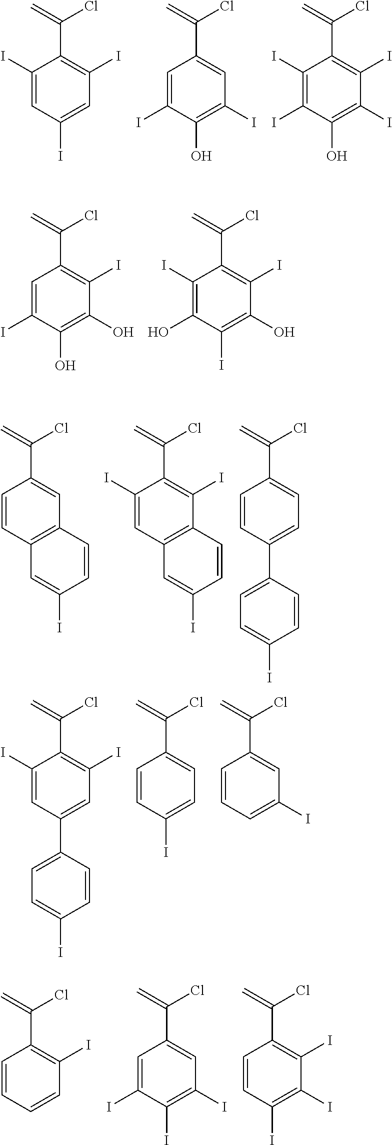

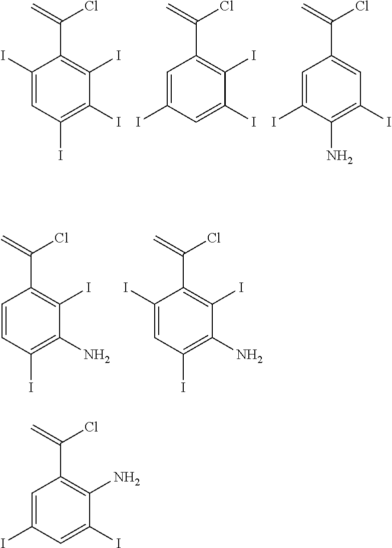

The compound (A) described above may be, for example, a compound represented by the following formula (1C). Although it is not particularly limited, the compound represented by the following formula (1C) is preferably used in combination with another compound (A) other than the compound represented by the formula (1C), as described below.

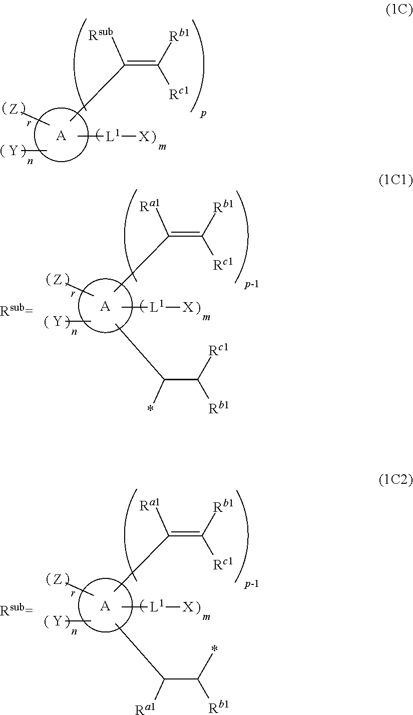

(In the formula (1C), the formula (1C1), and the formula (1C2),

X, L¹, Y, A, Z, p, m, n, and r are as defined in the formula (1);

R^{sub} represents the formula (1C1) or the formula (1C2);

each of R^{a1}, R^{b1}, and R^{c1} is independently H, I, F, Cl, Br, or an organic group having 1 to 60 carbon atoms and optionally having a substituent;

at least one of R^{a1}, R^{b1}, and R^{c1} is I, F, Cl, Br, or an organic group having 1 to 60 carbon atoms and optionally having a substituent;

p-1 is an integer of 0 or more; and

* is a site for binding with each formula.)

When the compound represented by the formula (1C) is used in a composition containing the compound (A) according to the present embodiment, the composition may contain the compound represented by the following formula (1C) in combination with another compound (A) other than the compound represented by the formula (1C). In this case, the composition is preferably prepared such that the compound represented by the formula (1C) is within a range of 1 ppm by mass or more and 10% by mass or less, more preferably within a range of 1 ppm by mass or more and 5% by mass or less, further preferably within a range of 1 ppm by mass or more and 3% by mass or less, and particularly preferably within a range of 1 ppm by mass or more and 1% by mass or less, based on the total compound (A). In the resin form after the formation of a resin made of starting materials including the composition thus prepared, the presence of a moiety containing X and a moiety consisting of Y or Z at a high density in the proximity area becomes the starting point for improving the sensitivity. Further, a local increase in the solubility of the resin leads to a reduction in the residue defect after development in a lithography process.

Examples of the compound (A) according to the present embodiment (above all, the compound represented by the formula (1C)) include compounds having the structures given below.

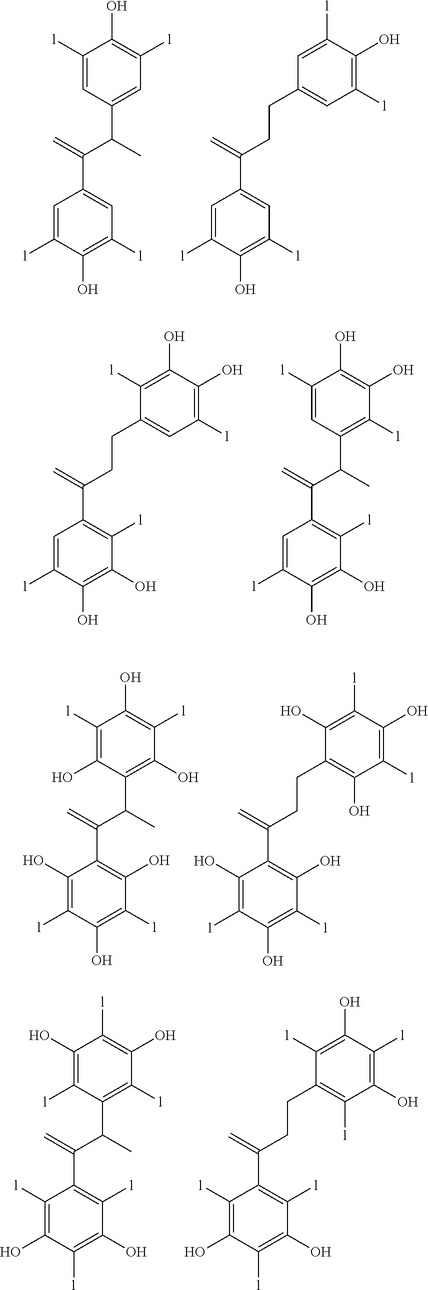

The compound (A) of the present embodiment may be used, for example, in combination with a compound represented by the following formula (1D).

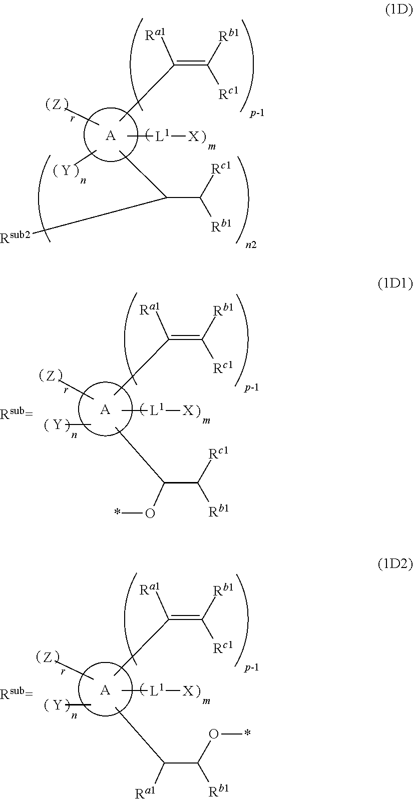

(In the formula (1D), the formula (1D1), or the formula (1D2),

X, L¹, Y, A, Z, p, m, n, and r are as defined in the formula (1);

R^{sub2} represents the formula (1D1) or the formula (1D2);

each of R^{a1}, R^{b1}, and R^{c1} is independently H, I, F, Cl, Br, or an organic group having 1 to 60 carbon atoms and optionally having a substituent;

at least one of R^{a1}, R^{b1}, and R^{c1} is I, F, Cl, Br, or an organic group having 1 to 60 carbon atoms and optionally having a substituent;

n2 represents an integer of 0 or more and 4 or less;

p-1 is an integer of 0 or more; and

* is a site for binding with an adjacent constitutional unit.)

When the compound represented by the formula (1D) is used in a composition containing the compound (A) of the present embodiment, the composition may contain the compound represented by the following formula (1D) in combination with another compound (A) other than the compound represented by the formula (1D). In this case, the composition is preferably prepared such that the compound represented by the formula (1D) is in a range of 1 ppm by mass or more and 10% by mass or less, more preferably in a range of 1 ppm by mass or more and 5% by mass or less, further preferably in a range of 1 ppm by mass or more and 3% by mass or less, and particularly preferably in a range of 1 ppm by mass or more and 1% by mass or less, based on the total compound (A). In the resin form in the case of forming a resin made of starting materials including the composition thus prepared, the coexistence of a moiety containing X and a moiety consisting of Y or Z at a high density in the proximity area becomes the starting point for improving the sensitivity. Further, a local increase in the solubility of the resin can lead to a reduction in the residue defect after development in a lithography process.

Examples of the compound (A) according to the present embodiment (above all, the compound represented by the formula (1D)) include compounds having the structures given below.

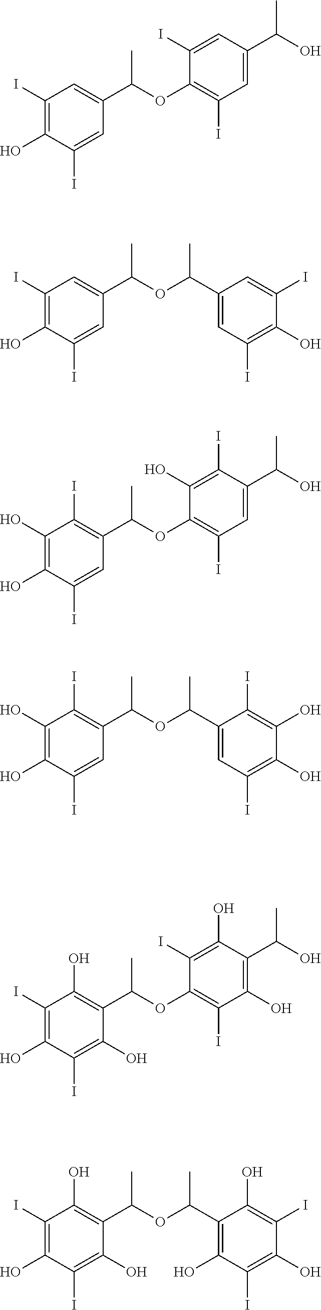 [SMILES: CC(O)c1cc(I)c(OC(C)c2cc(I)c(O)c(I)c2)c(I)c1 ; CC(OC(C)c1cc(I)c(O)c(I)c1)c1cc(I)c(O)c(I)c1 ; CC(O)c1cc(I)c(OC(C)c2cc(I)c(O)c(O)c2I)c(O)c1I ; CC(OC(C)c1cc(I)c(O)c(O)c1I)c1cc(I)c(O)c(O)c1I ; CC(O)c1c(O)c(I)c(OC(C)c2c(O)c(I)c(O)c(I)c2O)c(I)c1O ; CC(OC(C)c1c(O)c(I)c(O)c(I)c1O)c1c(O)c(I)c(O)c(I)c1O]

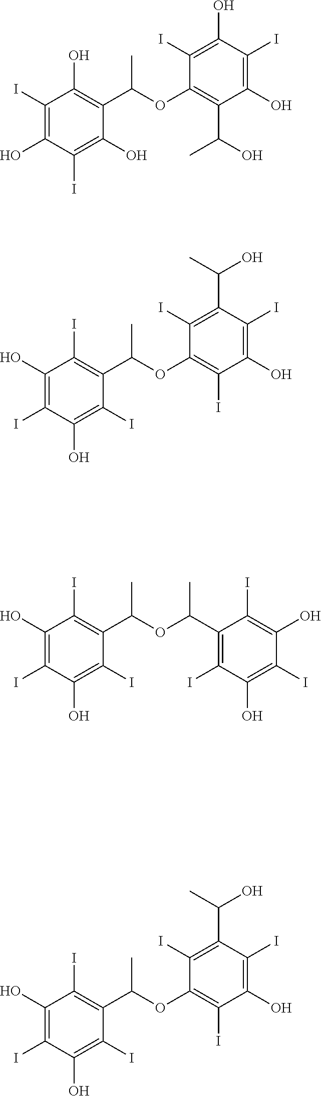 [SMILES: CC(O)c1c(O)c(I)c(O)c(I)c1OC(C)c1c(O)c(I)c(O)c(I)c1O ; CC(O)c1c(I)c(O)c(I)c(OC(C)c2c(I)c(O)c(I)c(O)c2I)c1I ; CC(OC(C)c1c(I)c(O)c(I)c(O)c1I)c1c(I)c(O)c(I)c(O)c1I ; CC(O)c1c(I)c(O)c(I)c(OC(C)c2c(I)c(O)c(I)c(O)c2I)c1I]

A compound represented by the following formula (1E) can be contained in the composition containing the compound (A) of the present embodiment. When the compound is used, the composition containing the compound (A) of the present embodiment preferably contains the compound represented by the formula (1E) in a range of 1 ppm by mass or more and 10% by mass or less, more preferably in a range of 1 ppm by mass or more and 5% by mass or less, further preferably in a range of 1 ppm by mass or more and 3% by mass or less, and particularly preferably in a range of 1 ppm by mass or more and 1% by mass or less, based on the total compound (A).

In the composition thus prepared, its stability tends to increase. The reason is not clear, but can be presumed that the equilibrium reaction of iodine atoms occurs between the compound (A) containing iodine and the compound (1E) containing no iodine and thus the composition is stabilized.

In this case, the composition preferably contains, as the compound (1E), a compound having a structure in which iodine atoms are eliminated from a compound exemplified as the compound (A) mentioned above, in combination.

In the composition thus prepared, its stability increases, leading to not only an increase in the storage stability, but also formation of a resin having stable properties, providing stable resist performance, and further, a reduction in the residue defect after development in a lithography process.

Examples of the method for using the compound represented by the formula (1E) in a range of 1 ppm by mass or more 10% by mass or less based on the compound (A) in the composition containing the compound (A) include, but are not particularly limited to, a method for adding the compound (1E) to the compound (A) and a method for producing the compound (1E) as a by-product during production of the compound (A).

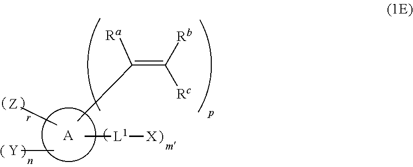

(In the formula (1E),

each X is independently F, Cl, Br, or an organic group having 1 to 30 carbon atoms and having 1 or more and 5 or less substituents selected from the group consisting of F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, F, Cl, Br, or an organic group having 1 to 60 carbon atoms and optionally having a substituent;

A is an organic group having 1 to 30 carbon atoms;

each Z is independently an alkoxy group, an ester group, an acetal group, or a carbonate ester group;

provided that none of X, L¹, Y, R^{a}, R^{b}, R^{c}, A, and Z contains I; and

p is an integer of 1 or more, m′ is an integer of 0 or more, n is an integer of 0 or more, and r is an integer of 0 or more.)

If the compound represented by the formula (1E) is contained in an amount more than 10% by mass based on the compound (A), the effect of improving the sensitivity may be reduced when a polymer containing the compound (A) is formed and used for lithography applications. On the other hand, when contained in an amount less than 1 ppm, the effect of improving aging stability may not be sufficiently exhibited.

m′ of the compound represented by the formula (1E) is preferably 0 to further increase the effect of aging stability.

Examples of the compound (1E) according to the present embodiment include compounds having the structures given below.

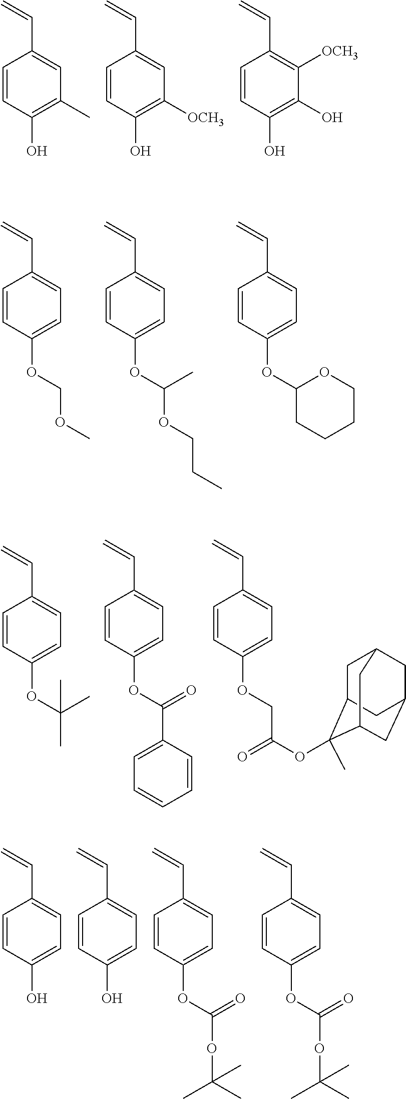

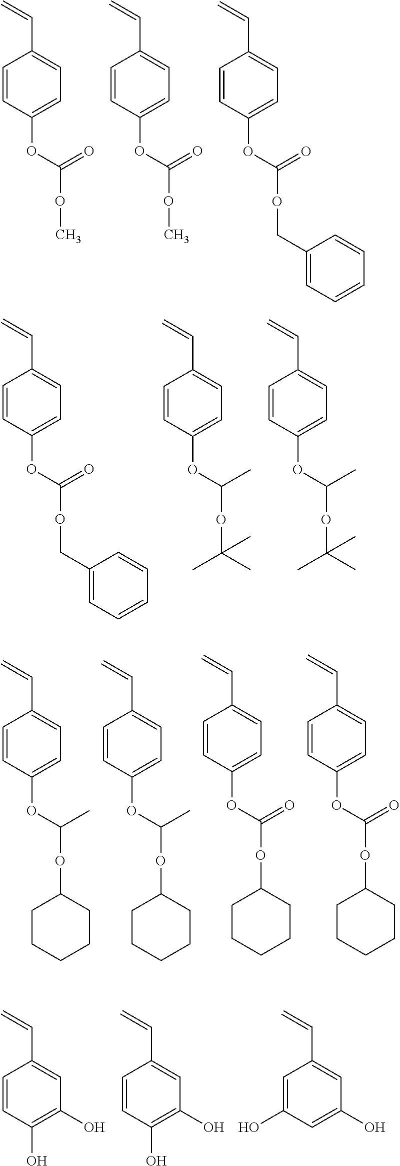

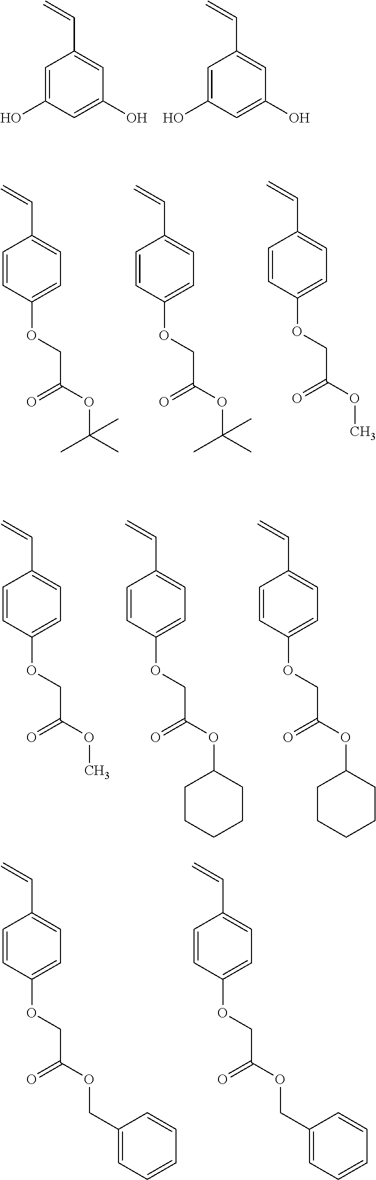

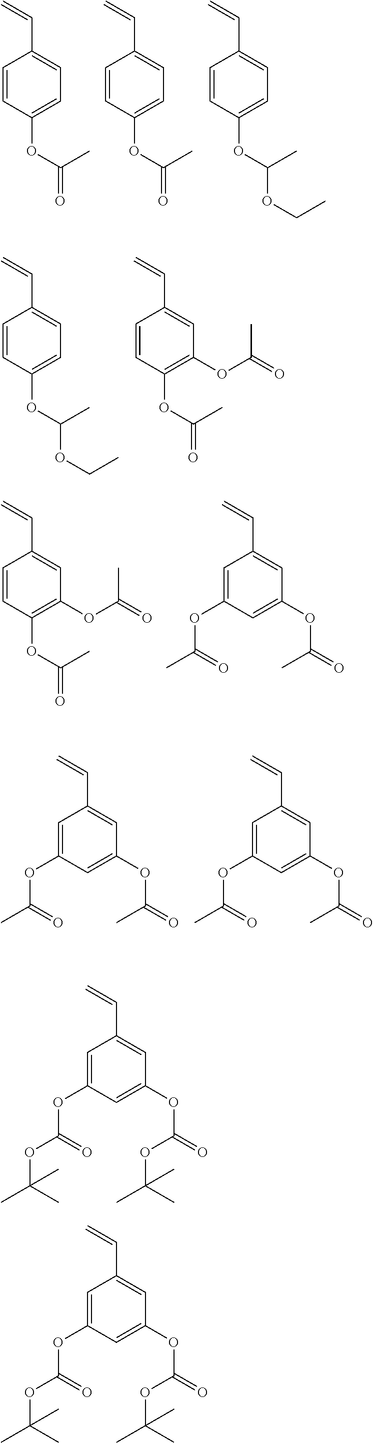

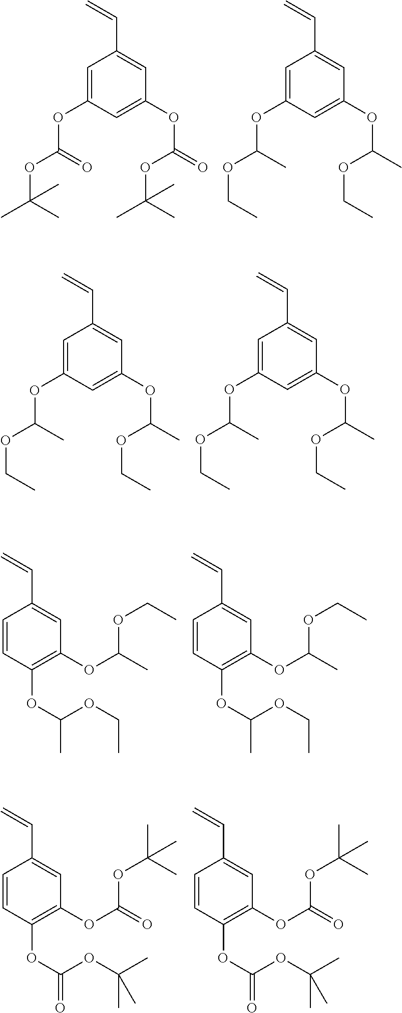

### [Method for Producing Compound (A)]

The compound represented by the formula (1) can be produced by various known synthetic methods.

As an example of the synthetic method, the synthesis can be carried out by introducing a halogen group, I, F, Cl, or Br, into a hydroxy group-containing aromatic aldehyde derivative, and then converting the aldehyde group into a vinyl group, but it is not particularly limited thereto. As another example of the synthetic method, a method for reacting iodine chloride in an organic solvent by carrying out iodination reaction on a hydroxybenzaldehyde derivative (e.g., Japanese Patent Laid-Open No. 2012-180326), a method for dropping iodine in an aqueous alkaline solution of phenol under alkaline conditions in the presence of βcyclodextrin (Japanese Patent Laid-Open No. 63-101342, Japanese Patent Laid-Open No. 2003-64012), or the like can be arbitrarily selected.

In the present embodiment, the iodination reaction through iodine chloride in an organic solvent is preferably used, particularly when introduction of a plurality of iodine atoms is intended. The compound (A) of the present embodiment can be synthesized by converting the aldehyde moiety of the synthesized iodine-introduced hydroxybenzaldehyde derivative into a vinyl group. As the method for converting the aldehyde moiety into a vinyl group, a Wittig reaction (e.g., the methods described in Synthetic Communications; Vol. 22; nb4; 1992 p513 and Synthesis; Vol. 49; nb. 23; 2017; p5217), a method for reacting malonic acid in the presence of a base (e.g., the methods described in Tetrahedron; Vol.46; nb.40; 2005; p6893, Tetrahedron; Vol.63; nb.4; 2007; p900, and US2004/118673), or the like can be arbitrarily used. As the synthetic method of the compound (A) of the present embodiment, for example, the method described in the above references can be arbitrarily used, but is not limited thereto.

The method for producing a compound represented by the formula (0) is shown below. Although the compound represented by the formula (0) includes both compounds containing no halogen and compounds containing halogen, for example, halogen may be introduced into a compound represented by the formula (0) containing no halogen such as a compound containing an amino group instead of halogen by a Sandmeyer reaction or the like to give a compound represented by the formula (1).

The method for producing the compound represented by the formula (0) according to the present embodiment preferably comprises,

a step of introducing an unsaturated double bond into a substituent Q of a compound represented by the following formula (S1) (hereinafter, may be referred to as the “double bond introduction step”). The production method may comprise a step of introducing a halogen atom into the compound represented by the following formula (S1) by reaction with a halogenating agent (hereinafter, may be referred to as the “halogen introduction step”).

In the production method, the order of the halogen introduction step and the double bond introduction step is not particularly limited, and either step may be carried out first.

Production of the compound represented by the formula (0) by the method enables the unsaturated double bond moiety which has low stability in production and requires careful handling (and the halogen group in the case of having a halogen) to be efficiently produced with relatively high stability and with high yield. In the case of comprising the halogen introduction step, the compound to be produced can be efficiently produced with relatively high stability and with high yield, even when the halogen group is an atom having a large atomic radius such as iodine.

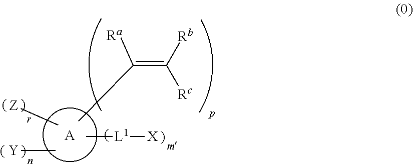

(In the formula (0),

each X is independently I, F, Cl, Br, or an organic group having 1 to 30 carbon atoms and having 1 or more and 5 or less substituents selected from the group consisting of I, F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, I, F, Cl, Br, or an organic group having 1 to 60 carbon atoms and optionally having a substituent;

A is an organic group having 1 to 30 carbon atoms;

each Z is independently an alkoxy group, an ester group, an acetal group, or a carbonate ester group; and

p is an integer of 1 or more, m′ is an integer of 0 or more, n is an integer of 0 or more, and r is an integer of 0 or more.)

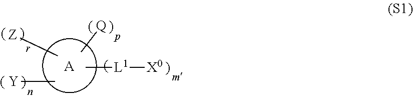

(In the formula (S1),

X⁰ is an organic group having 1 to 30 carbon atoms;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

A is an organic group having 1 to 30 carbon atoms;

each Z is independently an alkoxy group, an ester group, an acetal group, or a carbonate ester group;

Q is an organic group having 1 to 30 carbon atoms and having a hydroxyl group, an aldehyde group, a carboxyl group, or a ketone group; and

p is an integer of 1 or more, m′ is an integer of 0 or more, n is an integer of 0 or more, and r is an integer of 0 or more.)

Q is an organic group having 1 to 30 carbon atoms and having a hydroxyl group, an aldehyde group, a carboxyl group, or a ketone group, and when Q has an aldehyde group or a carboxyl group, the number of carbon atoms means the total number of carbon atoms including the number of carbon atoms of these functional groups. Q is preferably an organic group having 1 to 30 carbon atoms having a hydroxyl group, and preferably a hydroxymethyl group, from the viewpoint of preventing side reactions.

Examples of the step of introducing a halogen atom (halogen introduction step) include the aforementioned method for introducing a halogen group. Examples of the halogenating agent include, but are not particularly limited to, a iodinating agent such as iodine chloride, iodine, and N-iodosuccinimide; a fluorinating agent such as potassium fluoride and tetramethylammonium fluoride; a chlorinating agent such as thionyl chloride and dichloromethylmethyl ether; and a brominating agent such as a bromine molecule, carbon tetrabromide, and N-bromosuccinimide. Among them, a iodinating agent is preferable, and iodine chloride is more preferable.

The ratio of the halogenating agent to the compound represented by the formula (S1) in the step of introducing a halogen atom is preferably 1.2 mol times or more, more preferably 1.5 mol times or more, and further preferably 2.0 mol times or more.

The reaction temperature in the step of introducing a halogen atom is not particularly limited, but is preferably 40 to 80° C. The reaction time is not particularly limited, but is preferably 1 to 3 hours.

When Q is an organic group having 1 to 30 carbon atoms having a hydroxyl group, the production method according to the present embodiment may comprise a step of acidifying an alcohol and introducing an aldehyde group after the step of introducing a halogen atom. The oxidizing agent to be used in the oxidation is not particularly limited as long as it can introduce aldehyde, and examples thereof include manganese dioxide and chromium trioxide. The reaction temperature in the step of introducing an aldehyde group is not particularly limited, but is preferably 10 to 40° C. The reaction time is not particularly limited, but is preferably 1 to 6 hours.

In the step of introducing an unsaturated double bond into a substituent Q (double bond introduction step), the unsaturated double bond can be introduced by the Wittig reaction, the method for reacting malonic acid in the presence of a base, or the like, as mentioned above.

As the solvent to be used in the reaction, a commonly available solvent may be used. For example, an alcohol, an ether, a hydrocarbon, a halogenated solvent, or the like may be arbitrarily used within a range not inhibiting the above reaction. A plurality of solvent may be mixed and used within a range not inhibiting the above reaction. Since water inhibits the reaction, a dehydrated solvent is preferably used.

The reaction temperature and the reaction time depend on the substrate concentration and the catalyst to be used, but the reaction may typically be carried out at a reaction temperature of −20° C. to 100° C., a reaction time of 1 hour to 10 hours, and under a pressure of ordinary pressure, reduced pressure, or increased pressure. The reaction may be carried out by arbitrarily selecting a publicly known method such as a batch method, a semi-batch method, or a continuous method.

A polymerization inhibitor may be added in a series of reactions, and a commonly available commercial product may be used. Examples thereof include a nitroso compound such as 2,2,6,6-tetramethyl-4-hydroxypiperidine-1-oxyl, N-nitrosophenylhydroxylamine ammonium salt, N-nitrosophenylhydroxylamine aluminum salt, N-nitroso-N-(1-naphthyl)hydroxylamine ammonium salt, N-nitrosodiphenylamine, N-nitroso-N-methylaniline, nitrosonaphthol, p-nitrosophenol, and N,N′-dimethyl-p-nitrosoaniline; a sulfur containing compound such as phenothiazine, methylene blue, and 2-mercaptobenzimidazole; an amine such as N,N′-diphenyl-p-phenylenediamine, N-phenyl-N′-isopropyl-p-phenylenediamine, 4-hydroxydiphenylamine, and aminophenol; a quinone such as hydroxyquinoline, hydroquinone, methylhydroquinone, p-benzoquinone, and hydroquinone monomethyl ether; a phenol such as p-methoxyphenol, 2,4-dimethyl-6-t-butylphenol, catechol, 3-s-butylcatechol, 2,2-methylenebis-(6-t-butyl-4-methylphenol); an imide such as N-hydroxyphthalimide; an oxime such as cyclohexane oxime, p-quinone dioxime; and dialkyl thiodipropionate. The addition amount is, for example, 0.001 to 10 parts by mass, and preferably 0.01 to 1 part by mass, based on 100 parts by mass of a compound represented by the formula (S1).

The compound represented by the formula (0) obtained by the reaction can be isolated and purified as the desired high purity monomer by a separation and purification method by filtration, concentration, distillation, extraction, crystallization, recrystallization, column chromatography, activated carbon, or the like, which are known purification methods, or a combined method thereof.

### [Method for Producing Compound Formula (0)]

As the method for producing the compound formula (0), a production method in which the compound represented by the above formula (S1) is a compound represented by the following formula (SA1), the method comprising a step designated by the following A1 and a step designated by the following A2 can be selected.

A1) a step of obtaining a compound represented by the following formula (SA2) by using the compound represented by the above formula (SA1), and a compound represented by the following formula (RM1) or malononitrile

A2) a step of obtaining a compound represented by the formula (0) by using a compound represented by the formula (SA2) and a fluoride source

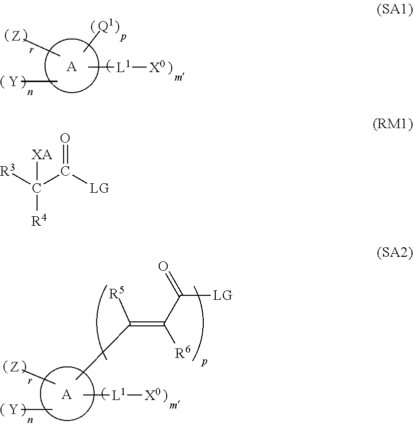

(In the formulas (SA1), (RM1), and (SA2),

X⁰, L¹, Y, A, Z, p, m′, n, and r are as defined in the formulas (S1) and (0);

Q¹ is aldehyde or ketone;

LG is a group selected from a hydroxy groups, an alkoxy group, a carbonate ester group, an acetal group, and a carboxy group, and the alkoxy group, the carbonate ester group, the acetal group, and the carboxy group contain an aliphatic group or an aromatic group optionally having a substituent having 1 to 60 carbon atoms;

R³ is a hydrogen group, or a carboxy group or ester group optionally having a substituent having 1 to 60 carbon atoms;

R⁴ is a hydrogen group;

each of R⁵ and R⁶ is independently H, F, Cl, Br, or an organic group having 1 to 60 carbon atoms and optionally having a substituent; and

XA is a group selected from a hydrogen group and a halogen group. R³ may form a cyclic structure by binding to LG.)

As mentioned above, step A1 is a step of obtaining the compound represented by the formula (SA2) by using the compound represented by the formula (SA1), and the compound represented by the formula (RM1) or malononitrile.

Specific examples of the compound represented by the formula (RM1) include a maleate derivative such as maleic acid, dimethyl maleate, diethyl maleate, dipropyl maleate, diisopropyl maleate, and maleic anhydride; and an acetate derivative such as ethyl acetate, propyl acetate, butyl acetate, ethyl achloroacetate, propyl achloroacetate, and butyl achloroacetate. RM1 is preferably a derivative selected from a malonic acid, a malonate derivative, and acetic acid derivative, and a acetate derivative.

In step A1, a method generally employed as a Knoevenagel reaction or a Doebner reaction may be used and, for example, the conditions described in Journal of Molecular Catalysis B:Enzymatic, 82, 92-95; 2012, Tetrahedron Letters, 46(40), 6893-6896; 2005, and the like may be used. Specifically, the compound according to the formula (SA2) can be obtained by reacting the compound represented by the formula (RM1) or malononitrile with a base in a solvent. In addition to a base, an acid may also be used in combination.

As the base, various known compounds may be used and, for example, a nitrogen-containing compound such as a nitrogen-containing cyclic compound containing a structure such as pyridine, piperidine, pyrrolidine, azole, diazole, triazole, and morpholine; and a tertiary amine such as tributylamine, trimethylamine, and trihydroxyethylamine can be arbitrarily used.

The acid that may be used in combination with the base is not particularly limited, and a weak acid such as acetic acid and propionic acid can be preferably used in combination.

The balance between acidity and basicity of the reaction system is not particularly limited, but the reaction is preferably carried out under acidic conditions when the compound of the present embodiment in which m is an integer of 1 or more is the intended compound.

In step A1, when LG is an alkoxy group, a carbonate ester group, an acetal group, or a carboxyl group, a compound represented by the formula (SA3) is preferably obtained by adding a reaction of further converting LG to a hydroxy group by a treatment such as hydrolysis. The treatment such as hydrolysis is not particularly limited as long as the LG group can be converted to a hydroxy group, and as one example of the reaction conditions, for example, a deprotection reaction can be carried out by using an acid such as hydrochloric acid, sulfuric acid, and paratoluenesulfonic acid as a catalyst in combination, under temperature conditions such as reflux. As another example of the reaction conditions, a deprotection reaction can be carried out by refluxing using an inorganic base such as sodium hydroxide and potassium hydrate or an organic base such as tertiary amine as the base under solvent conditions such as toluene and xylene.

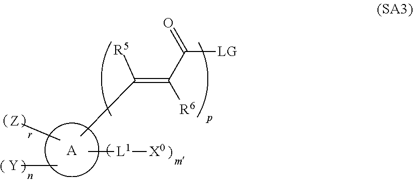

(In the formula (SA3),

X⁰, L¹, Y, A, Z, p, m′, n, and r are as defined in the formulas (S1) and (0); and

each of R⁵ and R⁶ is independently H, F, Cl, Br, or an organic group having 1 to 60 carbon atoms and optionally having a substituent.)

In step A1, the compound represented by the formula (SA2) may be obtained by further using a reducing agent. When a reducing agent is used to obtain the compound represented by the formula (SA2), RM1 having high stability can be used, which is advantageous in terms of conversion and purity. As the reducing agent, various materials can be used.

As the reducing agent, a wide variety of reducing agents which function under the reaction conditions of the present embodiment are used. Examples of suitable reducing agents include, but are not limited to, a metal hydride and a metal hydride complex compound. Specific examples include borane dimethylsulfide, diisobutylaluminum hydride, sodium borohydride, lithium borohydride, potassium borohydride, zinc borohydride, lithium tri-s-butylborohydride, potassium tri-s-butylborohydride, lithium triethylborohydride, lithium aluminum hydride, lithium tri-t-butoxyaluminum hydride, and sodium bis(methoxyethoxy)aluminum hydride.

The amount of the reducing agent used can be arbitrarily set according to, for example, the substrate, reducing agent, and reaction conditions to be used, without particular limitation. In general, it is suitably 1 to 500 parts by mass, and from the viewpoint of the yield, it is preferably 10 to 200 parts by mass, based on 100 parts by mass of the reaction raw materials.

In the above compound represented by the formula (S1), A is preferably benzene, toluene, or a heteroaromatic ring, in terms of the effect per mass with respect to the stability of the X group in the resin and the improvement of lithography performance such as the improvement of the sensitivity due to X group, the solubility in a developing solution of the resin when A is incorporated in the resin for lithography as a constitutional unit of the copolymer, and the effect of suppressing partial crystallinity in a resin matrix.

As the reaction solvent for the deprotection reaction, various solvents can be used without particular limitation as long as it is a solvent that can dissolve the compound of the above formula (SA2), and an alcohol solvent such as methanol, ethanol, propanol, and butanol; a ketone solvent such as cyclohexanone, cyclopentanone, MEK, and MIBK; a linear or cyclic ester solvent such as ethyl acetate, butyl acetate, ethyl propionate, isobutyl propionate, ethyl lactate, and gamma butyrolactone; an ether solvent such as diethyl ether; a glycol solvent such as diethylene glycol, PGMEA, and PGME; an aromatic solvent such as toluene and benzene; an amide solvent such as DMF; water; or the like can be arbitrarily used.

As mentioned above, step A2 is a step of subjecting the carboxyl group of the compound represented by the formula (SA2) or the carboxyl group and the ester group which are introduced into R⁵ to decarboxylation by using a fluoride source.

As the fluoride source, various compounds that generate fluorides can be used, and salts of quaternary amines and fluorides, such as tetrabutylamine fluoride, tetramethylamine fluoride, and tetrahydroxyethylamine fluoride; salts of metal cation species such as tetramethylaluminum and fluorides, salts of phosphonium such as tetraoctadecylphosphonium and fluorides; fluoride salts of alkali metals such as KF and NaF, and the like can be arbitrarily used.

In step A2, the compound according to the formula (1) can be obtained by subjecting the compound according to the formula (SA2) or the formula (SA3) to a decarboxylation reaction by using the fluoride source at a low temperature, the reaction temperature of 100° C. or less. For the formula (SA2) having a structure which may cause denaturation and degradation at high temperature depending on the selection of the core A, the functional group Z, the functional group Y, the L¹ group, and the X group, the compound represented by the formula (1) can be obtained at 80° C. or less, which is a further lowered temperature as the reaction temperature, or 60° C. or less, and more preferably 50° C. or less.

A polymerization inhibitor may be added in a series of reactions in step A2, and a commonly available commercial product may be used. Examples thereof include a nitroso compound such as 2,2,6,6-tetramethyl-4-hydroxypiperidine-1-oxyl, N-nitrosophenylhydroxylamine ammonium salt, N-nitrosophenylhydroxylamine aluminum salt, N-nitroso-N-(1-naphthyl)hydroxylamine ammonium salt, N-nitrosodiphenylamine, N-nitroso-N-methylaniline, nitrosonaphthol, p-nitrosophenol, and N,N′-dimethyl-p-nitrosoaniline; a sulfur containing compound such as phenothiazine, methylene blue, and 2-mercaptobenzimidazole; an amine such as N,N′-diphenyl-p-phenylenediamine, N-phenyl-N′-isopropyl-p-phenylenediamine, 4-hydroxydiphenylamine, and aminophenol; a quinone such as hydroxyquinoline, hydroquinone, methylhydroquinone, p-benzoquinone, and hydroquinone monomethyl ether; a phenol such as p-methoxyphenol, 2,4-dimethyl-6-t-butylphenol, catechol, 3-s-butylcatechol, 2,2-methylenebis-(6-t-butyl-4-methylphenol); an imide such as N-hydroxyphthalimide; an oxime such as cyclohexane oxime, p-quinone dioxime; and dialkyl thiodipropionate. The addition amount is, for example, 0.001 to 10 parts by mass, and preferably 0.01 to 1 part by mass, based on 100 parts by mass of the (meth)acrylic acid compound represented by the general formula (b).

The method for producing the compound represented by the following formula (1) comprises: a step of forming a compound represented by the following formula (SB1) by at least one of compounds represented by the following formula (SB2A) and the following formula (SB3A) obtained through a step designated by the following B1A and at least one of steps designated by the following B2A and B3A; and a double bond introduction step of introducing an unsaturated double bond into a substituent Qb of the compound represented by the formula (SB1).
B1A) a step of providing the following substrate SB1A comprising one or more amino groups, and a core B having an aldehyde group or a ketone group
B2A) a step of obtaining the compound represented by the following formula (SB2A) in which iodine is introduced into the core B
B3A) a step of obtaining the compound represented by the formula (SB3A) in which an amino group is substituted with a halogen group by a Sandmeyer reaction

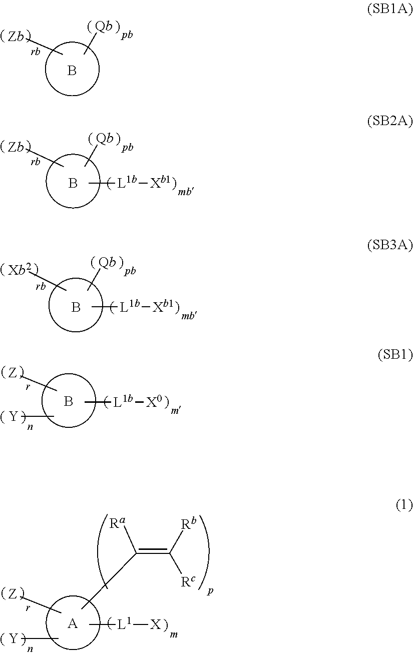

(In the formula (1),

each X is independently I, F, Cl, Br, or an organic group having 1 to 30 carbon atoms and having 1 or more and 5 or less substituents selected from the group consisting of I, F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;

each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, I, F, Cl, Br, or an organic group having 1 to 60 carbon atoms and optionally having a substituent;

A is an organic group having 1 to 30 carbon atoms;

each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and

p is an integer of 1 or more, m is an integer of 1 or more, n is an integer of 0 or more, and r is an integer of 0 or more.

In the formulas (SB1A), (SB2A), (SB3A), and (SB1),

Zb represents an amino group optionally having a substituent consisting of a hydrogen group or a hydrocarbon group having 1 to 30 carbon atoms and optionally having a substituent, rb represents an integer of 1 or more, Qb, L^{1b}, X^{b1}, B, pb, and mb′ have the same meanings as Q, L, X, A, p, and m in the formula (1), respectively. X^{b2} represents I, F, Cl, Br, or an organic group having 1 to 30 carbon atoms and having 1 or more and 5 or less substituents selected from the group consisting of I, F, Cl, and Br.)

In the step of introducing a double bond, an organic phosphorus compound and a base may be used. As the organic phosphorus compound, for example, an oxoacid of phosphorus, an oxoacid of alkylated phosphorus, and a phosphate can be used. Examples of the oxoacid of phosphorus include phosphoric acid and pyrophosphoric acid, examples of the oxoacid of alkylated phosphorus include dimethyl phosphinic acid and triethyl phosphate, and examples of the phosphate include diammonium hydrogen phosphate, but are not limited thereto. The organic phosphorus compound may be used not only alone but also in combination of two or more kinds. Examples of the base include an alkali metal hydride such as potassium hydride and sodium hydride, an alkali metal carbonate such as potassium carbonate and cesium carbonate, and an organic base such as a quaternary ammonium salt (tetramethyl ammonium hydroxide), alkoxide (sodium ethoxide and potassium t-butoxide (t-BuOK)), metal amide (lithium diisopropylamide (LDA), potassium hexamethyldisilazide (KHMDS), lithium 2,2,6,6,-tetramethylpiperidide (LiTMP), metal alkyl (alkyllithium and alkylaluminum), pyridine (pyridine and DMAP), and non-pyridine heterocyclic amine (DBU, DBN, and imidazole).

Among a step (B1A) of preparing a starting compound (SB1A) having an aromatic core B as A, at least one or more amino groups on the core B, and a group selected from at least one of an alcohol group, an aldehyde group as a carbonyl group, a ketone group, and a carboxyl group, a step (B2A) of obtaining the formula (SB2A) in which iodine is introduced into the core B, and further, a step (B3A) of obtaining the compound represented by the formula (SB3A) in which an amino group is substituted with a halogen group by a Sandmeyer reaction, a method including the step (B1A) and at least either one of the step (B2A) and the step (B3A) can be selected as another preferred method for obtaining the compound represented by the above formula (SA1).

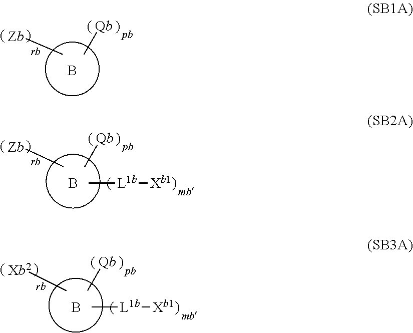

(In the formulas (SB1A), (SB2A), (SB3A), and (SA1A),

Zb represents an amino group optionally having a substituent consisting of a hydrogen group or a hydrocarbon group having 1 to 30 carbon atoms and optionally having a substituent;

rb represents an integer of 1 or more; and

Qb, L^{1b}, X^{b1}, B, pb, and mb′ have the same meanings as Q, L, X, A, p, and m in the formula (1), respectively. X^{b2} represents I, F, Cl, Br, or an organic group having 1 to 30 carbon atoms and having 1 or more and 5 or less substituents selected from the group consisting of I, F, Cl, and Br.)

That is, in the method for producing the compound represented by the above formula (SA1), the compound represented by the above formula (SA1) which is at least one of the compounds represented by the formula (SB2A) and the formula (SB3A) obtained through the step designated by B1A and at least one of the steps designated by B2A and B3A is preferably produced.

B1A) a step of providing a substrate SB1A comprising one or more amino groups, and a core B having an alcohol group, an aldehyde group, or a ketone group

B2A) a step of obtaining the compound represented by the formula (SB2A) in which iodine is introduced into the core B

B3A) a step of obtaining the compound represented by the formula (SB3A) in which an amino group is substituted with a halogen group by a Sandmeyer reaction

In this production method, the double bond introduction step (step B1A) and the halogen introduction step (step B2A or B3A) are conducted in the order presented.

According to the method described in the step (B2A), the reaction for introducing iodine into the compound represented by the formula (SB1A) (substrate SB1A) can be proceeded by at least allowing an iodinating agent to react with the compound represented by the formula (SB1A), and the intended compound can be obtained according to known reaction conditions for introducing iodine using the methods described in Non Patent Literatures such as Adv. Synth. Catal. 2007, 349, 1159-1172 and Organic Letters; Vol. 6; (2004); p. 2785-2788 and Patent Literatures such as U.S. Pat. Nos. 5,300,506, 5,434,154, US2009/281114, EP1439164, and WO 2006/101318. Examples of the iodinating agent that can be used include, but are not limited to, iodine compounds, monochloride iodine, N-iodosuccinimide, benzyltrimethylammonium dichloroiodate, tetraethylammonium iodide, tetranormalbutylammonium iodide, lithium iodide, sodium iodide, potassium iodide, 1-chloro-2-iodoethane, iodine silver fluoride, tert-butyl hypoiodite, 1,3-diiodo-5,5-dimethylhydantoin, iodine-morpholine complexes, trifluoroacetyl hypoiodite, iodine-iodic acid, iodine-periodic acid, iodine-hydrogen peroxide, 1-iodoheptafluoropropane, triphenylphosphate-methyl iodide, iodine-thallium (I) acetate, 1-chloro-2-iodoethane, and iodine-copper (II) acetate.

One or a plurality of additives can be added in the iodination reaction to promote the reaction and to suppress the by-products. Examples of the additive include an acid such as hydrochloric acid, sulfuric acid, nitric acid, phosphoric acid, acetic acid, p-toluenesulfonic acid, ferric chloride, aluminum chloride, copper chloride, antimony pentachloride, silver sulfate, silver nitrate, and silver trifluoroacetate; a base such as sodium hydroxide, potassium hydrate, lithium hydroxide, sodium carbonate, potassium carbonate, calcium carbonate, sodium bicarbonate, and potassium bicarbonate; an oxidizing agent such as ammonium cerium (IV) nitrate and sodium peroxodisulfate; an inorganic compound such as sodium chloride, potassium chloride, mercury (II) oxide, and cerium oxide; an organic compound such as acetic anhydride; and a porous material such as zeolite.

In the step (B2A), iodine is preferably introduced into the core B by at least using an iodine source and an oxidizing agent. Use of the iodine source and the oxidizing agent is preferable in terms of improving the reaction efficiency and the purity. Examples of the iodine source include the above iodinating agents. Examples of the oxidizing agent include periodic acid, hydrogen peroxide, and a predetermined additive (such as hydrochloric acid, sulfuric acid, nitric acid, and p-toluenesulfonic acid).

The core B in the substrate SB1A preferably has an aromatic ring structure optionally having a heteroatom in terms of the solubility in the developing solution. As the aromatic ring structure in the core B, at least any one of furan, thiophene, pyrrole, and indole is preferably contained in terms of a balance between the solubility in the developing solution and the effect of improving the sensitivity.

Although the reaction in the step (B2A) can be conducted neat without solvent, examples of the reaction solvent that can be used include a halogenated solvent such as dichloromethane, dichloroethane, chloroform, and carbon tetrachloride; an alkyl solvent such as hexane, cyclohexane, heptane, pentane, and octane; an aromatic hydrocarbon solvent such as benzene and toluene; an alcohol solvent such as methanol, ethanol, 1-propanol, 2-propanol, 1-butanol, and 2-butanol; an ether solvent such as diethyl ether, diisopropylether, and tetrahydrofuran; acetic acid, dimethylformamide, dimethylsulfoxide, and water.

The reaction temperature in the step (B2A) is not particularly limited, and the temperature may be anywhere from the freezing point to the boiling point of the solvent to be used in the reaction but is particularly preferably 0° C.-150° C.

The iodine substitution reaction into the compound represented by the formula (SB1A) in the step (B2A) can be proceeded by at least allowing an iodinating agent to react with the compound represented by the formula (SB1A), for example, the intended compound can be obtained by a Sandmeyer reaction using the method described in Chemistry—A European Journal, 24(55), 14622-14626; 2018, Synthesis (2007) (1), 81-84 and the like under known iodine substitution reaction conditions.

### (Method for Producing Compound Represented by Formula (1C))

As one example of the method for producing the compound represented by the formula (1C), the compound represented by the formula (1C) can be obtained by, when Ra is a hydrogen group in the method for producing the compound represented by the aforementioned formula (1), dimerizing the compound represented by the formula (1) obtained through the above production method. As the easiest method for dimerizing the compound represented by the formula (1), the compound (1) obtained is placed under high temperature conditions or basic conditions, so that an activity methylene moiety formed by elimination of the Ra group serves as a starting point and can progress dimerization.

The compound represented by the above formula (SA1) may be produced by a production method comprising a step designated by the following B1B, and at least either one of steps designated by the following B2B and B3B.
B1B) a step of providing the following substrate SB1B comprising one or more amino groups, and a core B having an aldehyde group or a ketone group
B2B) a step of obtaining a compound represented by the formula (SB2B) in which iodine is introduced into the core B
B3B) a step of obtaining a compound represented by the formula (SB3B) in which an amino group is substituted with a halogen group

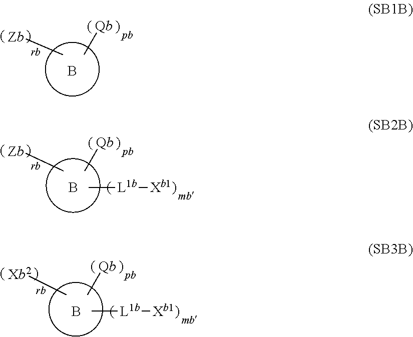

(In the formulas (SB1B), (SB2B), (SB3B), and (SA1B),

Zb represents an amino group optionally having a substituent consisting of a hydrogen group or a hydrocarbon group having 1 to 30 carbon atoms and optionally having a substituent, rb represents an integer of 1 or more, Qb, L^{1b}, X^{b1}, B, pb, and mb′ have the same meanings as Q, L, X, A, p, and m in the formula (1), respectively. X^{b2} represents I, F, Cl, Br, or an organic group having 1 to 30 carbon atoms and having 1 or more and 5 or less substituents selected from the group consisting of I, F, Cl, and Br.)

The method for producing the compound represented by the above formula (SA1) may further comprise a step designated by the following B4a. Comprising the step designated by the following B4a is preferable in terms of the reaction purity of the compound to be formed.

### B4a) Wittig Step

The Wittig step is a step of forming alkene by a Wittig reaction, and is a step of forming alkene from a carbonyl moiety having aldehyde or ketone using phosphorus ylide, although it is not limited. As the phosphorus ylide, for example, triphenyl alkyl phosphine bromide such as triphenyl methyl phosphine bromide that can form a stable phosphorus ylide can be used. Also, as the phosphorus ylide, a phosphonium salt can be reacted with a base to form a phosphorus ylide in the reaction system, which can be used in the above reaction. As the base, a conventionally known one can be used, and for example, an alkali metal salt of alkoxide can be arbitrarily used.

In the step designated by the above B2B, iodine may be introduced into the above core B by at least using an iodine source and an oxidizing agent. Use of the iodine source and the oxidizing agent is preferable in terms of the efficiency of a reaction and the purity.

The above core B preferably has an aromatic ring structure optionally having a heteroatom in terms of a balance between the solubility in the developing solution and the effect of improving the sensitivity.

The method for producing the compound represented by the following formula (1) is the method for producing the compound represented by the following formula (1), comprising: the halogen introduction step of introducing a halogen atom into the compound represented by the following formula (S1) by reaction with a halogenating agent; and the double bond introduction step of introducing an unsaturated double bond into the substituent Q, in which the step of introducing a double bond may use an organic phosphorus compound and a base.

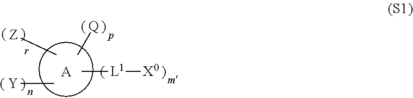

(In the formula (S1),

X⁰ is an organic group having 1 to 30 carbon atoms;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

A is an organic group having 1 to 30 carbon atoms;

each Z is independently an alkoxy group, an ester group, an acetal group, or a carbonate ester group;

Q is an organic group having 1 to 30 carbon atoms and having a hydroxyl group, an aldehyde group, a carboxyl group, or a ketone group; and

p is an integer of 1 or more, m′ is an integer of 0 or more, n is an integer of 0 or more, and r is an integer of 0 or more.)

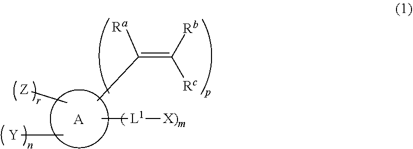

(In the formula (1),

each X is independently I, F, Cl, Br, or an organic group having 1 to 30 carbon atoms and having 1 or more and 5 or less substituents selected from the group consisting of I, F, Cl, and Br;

each L¹ is independently a single bond, an ether group, an ester group, a thioether group, an amino group, a thioester group, an acetal group, a phosphine group, a phosphone group, a urethane group, a urea group, an amide group, an imide group, or a phosphate group;

each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;

each of R^{a}, R^{b}, and R^{c} is independently H, I, F, Cl, Br, or an organic group having 1 to 60 carbon atoms and optionally having a substituent;

A is an organic group having 1 to 30 carbon atoms;

each Z is independently an alkoxy group, an ester group, an acetal group, or a carbonate ester group; and

p is an integer of 1 or more, m is an integer of 1 or more, n is an integer of 0 or more, and r is an integer of 0 or more.)

It is preferable that the compound in the present embodiment be obtained as a crude by the reaction described above and be then further subjected to purification, thereby removing the residual metal impurities. That is, it is preferable to avoid residual metal impurities derived from contamination by metal components which are used as a reaction aid during the production process of the compound or contaminated from a reaction vessel for production or other production equipment, from the viewpoint of preventing the deterioration of the resin with time, storage stability, and further, production yield due to processability, defects, and the like when the composition is formed into a resin and applied to a semiconductor production process.

The residual amounts of respective metal impurities are preferably less than 1 ppm, more preferably less than 100 ppb, further preferably less than 50 ppb, still more preferably less than 10 ppb, and most preferably less than 1 ppb, based on the resin. In particular, with respect to metal species such as Fe, Ni, Sb, W, and Al which are classified as transition metals, there is concern that the amount of residual metals of 1 ppm or more may cause the denaturation and deterioration of materials with time due to the interaction with the compound in the present embodiment. Further, there is also concern that the metal balance cannot be sufficiently reduced with the amount of residual metals of 1 ppm or more when a resin for semiconductor process is prepared using the compound prepared, which results in defects derived from residual metals in a semiconductor production process and reduction in yield due to performance deterioration.

The purification method is not particularly limited, but comprises a step of obtaining a solution (S) by dissolving the compound in the present embodiment in a solvent; and a step of extracting impurities in the compound in the present embodiment by bringing the obtained solution (S) into contact with an acidic aqueous solution (a first extraction step), wherein the solvent used in the step of obtaining the solution (S) contains an organic solvent that does not inadvertently mix with water.

According to the purification method, the contents of various metals that may be contained as impurities in the resin can be reduced.

More specifically, the compound in the present embodiment can be dissolved in an organic solvent that does not inadvertently mix with water to obtain the solution (S), and further, an extraction treatment can be carried out by bringing the solution (S) into contact with an acidic aqueous solution. Thereby, after the metals contained in the above solution (S) is transferred to the aqueous phase, the organic phase and the aqueous phase can be separated to obtain a resin having a reduced metal content.

The solvent that does not inadvertently mix with water used in the purification method is not particularly limited, but is preferably an organic solvent that is safely applicable to a semiconductor production process, and specifically it is an organic solvent having a solubility in water at room temperature of less than 30%, and more preferably is an organic solvent having a solubility of less than 20% and particularly preferably less than 10%. The amount of the organic solvent used is preferably 1 to 100 times the total mass of the resin to be used.

Specific examples of the solvent that does not inadvertently mix with water include, but are not limited to, ethers such as diethyl ether and diisopropyl ether, esters such as ethyl acetate, n-butyl acetate, and isoamyl acetate; ketones such as methyl ethyl ketone, methyl isobutyl ketone, ethyl isobutyl ketone, cyclohexanone, cyclopentanone, 2-heptanone, and 2-pentanone; glycol ether acetates such as ethylene glycol monoethyl ether acetate, ethylene glycol monobutyl ether acetate, propylene glycol monomethyl ether acetate (PGMEA), and propylene glycol monoethyl ether acetate; aliphatic hydrocarbons such as n-hexane and n-heptane; aromatic hydrocarbons such as toluene and xylene; and halogenated hydrocarbons such as methylene chloride and chloroform. Among them, toluene, 2-heptanone, cyclohexanone, cyclopentanone, methyl isobutyl ketone, propylene glycol monomethyl ether acetate, ethyl acetate, and the like are preferable, methyl isobutyl ketone, ethyl acetate, cyclohexanone, and propylene glycol monomethyl ether acetate are more preferable, and methyl isobutyl ketone and ethyl acetate are still more preferable. Since methyl isobutyl ketone, ethyl acetate, and the like have a relatively high saturation solubility and a relatively low boiling point of the compound of the present embodiment, the load in the case of industrially distilling off a solvent and in the step of removing the solvent by drying can be reduced. These solvents can be each used alone, or can also be used as a mixture of two or more kinds.

The acidic aqueous solution used in the purification method is arbitrarily selected from among aqueous solutions in which organic compounds or inorganic compounds are dissolved in water, generally known as acidic aqueous solutions. Examples of the acidic aqueous solution include, but are not limited to, aqueous mineral acid solutions in which mineral acids such as hydrochloric acid, sulfuric acid, nitric acid, and phosphoric acid are dissolved in water, or aqueous organic acid solutions in which organic acids such as acetic acid, propionic acid, oxalic acid, malonic acid, succinic acid, fumaric acid, maleic acid, tartaric acid, citric acid, methanesulfonic acid, phenolsulfonic acid, p-toluenesulfonic acid, and trifluoroacetic acid are dissolved in water. These acidic aqueous solutions can be each used alone, and can be also used in combination of two or more kinds. Among these acidic aqueous solutions, aqueous solutions of one or more mineral acids selected from the group consisting of hydrochloric acid, sulfuric acid, nitric acid and phosphoric acid, or aqueous solutions of one or more organic acids selected from the group consisting of acetic acid, propionic acid, oxalic acid, malonic acid, succinic acid, fumaric acid, maleic acid, tartaric acid, citric acid, methanesulfonic acid, phenolsulfonic acid, p-toluenesulfonic acid and trifluoroacetic acid are preferable, aqueous solutions of sulfuric acid, nitric acid, and carboxylic acids such as acetic acid, oxalic acid, tartaric acid and citric acid are more preferable, aqueous solutions of sulfuric acid, oxalic acid, tartaric acid and citric acid are further preferable, and an aqueous solution of oxalic acid is still more preferable. It is considered that polyvalent carboxylic acids such as oxalic acid, tartaric acid and citric acid coordinate with metal ions and provide a chelating effect, and thus there is a tendency that metals can be more effectively removed. Also, as for water used herein, it is preferable to use water, the metal content of which is small, such as ion exchanged water, according to the purpose of the purification method in the present embodiment.

The pH of the acidic aqueous solution used in the purification method is not particularly limited, but it is preferable to regulate the acidity of the aqueous solution in consideration of an influence on the above resin. Normally, the pH range is about 0 to 5, and is preferably about pH 0 to 3.

The amount of the acidic aqueous solution used in the purification method is not particularly limited, but it is preferable to regulate the amount from the viewpoint of reducing the number of extraction operations for removing metals and from the viewpoint of ensuring operability in consideration of the overall amount of fluid. From the above viewpoints, the amount of the acidic aqueous solution used is preferably 10 to 200% by mass, more preferably 20 to 100% by mass, based on 100% by mass of the solution (S).

In the purification method, by bringing the above acidic aqueous solution into contact with the above solution (S), metals can be extracted from the resin in the solution (S).

In the above purification method, the solution (S) can further contain an organic solvent that inadvertently mixes with water. When the solution (S) contains an organic solvent that inadvertently mixes with water, there is a tendency that the amount of the resin charged can be increased, also the fluid separability is improved, and purification can be carried out at a high reaction vessel efficiency. The method for adding the organic solvent that inadvertently mixes with water is not particularly limited. For example, any of a method involving adding it to the organic solvent-containing solution in advance, a method involving adding it to water or the acidic aqueous solution in advance, and a method involving adding it after bringing the organic solvent-containing solution into contact with water or the acidic aqueous solution may be employed. Among them, the method involving adding it to the organic solvent-containing solution in advance is preferable in terms of the workability of operations and the ease of managing the amount to be charged.

The organic solvent that inadvertently mixes with water used in the above purification method is not particularly limited, but is preferably an organic solvent that is safely applicable to a semiconductor production process. The amount of the organic solvent used that inadvertently mixes with water is not particularly limited as long as the solution phase and the aqueous phase separate, but is preferably 0.1 to 100 times, more preferably 0.1 to 50 times, and further preferably 0.1 to 20 times the total mass of the resin to be used.

Specific examples of the organic solvent that inadvertently mixes with water used in the above purification method include, but are not limited to, ethers such as tetrahydrofuran and 1,3-dioxolane; alcohols such as methanol, ethanol, and isopropanol; ketones such as acetone and N-methylpyrrolidone; aliphatic hydrocarbons such as glycol ethers such as ethylene glycol monoethyl ether, ethylene glycol monobutyl ether, propylene glycol monomethyl ether (PGME), and propylene glycol monoethyl ether. Among them, N-methylpyrrolidone, propylene glycol monomethyl ether and the like are preferable, and N-methylpyrrolidone and propylene glycol monomethyl ether are more preferable. These solvents can be each used alone, or can also be used as a mixture of two or more kinds.

The temperature when the extraction treatment is carried out is normally in the range of 20 to 90° C., and preferably 30 to 80° C. The extraction operation is carried out, for example, by thoroughly mixing the solution (S) and the acidic aqueous solution by stirring or the like and then leaving the obtained mixed solution to stand still. Thereby, metals contained in the solution (S) are transferred to the aqueous phase. Also, by this operation, the acidity of the solution is lowered, and the deterioration of the above resin can be suppressed.

By being left to stand still, the mixed solution is separated into an aqueous phase and a solution phase containing the resin and the solvent, and thus the solution phase is recovered by decantation or the like. The time for leaving the mixed solution to stand still is not particularly limited, but it is preferable to regulate the time for leaving the mixed solution to stand still from the viewpoint of attaining good separation of the solution phase containing the solvent and the aqueous phase. Normally, the time for leaving the mixed solution to stand still is 1 minute or longer, preferably 10 minutes or longer, and more preferably 30 minutes or longer. While the extraction treatment may be carried out only once, it is effective to repeat mixing, leaving-to-stand-still, and separating operations multiple times.

It is preferable that the purification method comprise the step of extracting impurities in the resin by further bringing the solution phase containing the above resin into contact with water after the first extraction step (the second extraction step). Specifically, for example, it is preferable that, after the above extraction treatment is carried out using an acidic aqueous solution, the solution phase that is extracted and recovered from the aqueous solution and that contains the resin and the solvent be further subjected to an extraction treatment with water. The extraction treatment with water is not particularly limited, and can be carried out, for example, by thoroughly mixing the solution phase and water by stirring or the like and then leaving the obtained mixed solution to stand still. The mixed solution after being left to stand still is separated into an aqueous phase and a solution phase containing the above resin and the solvent, and thus the solution phase can be recovered by decantation or the like.

Water used herein is preferably water, the metal content of which is small, such as ion exchanged water, according to the purpose of the present embodiment. While the extraction treatment may be carried out only once, it is effective to repeat mixing, leaving-to-stand-still, and separating operations multiple times. The proportions of both used in the extraction treatment, and temperature, time and other conditions are not particularly limited, and may be the same as those of the previous contact treatment with the acidic aqueous solution.

Water that is possibly present in the thus-obtained solution containing the resin and the solvent can be easily removed by performing vacuum distillation or a like operation. Also, if required, the concentration of the resin can be regulated to be any concentration by adding a solvent to the solution.

In the purification method of the compound according to the present embodiment, the compound can be purified by passing a solution obtained by dissolving the resin in a solvent through a filter.

According to the purification method according to the present embodiment, the content of various metals in the resin can be effectively and significantly reduced. The amount of these metal components can be measured by the method described in Examples, which will be mentioned later.

Note that “passing” in the present embodiment means that the above solution is passed from the outside of a filter through the inside of the filter, and then transferred to the outside of the filter again, and for example, an aspect in which the above solution is brought into contact merely on the surface of a filter and an aspect in which the above solution is transferred outside of the ion exchange resin while being brought into contact with the surface of a filter (that is, a mere contact aspect) are eliminated.

### [Filtering Purification Step (Passing Step)]

In the filter-passing step in the present embodiment, the filter used for removing the metals in a solution containing the resin and the solvent can be one which is normally commercially available as a filter for liquid filtration. The filtering precision of the filter is not particularly limited, but the nominal pore size of the filter is preferably 0.2 μm or less, more preferably less than 0.2 μm, further preferably 0.1 μm or less, still more preferably less than 0.1 μm, and even more preferably 0.05 μm or less. The lower limit value of the nominal pore size of the filter is not particularly limited, but is normally 0.005 μm. The nominal pore size herein refers to the nominal pore size indicating the separation performance of a filter and is the pore size determined by test methods defined by the manufacturer of the filter, such as a bubble point test, a mercury intrusion porosimetry test, and a standard particle capture test. In the case of using a commercial product, it is the value described in the catalog data of the manufacturer. By setting the nominal pore size to 0.2 μm or less, the content of the metals after the solution is passed through the filter once can be effectively reduced. In the present embodiment, the filter-passing step may be carried out twice or more to reduce the content of each metal in the solution.

As the form of the filter, a hollow fiber membrane filter, a membrane filter, a pleated membrane filter, and a filter packed with a filtering medium such as a non-woven fabric, cellulose, and diatomaceous earth can be used. Among the above, it is preferable that the filter be one or more selected from the group consisting of a hollow fiber membrane filter, a membrane filter, and a pleated membrane filter. In particular, in terms of highly fine filtering precision and a high filtration area as compared to other forms, a hollow fiber membrane filter is particularly preferably used.

Examples of the filter material include polyolefin such as polyethylene and polypropylene; a polyethylene resin in which a functional group having ion exchange capacity is applied by graft polymerization; a polar group-containing resin such as polyamide, polyester, and polyacrylonitrile; and a fluorine-containing resin such as fluorinated polyethylene (PTFE). Among the above, the filtering medium of the filter is preferably one or more selected from the group consisting of a filtering medium made of polyamide, a filtering medium made of polyolefin resin, and a filtering medium made of fluorine resin. From the viewpoint of an effect of reducing heavy metals such as chrome, polyamide is particularly preferable. From the viewpoint of avoiding the dissolution of metals from the filtering medium, a filter having a filter material other than sintered metals is preferably used.

Examples of the polyamide filter include (hereinafter, trademarks), but are not limited to, Polyfix nylon series manufactured by KITZ MICROFILTER CORPORATION., Ultipleat P-Nylon 66 and Ultipor N66 manufactured by Nihon Pall Ltd., and LifeASSURE PSN series and LifeASSURE EF series manufactured by 3M Japan Limited.

Examples of the polyolefin filter include, but are not limited to, Ultipleat, PE-Kleen and IonKleen manufactured by Nihon Pall Ltd., and Protego series, Microgard Plus HC10, and Optimizer D manufactured by Nihon Entegris G.K.

Examples of the polyester filter include, but are not limited to, Duraflow DFE manufactured by Central Filter Mfg Co Ltd., and a pleated type, PMC manufactured by Nihon Filter Co., Ltd.

Examples of the polyacrylonitrile filter include, but are not limited to, Ultrafilter AIP-0013D, ACP-0013D, and ACP-0053D manufactured by ADVANTEC TOYO KAISHA, LTD.

Examples of the fluorine resin filter include, but are not limited to, Emflon HTPFR manufactured by Nihon Pall Ltd., and LifeASSURE FA series manufactured by 3M Japan Limited.

These filters may be each used alone, or may also be used in combination of two or more kinds.

The above filter may contain an ion exchanger such as a cation exchange resin, a cation charge regulator that generates a zeta potential in an organic solvent solution to be filtered, or the like.

Examples of the filter containing an ion exchanger include, but are not limited to, Protego series manufactured by Nihon Entegris G.K. and KURANGRAFT manufactured by Kurashiki Textile Manufacturing Co., Ltd.

Examples of the filter containing a substance having a positive zeta potential, such as polyamide polyamine epichlorohydrin cationic resin, include (hereinafter, trademarks), but are not limited to, Zeta Plus 40QSH and Zeta Plus 020GN, or LifeASSURE EF series manufactured by 3M Japan Limited.

### [Applications of Compound (A)]

The compound (A) according to the present embodiment can increase the sensitivity to an exposure light source by being added to a composition for film formation as it is or as the polymer described below. The compound (A) or a polymer thereof is preferably used for photoresists.

### [Composition]

The composition of the present embodiment contains the compound (A). The content of the compound (A) in the present embodiment is preferably 90% by mass or more, more preferably 95% by mass or more, and further preferably 99% by mass or more.

As other preferred form of the composition of the present embodiment, it is preferable that the compound (A) at least include a compound represented by the formula (1) other than the formula (1C) and a compound represented by the formula (1C). The proportion of the monomer represented by the formula (1C) contained is preferably as little as 1 ppm by mass or more and 10% by mass or less, more preferably 20 ppm by mass or more and 2% by mass or less, and 50 ppm by mass or more and 1% by mass or less, based on the monomer represented by the formula (1).

By setting the content of the compound represented by the formula (1C) to the described range, the interaction between resins when being formed into a resin can be reduced, and by suppressing the crystalline due to the interaction between the resins after a film is formed using the resin, the locality in the solubility in the developing solution upon developing can be reduced at a molecular level from several to several tens of nanometers, the reduction in pattern quality, such as the line edge roughness and the residue defect, of a pattern which is formed in the pattern formation process in a series of lithography processes of exposure, post exposure bake, and development can be suppressed, and the resolution can be further improved.

The influence of these effects for lithography performance increases in the compound represented by the formula (1C), as a result that the hydrophilicity/hydrophobicity of the compound represented by the formula (1) and the compound represented by the formula (1C) each having the core A into which a halogen element, in particular, iodine, fluorine, or the like is introduced, is shifted to a compound having a hydroxystyrene skeleton in which no iodine and the like are introduced, and the polarization at the polar moieties is thus increased.

In the composition of the present embodiment, impurities containing K (potassium) are preferably 1 ppm by mass or less, more preferably 0.5 ppm by mass or less, further preferably 0.1 ppm by mass or less, and still more preferably 0.005 ppm by mass or less, in terms of element, based on the compound (A).

In the composition of the present embodiment, one or more elemental impurities selected from the group consisting of Mn (manganese), Al (aluminum), Si (silicon), and Li (lithium) (preferably, one or more elemental impurities selected from the group consisting of Mn and Al) are preferably 1 ppm or less, more preferably 0.5 ppm or less, further preferably 0.1 ppm or less, in terms of element, based on the compound (A).

The amount of K, Mn, Al, and the like is measured by inorganic elemental analysis (IPC-AES/IPC-MS). Examples of the inorganic element analyzer include “AG8900” manufactured by Agilent Technologies, Inc.

In the composition of the present embodiment, a phosphorus-containing compound is preferably 10 ppm or less, more preferably 8 ppm or less, and further preferably 5 ppm or less, based on the compound (A).

In the composition of the present embodiment, maleic acid is preferably 10 ppm or less, more preferably 8 ppm or less, and further preferably 5 ppm or less, based on the compound (A).

The amount of the phosphorus-containing compound and maleic acid is calculated from the area fraction of the GC chart and the peak intensity ratio between the target peak and the reference peak by gas chromatography mass spectrometry (GC-MS).

In the composition of the present embodiment, the amount of peroxide is preferably 10 ppm or less, more preferably 1 ppm or less, and further preferably 0.1 ppm or less, based on the compound (A).

The amount of peroxide is quantified by an ammonium ferrothiocyanate acid method (hereinafter, AFTA method) by adding trichloroacetic acid in a sample, then adding ammonium iron (II) sulfate and potassium thiocyanate, determining a calibration curve of peroxide which is known as a standard, and measuring the absorbance at a wavelength of 480 μm.

In the composition of the present embodiment, the moisture content is preferably 100,000 ppm or less, more preferably 20,000 ppm or less, further preferably 1,000 ppm or less, still more preferably 500 ppm or less, and still more preferably 100 ppm or less, based on the compound (A). The moisture content is measured by a Karl Fischer method (Karl Fischer moisture content measuring apparatus).

### [Polymer (A)]

The polymer (A) of the present embodiment contains a constitutional unit derived from the aforementioned compound (A). By containing the constitutional unit derived from the compound (A), the polymer (A) can increase the sensitivity to an exposure light source when being blended in a resist composition. In particular, the polymer (A) can exhibit sufficient sensitivity and can form good thin line patterns having a narrow line width even in the case of using an extreme ultraviolet ray as the exposure light source.

The conventional resist compositions were difficult to expand for actual semiconductor production, since their sensitivity to an exposure light source may be reduced over time due to storage or the like. However, according to the polymer (A) of the present embodiment, the stability of the resist composition is improved, and the reduction in the sensitivity to an exposure light source is suppressed even in the case of long-term storage.

The polymer (A) of the present embodiment contains the constitutional unit derived from the compound (A).

The constitutional unit derived from the compound (A) is the constitutional unit represented by the following formula (4).

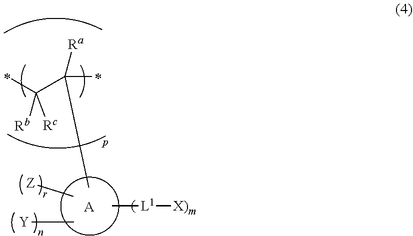

In the formula (4), X, L¹, Y, R^{a}, R^{b}, R^{c}, A, Z, p, m, n, and r are as defined in the formula (1).

The polymer (A) can be obtained by polymerizing the compound (A) of the present embodiment, or by copolymerizing the compound (A) and other monomers. For example, the polymer (A) can be used as a material for forming a film for lithography.

The constitutional unit derived from the compound (A) is preferably the constitutional unit represented by the following formula (5).

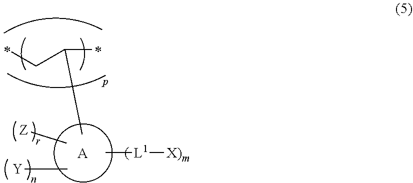

In the formula (5), X, L¹, Y, A, p, m, and n are as defined in the formula (1).

The constitutional unit derived from the compound (A) is more preferably the constitutional unit represented by the following formula (6).

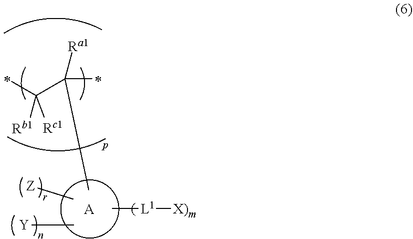

In the formula (6), X, L¹, Y, R^{a1}, R^{b1}, R^{c1}, A, Z, p, m, n, and r are as defined in the formula (1b).

The amount of the constitutional unit derived from the compound (A) is preferably 5 mol % or more, more preferably 8 mol % or more, and further preferably 10 mol % or more, based on the total amount of the monomer components of the polymer (A). The amount of the constitutional unit derived from the compound (A) is 100 mol % or less, preferably 80 mol % or less, more preferably 50 mol % or less, and further preferably 30 mol % or less, based on the total amount of the monomer components of the polymer (A).

As a preferred form of the polymer of the present embodiment, it is preferable that, as the constitutional unit of the polymer (A), the monomer represented by the compound (A) at least include a compound represented by the formula (1) other than the formula (1C) and a compound represented by the formula (1C). The proportion of the monomer represented by the formula (1C) contained is preferably as little as 10 ppm by mass or more and 10% by mass or less, more preferably 20 ppm or more and 2% by mass or less, and 50 ppm or more and 1% by mass or less, based on the monomer represented by the formula (1).

By setting the content of the compound represented by the formula (1C) to the described range, the interaction between resins when being formed into a resin can be reduced, and by suppressing the crystalline due to the interaction between the resins after a film is formed using the resin, the locality in the solubility in the developing solution upon developing can be reduced at a molecular level from several to several tens of nanometers, the reduction in pattern quality, such as the line edge roughness and the residue defect, of a pattern which is formed in the pattern formation process in a series of lithography processes of exposure, post exposure bake, and development can be suppressed, and the resolution can be further improved.

The influence of these effects for lithography performance increases in the monomer represented by the formula (1C), as a result that the hydrophilicity/hydrophobicity of the compound represented by the formula (1) and the compound represented by the formula (1C) each having the core A into which a halogen element, in particular, iodine, fluorine, or the like is introduced, is shifted to a compound having a hydroxystyrene skeleton in which no iodine and the like are introduced, and the polarization at the polar moieties is thus increased.

As the other monomers to be copolymerized with the compound (A), it is preferable to contain a polymerization unit that has an aromatic compound having an unsaturated double bond as a substituent, as a polymerization unit, and has a functional group for improving solubility in an alkaline developing solution by the action of an acid or a base.

Examples of other monomers include, but are not particularly limited to, those described in International Publication No. WO 2016/125782, International Publication No. WO 2015/115613, Japanese Patent Laid-Open No. 2015/117305, International Publication No. WO 2014/175275, and Japanese Patent Laid-Open No. 2012/162498, or a compound represented by the following formula (C1) or formula (C2). Among them, the compound represented by the following formula (C1) or formula (C2) is preferable.

From the viewpoint of the quality of the pattern shape after exposure and development in a lithography process, in particular, suppressing the roughness and the pattern collapse, it is preferable that the difference between the dissolution rate Rₘᵢₙ of the resin that becomes protrusions of a pattern during alkali development on unexposed portions during exposure in the alkaline developing solution and the dissolution rate Rₘₐₓ of the resin that becomes recesses of the pattern during alkali development on exposed portions during exposure in the alkaline developing solution be 3 or more orders of magnitude larger, and it is preferable that the difference in the dissolution rate due to the presence or absence of a protective group be large and the elimination rate of the protective group in post-exposure bake (PEB) and development be large. From these viewpoints, other monomers to be copolymerized with the compound (A) in the polymer (A) preferably have a constitutional unit represented by the following formula (C1).

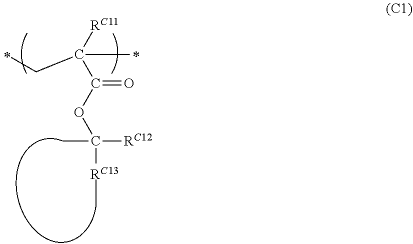

In the formula (C1),

R^{C11} is H or a methyl group;

R^{C12} is H or an alkyl group having 1 to 4 carbon atoms;

R^{C13} is taken together with a carbon atom to which R^{C13} is bonded to be a cycloalkyl group or heterocycloalkyl group having 4 to 20 carbon atoms; and

* is a site for binding with an adjacent constitutional unit.

R^{C12} is preferably H or an alkyl group having 1 to 3 carbon atoms, R^{C13} is preferably taken together with a carbon atom to which R^{C13} is bonded to be a cycloalkyl group or heterocycloalkyl group having 4 to 10 carbon atoms. The cycloalkyl group or heterocycloalkyl group of R^{C13} may have a substituent (e.g., an oxo group).

The amount of the constitutional unit represented by the formula (C1) is preferably 5 mol % or more, more preferably 10 mol % or more, and further preferably 20 mol % or more, based on the total amount of the monomer components of the polymer (A). The amount of the constitutional unit represented by the formula (C1) is preferably 90 mol % or less, more preferably 80 mol % or less, further preferably 70 mol % or less, based on the total amount of the monomer components of the polymer (A).

Other monomers to be copolymerized with the compound (A) in the polymer (A) are preferably the constitutional unit represented by the following formula (C2), from the viewpoint of the quality of the pattern shape after exposure and development in a lithography process, in particular, suppressing the roughness and the pattern collapse.

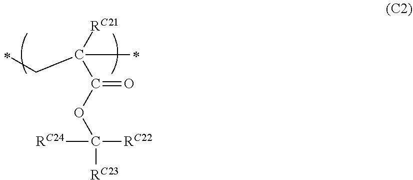

In the formula (C2),

R^{C21} is H or a methyl group;

each of R^{C22} and R^{C23} is independently an alkyl group having 1 to 4 carbon atoms;

R^{C24} is an alkyl group having 1 to 4 carbon atoms or a cycloalkyl group having 5 to 20 carbon atoms;

two or three of R^{C22}, R^{C23}, and R^{C24} are taken together with a carbon atom to which they are bonded to form an alicyclic structure having 3 to 20 carbon atoms; and

* is a site for binding with an adjacent constitutional unit.

R^{C22} is preferably an alkyl group having 1 to 3 carbon atoms, and R^{C24} is a cycloalkyl group having 5 to 10 carbon atoms. The above alicyclic structure formed by R^{C22}, R^{C23}, and R^{C24} may contain a plurality of rings such as an adamantyl group. The above alicyclic structure may have a substituent (e.g., a hydroxyl group and an alkyl group).

The amount of the constitutional unit represented by the formula (C2) is preferably 5 mol % or more, more preferably 10 mol % or more, and further preferably 20 mol % or more, based on the total amount of the monomer components of the polymer (A). The amount of the constitutional unit represented by the formula (C2) is preferably 80 mol % or less, more preferably 60 mol % or less, further preferably 40 mol % or less, based on the total amount of the monomer components of the polymer (A).

Examples of the monomer raw material of the constitutional unit represented by the formula (C2) include, but are not limited to, 2-methyl-2-(meth)acryloyloxyadamantane, 2-ethyl-2-(meth)acryloyloxyadamantane, 2-isopropyl-2-(meth)acryloyloxyadamantane, 2-n-propyl-2-(meth)acryloyloxyadamantane, 2-n-butyl-2-(meth)acryloyloxyadamantane, 1-methyl-1-(meth)acryloyloxycyclopentane, 1-ethyl-1-(meth)acryloyloxycyclopentane, 1-methyl-1-(meth)acryloyloxycyclohexane, 1-ethyl-1-(meth)acryloyloxycyclohexane, 1-methyl-1-(meth)acryloyloxycycloheptane, 1-ethyl-1-(meth)acryloyloxycycloheptane, 1-methyl-1-(meth)acryloyloxycyclooctane, 1-ethyl-1-(meth)acryloyloxycyclooctane, 2-ethyl-2-(meth)acryloyloxydecahydro-1,4:5,8-dimethanonaphtalene, and 2-ethyl-2-(meth)acryloyloxynorbornane. Commercial products may be used as these monomer.

Other monomers to be copolymerized with the compound (A) in the polymer (A) preferably have a constitutional unit represented by the following formula (C3).

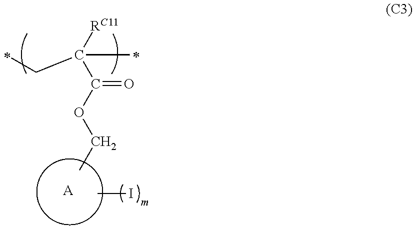

In the formula (C3), R^{C31} is H or a methyl group, and m, A, and * are as defined in the above formula (4).

Other monomers to be copolymerized with the compound (A) in the polymer (A) preferably have a constitutional unit represented by the following formula (C4).

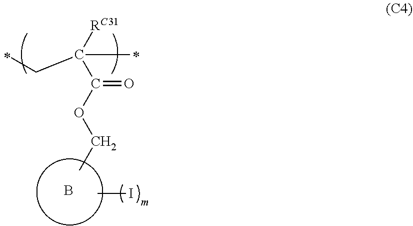

In the formula (C4), B represents an organic group containing an aromatic ring and having 5 to 30 carbon atoms, and R^{C31}, m, and * are as defined in the above formula (C3).

Other monomers to be copolymerized with the compound (A) in the polymer (A) preferably have a constitutional unit represented by the following formula (C5).

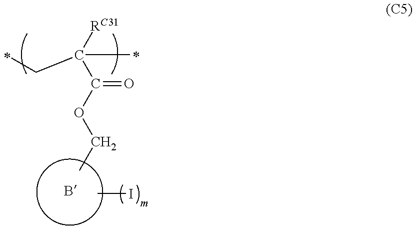

In the formula (C5), B′ represents an organic group containing an aromatic ring and having 5 to 30 carbon atoms, and R^{C31}, m, and * are as defined in the above formula (C3).

Other monomers to be copolymerized with the compound (A) in the polymer (A) are preferably the constitutional unit represented by the following formula (C6), from the viewpoint of the exposure sensitivity in pattern formation and the quality of the pattern shape after exposure and development in a lithography process, in particular, suppressing the roughness and the pattern collapse.

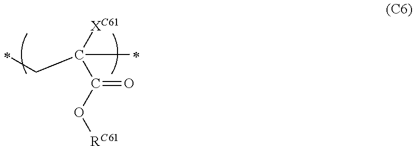

In the formula (C6),

X^{C61} is a hydroxyl group or a halogen group;

each R^{C61} is independently an alkyl group having 1 to 20 carbon atoms; and

* is a site for binding with an adjacent constitutional unit.

X^{C61} is preferably F, Cl, Br, or I, further preferably Cl or I, and still more preferably I. R^{C61} is preferably an alkyl group having 1 to 4 carbon atoms, and more preferably a methyl group.

The amount of the constitutional unit represented by the formula (C6) is preferably 20 mol % or more, more preferably 30 mol % or more, and further preferably 40 mol % or more, based on the total amount of the monomer components of the polymer (A). The amount of the constitutional unit represented by the formula (C6) is preferably 80 mol % or less, more preferably 70 mol % or less, further preferably 60 mol % or less, based on the total amount of the monomer components of the polymer (A).

Examples of the monomer raw material of the constitutional unit represented by the formula (C6) include, but are not limited to, methyl 2-chloroacrylate, ethyl 2-chloroacrylate, butyl 2-chloroacrylate, methyl 2-bromoacrylate, ethyl 2-bromoacrylate, butyl 2-bromoacrylate, methyl 2-iodoacrylate, ethyl 2-iodoacrylate, and butyl 2-iodoacrylate. Commercial products may be used as these monomer.

Next, the method for producing the polymer (A) will be described. The polymerization reaction is carried out by dissolving a monomer as a constitutional unit in a solvent and adding a polymerization initiator while heating or cooling. The reaction conditions can be inadvertently set depending on the kind of polymerization initiator, the initiation method such as heat and light, the temperature, the pressure, the concentration, the solvent, the additive, and the like. Examples of the polymerization initiator include a radical polymerization initiator such as azoisobutyronitrile and peroxide, and an anion polymerization initiator such as alkyllithium and a Grignard reagent.

As the solvent used in the polymerization reaction, a commonly available product can be used. For example, a wide variety of solvents such as alcohols, ethers, hydrocarbons, and halogenated solvents can be arbitrarily used within a range not inhibiting the reaction. Within the above range not inhibiting the reaction, a plurality of solvents can be used as a mixture.

The polymer (A) obtained in the polymerization reaction can be purified by a publicly known method. Specifically, ultrafiltration, crystallization, microfiltration, acid washing, water washing at an electrical conductivity of 10 mS/m or less, and extraction can be used in combination.

### [Composition for Film Formation]

The composition for film formation of the present embodiment contains the compound (A) or the polymer (A), and is the composition particularly suitable for lithography technology. The composition can be used for, without particular limitation, film formation purposes for lithography, for example, resist film formation purposes (that is, a “resist composition”). Furthermore, the composition can be used for upper layer film formation purposes (that is, a “composition for upper layer film formation”), intermediate layer formation purposes (that is, a “composition for intermediate layer formation”), underlayer film formation purposes (that is, a “composition for underlayer film formation”), and the like. According to the composition of the present embodiment, not only a film having high sensitivity can be formed, but also a good resist pattern shape can be imparted.

The composition for film formation of the present embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

The composition for film formation of the present embodiment contains the compound (A) or the polymer (A), and may contain other components such as a base material (B), a solvent (S), an acid generating agent (C), and an acid diffusion controlling agent (E), if required. Hereinafter, each of these components will be described.

### [Base Material (B)]

The “base material (B)” in the present embodiment is a compound (including a resin) other than the compound (A) or the polymer (A) and means a base material applied as a resist for g-ray, i-ray, KrF excimer laser (248 nm), ArF excimer laser (193 nm), extreme ultraviolet (EUV) lithography (13.5 nm) or electron beam (EB) (for example, a base material for lithography or a base material for resist). These base materials can be used as the base material (B) in the present embodiment without particular limitation. Examples of the base material (B) include a phenol novolac resin, a cresol novolac resin, a hydroxystyrene resin, a (meth)acrylic resin, and a hydroxystyrene-(meth)acrylic copolymer, a cycloolefin-maleic anhydride copolymer, a cycloolefin, a vinyl ether-maleic anhydride copolymer, and an inorganic resist material having a metallic element such as titanium, tin, hafnium, and zirconium, and derivatives thereof. Among them, from the viewpoint of the shape of a resist pattern to be obtained, preferable are a phenol novolac resin, a cresol novolac resin, a hydroxystyrene resin, a (meth)acrylic resin, a hydroxystyrene-(meth)acrylic copolymer, and an inorganic resist material having a metallic element such as titanium, tin, hafnium, and zirconium, and derivatives thereof.

Examples of the derivative include, but are not particularly limited to, those to which a dissociation group is introduced and those to which a crosslinkable group is introduced. The above derivative to which a dissociation group or a crosslinkable group is introduced can exhibit dissociation reaction or crosslinking reaction through the effect of light, acid or the like.

The “dissociation group” refers to a characteristic group that is cleaved to generate a functional group that alters solubility, such as an alkali soluble group. Examples of the alkali soluble group include, but are not particularly limited to, a phenolic hydroxy group, a carboxyl group, a sulfonic acid group, and a hexafluoroisopropanol group, and a phenolic hydroxy group and a carboxyl group are preferable, and a phenolic hydroxy group is particularly preferable.

The “crosslinking group” refers to a group that crosslinks in the presence of a catalyst or without a catalyst. Examples of the crosslinking group include, but are not particularly limited to, an alkoxy group having 1 to 20 carbon atoms, a group having an allyl group, a group having a (meth)acryloyl group, a group having an epoxy (meth)acryloyl group, a group having a hydroxy group, a group having a urethane (meth)acryloyl group, a group having a glycidyl group, and a group having a vinyl-containing phenylmethyl group.

### [Solvent (S)]

As the solvent in the present embodiment, a known solvent can be arbitrarily used as long as it can at least dissolve the compound (A) or the polymer (A) mentioned above. Examples of the solvent include, but are not particularly limited to, ethylene glycol monoalkyl ether acetates such as ethylene glycol monomethyl ether acetate, ethylene glycol monoethyl ether acetate, ethylene glycol mono-n-propyl ether acetate, and ethylene glycol mono-n-butyl ether acetate; ethylene glycol monoalkyl ethers such as ethylene glycol monomethyl ether and ethylene glycol monoethyl ether; propylene glycol monoalkyl ether acetates such as propylene glycol monomethyl ether acetate (PGMEA), propylene glycol monoethyl ether acetate, propylene glycol mono-n-propyl ether acetate, and propylene glycol mono-n-butyl ether acetate; propylene glycol monoalkyl ethers such as propylene glycol monomethyl ether (PGME) and propylene glycol monoethyl ether; lactate esters such as methyl lactate, ethyl lactate, n-propyl lactate, n-butyl lactate, and n-amyl lactate; aliphatic carboxylic acid esters such as methyl acetate, ethyl acetate, n-propyl acetate, n-butyl acetate, n-amyl acetate, n-hexyl acetate, methyl propionate, and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutylacetate, 3-methyl-3-methoxybutylacetate, butyl 3-methoxy-3-methylpropionate, butyl 3-methoxy-3-methylbutyrate, methyl acetoacetate, methyl pyruvate, and ethyl pyruvate; aromatic hydrocarbons such as toluene and xylene; ketones such as acetone, 2-butanone, 2-heptanone, 3-heptanone, 4-heptanone, cyclopentanone (CPN), and cyclohexanone (CHN); amides such as N,N-dimethylformamide, N-methylacetamide, N,N-dimethylacetamide, and N-methylpyrrolidone; and lactones such as y-lactone. The solvent used in the present embodiment is preferably a safe solvent, more preferably at least one selected from PGMEA, PGME, CHN, CPN, 2-heptanone, anisole, butyl acetate and ethyl lactate, and further preferably at least one selected from PGMEA, PGME, CHN, CPN and ethyl lactate.

In the composition for film formation of the present embodiment, the solid component concentration is not particularly limited, but is preferably 1 to 80% by mass, more preferably 1 to 50% by mass, further preferably 2 to 40% by mass, and still more preferably 2 to 10% by mass, based on the total mass of the composition for film formation.

### [Acid Generating Agent (C)]

It is preferable that the composition for film formation of the present embodiment contain one or more acid generating agents (C) that directly or indirectly generates an acid by radiation irradiation. Radiation is at least one selected from the group consisting of visible light, ultraviolet, excimer laser, electron beam, extreme ultraviolet (EUV), X-ray, and ion beam. The acid generating agent (C) is not particularly limited, and, for example, an acid generating agent described in International Publication No. WO 2013/024778 can be used. The acid generating agent (C) can be used alone or in combination of two or more kinds.

The amount of the acid generating agent (C) blended is preferably 0.001 to 49% by mass, more preferably 1 to 40% by mass, further preferably 3 to 30% by mass, and still more preferably 10 to 25% by mass, based on the total mass of the solid components. By using the acid generating agent (C) within the above range, a pattern profile with high sensitivity and low edge roughness tends to be obtained. In the present embodiment, the acid generation method is not particularly limited, as long as an acid is generated in the system. By using excimer laser instead of ultraviolet such as g-ray and i-ray, finer processing is possible, and also by using electron beam, extreme ultraviolet, X-ray, or ion beam as a high energy ray, further finer processing is possible.

### [Acid Diffusion Controlling Agent (E)]

The composition for film formation of the present embodiment may contain the acid diffusion controlling agent (E). The acid diffusion controlling agent (E) controls diffusion of an acid generated from an acid generating agent by radiation irradiation in a resist film to inhibit any unpreferable chemical reaction in an unexposed region or the like. By using the acid diffusion controlling agent (E), there is a tendency that the storage stability of the composition of the present embodiment can be improved. Also, by using the acid diffusion controlling agent (E), there is a tendency that not only the resolution of a film formed by using the composition of the present embodiment can be improved, but also the line width change of a resist pattern due to variation in the post exposure delay time before radiation irradiation and the post exposure delay time after radiation irradiation can also be suppressed, making the composition excellent in process stability. Examples of the acid diffusion controlling agent (E) include, but are not particularly limited to, a radiation degradable basic compound such as a nitrogen atom containing basic compound, a basic sulfonium compound, and a basic iodonium compound.

The acid diffusion controlling agent (E) is not particularly limited, and, for example, an acid diffusion controlling agent described in International Publication No. WO 2013/024778 can be used. The acid diffusion controlling agent (E) can be used alone or in combination of two or more kinds.

The amount of the acid diffusion controlling agent (E) blended is preferably 0.001 to 49% by mass, more preferably 0.01 to 10% by mass, further preferably 0.01 to 5% by mass, and further preferably 0.01 to 3% by mass, based on the total mass of the solid components. When the amount of the acid diffusion controlling agent (E) blended is within the above range, there is a tendency that a decrease in resolution, and deterioration of the pattern shape and the dimension fidelity or the like can be prevented. Moreover, even though the post exposure delay time from electron beam irradiation to heating after radiation irradiation becomes longer, deterioration of the shape of the pattern upper layer portion can be suppressed. When the amount of the acid diffusion controlling agent (E) blended is 10% by mass or less, there is a tendency that a decrease in sensitivity, and developability of the unexposed portion or the like can be prevented. Also, by using such an acid diffusion controlling agent, there is a tendency that the storage stability of a resist composition is improved, also along with improvement of the resolution, the line width change of a resist pattern due to variation in the post exposure delay time before radiation irradiation and the post exposure delay time after radiation irradiation can be suppressed, making the composition excellent in process stability.

### [Other Component (F)]

To the composition for film formation of the present embodiment, if required, as the other component (F), one kind or two or more kinds of various additives such as a cross-linking agent, a dissolution promoting agent, a dissolution controlling agent, a sensitizing agent, a surfactant, and an organic carboxylic acid or an oxo acid of phosphorus or derivative thereof can be added.

### [Crosslinking Agent]

The composition for film formation of the present embodiment may contain the cross-linking agent. The cross-linking agent may crosslink at least one of the compound (A), the polymer (A), and the base material (B). It is preferable that the crosslinking agent be an acid crosslinking agent capable of intramolecularly or intermolecularly crosslinking the base material (B) in the presence of the acid generated from the acid generating agent (C). Examples of such an acid crosslinking agent can include a compound having one or more groups capable of crosslinking the base material (B) (hereinafter, referred to as a “crosslinkable group”).

Examples of the crosslinkable group include: (i) a hydroxyalkyl group or a group derived therefrom, such as a hydroxy group, a hydroxyalkyl group (alkyl group having 1 to 6 carbon atoms), alkoxy having 1 to 6 carbon atoms (alkyl group having 1 to 6 carbon atoms) and acetoxy (alkyl group having 1 to 6 carbon atoms); (ii) a carbonyl group or a group derived therefrom, such as a formyl group and a carboxy (alkyl group having 1 to 6 carbon atoms); (iii) a nitrogenous group containing group such as a dimethylaminomethyl group, a diethylaminomethyl group, a dimethylolaminomethyl group, a diethylolaminomethyl group and a morpholinomethyl group; (iv) a glycidyl group containing group such as a glycidyl ether group, a glycidyl ester group and a glycidylamino group; (v) a group derived from an aromatic group such as an allyloxy having 1 to 6 carbon atoms (alkyl group having 1 to 6 carbon atoms) and an aralkyloxy having 1 to 6 carbon atoms (alkyl group having 1 to 6 carbon atoms) such as a benzyloxymethyl group and a benzoyloxymethyl group; and (vi) a polymerizable multiple bond containing group such as a vinyl group and an isopropenyl group. As the crosslinkable group of the crosslinking agent in the present embodiment, a hydroxyalkyl group, an alkoxyalkyl group and the like are preferable, and an alkoxymethyl group is particularly preferable.

The crosslinking agent having the crosslinkable group is not particularly limited, and, for example, an acid crosslinking agent described in International Publication No. WO 2013/024778 can be used. The crosslinking agent can be used alone or in combination of two or more kinds.

The amount of the cross-linking agent blended in the present embodiment is preferably 50% by mass or less, more preferably 40% by mass or less, further preferably 30% by mass or less, and further preferably 20% by mass or less, based on the total mass of the solid components.

### (Dissolution Promoting Agent)

The dissolution promoting agent is a component having a function of, when the solubility of a solid component is too low, increasing the solubility of the solid component in a developing solution to moderately increase the dissolution rate of the compound upon developing. As the dissolution promoting agent, those having a low molecular weight are preferable, and examples thereof include a phenolic compound having a low molecular weight. Examples of the phenolic compound having a low molecular weight include a bisphenol and a tris(hydroxyphenyl)methane. These dissolution promoting agents can be used alone or in mixture of two or more kinds.

The amount of the dissolution promoting agent blended, which is arbitrarily adjusted according to the kind of the above solid component to be used, is preferably 0 to 49% by mass of the total mass of the solid component, more preferably 0 to 5% by mass, further preferably 0 to 1% by mass, and particularly preferably 0% by mass.

### (Dissolution Controlling Agent)

The dissolution controlling agent is a component having a function of, when the solubility of a solid component is too high, controlling the solubility of the solid component in a developing solution to moderately decrease the dissolution rate upon developing. As such a dissolution controlling agent, the one which does not chemically change in steps such as calcination of resist coating, radiation irradiation, and development is preferable.

The dissolution controlling agent is not particularly limited, and examples thereof include an aromatic hydrocarbon such as phenanthrene, anthracene and acenaphthene; a ketone such as acetophenone, benzophenone and phenyl naphthyl ketone; and a sulfone such as methyl phenyl sulfone, diphenyl sulfone and dinaphthyl sulfone. These dissolution controlling agents can be used alone or in combination of two or more kinds.

The amount of the dissolution controlling agent blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Sensitizing Agent)

The sensitizing agent is a component having a function of absorbing irradiated radiation energy, transmitting the energy to the acid generating agent (C), and thereby increasing the acid production amount, and improving the apparent sensitivity of a resist. Examples of such a sensitizing agent can include, but are not particularly limited to, a benzophenone, a biacetyl, a pyrene, a phenothiazine and a fluorene. These sensitizing agents can be used alone or in combination of two or more kinds.

The amount of the sensitizing agent blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### (Surfactant)

The surfactant is a component having a function of improving coatability and striation of the composition of the present embodiment, and developability of a resist or the like. The surfactant may be any of anionic, cationic, nonionic, and amphoteric surfactants. Preferable examples of the surfactant include a nonionic surfactant. The nonionic surfactant has a good affinity with a solvent to be used in production of the composition of the present embodiment, and can further enhance the effects of the composition of the present embodiment. Examples of the nonionic surfactant include, but are not particularly limited to, a polyoxyethylene higher alkyl ether, a polyoxyethylene higher alkyl phenyl ether, and a higher fatty acid diester of polyethylene glycol. Examples of commercially available products of these surfactants include, hereinafter by trade name, EFTOP (manufactured by Jemco Inc.), MEGAFAC (manufactured by DIC Corporation), Fluorad (manufactured by Sumitomo 3M Limited), AsahiGuard, Surflon (hereinbefore, manufactured by Asahi Glass Co., Ltd.), Pepole (manufactured by Toho Chemical Industry Co., Ltd.), KP (manufactured by Shin-Etsu Chemical Co., Ltd.), and Polyflow (manufactured by Kyoeisha Chemical Co., Ltd.).

The amount of the surfactant blended, which is arbitrarily adjusted according to the kind of the above solid component to be used, is preferably 0 to 49% by mass of the total mass of the solid component, more preferably 0 to 5% by mass, further preferably 0 to 1% by mass, and particularly preferably 0% by mass.

### (Organic Carboxylic Acid or Oxo Acid of Phosphorus or Derivative Thereof)

For the purpose of prevention of sensitivity deterioration or improvement of a resist pattern shape and post exposure delay stability or the like, and as an additional optional component, the composition of the present embodiment can contain an organic carboxylic acid or an oxo acid of phosphorus or derivative thereof. The organic carboxylic acid or the oxo acid of phosphorus or derivative thereof can be used in combination with the acid diffusion controlling agent, or may be used alone. Examples of suitable organic carboxylic acids include malonic acid, citric acid, malic acid, succinic acid, benzoic acid and salicylic acid. Examples of the oxo acid of phosphorus or derivative thereof include phosphoric acid or derivative thereof such as ester including phosphoric acid, di-n-butyl phosphate and diphenyl phosphate; phosphonic acid or derivative thereof such as ester including phosphonic acid, dimethyl phosphonate, di-n-butyl phosphonate, phenylphosphonic acid, diphenyl phosphonate and dibenzyl phosphonate; and phosphinic acid and derivative thereof such as ester including phosphinic acid and phenylphosphinic acid. Among them, phosphonic acid is particularly preferable.

The organic carboxylic acid or the oxo acid of phosphorus or derivative thereof can be used alone or in combination of two or more kinds. The amount of the organic carboxylic acid or the oxo acid of phosphorus or derivative thereof blended, which is arbitrarily adjusted according to the kind of the above compound to be used, is preferably 0 to 49% by mass of the total mass of the solid component, more preferably 0 to 5% by mass, further preferably 0 to 1% by mass, and particularly preferably 0% by mass.

### [Other Additive]

Furthermore, the composition of the present embodiment can contain one kind or two kinds or more of additives other than the components mentioned above, if required. Examples of such an additive include a dye, a pigment and an adhesion aid. For example, when the composition is blended with a dye or a pigment, a latent image of the exposed portion is visualized and influence of halation upon exposure can be alleviated, which is preferable. Also, when the composition is blended with an adhesion aid, adhesiveness to a substrate can be improved, which is preferable. Furthermore, examples of the other additive include a halation preventing agent, a storage stabilizing agent, a defoaming agent and a shape improving agent. Specific examples thereof include 4-hydroxy-4′-methylchalkone.

In the composition of the present embodiment, the total content of the optional component (F) can be 0 to 99% by mass of the total mass of the solid component, and is preferably 0 to 49% by mass, more preferably 0 to 10% by mass, further preferably 0 to 5% by mass, still more preferably 0 to 1% by mass, and particularly preferably 0% by mass.

### [Resist Pattern Formation Method]

The resist pattern formation method of the present embodiment comprises:

a step of forming a resist film on a substrate by using a composition for film formation containing the compound (A) or the polymer (A);

a step of exposing a pattern on the resist film; and

a step of subjecting the resist film to a development treatment after exposure.

Examples of the coating method in the step of forming a resist film include, but are not particularly limited to, spin coating, dip coating, and roll coating. Examples of the substrate include, but are not particularly limited to, a silicon wafer, metal, plastic, glass, and ceramic. After formation of the resist film, a heat treatment may be carried out at a temperature of about 50° C. to 200° C. The film thickness of the resist film is not particularly limited and is for example, 50 nm to 1 μm.

In the exposure step, exposure may be carried out via a predetermined mask pattern, or shot exposure may be carried out without masking. The thickness of the coating film is, for example, 0.1 to 20 μm, and preferably about 0.3 to 2 μm. Lights of various wavelengths such as ultraviolet ray, X-ray can be utilized for exposure, and for example, far ultraviolet ray such as F2 excimer laser (wavelength: 157 nm), ArF excimer laser (wavelength: 193 nm), and KrF excimer laser (wavelength: 248 nm), extreme ultraviolet ray (wavelength: 13 n), X-ray, and electron beam can be arbitrarily selected and used, as a light source. Among them, extreme ultraviolet ray is preferable. The exposure conditions such as the exposure amount are arbitrarily selected depending on the composition of the above resin and/or compound blended, the kind of each additive, and the like.

In the present embodiment, to stably form a fine pattern with a high degree of accuracy, the resist film is preferably subjected to a heat treatment at a temperature of 50 to 200° C. for 30 seconds or more, after exposure. In this case, variation of sensitivity depending on the kind of substrate may increase at a temperature lower than 50° C. Thereafter, development is carried out with an alkaline developing solution under the conditions of normally at 10 to 50° C. for 10 to 200 seconds, and preferably 20 to 25° C. for 15 to 90 seconds, resulting in formation of a predetermined resist pattern.

The above alkaline developing solution used is an alkaline aqueous solution in which an alkaline compound such as an alkali metal hydroxide, aqueous ammonia, an alkylamine, an alkanolamine, a heterocyclic amine, a tetraalkylammonium hydroxide, choline, 1,8-diazabicyclo-[5.4.0]-7-undecene, and 1,5-diazabicyclo-[4.3.0]-5-nonene is dissolved such that the concentration is normally 1 to 10% by mass and preferably 1 to 3% by mass. Into the above developing solution comprising the alkaline aqueous solution, a water-soluble organic solvent and a surfactant can be arbitrarily added.

The composition of the present embodiment can also be used as an optical component forming composition applying lithography technology. The optical component is used in the form of a film or a sheet and is also useful as a plastic lens (a prism lens, a lenticular lens, a microlens, a Fresnel lens, a viewing angle control lens, a contrast improving lens, etc.), a phase difference film, a film for electromagnetic wave shielding, a prism, an optical fiber, a solder resist for flexible printed wiring, a plating resist, an interlayer insulating film for multilayer printed circuit boards, a photosensitive optical waveguide, a liquid crystal display, an organic electroluminescent (EL) display, an optical semiconductor (LED) element, a solid state image sensing element, an organic thin film solar cell, a dye sensitized solar cell, and an organic thin film transistor (TFT). The composition can be suitably utilized as an embedded film and a smoothed film on a photodiode, a smoothed film in front of or behind a color filter, a microlens, and a smoothed film and a conformal film on a microlens, all of which are components of a solid state image sensing element, to which high refractive index is particularly demanded.

Also, the composition of the present embodiment can be used as a patterning material for lithography applications. For lithography process applications, the composition can be used for various applications such as a semiconductor, a liquid crystal display panel, a display panel using OLED, a power device, and CCD and other sensors. In particular, for semiconductors and integrated circuits on devices, the composition of the present embodiment can be suitably utilized in the process of forming a device element on a silicon wafer, for the purpose of constructing a semiconductor element and other devices by forming a pattern on an insulating film on the substrate side by etching based on the pattern formed on the upper surface side of the insulating layer such as a silicon oxide film and other oxide films utilizing the composition of the present embodiment, further laminating a metal film or a semiconductor material based on the formed insulating film pattern, and forming a circuit pattern.

### Second Embodiment

Hereinafter, the second embodiment of the present invention will be described. The second embodiment relates to the method for producing an iodine-containing vinyl monomer having the formula (1) described later, and preferably relates to the method for producing an iodine-containing hydroxystyrene. The method for producing the second embodiment can be utilized as the method for producing the compound of the first embodiment.

### [Method for Producing Iodine-Containing Vinyl Monomer Represented by Formula (1)]

The second embodiment is the method for producing the iodine-containing vinyl monomer having the following formula (1), preferably the iodine-containing hydroxystyrene.

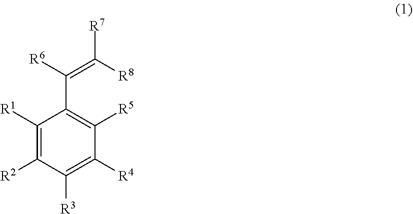

(In the formula (1), each of R¹ to R⁵ is independently H, OH, OCH₃, a halogen, or a linear or branched alkyl, each of R⁶ to R⁸ is independently H, OH, OCH₃, a halogen, or a cyano group, provided that at least one of R¹ to R⁵ is OH and at least one of them is iodine.)

Examples of the hydroxystyrene produced by the method of the present embodiment include, but are not limited to, iodine-containing 2-hydroxystyrene, iodine-containing 3-hydroxystyrene, iodine-containing 4-hydroxystyrene, iodine-containing 3-methoxy-4-hydroxystyrene, iodine-containing 3,5-dimethoxy-4-hydroxystyrene, iodine-containing 2,3-dihydroxystyrene, iodine-containing 2,4-dihydroxystyrene, iodine-containing 2,5-dihydroxystyrene, iodine-containing 2,6-dihydroxystyrene, iodine-containing 3,4-dihydroxystyrene, iodine-containing 3,5-dihydroxystyrene, iodine-containing 2,3,4-trihydroxystyrene, iodine-containing 2,4,6-trihydroxystyrene, iodine-containing 3,4,5-trihydroxystyrene, and iodine-containing α-cyano-4-hydroxystyrene. At least one iodine atom is introduced, and two or more iodine atoms are preferably introduced. At least one OH is introduced, and two or more OH are preferably introduced.

Specific examples of the hydroxystyrene produced by the method of the present embodiment include, but are not limited to, the followings.

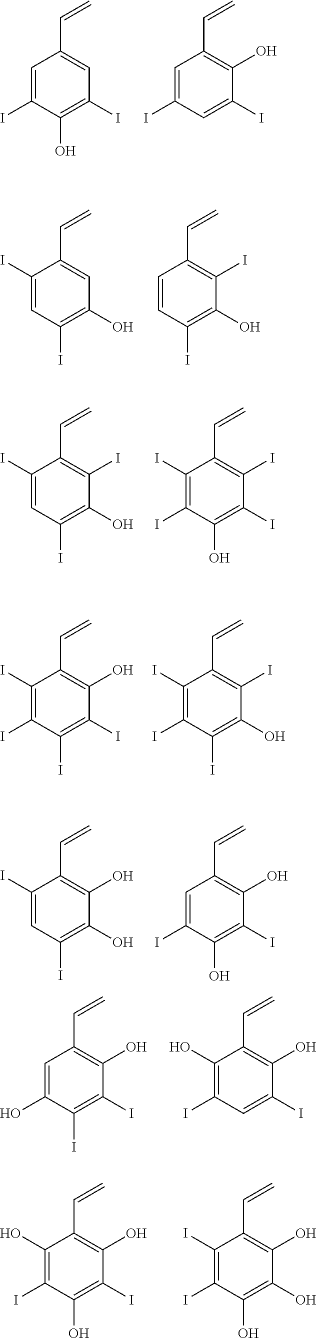

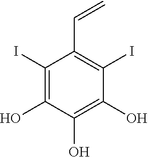

### (Iodine-Containing Alcohol Substrate)

The iodine-containing alcohol substrate used in the present invention is an iodine-containing alcohol substrate having the formula (1-1):

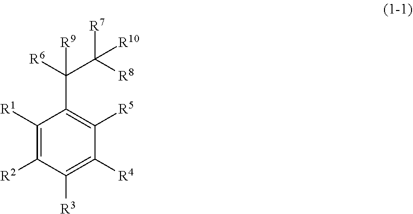

wherein, each of R¹ to R⁵ is independently H, OH, OCH₃, a halogen, or a linear or branched alkyl, each of R⁶ to R¹⁰ is independently H, OH, OCH₃, a halogen, or a cyano group, provided that at least one of R¹ to R⁵ is OH and at least one of them is iodine, and one of R⁶ to R¹⁰ is OH or OCH₃.

Examples of suitable iodine-containing alcohol substrates include, but are not limited to, iodine-containing 2-(1-hydroxyethyl)phenol, iodine-containing 3-(1-hydroxyethyl)phenol, iodine-containing 4-(1-hydroxyethyl)phenol, iodine-containing 4-(1-hydroxyethyl)-1-methoxyphenol, iodine-containing 4-(1-hydroxyethyl)-2,6-dimethoxyphenol, iodine-containing 3-(1-hydroxyethyl)benzene-1,2-diol, iodine-containing 4-(1-hydroxyethyl)benzene-1,3-diol, iodine-containing 2-(1-hydroxyethyl)benzene-1,4-diol, iodine-containing 6-(1-hydroxyethyl)benzene-1,5-diol, iodine-containing 4-(1-hydroxyethyl)benzene-1,2-diol, iodine-containing 5-(1-hydroxyethyl)benzene-1,3-diol, iodine-containing 4-(1-hydroxyethyl)benzene-1,2,3-triol, iodine-containing 2-(1-hydroxyethyl)benzene-1,3,5-triol, iodine-containing 5-(1-hydroxyethyl)benzene-1,2,3-triol, iodine-containing 2-(1-cyano-1-hydroxyethyl)phenol, iodine-containing 2-(2-hydroxyphenyl)ethanol, iodine-containing 2-(3-hydroxyphenyl)ethanol, iodine-containing 2-(4-hydroxyphenyl)ethanol, iodine-containing 2-(3-methoxy-4-hydroxyphenyl)ethanol, iodine-containing 2-(3,5-dimethoxy-4-hydroxyphenyl)ethanol, iodine-containing 2-(2,3-dihydroxyphenyl)ethanol, iodine-containing 2-(2,4-dihydroxyphenyl)ethanol, iodine-containing 2-(2,5-dihydroxyphenyl)ethanol, iodine-containing 2-(2,6-dihydroxyphenyl)ethanol, iodine-containing 2-(3,4-dihydroxyphenyl)ethanol, iodine-containing 2-(3,5-dihydroxyphenyl)ethanol, iodine-containing 2-(2,3,4-trihydroxyphenyl)ethanol, iodine-containing 2-(2,4,6-trihydroxyphenyl)ethanol, iodine-containing 2-(3,4,5-trihydroxyphenyl)ethanol, and iodine-containing 1-cyano-2-(4-hydroxyphenyl)ethanol. At least one iodine atom is introduced, and two or more iodine atoms are preferably introduced. At least one OH is introduced, and two or more OH are preferably introduced. OH may be substituted with OMe.

Specific examples of the iodine-containing alcohol substrate used in the present invention include, but are not limited to the followings.

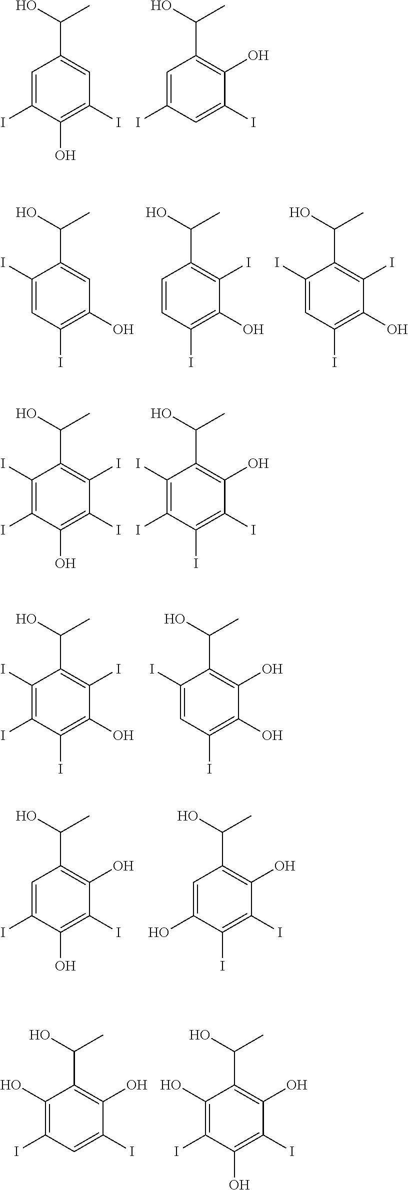

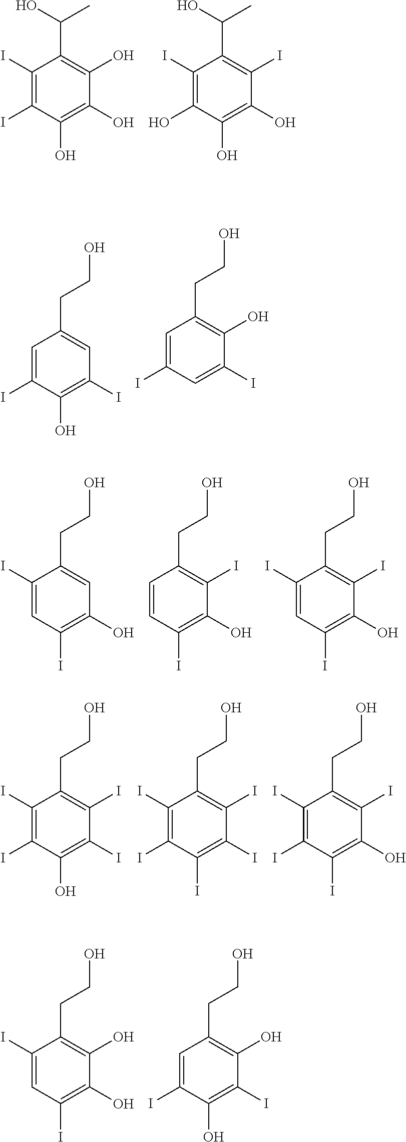

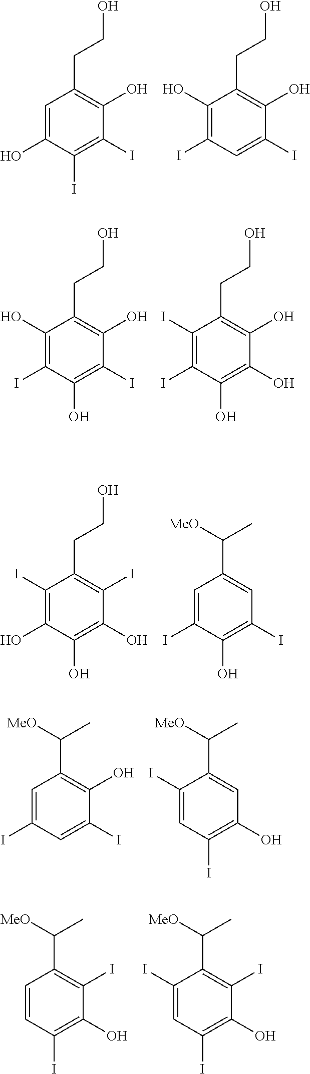

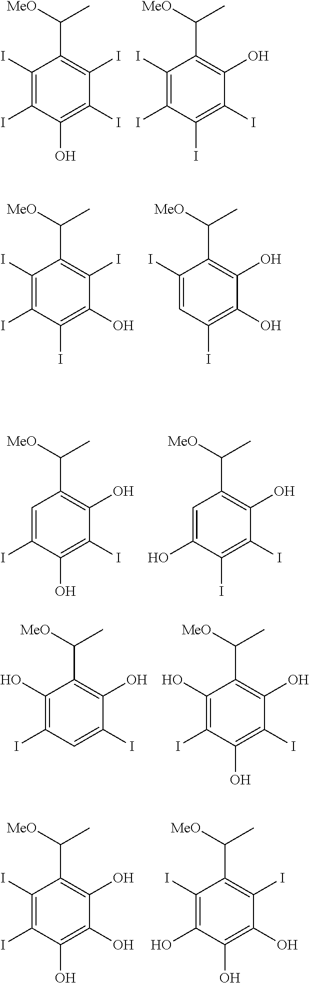

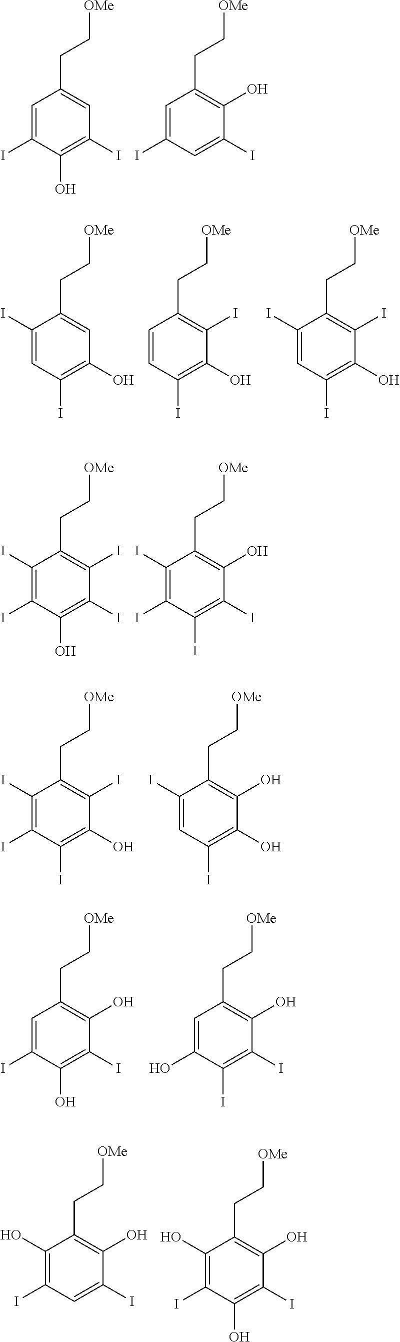

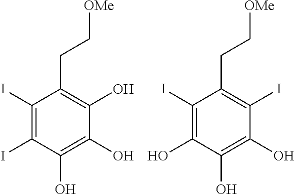

These iodine-containing alcohol substrates can be obtained by many methods, but is preferably obtained by the method described later, from the viewpoint of the availability and yield of the raw material.

The method for producing the iodine-containing vinyl monomer represented by the formula (1) comprises:
a) a step of providing an iodine-containing alcohol substrate having the formula (1-1); and
b) a step of dehydrating the iodine-containing alcohol substrate.

As the solvent that may be used in the dehydrating step, a wide variety of solvents including a polar aprotic solvent and protic polar solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, it is preferably 100 to 2000 parts by mass, based on 100 parts by mass of the reaction raw materials.

As the catalyst that may be used in the dehydrating step, a wide variety of catalysts which function under the reaction conditions of the present embodiment are used. An acid catalyst is preferable. Examples of suitable acid catalysts include, but are not limited to, an inorganic acid such as hydrochloric acid, sulfuric acid, phosphoric acid, hydrobromic acid, and hydrofluoric acid; an organic acid such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; a Lewis acid such as zinc chloride, aluminum chloride, iron chloride, and boron trifluoride; and a solid acid such as tungstosilicic acid, tungstophosphoric acid, silicomolybdic acid, and phosphomolybdic acid. These acid catalysts are used alone as one kind or in combination of two or more kinds. Among them, organic acids and solid acids are preferable from the viewpoint of production, and it is preferable to use hydrochloric acid or sulfuric acid from the viewpoint of production such as easy availability and handleability.

The amount of the catalyst used can be arbitrarily set according to, for example, the substrate, catalyst, and reaction conditions to be used, without particular limitation. In general, it is suitably 0.0001 to 100 parts by mass, and from the viewpoint of the yield, preferably 0.001 to 10 parts by mass, based on 100 parts by mass of the reaction raw materials.

As the polymerization inhibitor that may be used in the dehydrating step, a wide variety of polymerization inhibitors which function under the reaction conditions of the present embodiment are used. The polymerization inhibitor is effective, but is not an essential component. Examples of suitable polymerization inhibitors include, but are not limited to, hydroquinone, hydroquinone monomethyl ether, 4-tert-butylcatechol, phenothiazine, and an n-oxyl (nitroxide) inhibitor, for example, Prostab(R) 5415 (bis(1-oxyl-2,2,6,6-tetramethylpiperidin-4-yl)sebacate which is commercially available from Ciba Specialty Chemicals, Tarrytown, N.Y., CAS #2516-92-9), 4-hydroxy-TEMPO (4-hydroxy-2,2,6,6-tetramethylpiperidin-1-yloxy, which is commercially available from TCI, CAS #2226-96-2), and Uvinul(R) 4040P (1,6-hexamethylene-bis(N-formyl-N-(1-oxyl-2,2,6,6-tetramethylpiperidin-4-yl)amine) which is commercially available from BASF Corp., Worcester, Mass.

The amount of the polymerization inhibitor used can be arbitrarily set according to, for example, the substrate, catalyst, and reaction conditions to be used, without particular limitation. In general, it is suitably 0.0001 to 100 parts by mass, and from the viewpoint of the yield, it is preferably 0.001 to 10 parts by mass, based on 100 parts by mass of the reaction raw materials.

As the polymerization retarder that may be used in the dehydrating step, a wide variety of polymerization retarders which function under the reaction conditions of the present embodiment are used. The polymerization retarder is effective, but is not an essential component. A polymerization retardant is effectively used by combining with the polymerization inhibitor. The polymerization retardant is well-known in the art as a compound that can delay the polymerization reaction, but cannot prevent all polymerization from occurring. A typical retardant is an aromatic nitro compound such as dinitro-ortho-cresol (DNOC) and dinitro butyl phenol (DNBP). The method for producing the polymerization retardant is common and well-known in the art (e.g., see U.S. Pat. Nos. 6,339,177; Park et al., Polymer (Korea) (1988), 12(8), 710-19), and its use in the control of styrene polymerization is well documented (e.g., see Bushby et al., Polymer (1998), 39(22), 5567-5571).

The amount of the polymerization retarder used can be arbitrarily set according to, for example, the substrate, catalyst, and reaction conditions to be used, without particular limitation. In general, it is suitably 0.0001 to 100 parts by mass, and from the viewpoint of the yield, preferably 0.001 to 10 parts by mass, based on 100 parts by mass of the reaction raw materials.

### (Reaction Conditions)

The reaction mixture is formed by adding the iodine-containing alcohol substrate having the formula (1-1), the catalyst, and the solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 10° C. to 190° C. is preferable, a temperature of 25° C. to 150° C. is more preferable, and a temperature of 50° C. to 100° C. is further preferable.

In the reaction using 1-(4-hydroxy-3,5-diiodophenyl)ethanol as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation. In the reaction using 1-(4-hydroxy-3,5-diiodophenyl)ethanol as the substrate, the preferred reaction pressure is reduced pressure to normal pressure, and reduced pressure is preferable.

From the viewpoint of the reaction rate, the reaction is preferably performed while removing low boiling products such as water to be produced and methanol from the reaction system. As the method for removing low boiling products, a conventionally known suitable method can be used and conducted. For example, they can be removed using evaporation, and are preferably removed by using evaporation at reduced pressure.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 1-(4-hydroxy-3,5-diiodophenyl)ethanol as the substrate, the preferred reaction time range is 15 minutes to 600 minutes.

Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

### [Production Method (i) of Iodine-Containing Alcohol Substrate Represented by Formula (1-1)]

The iodine-containing ketone substrate used in the production of the formula (1-1) is an iodine-containing ketone substrate having the formula (1-2):

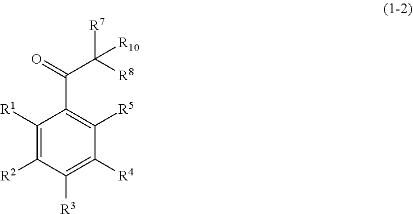

wherein, each of R¹ to R⁵ is independently H, OH, OCH₃, a halogen, or a linear or branched alkyl, each of R⁷, R⁸, and R¹⁰ is independently H, OH, OCH₃, a halogen, or a cyano group, provided that at least one of R¹ to R⁵ is OH and at least one of them is iodine.

Examples of suitable iodine-containing ketone substrates include, but are not limited to, iodine-containing 2-hydroxyphenylmethylketone, iodine-containing 3-hydroxyphenylmethylketone, iodine-containing 4-hydroxyphenylmethylketone, iodine-containing 3-methoxy-4-hydroxyphenylmethylketone, iodine-containing 3,5-dimethoxy-4-hydroxyphenylmethylketone, iodine-containing 2,3-dihydroxyphenylmethylketone, iodine-containing 2,4-dihydroxyphenylmethylketone, iodine-containing 2,5-dihydroxyphenylmethylketone, iodine-containing 2,6-dihydroxyphenylmethylketone, iodine-containing 3,4-dihydroxyphenylmethylketone, iodine-containing 3,5-dihydroxyphenylmethylketone, iodine-containing 2,3,4-trihydroxyphenylmethylketone, iodine-containing 2,4,6-trihydroxyphenylmethylketone, iodine-containing 3,4,5-trihydroxyphenylmethylketone, and iodine-containing 4-hydroxyphenylα-cyanomethylketone. At least one iodine atom is introduced, and two or more iodine atoms are preferably introduced. At least one OH is introduced, and two or more OH are preferably introduced.

Specific examples of the iodine-containing ketone substrate used in the present invention include, but are not limited to the followings.

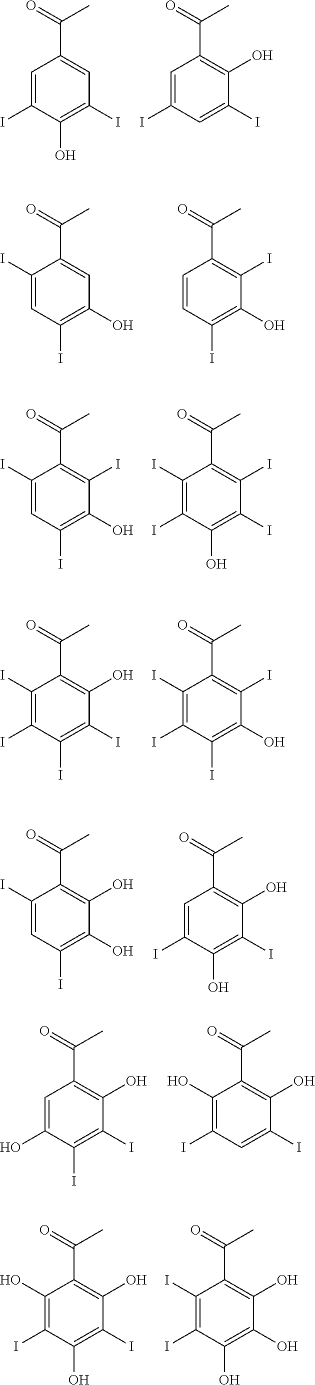

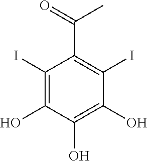

These iodine-containing ketone substrates can be obtained by many methods, but is preferably obtained by the method described later, from the viewpoint of the availability and yield of the raw material.

The method for producing the iodine-containing alcohol substrate represented by the formula (1-1) comprises:
c) a step of providing an iodine-containing ketone substrate having the formula (1-2); and
d) a step of reducing the iodine-containing ketone substrate.

As the solvent that may be used in the reducing step, a wide variety of solvents including a polar aprotic solvent and protic polar solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable, and from the viewpoint of suppressing side reactions, a mixture of a polar aprotic solvent and a polar protic solvent is preferable. As the polar protic solvent, water, or an alcohol solvent such as methanol, ethanol, propanol, and butanol is further preferable. The solvent is effective, but is not an essential component. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

As the reducing agent that may be used in the reducing step, a wide variety of reducing agents which function under the reaction conditions of the present embodiment are used. Examples of suitable reducing agents include, but are not limited to, a metal hydride and a metal hydride complex compound, such as borane dimethylsulfide, diisobutylaluminum hydride, sodium borohydride, lithium borohydride, potassium borohydride, zinc borohydride, lithium tri-s-butylborohydride, potassium tri-s-butylborohydride, lithium triethylborohydride, lithium aluminum hydride, lithium tri-t-butoxyaluminum hydride, and sodium bis(methoxyethoxy)aluminum hydride.

The amount of the reducing agent used can be arbitrarily set according to, for example, the substrate, reducing agent, and reaction conditions to be used, without particular limitation. In general, it is suitably 1 to 500 parts by mass, and from the viewpoint of the yield, it is preferably 10 to 200 parts by mass, based on 100 parts by mass of the reaction raw materials.

As a quenching agent, a wide variety of quenching agents which function under the reaction conditions of the present embodiment are used. The quenching agent has a function of deactivating the reducing agent. The quenching agent is effective, but is not an essential component. Examples of suitable quenching agents include, but are not limited to, ethanol, aqueous ammonium chloride solution, water, hydrochloric acid, and sulfuric acid.

The amount of the quenching agent used can be arbitrarily set according to the amount of the reducing agent to be used, without particular limitation. In general, it is suitably 1 to 500 parts by mass, and from the viewpoint of the yield, it is preferably 50 to 200 parts by mass, based on 100 parts by mass of the reducing agents.

### (Reaction Conditions)

The reaction mixture is formed by adding the iodine-containing ketone substrate having the formula (1-2), the reducing agent, and the solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the reducing agent, and the desired yield. In general, a temperature of 0° C. to 200° C. is suitable, and from the viewpoint of the yield, a temperature of 0° C. to 100° C. is preferable, a temperature of 0° C. to 70° C. is more preferable, and a temperature of 0° C. to 50° C. is further preferable.

In the reaction using 4′-hydroxy-3′,5′-diiodoacetophenone as the substrate, the temperature range is preferably 0° C. to 100° C.

The reaction pressure is not particularly limited. The preferred range varies according to the concentration of the substrate, the stability of the formed product, the selection of the reducing agent, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxy-3′,5′-diiodoacetophenone as the substrate, the preferred reaction pressure is reduced pressure to normal pressure, and reduced pressure is preferable.

The reaction time is not particularly limited. The preferred range varies according to the concentration of the substrate, the stability of the formed product, the selection of the reducing agent, and the desired yield. However, the majority of the reactions are carried out for less than 6 hours, and the reaction time is typically 15 minutes to 600 minutes.

In the reaction using 4′-hydroxy-3′,5′-diiodoacetophenone as the substrate, the preferred reaction time range is 15 minutes to 600 minutes.

Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity compound by a separation and purification method by filtration, concentration, distillation, extraction, crystallization, recrystallization, column chromatography, activated carbon, or the like, which are well-known purification methods in the art, or a combined method thereof.

### [Production Method (ii) of Iodine-Containing Alcohol Substrate Represented by Formula (1-1)]

The alcohol substrate used in the production of the formula (1-1) is an alcohol substrate having the formula (1-3):

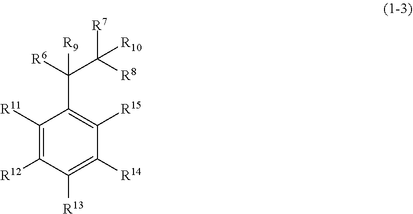

wherein, each of R¹¹ to R¹⁵ is independently H, OH, OCH₃, or a linear or branched alkyl, each of R⁶ to R¹⁰ is independently H, OH, OCH₃, a halogen, or a cyano group, provided that at least one of R¹¹ to R¹⁵ is OH and one of R⁶ to R¹⁰ is OH or OCH₃.

Examples of suitable alcohol substrates include, but are not limited to, 2-(1-hydroxyethyl)phenol, 3-(1-hydroxyethyl)phenol, 4-(1-hydroxyethyl)phenol, 4-(1-hydroxyethyl)-1-methoxyphenol, 4-(1-hydroxyethyl)-2,6-dimethoxyphenol, 3-(1-hydroxyethyl)benzene-1,2-diol, 4-(1-hydroxyethyl)benzene-1,3-diol, 2-(1-hydroxyethyl)benzene-1,4-diol, 6-(1-hydroxyethyl)benzene-1,5-diol, 4-(1-hydroxyethyl)benzene-1,2-diol, 5-(1-hydroxyethyl)benzene-1,3-diol, 4-(1-hydroxyethyl)benzene-1,2,3-triol, 2-(1-hydroxyethyl)benzene-1,3,5-triol, 5-(1-hydroxyethyl)benzene-1,2,3-triol, 2-(1-cyano-1-hydroxyethyl)phenol, 2-(2-hydroxyphenyl)ethanol, 2-(3-hydroxyphenyl)ethanol, 2-(4-hydroxyphenyl)ethanol, 2-(3-methoxy-4-hydroxyphenyl)ethanol, 2-(3,5-dimethoxy-4-hydroxyphenyl)ethanol, 2-(2,3-dihydroxyphenyl)ethanol, 2-(2,4-dihydroxyphenyl)ethanol, 2-(2,5-dihydroxyphenyl)ethanol, 2-(2,6-dihydroxyphenyl)ethanol, 2-(3,4-dihydroxyphenyl)ethanol, 2-(3,5-dihydroxyphenyl)ethanol, 2-(2,3,4-trihydroxyphenyl)ethanol, 2-(2,4,6-trihydroxyphenyl)ethanol, 2-(3,4,5-trihydroxyphenyl)ethanol, and 1-cyano-2-(4-hydroxyphenyl)ethanol. At least one OH is introduced, and two or more OH are preferably introduced. OH may be substituted with OMe.

Specific examples of the alcohol substrate used in the present embodiment include, but are not limited to the followings.

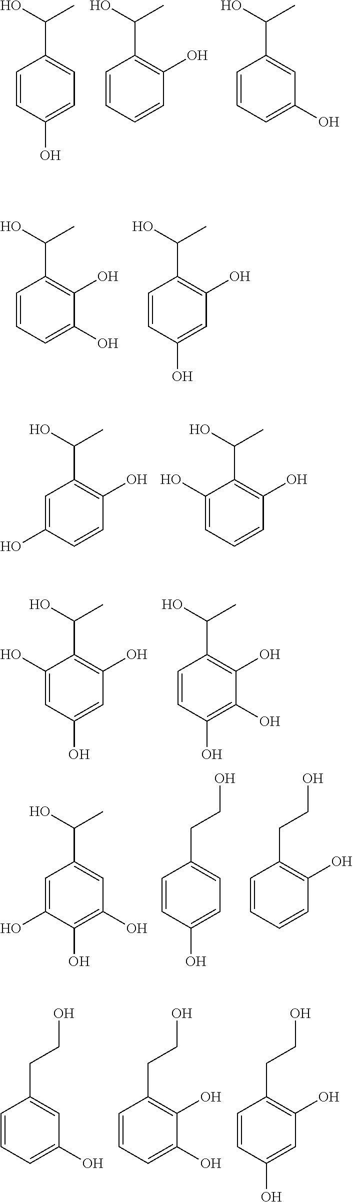

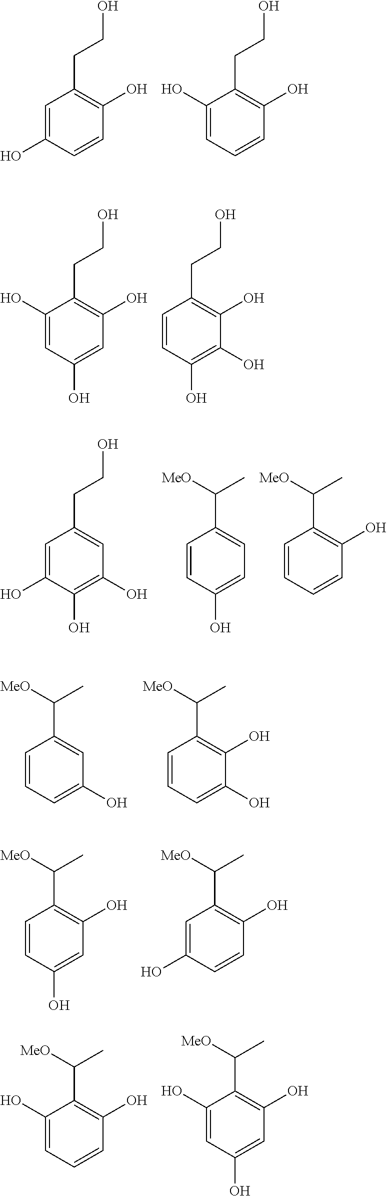

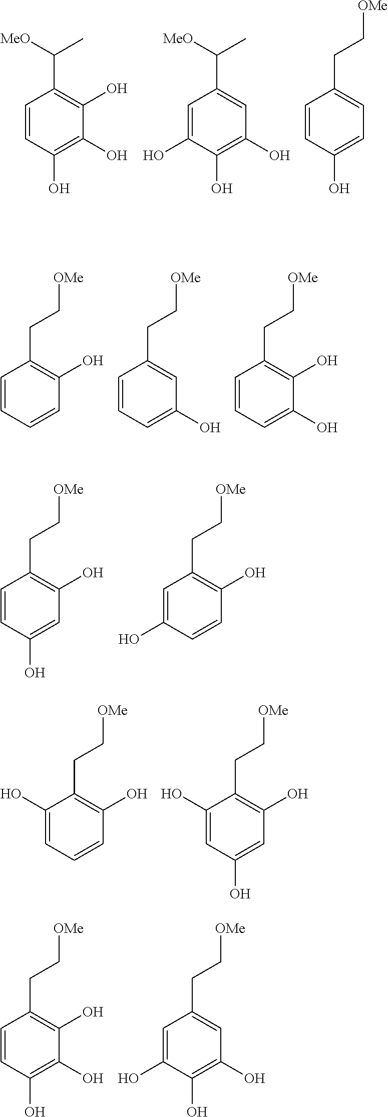

These alcohol substrates can be obtained by many methods, but is preferably obtained by the method described later, from the viewpoint of the availability and yield of the raw material.

The method for producing the iodine-containing alcohol substrate represented by the formula (1-1) comprises:
e) a step of providing an alcohol substrate having the formula (1-3); and
f) a step of introducing iodine into the alcohol substrate.

As the solvent that may be used in the iodine-introducing step, a wide variety of solvents including a polar aprotic solvent and protic polar solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar protic solvent or a mixture thereof is preferable, and from the viewpoint of suppressing side reactions, a mixture of a polar protic solvent and water is preferable. The solvent is effective, but is not an essential component. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

### (Reaction Conditions)

The reaction mixture is formed by adding the alcohol substrate having the formula (1-3), the catalyst, and the solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable, a temperature of 0° C. to 70° C. is more preferable, and a temperature of 0° C. to 50° C. is further preferable.

In the reaction using 1-(4-hydroxyphenyl)ethanol as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 1-(4-hydroxyphenyl)ethanol as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 1-(4-hydroxyphenyl)ethanol as the substrate, the preferred reaction time range is 15 minutes to 600 minutes.

Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity compound by a separation and purification method by filtration, concentration, distillation, extraction, crystallization, recrystallization, column chromatography, activated carbon, or the like, which are well-known purification methods in the art, or a combined method thereof.

### [Production Method) of Iodine-Containing Ketone Substrate Represented by Formula (1-2)]

The ketone substrate used in the production of the formula (1-2) is a ketone substrate having the formula (1-4):

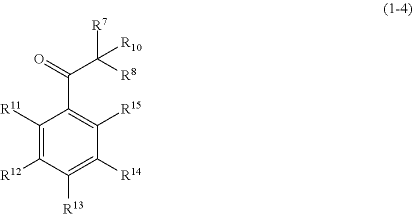

wherein, each of R¹¹ to R¹⁵ is independently H, OH, OCH₃, or a linear or branched alkyl, each of R⁷ to R⁸ and R¹⁰ is independently H, OH, OCH₃, a halogen, or a cyano group, provided that at least one of R¹¹ to R¹⁵ is OH.

Examples of suitable ketone substrates include, but are not limited to, 2-hydroxyphenylmethylketone, 3-hydroxyphenylmethylketone, 4-hydroxyphenylmethylketone, 3-methoxy-4-hydroxyphenylmethylketone, 3,5-dimethoxy-4-hydroxyphenylmethylketone, 2,3-dihydroxyphenylmethylketone, 2,4-dihydroxyphenylmethylketone, 2,5-dihydroxyphenylmethylketone, 2,6-dihydroxyphenylmethylketone, 3,4-dihydroxyphenylmethylketone, 3,5-dihydroxyphenylmethylketone, 2,3,4-trihydroxyphenylmethylketone, 2,4,6-trihydroxyphenylmethylketone, 3,4,5-trihydroxyphenylmethylketone, and 4-hydroxyphenylα-cyanomethylketone.

Specific examples of the ketone substrate used in the present embodiment include, but are not limited to the followings.

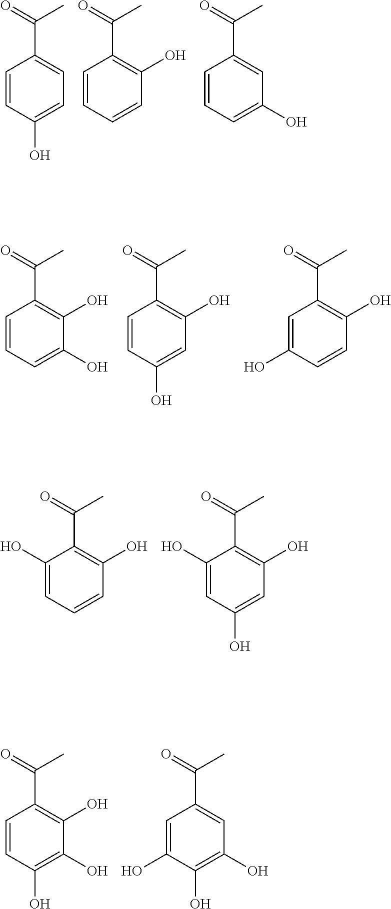

These ketone substrates can be obtained by many methods.

The method for producing the iodine-containing ketone substrate represented by the formula (1-2) comprises:
g) a step of providing a ketone substrate having the formula (1-4); and
h) a step of introducing iodine into the ketone substrate.

As the solvent that may be used in the iodine-introducing step, a wide variety of solvents including a polar aprotic solvent and protic polar solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar protic solvent or a mixture thereof is preferable, and from the viewpoint of suppressing side reactions, a mixture of a polar protic solvent and water is preferable. The solvent is effective, but is not an essential component. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

### (Reaction Conditions)

The reaction mixture is formed by adding the ketone substrate having the formula (1-4), the catalyst, and the solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 0° C. to 100° C. is preferable, a temperature of 0° C. to 70° C. is more preferable, and a temperature of 0° C. to 50° C. is further preferable.

In the reaction using 4′-hydroxyacetophenone as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxyacetophenone as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 4′-hydroxyacetophenone as the substrate, the preferred reaction time range is 15 minutes to 600 minutes.

Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity compound by a separation and purification method by filtration, concentration, distillation, extraction, crystallization, recrystallization, column chromatography, activated carbon, or the like, which are well-known purification methods in the art, or a combined method thereof.

### [Production Method) of Alcohol Substrate Represented by Formula (1-3)]

The ketone substrate used in the production of the formula (1-3) is a ketone substrate having the aforementioned formula (1-4).

The method for producing the alcohol substrate represented by the formula (1-3) comprises:
i) a step of providing a ketone substrate having the formula (1-4); and
j) a step of reducing the ketone substrate.

As the solvent that may be used in the reducing step, a wide variety of solvents including a polar aprotic solvent and protic polar solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable, and from the viewpoint of suppressing side reactions, a mixture of a polar aprotic solvent and a polar protic solvent is preferable. As the polar protic solvent, water, or an alcohol solvent such as methanol, ethanol, propanol, and butanol is further preferable. The solvent is effective, but is not an essential component. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, reducing agent, and reaction conditions to be used, without particular limitation. In general, it is suitably 0 to 10000 parts by mass, and from the viewpoint of the yield, it is preferably 100 to 2000 parts by mass, based on 100 parts by mass of the reaction raw materials.

As the reducing agent, a wide variety of reducing agents which function under the reaction conditions of the present embodiment are used. Examples of suitable reducing agents include, but are not limited to, a metal hydride and a metal hydride complex compound, such as borane dimethylsulfide, diisobutylaluminum hydride, sodium borohydride, lithium borohydride, potassium borohydride, zinc borohydride, lithium tri-s-butylborohydride, potassium tri-s-butylborohydride, lithium triethylborohydride, lithium aluminum hydride, lithium tri-t-butoxyaluminum hydride, and sodium bis(methoxyethoxy)aluminum hydride.

The amount of the reducing agent used can be arbitrarily set according to, for example, the substrate, reducing agent, and reaction conditions to be used, without particular limitation. In general, it is suitably 1 to 500 parts by mass, and from the viewpoint of the yield, it is preferably 10 to 200 parts by mass, based on 100 parts by mass of the reaction raw materials.

As a quenching agent, a wide variety of quenching agents which function under the reaction conditions of the present embodiment are used. The quenching agent has a function of deactivating the reducing agent. The quenching agent is effective, but is not an essential component. Examples of suitable quenching agents include, but are not limited to, ethanol, aqueous ammonium chloride solution, water, hydrochloric acid, and sulfuric acid.

The amount of the quenching agent used can be arbitrarily set according to the amount of the reducing agent to be used, without particular limitation. In general, it is suitably 1 to 500 parts by mass, and from the viewpoint of the yield, it is preferably 50 to 200 parts by mass, based on 100 parts by mass of the reaction raw materials.

### (Reaction Conditions)

The reaction mixture is formed by adding the ketone substrate having the formula (1-4), the reducing agent, and the solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the reducing agent, and the desired yield. In general, a temperature of 0° C. to 200° C. is suitable, and from the viewpoint of the yield, a temperature of 0° C. to 100° C. is preferable, a temperature of 0° C. to 70° C. is more preferable, and a temperature of 0° C. to 50° C. is further preferable.

In the reaction using 4′-hydroxyacetophenone as the substrate, the temperature range is preferably 0° C. to 100° C.

The reaction pressure is not particularly limited. The preferred range varies according to the concentration of the substrate, the stability of the formed product, the selection of the reducing agent, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4′-hydroxyacetophenone as the substrate, the preferred reaction pressure is reduced pressure to normal pressure, and reduced pressure is preferable.

The reaction time is not particularly limited. The preferred range varies according to the concentration of the substrate, the stability of the formed product, the selection of the reducing agent, and the desired yield. However, the majority of the reactions are carried out for less than 6 hours, and the reaction time is typically 15 minutes to 600 minutes.

In the reaction using 4′-hydroxyacetophenone as the substrate, the preferred reaction time range is 15 minutes to 600 minutes.

Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity compound by a separation and purification method by filtration, concentration, distillation, extraction, crystallization, recrystallization, column chromatography, activated carbon, or the like, which are well-known purification methods in the art, or a combined method thereof.

### [Method for Producing Iodine-Containing Acetylated Vinyl Monomer Represented by Formula (2)]

The present embodiment is a method for producing an iodine-containing acetylated vinyl monomer, specifically, an iodine-containing acetoxystyrene having the formula (2):

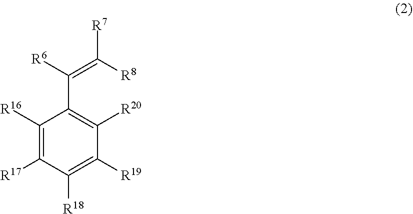

wherein, each of R¹⁶ to R²⁰ is independently H, OH, OCH₃, OAc, a halogen, or a linear or branched alkyl, each of R⁶ to R⁸ is independently H, OH, OCH₃, a halogen, or a cyano group, provided that at least one of R¹⁶ to R²⁰ is OAc and at least one of them is iodine.

Examples of the iodine-containing acetylated vinyl monomer produced by the method of the present embodiment include, but are not limited to, iodine-containing 2-acetoxystyrene, iodine-containing 3-acetoxystyrene, iodine-containing 4-acetoxystyrene, iodine-containing 3-methoxy-4-acetoxystyrene, iodine-containing 3,5-dimethoxy-4-acetoxystyrene, iodine-containing 2,3-acetoxystyrene, iodine-containing 2,4-acetoxystyrene, iodine-containing 2,5-acetoxystyrene, iodine-containing 2,6-acetoxystyrene, iodine-containing 3,4-acetoxystyrene, iodine-containing 3,5-acetoxystyrene, iodine-containing 2,3,4-triacetoxystyrene, iodine-containing 2,4,6-triacetoxystyrene, iodine-containing 3,4,5-triacetoxystyrene, and iodine-containing α-cyano-4-acetoxystyrene. At least one iodine atom is introduced, and two or more iodine atoms are preferably introduced. At least one OAc is introduced, and two or more OAc are preferably introduced.

Specific examples of the iodine-containing acetylated vinyl monomer produced by the method of the present embodiment include, but are not limited to, the followings:

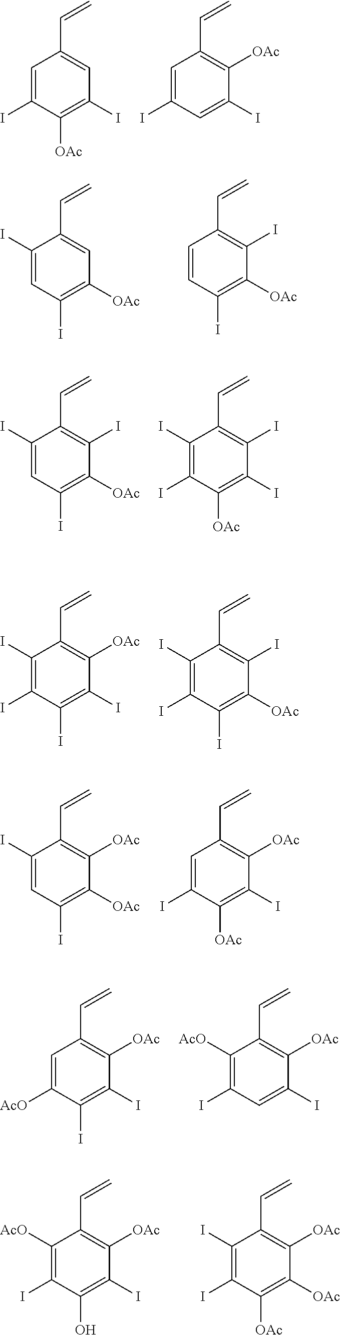

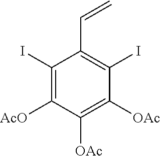

wherein Ac represents an acetyl group.

The method for producing the iodine-containing acetylated vinyl monomer (iodine-containing acetoxystyrene) represented by the formula (2) comprises:
k) a step of providing the iodine-containing vinyl monomer (iodine-containing hydroxystyrene) having the formula (1); and
l) a step of acetylating the iodine-containing hydroxystyrene.

As the solvent that may be used in the acetylating step, a wide variety of solvents including a polar aprotic solvent and protic polar solvent are used. A single protic polar solvent or a single polar aprotic solvent can be used. Furthermore, a mixture of polar aprotic solvents, a mixture of protic polar solvents, a mixture of a polar aprotic solvent and a protic polar solvent, and a mixture of an aprotic or protic solvent and a nonpolar solvent can be used, and a polar aprotic solvent or a mixture thereof is preferable. The solvent is effective, but is not an essential component. Examples of suitable polar aprotic solvents include, but are not limited to, an ether solvent such as diethyl ether, tetrahydrofuran, dimethoxyethane, diglyme, and triglyme; an ester solvent such as ethyl acetate and γ-butyrolactone; a nitrile solvent such as acetonitrile; a hydrocarbon solvent such as toluene and hexane; an amide solvent such as N,N-dimethylformamide, 1-methyl-2-pyrrolidinone, N,N-dimethylacetamide, hexamethylphosphoramide, hexamethylphosphorous triamide; and dimethylsulfoxide. Dimethylsulfoxide is preferable. Examples of suitable protic polar solvents include, but are not limited to, water, and an alcohol solvent such as methanol, ethanol, propanol, and butanol, di(propylene glycol) methyl ether, di(ethylene glycol) methyl ether, 2-butoxyethanol, ethylene glycol, 2-methoxyethanol, propyleneglycol methylether, n-hexanol, and n-butanol.

The amount of the solvent used can be arbitrarily set according to, for example, the substrate, catalyst, and reaction conditions to be used, without particular limitation. In general, it is suitably 0 to 10000 parts by mass, and from the viewpoint of the yield, preferably 100 to 2000 parts by mass, based on 100 parts by mass of the reaction raw materials.

As an acetylating agent, a wide variety of acetylating agents which function under the reaction conditions of the present embodiment are used.

Examples of suitable acetylating agents include, but are not limited to, acetic anhydride, acetyl halide, and acetic acid, and acetic anhydride is preferable.

As the catalyst that may be used in the acetylating step, a wide variety of acetylation catalysts which function under the reaction conditions of the present embodiment are used. An acid catalyst or a base catalyst is preferable.

Examples of suitable acid catalysts include, but are not limited to, an inorganic acid such as hydrochloric acid, sulfuric acid, phosphoric acid, hydrobromic acid, and hydrofluoric acid; an organic acid such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; a Lewis acid such as zinc chloride, aluminum chloride, iron chloride, and boron trifluoride; and a solid acid such as tungstosilicic acid, tungstophosphoric acid, silicomolybdic acid, and phosphomolybdic acid. These acid catalysts are used alone as one kind or in combination of two or more kinds. Among them, organic acids and solid acids are preferable from the viewpoint of production, and it is preferable to use hydrochloric acid or sulfuric acid from the viewpoint of production such as easy availability and handleability.

Examples of suitable base catalysts include, but are not limited to, an amine-containing catalyst such as pyridine and ethylenediamine, and non-amine basic catalyst such as a metal salt, and in particular, potassium salt or acetate is preferable. Examples of suitable catalysts include, but are not limited to, potassium acetate, potassium carbonate, potassium hydrate, sodium acetate, sodium carbonate, sodium hydroxide, and magnesium oxide.

All non-amine base catalysts of the present embodiment are commercially available from, for example, EM Science (Gibbstown) or Aldrich (Milwaukee).

The amount of the catalyst used can be arbitrarily set according to, for example, the substrate, catalyst, and reaction conditions to be used, without particular limitation. In general, it is suitably 1 to 5000 parts by mass, and from the viewpoint of the yield, preferably 50 to 3000 parts by mass, based on 100 parts by mass of the reaction raw materials.

As the polymerization inhibitor that may be used in the acetylating step, a wide variety of polymerization inhibitors which function under the reaction conditions of the present embodiment are used. The polymerization inhibitor is effective, but is not an essential component. Examples of suitable polymerization inhibitors include, but are not limited to, hydroquinone, hydroquinone monomethyl ether, 4-tert-butylcatechol, phenothiazine, and an n-oxyl (nitroxide) inhibitor, for example, Prostab(R) 5415 (bis(1-oxyl-2,2,6,6-tetramethylpiperidin-4-yl)sebacate which is commercially available from Ciba Specialty Chemicals, Tarrytown, N.Y., CAS #2516-92-9), 4-hydroxy-TEMPO (4-hydroxy-2,2,6,6-tetramethylpiperidin-1-yloxy, which is commercially available from TCI, CAS #2226-96-2), and Uvinul(R) 4040P (1,6-hexamethylene-bis(N-formyl-N-(1-oxyl-2,2,6,6-tetramethylpiperidin-4-yl)amine) which is commercially available from BASF Corp., Worcester, Mass.

The amount of the polymerization inhibitor used can be arbitrarily set according to, for example, the substrate, catalyst, and reaction conditions to be used, without particular limitation. In general, it is suitably 0.0001 to 100 parts by mass, and from the viewpoint of the yield, it is preferably 0.001 to 10 parts by mass, based on 100 parts by mass of the reaction raw materials.

As the polymerization retarder that may be used in the acetylating step, a wide variety of polymerization retarders which function under the reaction conditions of the present embodiment are used. The polymerization retarder is effective, but is not an essential component. A polymerization retardant is effectively used by combining with the polymerization inhibitor. The polymerization retardant is well-known in the art as a compound that can delay the polymerization reaction, but cannot prevent all polymerization from occurring. A typical retardant is an aromatic nitro compound such as dinitro-ortho-cresol (DNOC) and dinitro butyl phenol (DNBP). The method for producing the polymerization retardant is common and well-known in the art (e.g., see U.S. Pat. No. 6,339,177; Park et al., Polymer (Korea) (1988), 12(8), 710-19), and its use in the control of styrene polymerization is well documented (e.g., see Bushby et al., Polymer (1998), 39(22), 5567-5571).

The amount of the polymerization retarder used can be arbitrarily set according to, for example, the substrate, catalyst, and reaction conditions to be used, without particular limitation. In general, it is suitably 0.0001 to 100 parts by mass, and from the viewpoint of the yield, preferably 0.001 to 10 parts by mass, based on 100 parts by mass of the reaction raw materials.

### (Reaction Conditions)

The reaction mixture is formed by adding the iodine-containing hydroxystyrene having the formula (1), the catalyst, and the solvent to a reactor. Any suitable reactor is used.

The reaction may be carried out by arbitrarily selecting a known method such as a batch method, a semi-batch method, or a continuous method.

The reaction temperature is not particularly limited. The preferred range varies according to the concentration of the substrate, the stability of the formed product, the selection of the catalyst, and the desired yield. In general, a temperature of 0° C. to 200° C. is suitable, and from the viewpoint of the yield, a temperature of 10° C. to 190° C. is preferable, a temperature of 25° C. to 150° C. is more preferable, and a temperature of 50° C. to 100° C. is further preferable.

In the reaction using 4-hydroxy-3,5-diiodostyrene as the substrate, the temperature range is preferably 0° C. to 100° C.

The reaction pressure is not particularly limited. The preferred range varies according to the concentration of the substrate, the stability of the formed product, the selection of the catalyst, and the desired yield. The pressure can be adjusted by using an inert gas such as nitrogen, or by using a suction pump or the like. In the reaction at high pressure, a conventional pressure reactor comprising a shaking vessel, a rocker vessel, and a stirred autoclave is used, without limitation.

In the reaction using 4-hydroxy-3,5-diiodostyrene as the substrate, the preferred reaction pressure is reduced pressure to normal pressure, and reduced pressure is preferable.

The reaction time is not particularly limited. The preferred range varies according to the concentration of the substrate, the stability of the formed product, the selection of the catalyst, and the desired yield. However, the majority of the reactions are carried out for less than 6 hours, and the reaction time is typically 15 minutes to 600 minutes.

In the reaction using 4-hydroxy-3,5-diiodostyrene as the substrate, the preferred reaction time range is 15 minutes to 600 minutes.

Isolation and purification can be conducted by using a conventionally known suitable method after terminating the reaction. For example, the reaction mixture is poured in ice water and extracted in a solvent such as ethyl acetate or diethyl ether. Then, the product is recovered by removing the solvent using evaporation at reduced pressure. The product can be isolated and purified as the desired high purity monomer by a separation and purification method by filtration, concentration, distillation, extraction, crystallization, recrystallization, column chromatography, activated carbon, or the like, which are well-known purification methods in the art, or a combined method thereof.

According to the production method of the present embodiment, the iodine-containing hydroxystyrene and acetylated derivatives thereof can be produced with low-cost raw materials, under mild conditions, and in high yield.

The obtained iodine-containing hydroxystyrene and acetylated derivatives thereof are suitably used as the raw material monomer for the resist composition for lithography by extreme ultraviolet ray. Also, they are useful in a wide variety of industrial applications including various semiconductor materials and electronic materials.

### EXAMPLE

Hereinafter, the present invention will be described in further detail with reference to Examples and Comparative Examples, but the present invention is not limited by these Examples in any way.

### [Measurement Method]

### [Nuclear Magnetic Resonance (NMR)]

The structure of compounds was verified by carrying out NMR measurement under the following conditions using a nucleus magnetic resonance apparatus “Advance 600 II spectrometer” (product name: manufactured by Bruker).

### [¹H-NMR Measurement]

Frequency: 400 MHz

Solvent: CDCl₃, or d₆-DMSO

Internal standard: TMS

Measurement temperature: 23° C.

### [¹³C-NMR Measurement]

Frequency: 500 MHz

Solvent: CDCl₃, or d₆-DMSO

Internal standard: TMS

Measurement temperature: 23° C.

### [Inorganic Element Content]

The metal content contained in the compounds prepared in Examples and Comparative Examples was measured using an inorganic element analyzer (ICP-AES/ICP-MS) “AG8900” (product name, manufactured by Agilent Technologies, Inc.).

### [Organic Impurity Content]

The organic impurity content contained in the compounds prepared in Examples and Comparative Examples was calculated from the area fraction of the GC chart and the peak intensity ratio between the target peak and the reference peak by gas chromatography mass spectrometry (GC-MS).

### Example A1

### Synthesis of Compound A1 Represented by Formula (M1)

Using a 200 mL glass flask as a reaction vessel, 4.96 g (40 mmol) of 4-hydroxybenzyl alcohol was dissolved using butanol as a solvent, a 20% by mass aqueous iodine chloride solution (81.2 g, 100 mmol) was added dropwise thereto at 50° C. over 60 minutes, and then the mixture was stirred at 50° C. for 2 hours to allow 4-hydroxybenzyl alcohol to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 12.1 g of a white solid. The white solid sample was analyzed by liquid chromatography-mass spectrometry (LC-MS) and, as a result, confirmed to be 4-hydroxy-3,5-diiodobenzyl alcohol.

MnO₂ (3.4 g, 40 mmol) was added to a methylene chloride solvent and stirred, which was then stirred for 1 hour while a 50% by mass solution in which the total amount of the synthesized 4-hydroxy-3,5-diiodobenzyl alcohol was dissolved in methylene chloride was added dropwise and stirred at room temperature for 4 hours, and then the reaction solution was filtered off and the solvent was distilled off to obtain 4-hydroxy-3,5-diiodobenzaldehyde.

A solution in which dimethyl malonate (5.3 g, 40 mmol) and the total amount of 4-hydroxy-3,5-diiodobenzaldehyde synthesized above was dissolved in a DMF solvent was prepared and then stirred for 1 hour while a solution in which ethylenediamine (0.3 g) was dissolved in DMF was added dropwise, and the mixture was allowed to react for 6 hours with stirring while controlling the liquid temperature at 150° C. in an oil bath. Thereafter, ethyl acetate and water was added, a 2 mol/L aqueous HCl solution was added to control pH to 4 or less, and then a separation operation was carried out to collect the organic phase. Further, the obtained organic phase was washed by being subjected to a separation operation with a 2 mol/L aqueous sodium carbonate solution, water, and brine in the order presented, and then purified by a filter, and the solvent was distilled off from the organic phase to obtain 8.1 g of the compound A1 (4-hydroxy-3,5-diiodostyrene (a compound represented by the following formula (M1))). The inorganic element content and the organic impurity content were measured by the aforementioned method, and the results are shown in Table 1.

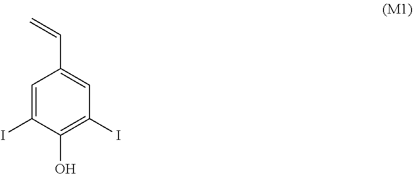

### Example A2

### Synthesis of Compound A2 Represented by Formula (M2)

In a 2 L flask, 400 mL of dichloromethane, 41 g of the obtained compound A1, 16.2 g of triethylamine, and 0.7 g of N-(4-pyridyl)dimethylamine (DMAP) was dissolved in a nitrogen flow. 33.6 g of di-tert-butyl dicarbonate was dissolved in 100 mL of dichloromethane, which was added dropwise into the above 2 L flask with stirring, and the mixture was stirred at room temperature for 3 hours. Thereafter, water washing was conducted three times by a separation operation using 100 mL of water, the solvent was distilled off from the obtained organic phase, original components were removed by silica gel chromatography using dichloromethane/hexane, and the solvent was further distilled off to obtain 4.5 g of a BOC group-substituted compound (a compound represented by the following formula (M2), hereinafter, also referred to as the “compound A2”) of the compound A1, as the target component. The inorganic element content and the organic impurity content were measured by the aforementioned method, and the results are shown in Table 1.

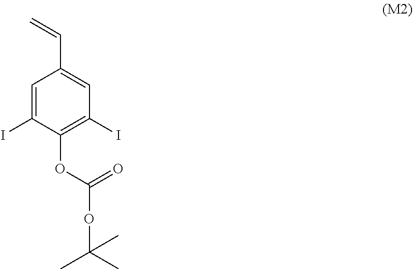 [SMILES: C=Cc1cc(I)c(OC(=O)OC(C)(C)C)c(I)c1]

### Example A3

### Synthesis of Compound A3 Represented by Formula (M3)

In a 2 L flask, 400 mL of dichloromethane, 41 g of the obtained compound A1, 16.2 g of triethylamine, and 0.7 g of N-(4-pyridyl)dimethylamine (DMAP) was dissolved in a nitrogen flow. 20.7 g of dimethyl dicarbonate was dissolved in 100 mL of dichloromethane, which was added dropwise into the above 2 L flask with stirring, and the mixture was stirred at room temperature for 3 hours. Thereafter, water washing was conducted three times by a separation operation using 100 mL of water, the solvent was distilled off from the obtained organic phase, original components were removed by silica gel chromatography using dichloromethane/hexane, and the solvent was further distilled off to obtain 4.5 g of a BOC group-substituted compound (a compound represented by the following formula (M3), hereinafter, also referred to as the “compound A3”) of the compound A1, as the target component. The inorganic element content and the organic impurity content were measured by the aforementioned method, and the results are shown in Table 1.

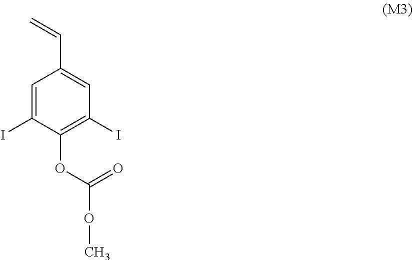

### Example A4

### Synthesis of Compound A4 Represented by Formula (M4)

In a 2 L flask, 400 mL of dichloromethane, 41 g of the obtained compound A1, 16.2 g of triethylamine, and 0.7 g of N-(4-pyridyl)dimethylamine (DMAP) was dissolved in a nitrogen flow. 20.7 g of dibenzyl dicarbonate was dissolved in 100 mL of dichloromethane, which was added dropwise into the above 2 L flask with stirring, and the mixture was stirred at room temperature for 3 hours. Thereafter, water washing was conducted three times by a separation operation using 100 mL of water, the solvent was distilled off from the obtained organic phase, original components were removed by silica gel chromatography using dichloromethane/hexane, and the solvent was further distilled off to obtain 4.5 g of a BOC group-substituted compound (a compound represented by the following formula (M4), hereinafter, also referred to as the “compound A4”) of the compound A1, as the target component. The inorganic element content and the organic impurity content were measured by the aforementioned method, and the results are shown in Table 1.

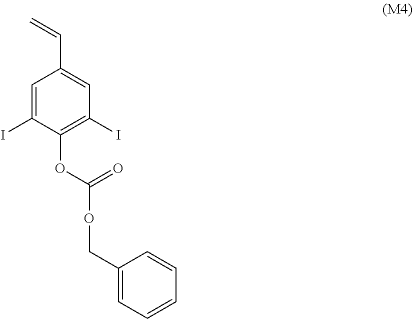 [SMILES: C=Cc1cc(I)c(OC(=O)OCc2ccccc2)c(I)c1]

### Example A5

### Synthesis of Compound A5 Represented by Formula (M5)

Using a 200 mL glass flask as a reaction vessel, 5.6 g (40 mmol) of 3,4-dihydroxybenzyl alcohol was dissolved using butanol as a solvent, a 20% by mass aqueous iodine chloride solution (81.2 g, 100 mmol) was added dropwise thereto at 50° C. over 60 minutes, and then the mixture was stirred at 50° C. for 2 hours to allow 3,4-dihydroxybenzyl alcohol to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 11.3 g of a white solid. The white solid sample was analyzed by liquid chromatography-mass spectrometry (LC-MS) and, as a result, confirmed to be 3,4-dihydroxy-2,5-diiodobenzyl alcohol.

MnO₂ (3.4 g, 40 mmol) was added to a methylene chloride solvent and stirred, which was then stirred for 1 hour while a 50% by mass solution in which the total amount of the synthesized 3,4-dihydroxy-2,5-diiodobenzyl alcohol was dissolved in methylene chloride was added dropwise and stirred at room temperature for 4 hours, and then the reaction solution was filtered off and the solvent was distilled off to obtain 3,4-dihydroxy-2,5-diiodobenzaldehyde.

A solution in which dimethyl malonate (5.3 g, 40 mmol) and the total amount of 3,4-dihydroxy-2,5-diiodobenzaldehyde synthesized above was dissolved in a DMF solvent was prepared and then stirred for 1 hour while a solution in which ethylenediamine (0.3 g) was dissolved in DMF was added dropwise, and the mixture was allowed to react for 6 hours with stirring while controlling the liquid temperature at 150° C. in an oil bath. Thereafter, ethyl acetate and water was added, a 2 mol/L aqueous HCl solution was added to control pH to 4 or less, and then a separation operation was carried out to collect the organic phase. Further, the obtained organic phase was washed by being subjected to a separation operation with a 2 mol/L aqueous sodium carbonate solution, water, and brine in the order presented, and then purified by a filter, and the solvent was distilled off from the organic phase to obtain 7.8 g of the compound A5 (3,4-dihydroxy-2,5-diiodostyrene (a compound represented by the following formula (M5))). The inorganic element content and the organic impurity content were measured by the aforementioned method, and the results are shown in Table 1.

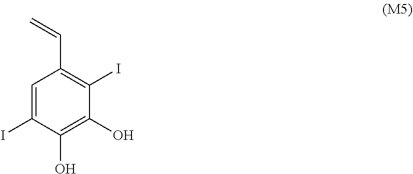

### Example A6

### Synthesis of Compound A6 Represented by Formula (M6)

Using a 200 mL glass flask as a reaction vessel, 5.6 g (40 mmol) of 3,5-dihydroxybenzyl alcohol was dissolved using butanol as a solvent, a 20% by mass aqueous iodine chloride solution (105.6 g, 130 mmol) was added dropwise thereto at 50° C. over 60 minutes, and then the mixture was stirred at 50° C. for 2 hours to allow 3,5-dihydroxybenzyl alcohol to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 14.4 g of a white solid. The white solid sample was analyzed by liquid chromatography-mass spectrometry (LC-MS) and, as a result, confirmed to be 3,5-dihydroxy-2,4,6-triiodobenzyl alcohol.

MnO₂ (3.4 g, 40 mmol) was added to a methylene chloride solvent and stirred, which was then stirred for 1 hour while a 50% by mass solution in which the total amount of the synthesized 3,5-dihydroxy-2,4,6-triiodobenzyl alcohol was dissolved in methylene chloride was added dropwise and stirred at room temperature for 4 hours, and then the reaction solution was filtered off and the solvent was distilled off to obtain 3,5-dihydroxy-2,4,6-triiodobenzaldehyde.

A solution in which dimethyl malonate (5.3 g, 40 mmol) and the total amount of 3,4-dihydroxy-2,5-diiodobenzaldehyde synthesized above was dissolved in a DMF solvent was prepared and then stirred for 1 hour while a solution in which ethylenediamine (0.3 g) was dissolved in DMF was added dropwise, and the mixture was allowed to react for 6 hours with stirring while controlling the liquid temperature at 150° C. in an oil bath. Thereafter, ethyl acetate and water was added, a 2 mol/L aqueous HCl solution was added to control pH to 4 or less, and then a separation operation was carried out to collect the organic phase. Further, the obtained organic phase was washed by being subjected to a separation operation with a 2 mol/L aqueous sodium carbonate solution, water, and brine in the order presented, and then purified by a filter, and the solvent was distilled off from the organic phase to obtain 9.8 g of the compound A6 (3,5-dihydroxy-2,4,6-triiodostyrene (a compound represented by the following formula (M6))). The inorganic element content and the organic impurity content were measured by the aforementioned method, and the results are shown in Table 1.

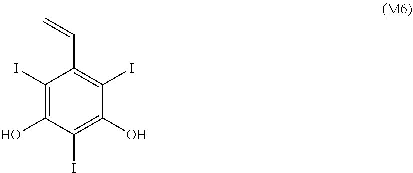

### Example A7

### Synthesis of Compound A7 Represented by Formula (M7)

In a container (internal capacity: 200 mL) equipped with a stirrer, a condenser tube, and a burette, 4.61 g (12.4 mmol) of the compound A1 obtained in the above Example A1 and 2.42 g (12.4 mmol) of tert-butyl bromoacetate were charged to 100 mL of acetone, 1.71 g (12.4 mmol) of potassium carbonate and 0.4 g of 18-crown-6 (IUPAC name: 1,4,7,10,13,16-hexaoxacyclooctadecane) was added, and the contents were reacted by being stirred under reflux for 3 hours to obtain a reaction liquid. Next, the reaction solution was concentrated, and the reaction product was precipitated by the addition of 100 g of pure water to the concentrate, cooled to room temperature, and then filtered to separate solid matter.

The obtained solid matter was filtered and then dried, and then the solid matter was separated and purified by column chromatography to obtain 3.2 g of the compound A7 (a compound represented by the following formula (M7)). Furthermore, the inorganic element content and the organic impurity content were measured by the aforementioned method, and the results are shown in Table 1.

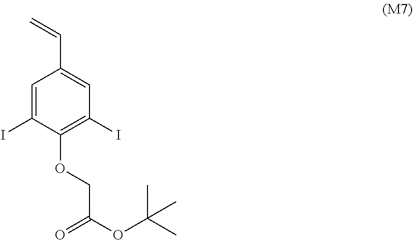 [SMILES: C=Cc1cc(I)c(OCC(=O)OC(C)(C)C)c(I)c1]

### Example A8

### Synthesis of Compound A8 Represented by Formula (M8)

3,5-diiodo4-hydroxybenzaldehyde was obtained by the same process as described in Example A1. Specifically, the method described below was used.

### (Step 1) Iodination Reaction

Using a 200 mL glass flask as a reaction vessel, 5.52 g (40 mmol) of 4-hydroxybenzalcohol was dissolved using butanol as a solvent, a 20% by mass aqueous iodine chloride solution (81.2 g, 100 mmol) was added dropwise thereto at 50° C. over 60 minutes, and then the mixture was stirred at 50° C. for 2 hours to allow 4-hydroxybenzalcohol to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 15.3 g of a white solid. The white solid sample was analyzed by liquid chromatography-mass spectrometry (LC-MS) and, as a result, confirmed to be 4-hydroxy-3,5-diiodobenzyl alcohol.

### (Step 2) Oxidation Reaction

MnO₂ (3.4 g, 40 mmol) was added to a methylene chloride solvent and stirred, which was then stirred for 1 hour while a 50% by mass solution in which the total amount of the synthesized 4-hydroxy-3,5-diiodobenzyl alcohol was dissolved in methylene chloride was added dropwise and stirred at room temperature for 4 hours, and then the reaction solution was filtered off and the solvent was distilled off to obtain 14.5 g of 4-hydroxy-3,5-diiodobenzaldehyde.

### (Step 3) Malonic Acid Addition Reaction

Using a 200 mL eggplant flask equipped with a Dienstark reflux tube, 14.6 g (38 mmol) of 3,5-diiodo4-hydroxybenzaldehyde was mixed with dimethyl malonate (10.6 g, 80 mmol), piperidine (3.4 g, 40 mmol), acetic acid (2.4 g, 40 mmol), and 40 mL of benzene, and allowed to react for 3 hours under reflux conditions. The obtained reaction solution was washed with 20 mL of a 5% by mass aqueous HCl solution, and then washed with a 5% aqueous NaHCO₃ solution. The obtained organic phase was dried over magnesium sulfate, and then concentrated under reduced pressure to obtain 15.8 g of a reaction product M8-CINMe.

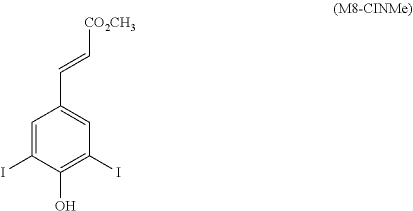 [SMILES: COC(=O)/C=C/c1cc(I)c(O)c(I)c1]

### (M8-CINMe)

### (Step 4) Hydrolysis Reaction

Using a 1 L eggplant flask equipped with a reflux tube, hydrochloric acid (6N, 131 mL) and acetic acid (131 mL) were added to 38 mmol of the product M8-CINMe obtained above, and the mixture was refluxed for 48 hours. Thereafter, 6M, 500 mL of NaOH aq. was added, and the mixture was extracted with 250 mL of ethyl acetate to recover the organic phase consisting of ethyl acetate. The obtained organic phase was subjected to a dehydration treatment with magnesium sulfate, and the filtrate filtered thereafter was concentrated under reduced pressure to obtain 15.2 g of a cinnamic acid derivative M8-CIN.

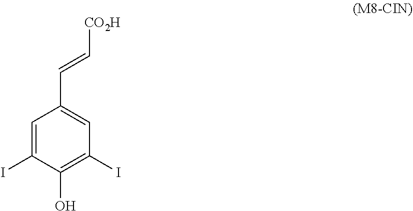 [SMILES: O=C(O)/C=C/c1cc(I)c(O)c(I)c1]

### (Step 5) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 40 mmol of the cinnamic acid derivative M8-CIN prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 14.4 g of a compound (M8-OH) represented by the formula (M8-OH).

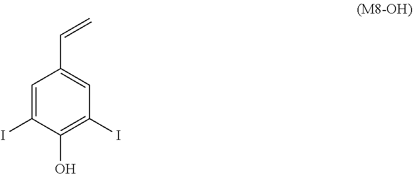

### (Step 6) Acetyl Protecting Group Introduction Reaction

Using a 1 L eggplant flask, 6.1 g (60 mmol) of acetic anhydride, 6.0 g (60 mmol) of triethylamine, 0.8 g (6 mmol) of DMAP, and 350 mL of a solvent (dichloromethane), which were cooled to 4° C. with ice water, were put thereinto, and stirred and dissolved to prepare a reaction solution. Under cooling with ice at 4° C., 14.4 g, 37 mmol of the compound M8-OH prepared in the previous step was dissolved in 50 mL of dichloromethane to prepare a solution of the compound M8-OH, which was added to the solution prepared in the 1 L eggplant flask over 30 minutes. Thereafter, the mixture was stirred at 4° C. for 2 hours to allow the reaction to proceed sufficiently and washed with 400 mL of ice water and 400 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 14.8 g of the target compound A8 represented by the formula (M8). The yield was 90% by mass.

The following peaks were found by carrying out ¹H-NMR measurement under the above measurement conditions, and the compound was confirmed to have the above chemical structure.

δ (ppm) (d6-DMSO): 2.3 (3H, —CH3), 7.7 (2H, Ph), 6.7 (1H, —CH═), 5.3 (1H, ═CH2), 5.7 (1H, ═CH2)

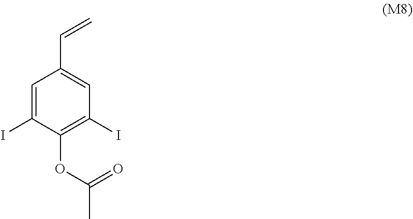

### Example A9

### Synthesis of Compound A9 Represented by Formula (M9)

The compound A9 represented by the formula (M9) was synthesized by the method described below.

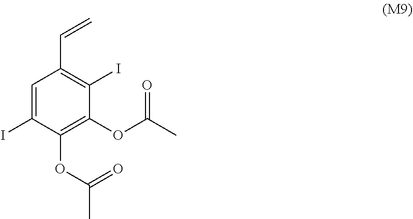 [SMILES: C=Cc1cc(I)c(OC(C)=O)c(OC(C)=O)c1I]

### (Step 1)

Using a 200 mL glass flask as a reaction vessel, 5.52 g (40 mmol) of 3,4-dihydroxybenzaldehyde was dissolved using methanol as a solvent, and then a 20% by mass aqueous iodine chloride solution (81.2 g, 100 mmol) was added dropwise thereto under ice cold conditions over 60 minutes. Furthermore, 4.90 g (20 mmol) of a 71.9% by mass aqueous iodic acid solution was added dropwise under ice cold conditions in a range where the liquid temperature was 8° C. or less over 30 minutes. Thereafter, the mixture was stirred at 40° C. for 3 hours to react 3,4-dihydroxybenzaldehyde with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 15.3 g of a white solid. The white solid sample was analyzed by liquid chromatography-mass spectrometry (LC-MS) and, as a result, confirmed to be 2,5-diiodo-3,4-dihydroxybenzaldehyde.

### (Step 2)

Using a 200 mL eggplant flask equipped with a Dienstark reflux tube, 15.3 g (39 mmol) of 2,5-diiodo-3,4-dihydroxybenzaldehyde was mixed with malononitrile (3.97 g, 60 mmol), piperidine (3.4 g, 40 mmol), acetic acid (2.4 g, 40 mmol), and 40 mL of benzene, and the mixture was allowed to react for 3 hours under reflux conditions. The obtained reaction solution was washed with 20 mL of a 5% by mass aqueous HCl solution, and then washed with a 5% aqueous NaHCO₃ solution. The obtained organic phase was dried over magnesium sulfate, and then concentrated under reduced pressure to obtain a reaction product represented by the following formula M9-CN.

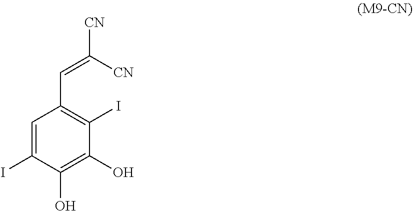 [SMILES: N#CC(C#N)=Cc1cc(I)c(O)c(O)c1I]

### (Step 3)

Using a 1 L eggplant flask equipped with a reflux tube, hydrochloric acid (6N, 131 mL) and acetic acid (131 mL) were added to 39 mmol of the product M9-CN obtained above, and the mixture was refluxed for 48 hours. Thereafter, 6M, 500 mL of NaOH aq. was added, and the mixture was extracted with 250 mL of ethyl acetate to recover the organic phase consisting of ethyl acetate. The obtained organic phase was subjected to a dehydration treatment with magnesium sulfate, and the filtrate filtered thereafter was concentrated under reduced pressure to obtain 16.4 g (38 mmol) of a cinnamic acid derivative represented by the following (M9-CA).

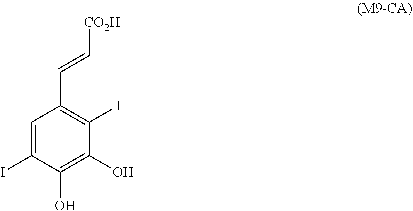 [SMILES: O=C(O)/C=C/c1cc(I)c(O)c(O)c1I]

### (Step 4) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 38 mmol of the cinnamic acid derivative M9-CA prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 14.4 g (37 mmol) of a compound (M9-OH) represented by the formula (M9-OH).

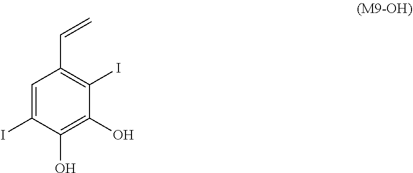

### (Step 5) Acetyl Protecting Group Introduction Reaction

Using a 1 L eggplant flask, 6.1 g (60 mmol) of acetic anhydride, 6.0 g (60 mmol) of triethylamine, 0.8 g (6 mmol) of DMAP, and 350 mL of a solvent (dichloromethane), which were cooled to 4° C. with ice water, were put thereinto, and stirred and dissolved to prepare a reaction solution. Under cooling with ice at 4° C., 14.4 g, 37 mmol of the compound M9-OH prepared in the previous step was dissolved in 50 mL of dichloromethane to prepare a solution of the compound M9-OH, which was added to the solution prepared in the 1 L eggplant flask over 30 minutes. Thereafter, the mixture was stirred at 4° C. for 2 hours to allow the reaction to proceed sufficiently and washed with 400 mL of ice water and 400 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 16.5 g of the target compound A9 represented by the formula (M9). The yield was 88% by mass.

The following peaks were found by carrying out ¹H-NMR measurement under the above measurement conditions, and the compound was confirmed to have the following chemical structure.

δ (ppm) (d6-DMSO): 2.3 (6H, —CH3), 7.4 (1H, Ph), 7.4 (1H, —CH═), 5.6 (1H, ═CH2), 5.7 (1H, ═CH2)

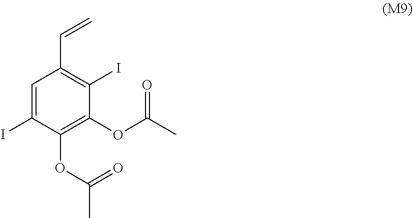 [SMILES: C=Cc1cc(I)c(OC(C)=O)c(OC(C)=O)c1I]

### Example A10

### Synthesis of Compound A10 Represented by Formula (M10)

The compound A10 represented by the formula (M10) was synthesized by the method described below.

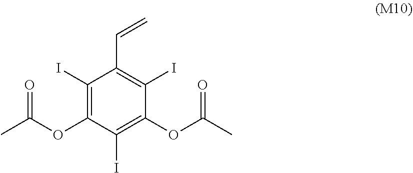 [SMILES: C=Cc1c(I)c(OC(C)=O)c(I)c(OC(C)=O)c1I]

### (Step 1)

Using a 200 mL glass flask as a reaction vessel, 5.52 g (40 mmol) of 3,5-dihydroxybenzaldehyde was dissolved using methanol as a solvent, and then a 20% by mass aqueous iodine chloride solution (121.8 g, 150 mmol) was added dropwise thereto under ice cold conditions over 90 minutes. Furthermore, 7.45 g (30 mmol) of a 71.9% by mass aqueous iodic acid solution was added dropwise under ice cold conditions in a range where the liquid temperature was 8° C. or less over 30 minutes. Thereafter, the mixture was stirred at 40° C. for 3 hours to allow 3,5-dihydroxybenzaldehyde to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 20.1 g of a white solid. The white solid sample was analyzed by liquid chromatography-mass spectrometry (LC-MS) and, as a result, confirmed to be 2,4,6-triiodo-3,5-dihydroxybenzaldehyde.

### (Step 2)

Using a 200 mL eggplant flask equipped with a Dienstark reflux tube, 20.1 g (39 mmol) of 2,4,6-triiodo-3,5-dihydroxybenzaldehyde was mixed with malonic acid (15.6 g, 150 mmol), piperidine (12.8 g, 150 mmol), acetic acid (90 g, 150 mmol), and 40 mL of benzene, and allowed to react for 3 hours under reflux conditions. The obtained reaction solution was washed with 20 mL of a 5% by mass aqueous HCl solution, and then washed with a 5% aqueous NaHCO₃ solution. The obtained organic phase was dried over magnesium sulfate, and then concentrated under reduced pressure to obtain 20.6 g of a cinnamic acid derivative (M10-CA).

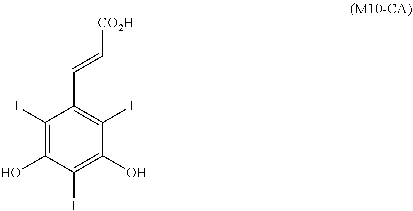 [SMILES: O=C(O)/C=C/c1c(I)c(O)c(I)c(O)c1I]

### (Step 3) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 20.6 g (37 mmol) of the cinnamic acid derivative M10-CA prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 18.0 g (35 mmol) of a compound (M10-OH) represented by the formula (M10-OH).

### (Step 4) Acetyl Protecting Group Introduction Reaction

Using a 1 L eggplant flask, 6.1 g (60 mmol) of acetic anhydride, 6.0 g (60 mmol) of triethylamine, 0.8 g (6 mmol) of DMAP, and 350 mL of a solvent (dichloromethane), which were cooled to 4° C. with ice water, were put thereinto, and stirred and dissolved to prepare a reaction solution. Under cooling with ice at 4° C., 18.0 g, 35 mmol of the compound M10-OH prepared in the previous step was dissolved in 50 mL of dichloromethane to prepare a solution of the compound M10-OH, which was added to the solution prepared in the 1 L eggplant flask over 30 minutes. Thereafter, the mixture was stirred at 4° C. for 2 hours to allow the reaction to proceed sufficiently and washed with 400 mL of ice water and 400 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 20.3 g of the target compound M10. The yield was 85% by mass.

The following peaks were found by carrying out ¹H-NMR measurement under the above measurement conditions, and the compound was confirmed to have the following chemical structure.

δ (ppm) (d6-DMSO): 2.3 (6H, —CH3), 7.4 (1H, —CH═), 5.6 (1H, ═CH2), 5.7 (1H, ═CH2)

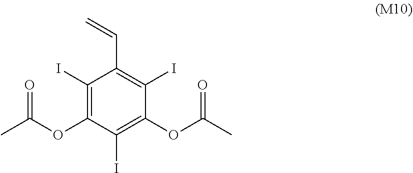 [SMILES: C=Cc1c(I)c(OC(C)=O)c(I)c(OC(C)=O)c1I]

### Example A11 and Example A12

### Synthesis of Compound A11 Represented by Formula (M11) and Compound A12 Represented by Formula (M12)

The compound A11 represented by the formula (M11) and the compound A12 represented by the formula (12) were synthesized by the method described below.

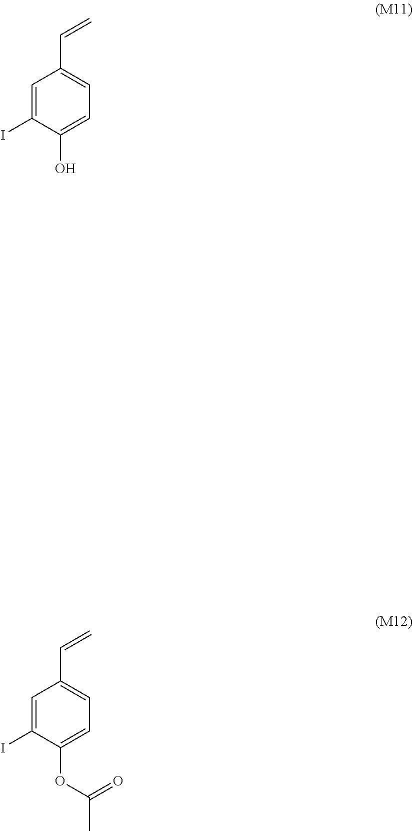

### (Step 1) Iodination Reaction

Using a 200 mL glass flask as a reaction vessel, 5.45 g (40 mmol) of 4-hydroxybenzyl alcohol was dissolved using butanol as a solvent, a 20% by mass aqueous iodine chloride solution (40.6 g, 50 mmol) was added dropwise thereto at 50° C. over 60 minutes, and then the mixture was stirred at 50° C. for 2 hours to allow 4-hydroxybenzaldehyde to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 10.3 g of a white solid. The white solid sample was analyzed by liquid chromatography-mass spectrometry (LC-MS) and, as a result, confirmed to be 4-hydroxy-3-iodobenzyl alcohol.

### (Step 2) Oxidation Reaction

MnO₂ (3.4 g, 40 mmol) was added to a methylene chloride solvent and stirred, which was then stirred for 1 hour while a 50% by mass solution in which the total amount of the synthesized 4-hydroxy-3-iodobenzyl alcohol was dissolved in methylene chloride was added dropwise and stirred at room temperature for 4 hours, and then the reaction solution was filtered off and the solvent was distilled off to obtain 14.5 g of 4-hydroxy-3-iodobenzaldehyde.

### (Step 3) Malonic Acid Addition Reaction

Using a 200 mL eggplant flask equipped with a Dienstark reflux tube, 14.6 g (38 mmol) of 4-iodo-3-hydroxybenzaldehyde was mixed with dimethyl malonate (10.6 g, 80 mmol), piperidine (3.4 g, 40 mmol), acetic acid (2.4 g, 40 mmol), and 40 mL of benzene, and allowed to react for 3 hours under reflux conditions. The obtained reaction solution was washed with 20 mL of a 5% by mass aqueous HCl solution, and then washed with a 5% aqueous NaHCO₃ solution. The obtained organic phase was dried over magnesium sulfate, and then concentrated under reduced pressure to obtain 13.4 g of a reaction product M11-CINMe.

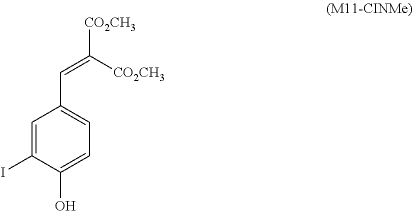 [SMILES: COC(=O)C(=Cc1ccc(O)c(I)c1)C(=O)OC]

### (Step 4) Hydrolysis Reaction

Using a 1 L eggplant flask equipped with a reflux tube, hydrochloric acid (6N, 131 mL) and acetic acid (131 mL) were added to 13.4 g (37 mmol) of the product M11-CINMe obtained above, and the mixture was refluxed for 48 hours. Thereafter, 6M, 500 mL of NaOH aq. was added, and the mixture was extracted with 250 mL of ethyl acetate to recover the organic phase consisting of ethyl acetate. The obtained organic phase was subjected to a dehydration treatment with magnesium sulfate, and the filtrate filtered thereafter was concentrated under reduced pressure to obtain 10.4 g of a cinnamic acid derivative MA11-CA.

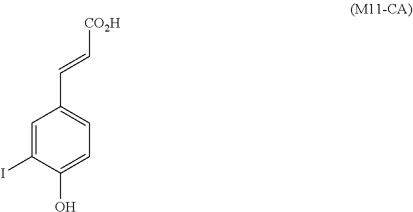 [SMILES: O=C(O)/C=C/c1ccc(O)c(I)c1]

### (Step 5) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 10.4 g (36 mmol) of the cinnamic acid derivative M11-CA prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 8.6 g of a compound (A11) represented by the formula (M11).

The following peaks were found by carrying out ¹H-NMR measurement under the above measurement conditions, and the compound was confirmed to have the following chemical structure.

δ (ppm) (d6-DMSO): 2.3 (3H, —CH3), 7.7 (2H, Ph), 6.7 (1H, —CH═), 5.3 (1H, ═CH2), 5.7 (1H, ═CH2)

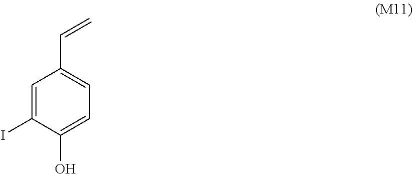

### (Step 6) Acetyl Protecting Group Introduction Reaction

Using a 1 L eggplant flask, 6.1 g (60 mmol) of acetic anhydride, 6.0 g (60 mmol) of triethylamine, 0.8 g (6 mmol) of DMAP, and 350 mL of a solvent (dichloromethane), which were cooled to 4° C. with ice water, were put thereinto, and stirred and dissolved to prepare a reaction solution. Under cooling with ice at 4° C., 8.6 g, 36 mmol of the compound A11 prepared in the previous step was dissolved in 50 mL of dichloromethane to prepare a solution of the compound A11, which was added to the solution prepared in the 1 L eggplant flask over 30 minutes. Thereafter, the mixture was stirred at 4° C. for 2 hours to allow the reaction to proceed sufficiently and washed with 400 mL of ice water and 400 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 10.0 g of the target compound A12 represented by the formula (M12). The yield was 88% by mass.

The following peaks were found by carrying out ¹H-NMR measurement under the above measurement conditions, and the compound was confirmed to have the following chemical structure.

δ (ppm) (d6-DMSO): 2.3 (3H, —CH3), 7.7 (2H, Ph), 6.7 (1H, —CH═), 5.3 (1H, ═CH2), 5.7 (1H, ═CH2)

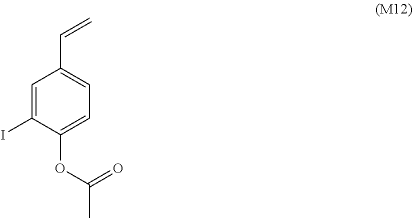

### Example A13 and Example A14

### Synthesis of Compound A13 and Compound A14 Represented by Formula (M13) and Formula (M14)

The compound A13 represented by the formula (M13) and the compound A14 represented by the formula (M14) were synthesized by the method described below.

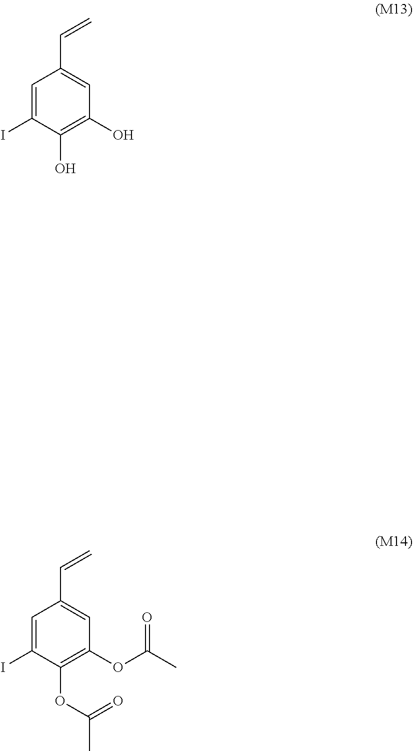 [SMILES: C=Cc1cc(O)c(O)c(I)c1 ; C=Cc1cc(I)c(OC(C)=O)c(OC(C)=O)c1]

### (Step 1)

Using a 200 mL glass flask as a reaction vessel, 5.52 g (40 mmol) of 3,4-dihydroxybenzaldehyde was dissolved using methanol as a solvent, and then a 20% by mass aqueous iodine chloride solution (40.6 g, 50 mmol) was added dropwise thereto under ice cold conditions over 60 minutes. Furthermore, 2.45 g (10 mmol) of a 71.9% by mass aqueous iodic acid solution was added dropwise under ice cold conditions in a range where the liquid temperature was 8° C. or less over 30 minutes. Thereafter, the mixture was stirred at 40° C. for 3 hours to react 3,4-dihydroxybenzaldehyde with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 10.2 g of a white solid. The white solid sample was analyzed by liquid chromatography-mass spectrometry (LC-MS) further using silica gel chromatography and, as a result, confirmed to be 2-iodo-3,4-dihydroxybenzaldehyde.

### (Step 2)

Using a 200 mL eggplant flask equipped with a Dienstark reflux tube, 10.3 g (39 mmol) of 2-iodo-3,4-dihydroxybenzaldehyde was mixed with malononitrile (3.97 g, 60 mmol), piperidine (3.4 g, 40 mmol), acetic acid (2.4 g, 40 mmol), and 40 mL of benzene, and the mixture was allowed to react for 3 hours under reflux conditions. The obtained reaction solution was washed with 20 mL of a 5% by mass aqueous HCl solution, and then washed with a 5% by mass aqueous NaHCO₃ solution. The obtained organic phase was dried over magnesium sulfate, and then concentrated under reduced pressure to obtain 11.9 g of a reaction product (M13-CINMe).

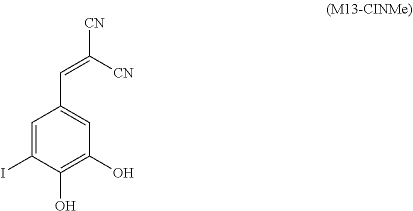 [SMILES: N#CC(C#N)=Cc1cc(O)c(O)c(I)c1]

### (Step 3)

Using a 1 L eggplant flask equipped with a reflux tube, hydrochloric acid (6N, 131 mL) and acetic acid (131 mL) were added to 11.9 (38 mmol) of the product obtained above, and the mixture was refluxed for 48 hours. Thereafter, 6M, 500 mL of NaOH aq. was added, and the mixture was extracted with 250 mL of ethyl acetate to recover the organic phase consisting of ethyl acetate. The obtained organic phase was subjected to a dehydration treatment with magnesium sulfate, and the filtrate filtered thereafter was concentrated under reduced pressure to obtain 11.6 g of a cinnamic acid derivative (M13-CA).

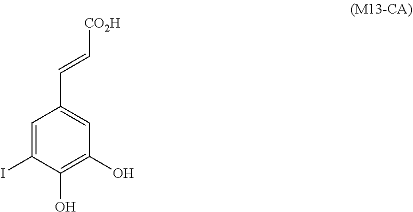 [SMILES: O=C(O)/C=C/c1cc(O)c(O)c(I)c1]

### (Step 4) Decarboxylation Reaction

Using a 1 L eggplant flask, a solution in which 0.023 g (0.4 mmol) of potassium fluoride trihydrate was dissolved in a mixed solution of 4 mL of acetic acid and 16 mL of dimethylsulfoxide was slowly added to a solution in which 11.6 g (38 mmol) of the cinnamic acid derivative prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 9.1 g of a compound A13 represented by the formula (M13).

The following peaks were found by carrying out ¹H-NMR measurement under the above measurement conditions, and the compound was confirmed to have the chemical structure of the compound A13.

δ (ppm) (d6-DMSO): 9.5 (1H, OH), 9.6 (1H, OH), 7.0 (2H, Ph), 6.7 (1H, —CH═), 5.3 (1H, ═CH2), 5.7 (1H, ═CH2)

### (Step 5) Acetyl Protecting Group Introduction Reaction

Using a 1 L eggplant flask, 6.1 g (60 mmol) of acetic anhydride, 6.0 g (60 mmol) of triethylamine, 0.8 g (6 mmol) of DMAP, and 350 mL of a solvent (dichloromethane), which were cooled to 4° C. with ice water, were put thereinto, and stirred and dissolved to prepare a reaction solution. Under cooling with ice at 4° C., 9.1 g, 35 mmol of the compound A13 prepared in the previous step was dissolved in 50 mL of dichloromethane to prepare a solution of the compound A13, which was added to the solution prepared in the 1 L eggplant flask over 30 minutes. Thereafter, the mixture was stirred at 4° C. for 2 hours to allow the reaction to proceed sufficiently and washed with 400 mL of ice water and 400 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 12.1 g of the target compound A14 represented by the formula (M14).

The following peaks were found by carrying out ¹H-NMR measurement under the above measurement conditions, and the compound was confirmed to have the chemical structure of the compound A14.

δ (ppm) (d6-DMSO): 2.3 (6H, —CH3), 7.7 (2H, Ph), 6.7 (1H, —CH═), 5.3 (1H, ═CH2), 5.7 (1H, ═CH2)

### Example A15 and Example A16

### Synthesis of Compound A15 and Compound A16 Represented by Formula (M15) and Formula (M16)

The compound A15 represented by the formula (M15) and the compound A16 represented by the formula (M16) were synthesized by the method described below.

(Step 1) Formation of 4-iodo-3,5-dihydroxybenzaldehyde

Using a 200 mL glass flask as a reaction vessel, 5.52 g (40 mmol) of 3,5-dihydroxybenzaldehyde was dissolved using methanol as a solvent, and then a 20% by mass aqueous iodine chloride solution (40.6 g, 50 mmol) was added dropwise thereto under ice cold conditions over 60 minutes. Furthermore, 2.45 g (10 mmol) of a 71.9% by mass aqueous iodic acid solution was added dropwise under ice cold conditions in a range where the liquid temperature was 8° C. or less over 30 minutes. Thereafter, the mixture was stirred at 40° C. for 3 hours to allow 3,5-dihydroxybenzaldehyde to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 10.2 g of a white solid. The white solid sample was analyzed by liquid chromatography-mass spectrometry (LC-MS) and, as a result, confirmed to be 4-iodo-3,5-dihydroxybenzaldehyde.

### (Step 2) Formation of Cinnamic Acid Derivative Structure

Using a 200 mL eggplant flask equipped with a Dienstark reflux tube, 10.3 g (39 mmol) of 4-iodo-3,5-dihydroxybenzaldehyde was mixed with malonic acid (6.24 g, 60 mmol), piperidine (3.4 g, 40 mmol), acetic acid (2.4 g, 40 mmol), and 40 mL of benzene, and allowed to react for 3 hours under reflux conditions. The obtained reaction solution was washed with 20 mL of a 5% by mass aqueous HCl solution, and then washed with a 5% aqueous NaHCO₃ solution. The obtained organic phase was dried over magnesium sulfate, and then concentrated under reduced pressure to obtain 11.7 g of a reaction product (M15-CA) consisting of a cinnamic acid derivative.

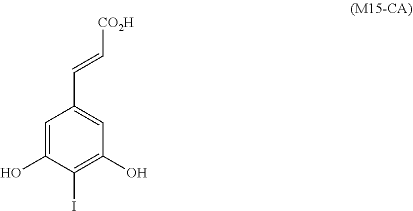 [SMILES: O=C(O)/C=C/c1cc(O)c(I)c(O)c1]

### (Step 3) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 11.7 g (38 mmol) of the cinnamic acid derivative M15-CA prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 9.4 g of a compound (A15) represented by the formula (M15).

The following peaks were found by carrying out ¹H-NMR measurement under the above measurement conditions, and the compound was confirmed to have the following chemical structure.

δ (ppm) (d6-DMSO): 11.6 (2H, OH), 6, 0 (2H, Ph), 6.7 (1H, —CH═), 5.3 (1H, ═CH2), 5.7 (1H, ═CH2)

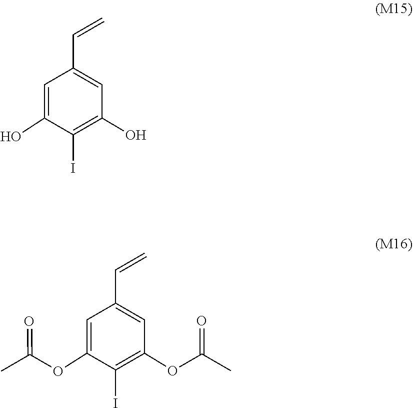 [SMILES: C=Cc1cc(O)c(I)c(O)c1 ; C=Cc1cc(OC(C)=O)c(I)c(OC(C)=O)c1]

### (Step 4) Acetyl Protecting Group Introduction Reaction

Using a 1 L eggplant flask, 6.1 g (60 mmol) of acetic anhydride, 6.0 g (60 mmol) of triethylamine, 0.8 g (6 mmol) of DMAP, and 350 mL of a solvent (dichloromethane), which were cooled to 4° C. with ice water, were put thereinto, and stirred and dissolved to prepare a reaction solution. Under cooling with ice at 4° C., 9.4 g, 36 mmol of the compound A15 prepared in the previous step was dissolved in 50 mL of dichloromethane to prepare a solution of the compound A15, which was added to the solution prepared in the 1 L eggplant flask over 30 minutes. Thereafter, the mixture was stirred at 4° C. for 2 hours to allow the reaction to proceed sufficiently and washed with 400 mL of ice water and 400 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 12.3 g of the target compound A16.

The following peaks were found by carrying out ¹H-NMR measurement under the above measurement conditions, and the compound was confirmed to have the chemical structure of the compound A16.

δ (ppm) (d6-DMSO): 2.3 (6H, —CH3), 6.7 (2H, Ph), 6.7 (1H, —CH═), 5.3 (1H, ═CH2), 5.7 (1H, ═CH2)

### Synthesis Example ACL1

### Synthesis of Compound MCL1 Represented by Formula (MCL1)

The compound MCL1 represented by the formula (MCL1) was synthesized by the method described below.

(Step 1) Diiodination of 4-hydroxyacetophenone

Using a 200 mL glass flask as a reaction vessel, 6.1 g (45 mmol) of 4-hydroxyacetophenone was dissolved using butanol as a solvent, a 20% by mass aqueous iodine chloride solution (81.2 g, 100 mmol) was added dropwise thereto at 50° C. over 60 minutes, and then the mixture was stirred at 50° C. for 2 hours to allow 4-hydroxyacetophenone to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 16.3 g of a white solid. The white solid sample was analyzed by liquid chromatography-mass spectrometry (LC-MS) and, as a result, confirmed to be 4-hydroxy-3,5-diiodoacetophenone.

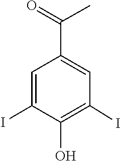

### (Raw Material: Acetophenone Derivative) Mw 387.94

### (Step 2) Introduction of Chloro Group at a Position

In a flask, 0.6 g (6.1 mmol) of CuCl, 1.3 g (13 mmol) of triethylamine, 5.2 g (34 mmol) of POC13 (phosphorus(V) oxychloride), and 15 mL of heptane were stirred at 25° C., and 16.3 g (42 mmol) of 4-hydroxy-3,5-diiodoacetophenone prepared in step 1 was added thereto and dissolved. The mixture was heated until the solution temperature reached 100° C., allowed to react for 20 hours, and cooled to 45° C., and 25 mL of pure water was added dropwise to terminate the reaction. After the aqueous layer was removed, the reaction mixture was washed with pure water (10 mL) and brine (10 mL) by a separation treatment, and then subjected to a dehydration treatment by adding magnesium sulfate. The filtrate after filtration was concentrated to obtain a compound according to the formula (MCL1).

The following peaks were found by carrying out ¹H-NMR measurement under the above measurement conditions, and the compound was confirmed to have the following chemical structure.

δ (ppm) (d6-DMSO): 9.6 (1H, —OH), 7.5 (2H, Ph), 5.4 (1H, ═CH2), 5.7 (1H, ═CH2)

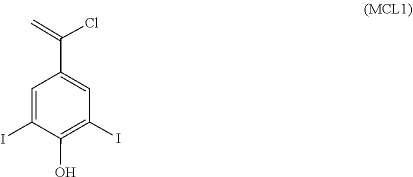

### (Target Substance) Mw 406.39

### Synthesis Example AD1

### Synthesis of Compound MD1 Represented by Formula (MD1)

The compound MD1 represented by the formula (MD1) was synthesized by the method described below.

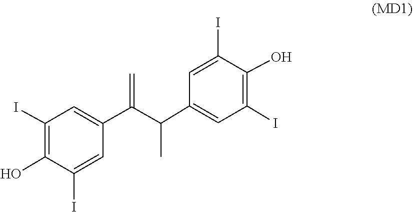 [SMILES: C=C(c1cc(I)c(O)c(I)c1)C(C)c1cc(I)c(O)c(I)c1]

The reactor was charged with 15.6 g of 1-(4-hydroxy-3,5-diiodophenyl)ethanol, 0.12 g of concentrated sulfuric acid, 0.04 g of 4-hydroxy-2,2,6,6-tetramethylpiperidine 1-oxyl free radical, and 1.60 mL of DMSO, and stirring was initiated. Subsequently, reduced pressure conditions were regulated to reflux at 120° C. using a Dienstark and a condenser, and blowing of air into the reaction solution at a flow rate of 1 mL/minute was initiated. The moisture recovered in the Dienstark was arbitrarily discharged to the outside of the system. Subsequently, the reactor was immersed in a water bath at 90° C., and stirring was continued over 30 hours. Subsequently, the reactor was immersed in a water bath at 25° C. to cool the reaction solution. Subsequently, the reaction solution was gradually added to 400 g of an aqueous sodium hydrogen sulfite solution with a mass percent concentration of 0.1 with vigorous stirring and mixed. Subsequently, the precipitate was filtered off with a suction filter, compressed, and washed with 200 mL of an aqueous methanol solution with a volume percent concentration of 33.3. After only main components were isolated from the obtained precipitate by column purification, the solvent was distilled off by evaporation, and the obtained solid was dried in vacuo at 40° C. to obtain 9.7 g of a white solid. The yield was 66%.

As a result of the analysis by liquid chromatography-mass spectrometry (LC-MS), the molecular weight was found to be 743.9, and the white solid was confirmed to be the compound MD1 represented by the formula (MD1).

The following peaks were found by carrying out ¹H-NMR measurement under the above measurement conditions, and the compound was confirmed to have the chemical structure of the compound MD1.

δ (ppm) (d6-DMSO): 9.6 (2H, OH), 7.5 (2H, Ph), 7.9 (2H, Ph), 3.5 (1H, —CH—), 1.3 (3H, —CH3), 4.9 (1H, ═CH2), 5.3 (1H, ═CH2)

### Synthesis Example AD2

### Synthesis of Compound MD2 Represented by Formula (MD2)

The compound MD2 represented by the formula (MD2) was synthesized by the method described below.

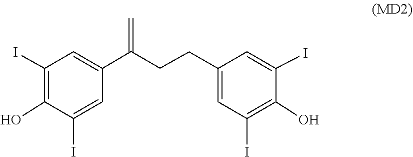 [SMILES: C=C(CCc1cc(I)c(O)c(I)c1)c1cc(I)c(O)c(I)c1]

The reactor was charged with 15.6 g of 1-(4-hydroxy-3,5-diiodophenyl)ethanol, 0.12 g of concentrated sulfuric acid, 0.2 g of 4-methoxyphenol, and 150 mL of toluene, and stirring was initiated. Subsequently, blowing of air into the reaction solution at a flow rate of 1 mL/minute was initiated under reflux conditions at 113° C. using a Dienstark and a condenser. The moisture recovered in the Dienstark was arbitrarily discharged to the outside of the system. Subsequently, the reactor was immersed in a water bath at 90° C., and stirring was continued over 30 hours. Subsequently, the reactor was immersed in a water bath at 25° C. to cool the reaction solution. Subsequently, the reaction solution was gradually added to 400 g of an aqueous sodium hydrogen sulfite solution with a mass percent concentration of 0.1 with vigorous stirring and mixed. Subsequently, the precipitate was filtered off with a suction filter, compressed, and washed with 200 mL of an aqueous methanol solution with a volume percent concentration of 33.3. After only main components were isolated from the obtained precipitate by column purification, the solvent was distilled off by evaporation, and the obtained solid was dried in vacuo at 40° C. to obtain 5.9 g of a white solid. The yield was 41%.

As a result of the analysis by liquid chromatography-mass spectrometry (LC-MS), the molecular weight was found to be 743.89, and the white solid was confirmed to be the compound MD2 represented by the formula (MD2).

The following peaks were found by carrying out ¹H-NMR measurement under the above measurement conditions, and the compound was confirmed to have the chemical structure of the compound MD2.

δ (ppm) (d6-DMSO): 9.6 (2H, OH), 7.5 (2H, Ph), 7.6 (2H, Ph), 2.3 (2H, —CH2-), 2.6 (2H, —CH2-), 4.9 (1H, ═CH2), 5.3 (1H, ═CH2)

### Synthesis Example AD3

### Synthesis of Compound MD3 Represented by Formula (MD3)

The compound MD3 represented by the formula (MD3) was synthesized by the method described below.

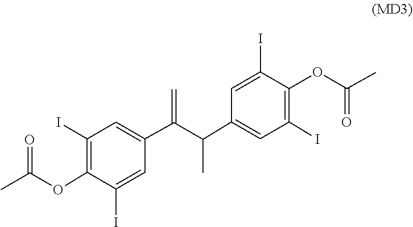 [SMILES: C=C(c1cc(I)c(OC(C)=O)c(I)c1)C(C)c1cc(I)c(OC(C)=O)c(I)c1]

The following steps were conducted using the compound MD1 synthesized above.

Using a 1 L eggplant flask, 1.57 g of acetic anhydride, 1.53 g of triethylamine, 0.19 g of DMAP, and 35 mL of a solvent (dichloromethane), which were cooled to 4° C. with ice water, were put thereinto, and stirred and dissolved to prepare a reaction solution. Under cooling with ice at 4° C., 7.43 g, 10 mmol of the compound MD1 prepared in the previous step was dissolved in 10 mL of dichloromethane to prepare a solution of the compound MD1, which was added to the solution prepared in the 1 L eggplant flask over 30 minutes. Thereafter, the mixture was stirred at 4° C. for 2 hours to allow the reaction to proceed sufficiently and washed with 40 mL of ice water and 40 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 6.7 g of the target compound MD3.

The following peaks were found by carrying out ¹H-NMR measurement under the above measurement conditions, and the compound was confirmed to have the chemical structure of the compound MD3.

δ (ppm) (d6-DMSO): 2.3 (6H, —CH3), 7.7 (2H, Ph), 8.0 (2H, Ph), 1.3 (3H, —CH3), 3.4 (1H, —CH—), 4.9 (1H, ═CH2), 5.3 (1H, ═CH2)

### Synthesis Example AD4

### Synthesis of Compound MD4 Represented by Formula (MD4)

The compound MD4 represented by the formula (MD4) was synthesized by the method described below.

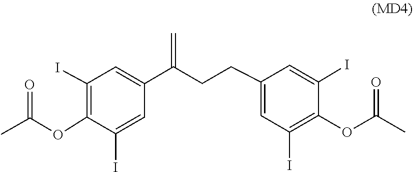 [SMILES: C=C(CCc1cc(I)c(OC(C)=O)c(I)c1)c1cc(I)c(OC(C)=O)c(I)c1]

The following steps were conducted using the compound MD2 synthesized above.

Using a 1 L eggplant flask, 1.57 g of acetic anhydride, 1.53 g of triethylamine, 0.19 g of DMAP, and 35 mL of a solvent (dichloromethane), which were cooled to 4° C. with ice water, were put thereinto, and stirred and dissolved to prepare a reaction solution. Under cooling with ice at 4° C., 7.43 g, 10 mmol of the compound MD2 prepared in the previous step was dissolved in 10 mL of dichloromethane to prepare a solution of the compound MD2, which was added to the solution prepared in the 1 L eggplant flask over 30 minutes. Thereafter, the mixture was stirred at 4° C. for 2 hours to allow the reaction to proceed sufficiently and washed with 40 mL of ice water and 40 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 7.1 g of the target compound MD4.

The following peaks were found by carrying out ¹H-NMR measurement under the above measurement conditions, and the compound was confirmed to have the chemical structure of the compound MD4.

δ (ppm) (d6-DMSO): 2.3 (6H, —CH3), 7.7 (4H, Ph), 2.6 (2H, —CH2-), 2.3 (2H, —CH2), 5.0 (1H, ═CH2), 5.3 (1H, ═CH2)

### Example AH1

### Synthesis of Compound Represented by Formula (MH1)

### (Step 1)

Using a 200 mL glass flask as a reaction vessel, 6.24 g (44 mmol) of BF3.OEt2 complex was added to 4.28 g (40 mmol) of 3-pyridinecarbaldehyde, 2,2,6,6-tetramethylpiperidinylmagnesium chloride lithium chloride complex (100 mL, 44 mmol, 1.2 M/THF)) was added thereto at −40° C. and stirred for 30 minutes, a solution in which (20 g, 80 mmol) was dissolved in 80 mL of THF was slowly added dropwise, and then the mixture was warmed to 25° C. and stirred. Thereafter, the mixture was washed with 180 mL of a saturated aqueous NH4Cl solution, further washed with 20 mL of an aqueous ammonium solution and 40 mL of an aqueous Na2SO3 solution, and extracted with diethyl ether. Sodium sulfate was added, drying was performed, and then the obtained diethyl ether solution was concentrated and purified by silica gel chromatography to obtain 5.7 g of 2-iodo-3-pyridinecarbaldehyde as the target substance.

### (Step 2) Wittig

Using a 200 mL glass flask as a reaction vessel, 6.4 g (16.8 mmol) of triphenylphosphonium methyl bromide and 20 mL of toluene were put thereinto and dissolved. After a KTB solution in which 2.2 g (19.6 mmol) of potassium tert-butoxide was dissolved in 9 mL of THF was prepared, the KTB solution was added dropwise into a toluene solution which was put in an ice bath while the temperature was regulated to 0° C. or less, and then the mixture was stirred for 30 minutes as it was. While the temperature was further regulated to 0° C. or less, a solution in which 4.66 g (20.0 mmol) of 2-iodo-3-pyridinecarbaldehyde was dissolved in 15 mL of toluene was added dropwise, and then the mixture was stirred for 4 hours as it was. Thereafter, the mixture was further washed with 10 mL of water, 10 mL of 10% sodium bisulfite, 10 mL of 5% sodium bicarbonate water, and 10 mL of pure water, in the order presented. 8.1 g of 3-vinyl-2-iodopyridine represented by the formula (MH1) as the target substance was isolated using a silica gel column.

The following peaks were found by carrying out ¹H-NMR measurement under the above measurement conditions, and the compound was confirmed to have the chemical structure of the compound represented by the formula (MH1).

δ (ppm) (d6-DMSO): 8.0 (1H, Pyridine), 7.4 (1H, Pyridine), 7.2 (1H, Pyridine), 7.1 (1H, —CH═), 5.9 (1H, ═CH2), 5.4 (1H, ═CH2)

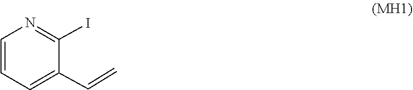

### Example AH2

### Synthesis of Compound Represented by Formula (MH2)

### (Step 1)

Using a 200 mL glass flask as a reaction vessel, 4.56 g (40 mmol) of 5-oxooxolane-3-carbaldehyde was dissolved in 20 mL of THF, a lithiumdiisopropylamide.THF solution (22 mL, 44 mmol, 2 mol/L) was added thereto at −40° C. and stirred for 30 minutes, a solution in which 12 (20 g, 80 mmol) was dissolved in 80 mL of THF was slowly added dropwise, and then the mixture was warmed to 25° C. and stirred. Thereafter, 3 mL of isopropanol was slowly added dropwise and then the mixture was further stirred for 30 minutes. Thereafter, the mixture was washed with 180 mL of a saturated aqueous NH4Cl solution, further washed with 20 mL of an aqueous ammonium solution and 40 mL of an aqueous Na2SO3 solution, and extracted with diethyl ether. Sodium sulfate was added, drying was performed, and then the obtained diethyl ether solution was concentrated and purified by silica gel chromatography to obtain 6.7 g of a compound represented by the formula (MH2-AL) as the target substance.

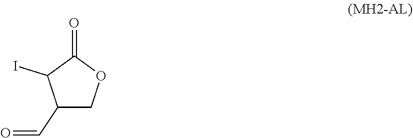

### (Step 2) Wittig

Using a 200 mL glass flask as a reaction vessel, 6.4 g (16.8 mmol) of triphenylphosphonium methyl bromide and 20 mL of toluene were put thereinto and dissolved. After a KTB solution in which 2.2 g (19.6 mmol) of potassium tert-butoxide was dissolved in 9 mL of THF was prepared, the KTB solution was added dropwise into a toluene solution which was put in an ice bath while the temperature was regulated to 0° C. or less, and then the mixture was stirred for 30 minutes as it was. While the temperature was further regulated to 0° C. or less, a solution in which 4.8 g (20.0 mmol) of 2-iodo-5-oxooxolane-3-carbaldehyde was dissolved in 15 mL of toluene was added dropwise, and then the mixture was stirred for 4 hours as it was. Thereafter, the mixture was further washed with 10 mL of water, 10 mL of 10% sodium bisulfite, 10 mL of 5% sodium bicarbonate water, and 10 mL of pure water, in the order presented. 8.1 g of the compound represented by the formula (MH2) as the target substance was isolated using a silica gel column.

The following peaks were found by carrying out ¹H-NMR measurement under the above measurement conditions, and the compound was confirmed to have the chemical structure of the compound represented by the formula (MH2).

δ (ppm) (d6-DMSO): 4.9 (1H, —CH (I)—), 2.93 (1H, —CH (C)), 4.4 (2H, —CH2-O), 5.7 (1H, —CH═), 5.0 (1H, ═CH2), 5.1 (1H, ═CH2)

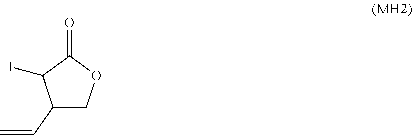

### Synthesis Example B1

### Synthesis of 1,3-divinylbenzene

### (Malonic Acid Addition Reaction Step)

Using a 200 mL eggplant flask equipped with a Dienstark reflux tube, 5.36 g (40 mmol) of 1,3-benzenedicarboxy aldehyde was mixed with malonic acid (10.4 g, 100 mmol), piperidine (6.8 g, 80 mmol), acetic acid (4.8 g, 80 mmol), and 80 mL of benzene, and allowed to react for 3 hours under reflux conditions. The obtained reaction solution was washed with 40 mL of a 5% by mass aqueous HCl solution, and then washed with a 5% aqueous NaHCO₃ solution. The obtained organic phase was dried over magnesium sulfate, and then concentrated under reduced pressure to obtain 8.3 g of a reaction product consisting of a cinnamic acid derivative.

### (Decarboxylation Reaction Step)

Using a 1 L eggplant flask, a solution in which 0.13 g (0.4 mmol) of tetrabutylammonium fluoride trihydrate was dissolved in 20 mL of dimethylsulfoxide was slowly added to a solution in which 8.3 g (38 mmol) of the cinnamic acid derivative prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 4.8 g of 1,3-divinylbenzene.

The following peaks were found by carrying out ¹H-NMR measurement under the above measurement conditions, and the compound was confirmed to have the following chemical structure.

δ (ppm) (d6-DMSO): 7.2 (1H, Ph), 7.5 (2H, Ph), 6.7 (1H, Ph), 6.7 (2H, —CH═), 5.3 (2H, ═CH2), 5.7 (2H, ═CH2)

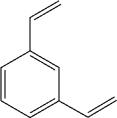

### Synthesis Example B2

### Synthesis of 1,4-divinylbenzene

4.7 g of 1,4-divinylbenzene was obtained in the same manner as Synthesis Example of 1,3-divinylbenzene except for using 5.36 g of 1,4-benzenedicarboxylaldehyde instead of 1,3-benzenedicarboxylaldehyde.

The following peaks were found by carrying out ¹H-NMR measurement under the above measurement conditions, and the compound was confirmed to have the following chemical structure.

δ (ppm) (d6-DMSO): 7.3 (4H, Ph), 6.7 (2H, —CH═), 5.3 (2H, ═CH2), 5.7 (2H, ═CH2)

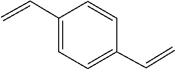

### Synthesis Example B3

### Synthesis of 4-vinylbiphenyl

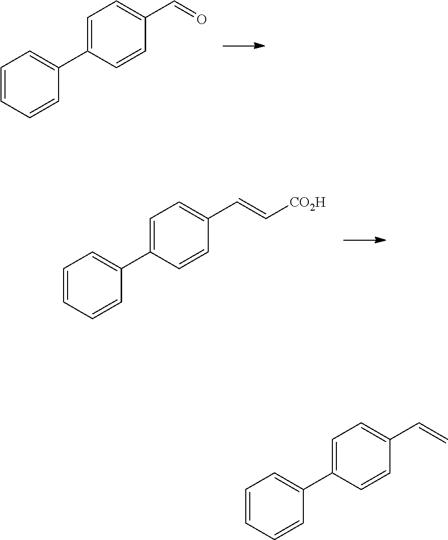

5.7 g of 4-vinylbiphenyl was obtained in the same manner as Synthesis Example of 1,3-divinylbenzene except for using 7.3 g of 4-phenylbenzaldehyde instead of 1,3-benzenedicarboxylaldehyde.

The following peaks were found by carrying out ¹H-NMR measurement under the above measurement conditions, and the compound was confirmed to have the chemical structure of 4-vinylbiphenyl.

δ (ppm) (d6-DMSO): 7.4 (1H, Ph), 7.5 (4H, Ph), 7.6 (2H, Ph), 7.8 (2H, Ph), 6.7 (2H, —CH═), 5.3 (2H, ═CH2), 5.7 (2H, ═CH2)

### Synthesis Example B4

### Synthesis of 2-vinylfuran

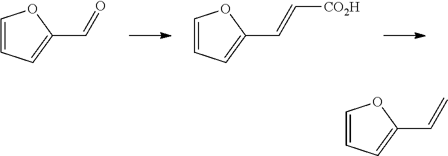

3.1 g of 2-vinylfuran was obtained in the same manner as Synthesis Example of 1,3-divinylbenzene except for using 3.9 g of 2-furanaldehyde instead of 1,3-benzenedicarboxylaldehyde.

The following peaks were found by carrying out ¹H-NMR measurement under the above measurement conditions, and the compound was confirmed to have the chemical structure of 2-vinylfuran.

δ (ppm) (d6-DMSO): 7.7(1H, —CH═), 6.5 (1H, —CH═), 7.0 (1H, —CH═), 6.6 (1H, —CH═), 5.8, 5.4 (1H, ═CH2)

### Synthesis Example B5

### Synthesis of 2-vinylthiophene

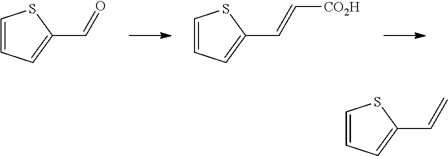

3.5 g of 2-vinylthiophene was obtained in the same manner as Synthesis Example of 1,3-divinylbenzene except for using 4.5 g of thiophene-2-aldehyde instead of 1,3-benzenedicarboxylaldehyde.

The following peaks were found by carrying out ¹H-NMR measurement under the above measurement conditions, and the compound was confirmed to have the chemical structure of 2-vinylthiophene.

δ (ppm) (d6-DMSO): 7.7 (1H, —CH═), 7.0 (1H, —CH═), 7.0 (1H, —CH═), 6.6 (1H, —CH═), 5.4 (1H, ═CH2), 5.9 (1H, ═CH2)

### Synthesis Example B6

### Synthesis of 3-vinylfuran

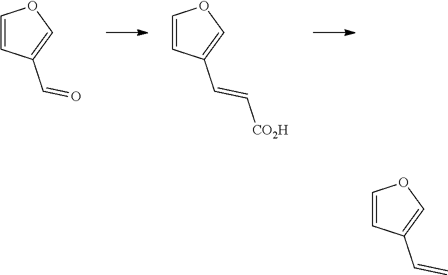

2.6 g of 3-vinylfuran was obtained in the same manner as Synthesis Example of 1,3-divinylbenzene except for using 3.9 g of 3-furanaldehyde instead of 1,3-benzenedicarboxylaldehyde.

The following peaks were found by carrying out ¹H-NMR measurement under the above measurement conditions, and the compound was confirmed to have the chemical structure of 3-vinylfuran.

δ (ppm) (d6-DMSO): 7.3 (1H, —CH═), 8.2 (1H, —CH═), 6.8 (1H, —CH═), 7.1 (1H, —CH═), 5.9 (1H, ═CH2), 5.4 (1H, ═CH2)

### Synthesis Example B7

### Synthesis of 3-vinylthiophene

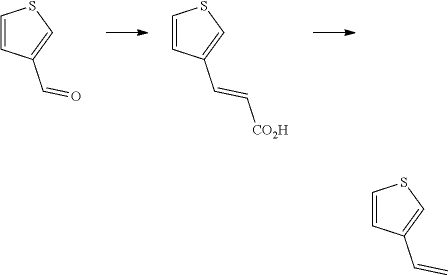

2.9 g of 3-vinylthiophene was obtained in the same manner as Synthesis Example of 1,3-divinylbenzene except for using 4.5 g of thiophene-3-aldehyde instead of 1,3-benzenedicarboxylaldehyde.

The following peaks were found by carrying out ¹H-NMR measurement under the above measurement conditions, and the compound was confirmed to have the chemical structure of 3-vinylthiophene.

δ (ppm) (d6-DMSO): 7.8 (1H, —CH═), 7.7 (1H, —CH═), 7.2 (1H, —CH═), 7.1 (1H, —CH═), 5.4 (1H, ═CH2), 5.9 (1H, ═CH2)

### Example AZ1

### Synthesis of Compound AZ1 Represented by Formula (MZ1)

Using a 200 mL glass flask as a reaction vessel, 4.9 g (40 mmol) of isopropylbenzene was dissolved using butanol as a solvent, a 20% by mass aqueous iodine chloride solution (121.8 g, 150 mmol) was added dropwise thereto at 50° C. over 60 minutes, and then the mixture was stirred at 50° C. for 2 hours to allow isopropylbenzene to react with iodine chloride. An aqueous sodium bisulfate solution was added to the reaction solution after the reaction, the mixture was stirred for 1 hour, and then the liquid temperature was cooled to 10° C. The precipitate precipitated by cooling was filtered off, washed, and dried to obtain 16.3 g of a white solid. The white solid sample was analyzed by liquid chromatography-mass spectrometry (LC-MS) and, as a result, confirmed to be 2,4,6-triiodoisopropylbenzene.

MnO₂ (3.4 g, 40 mmol) was added to a methylene chloride solvent and stirred, which was then stirred for 1 hour while a 50% by mass solution in which the total amount of the synthesized 2,4,6-triiodoisopropylbenzene was dissolved in methylene chloride was added dropwise and stirred at room temperature for 4 hours, and then the reaction solution was filtered off and the solvent was distilled off to obtain 16.1 g (31 mmol) of 2,4,6-triiodo-1′-hydroxyisopropylbenzene.

Using a 500 mL glass flask attached with a Dienstark tube as a reaction vessel, the total amount of the obtained 2,4,6-triiodo-1′-hydroxyisopropylbenzene was dissolved in a toluene solvent, and 0.6 g (6 mmol) of a concentrated sulfuric acid was added dropwise thereto with stirring, which were allowed to react under reflux conditions for 4 hours to obtain 13.3 g of the compound AZ1 (αmethyl-2,4,6-triiodostyrene (a compound represented by the formula (MZ1))). The inorganic element content and the organic impurity content were measured by the aforementioned method, and the results are shown in Table 1.

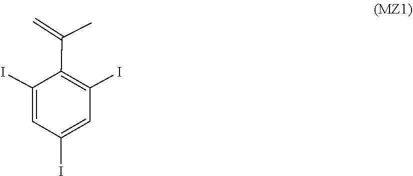

### Example AZ2

### Synthesis of Compound AZ2 Represented by Formula (MZ2)

Using a 200 mL glass flask as a reaction vessel, MeCN (80 mL), p-toluenesulfonic acid.H₂O (22.82 g, 120 mmol), and 3.3 g (20 mmol) of 2,4,6-triaminophenyl-1-ethanone were added thereinto. The obtained suspension solution was cooled to 0 to 5° C., and then a solution in which NaNO₂ (4.14 g, 60 mmol) was dissolved in water (9 mL) and a solution in which KI (12.5 g, 75 mmol) was dissolved in water (9 mL) was added thereto. After stirring at 0 to 5° C. for 10 minutes, the mixture was warmed to room temperature and stirred for 2 hours at the same temperature. Water (350 mL) was added to the reaction solution and the pH thereof was regulated to 9 with an aqueous 1M NaHCO₃ solution. Furthermore, an aqueous 2M Na₂S₂O₃ solution (40 mL) was added and then the mixture was extracted with EtOAc. The obtained organic layer was concentrated under reduced pressure, and then purified by silica gel chromatography (n-hexane:EtOAc=10:1) to obtain 8.5 g of 2′,4′,6′-triiodoacetophenone. (Yield 86%)

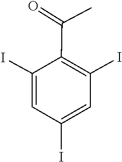

### Step 2: Styrenation Reaction

Using a 200 mL glass flask as a reaction vessel, 6.4 g (16.8 mmol) of triphenylphosphonium methyl bromide and 20 mL of toluene were put thereinto and dissolved. After a KTB solution in which 2.2 g (19.6 mmol) of potassium tert-butoxide was dissolved in 9 mL of THF was prepared, the KTB solution was added dropwise into a toluene solution which was put in an ice bath while the temperature was regulated to 0° C. or less, and then the mixture was stirred for 30 minutes as it was. While the temperature was further regulated to 0° C. or less, a solution in which 8.5 g (17.1 mmol) of 2′,4′,6′-triiodoacetophenone was dissolved in 15 mL of toluene was added dropwise, and then the mixture was stirred for 4 hours as it was. Thereafter, the mixture was further washed with 10 mL of water, 10 mL of 10% sodium bisulfite, 10 mL of 5% sodium bicarbonate water, and 10 mL of pure water, in the order presented. 5.9 g of the compound AZ2 (2,4,6-triiodophenyl-1-isopropene (a compound represented by the formula (MZ2))) as the target substance was isolated using a silica gel column.

The following peaks were found by carrying out ¹H-NMR measurement under the above measurement conditions, and the compound was confirmed to have the chemical structure of 2,4,6-triiodophenyl-1-isopropene.

δ (ppm) (d6-DMSO): 7.9 (2H, Ph), 2.1 (1H, —CH═), 5.1 (2H, ═CH2)

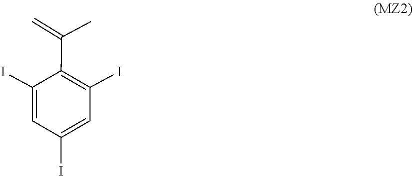

### Example AZ3

### Synthesis of Compound AZ3 Represented by Formula (MZ3)

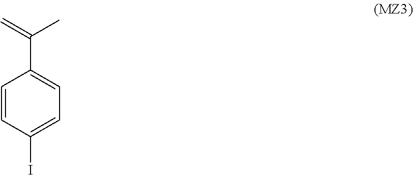

Step 1: Sandmeyer (Synthesis of 4′-iodoacetophenone)

Using a 200 mL glass flask as a reaction vessel, MeCN (80 mL), p-toluenesulfonic acid.H₂O (11.41 g, 60 mmol), and 4′-aminoacetophenone (2.70 g, 20 mmol) were added thereinto. The obtained suspension solution was cooled to 0 to 5° C., and then a solution in which NaNO₂ (2.76 g, 40 mmol) was dissolved in water (6 mL) and a solution in which KI (8.3 g, 50 mmol) was dissolved in water (6 mL) was added thereto. After stirring at 0 to 5° C. for 10 minutes, the mixture was warmed to room temperature and stirred for 2 hours at the same temperature. Water (350 mL) was added to the reaction solution and the pH thereof was regulated to 9 with an aqueous 1M NaHCO₃ solution. Furthermore, an aqueous 2M Na₂S₂O₃ solution (40 mL) was added and then the mixture was extracted with EtOAc. The obtained organic layer was concentrated under reduced pressure, and then purified by silica gel chromatography (n-hexane:EtOAc=10:1) to obtain 4.38 g of 4′-iodoacetophenone. (Yield 89%)

### Step 2: Styrenation Reaction

Using a 200 mL glass flask as a reaction vessel, 6.4 g (16.8 mmol) of triphenylphosphonium methyl bromide and 20 mL of toluene were put thereinto and dissolved. After a KTB solution in which 2.2 g (19.6 mmol) of potassium tert-butoxide was dissolved in 9 mL of THF was prepared, the KTB solution was added dropwise into a toluene solution which was put in an ice bath while the temperature was regulated to 0° C. or less, and then the mixture was stirred for 30 minutes as it was. While the temperature was further regulated to 0° C. or less, a solution in which 4.2 g (17.1 mmol) of 4′-iodoacetophenone was dissolved in 15 mL of toluene was added dropwise, and then the mixture was stirred for 4 hours as it was. Thereafter, the mixture was further washed with 10 mL of water, 10 mL of 10% sodium bisulfite, 10 mL of 5% sodium bicarbonate water, and 10 mL of pure water, in the order presented. 3.1 g of 1-iodo-4-isopropenylbenzene as the target substance was isolated using a silica gel column.

The following peaks were found by carrying out ¹H-NMR measurement under the above measurement conditions, and the compound was confirmed to have the chemical structure of the compound AZ3.

δ (ppm) (d6-DMSO): 7.3 (2H, Ph), 7.7 (2H, Ph), 2.1 (3H, —CH3), 5.1 (1H, ═CH2), 5.3 (1H, ═CH2)

### Example AZ4

### Synthesis of Compound MZ4 Represented by Formula (MZ4)

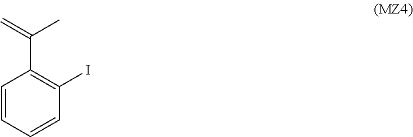

2.9 g of 1-iodo-2-isopropenylbenzene as the target substance was isolated in the same manner except for using 2′-aminoacetophenone (2.70 g, 20 mmol) instead of 4′-aminoacetophenone in Example AZ3.

The following peaks were found by carrying out ¹H-NMR measurement under the above measurement conditions, and the compound was confirmed to have the chemical structure of the compound MZ4.

δ (ppm) (d6-DMSO): 7.7 (1H, Ph), 7.5 (1H, Ph), 7.4 (1H, Ph), 7.1 (1H, Ph), 2.1 (3H, —CH3), 5.1 (2H, ═CH2)

### Example AZ5

### Synthesis of Compound MZ5 Represented by Formula (MZ5)

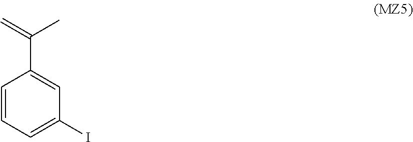

2.6 g of 1-iodo-3-isopropenylbenzene as the target substance was isolated in the same manner except for using 3′-aminoacetophenone (2.70 g, 20 mmol) instead of 4′-aminoacetophenone in Example AZ3.

The following peaks were found by carrying out ¹H-NMR measurement under the above measurement conditions, and the compound was confirmed to have the chemical structure.

δ (ppm) (d6-DMSO): 7.7 (2H, Ph), 7.0 (1H, Ph), 6.8 (1H, Ph), 2.1 (1H, —CH═), 5.0 (1H, ═CH2), 5.3 (1H, ═CH2)

### Example AZ6

### Synthesis of Compound MZ6 Represented by Formula (MZ6)

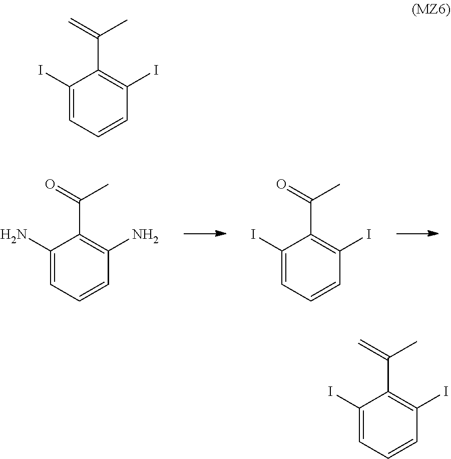

Step 1: Sandmeyer (Synthesis of 2′,6′-diiodoacetophenone)

Using a 200 mL glass flask as a reaction vessel, MeCN (80 mL), p-toluenesulfonic acid.H₂O (11.41 g, 60 mmol), and 2′,6′-diaminoacetophenone (3.0 g, 20 mol) were added thereinto. The obtained suspension solution was cooled to 0 to 5° C., and then a solution in which NaNO₂ (5.52 g, 80 mmol) was dissolved in water (6 mL) and a solution in which KI (16.6 g, 100 mmol) was dissolved in water (12 mL) was added thereto. After stirring at 0 to 5° C. for 10 minutes, the mixture was warmed to room temperature and stirred for 2 hours at the same temperature. Water (350 mL) was added to the reaction solution and the pH thereof was regulated to 9 with an aqueous 1M NaHCO₃ solution. Furthermore, an aqueous 2M Na₂S₂O₃ solution (40 mL) was added and then the mixture was extracted with EtOAc. The obtained organic layer was concentrated under reduced pressure, and then purified by silica gel chromatography (n-hexane:EtOAc=10:1) to obtain 6.7 g of 2′,6′-diiodoacetophenone. (Yield 90%)

### Step 2: Wittig

Using a 200 mL glass flask as a reaction vessel, 6.4 g (16.8 mmol) of triphenylphosphonium methyl bromide and 20 mL of toluene were put thereinto and dissolved. After a KTB solution in which 2.2 g (19.6 mmol) of potassium tert-butoxide was dissolved in 9 mL of THF was prepared, the KTB solution was added dropwise into a toluene solution which was put in an ice bath while the temperature was regulated to 0° C. or less, and then the mixture was stirred for 30 minutes as it was. While the temperature was further regulated to 0° C. or less, a solution in which 6.7 g (18.0 mmol) of 4′-iodoacetophenone was dissolved in 15 mL of toluene was added dropwise, and then the mixture was stirred for 4 hours as it was. Thereafter, the mixture was further washed with 10 mL of water, 10 mL of 10% sodium bisulfite, 10 mL of 5% sodium bicarbonate water, and 10 mL of pure water, in the order presented. 6.3 g of 1,3-diiodo-2-isopropenylbenzene as the target substance was isolated using a silica gel column.

The following peaks were found by carrying out ¹H-NMR measurement under the above measurement conditions, and the compound was confirmed to have the chemical structure of 1,3-diiodo-2-isopropenylbenzene.

δ (ppm) (d6-DMSO): 7.7 (2H, Ph), 6.8 (1H, Ph), 2.1 (1H, —CH═), 5.1 (1H, ═CH2), 5.2 (1H, ═CH2)

### Example AZ7

### Synthesis of Compound MZ7 Represented by Formula (MZ7)

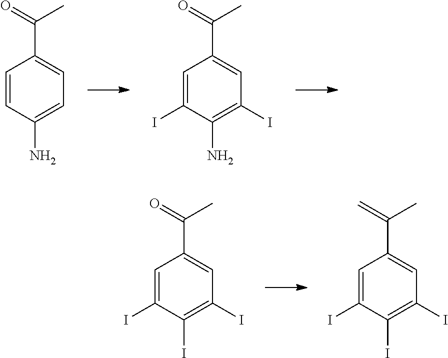

### Step 1: Iodine Source+Oxidizing Agent/Methanol (Diiodination)

After 4.0 g (29.6 mmol) of 4-aminoacetophenone was dissolved in 20 mL of toluene, 7.6 g (90 mmol) of NaHCO_{3/100} mL of water was added, 18.0 g (70.8 mmol) of 12 was added, and the mixture was stirred at 25° C. for 20 hours. Thereafter, 40 mL of an aqueous saturated Na2SO3 solution was added, the mixture was stirred for 10 minutes, 120 mL of ethyl acetate and 10 mL of pure water were added, and the ethyl acetate phase was extracted. The extracted ethyl acetate phase was washed with brine, magnesium sulfate was added and stirred, and the mixture was dried overnight. After magnesium sulfate was filtered off, the filtrate was concentrated and separated by chromatography to obtain 11.1 g of 3,5-diiodo-4-aminoacetophenone as the target substance.

Step 2: Iodine Substitution Reaction (Synthesis of 3′,4′,5′-triiodoacetophenone)

Using a 200 mL glass flask as a reaction vessel, MeCN (80 mL), p-toluenesulfonic acid.H₂O (11.41 g, 60 mmol), and 3,5-diiodo-4-aminoacetophenone (7.73 g, 20 mmol) were added thereinto. The obtained suspension solution was cooled to 0 to 5° C., and then a solution in which NaNO₂ (2.76 g, 40 mmol) was dissolved in water (6 mL) and a solution in which KI (8.3 g, 50 mmol) was dissolved in water (12 mL) was added thereto. After stirring at 0 to 5° C. for 10 minutes, the mixture was warmed to room temperature and stirred for 2 hours at the same temperature. Water (350 mL) was added to the reaction solution and the pH thereof was regulated to 9 with an aqueous 1M NaHCO₃ solution. Furthermore, an aqueous 2M Na₂S₂O₃ solution (40 mL) was added and then the mixture was extracted with EtOAc. The obtained organic layer was concentrated under reduced pressure, and then purified by silica gel chromatography (n-hexane:EtOAc=10:1) to obtain 9.0 g of 3′,4′,5′-triiodoacetophenone.

### Step 3: Styrenation Reaction

Using a 200 mL glass flask as a reaction vessel, 6.4 g (16.8 mmol) of triphenylphosphonium methyl bromide and 20 mL of toluene were put thereinto and dissolved. After a KTB solution in which 2.2 g (19.6 mmol) of potassium tert-butoxide was dissolved in 9 mL of THF was prepared, the KTB solution was added dropwise into a toluene solution which was put in an ice bath while the temperature was regulated to 0° C. or less, and then the mixture was stirred for 30 minutes as it was. While the temperature was further regulated to 0° C. or less, a solution in which 9.0 g (18.1 mmol) of 3′,4′,5′-triiodoacetophenone was dissolved in 15 mL of toluene was added dropwise, and then the mixture was stirred for 4 hours as it was. Thereafter, the mixture was further washed with 10 mL of water, 10 mL of 10% sodium bisulfite, 10 mL of 5% sodium bicarbonate water, and 10 mL of pure water, in the order presented. 5.5 g of 3,4,5-triiodo-4-isopropenylbenzene as the target substance was isolated using a silica gel column.

The following peaks were found by carrying out ¹H-NMR measurement under the above measurement conditions, and the compound was confirmed to have the following chemical structure.

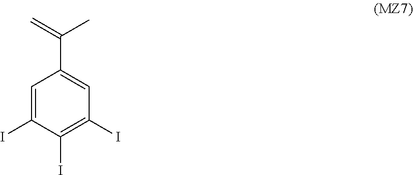

δ (ppm) (d6-DMSO): 7.4 (2H, Ph), 2.1 (1H, —CH═), 5.0 (1H, ═CH2), 5.3 (1H, ═CH2)

### Example AZ8

### Synthesis of Compound MZ8 Represented by Formula (MZ8)

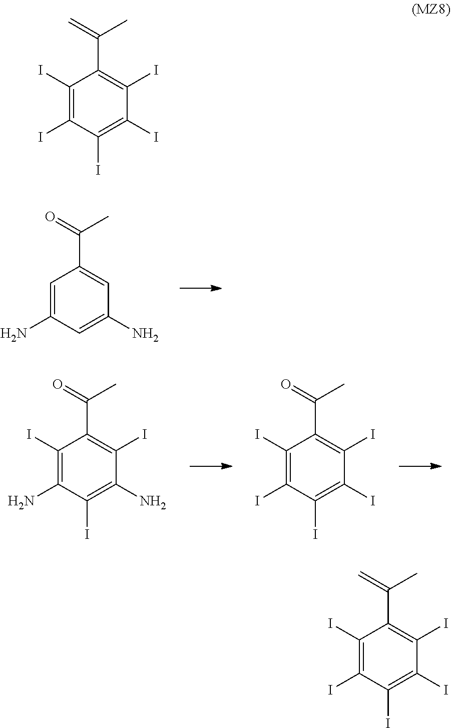

### Step 1: Diiodination by Iodine Introduction Reaction

After 4.5 g (29.6 mmol) of 3,5-diaminoacetophenone was dissolved in 20 mL of toluene, 11.4 g (135 mmol) of NaHCO_{3/100} mL of water was added, 27.0 g (106.2 mmol) of 12 was added, and the mixture was stirred at 25° C. for 20 hours. Thereafter, 40 mL of an aqueous saturated Na2SO3 solution was added, the mixture was stirred for 10 minutes, 120 mL of ethyl acetate and 10 mL of pure water were added, and the ethyl acetate phase was extracted. The extracted ethyl acetate phase was washed with brine, magnesium sulfate was added and stirred, and the mixture was dried overnight. After magnesium sulfate was filtered off, the filtrate was concentrated and separated by chromatography to obtain 14.4 g of 2,4,6-triiodo-3,5-diaminoacetophenone as the target substance.

### Step 2: Iodine Substitution Reaction

Using a 200 mL glass flask as a reaction vessel, MeCN (80 mL), p-toluenesulfonic acid.H₂O (22.82 g, 120 mmol), and 2,4,6-triiodo-3,5-diaminoacetophenone (10.6 g, 20 mmol) were added thereinto. The obtained suspension solution was cooled to 0 to 5° C., and then a solution in which NaNO₂ (5.52 g, 80 mmol) was dissolved in water (12 mL) and a solution in which KI (16.6 g, 100 mmol) was dissolved in water (12 mL) was added thereto. After stirring at 0 to 5° C. for 10 minutes, the mixture was warmed to room temperature and stirred for 2 hours at the same temperature. Water (350 mL) was added to the reaction solution and the pH thereof was regulated to 9 with an aqueous 1M NaHCO₃ solution. Furthermore, an aqueous 2M Na₂S₂O₃ solution (40 mL) was added and then the mixture was extracted with EtOAc. The obtained organic layer was concentrated under reduced pressure, and then purified by silica gel chromatography (n-hexane:EtOAc=10:1) to obtain 12.8 g of 2′,3′,4′,5′,6′-pentaiodoacetophenone.

### Step 3: Styrenation Reaction

Using a 200 mL glass flask as a reaction vessel, 6.4 g (16.8 mmol) of triphenylphosphonium methyl bromide and 20 mL of toluene were put thereinto and dissolved. After a KTB solution in which 2.2 g (19.6 mmol) of potassium tert-butoxide was dissolved in 9 mL of THF was prepared, the KTB solution was added dropwise into a toluene solution which was put in an ice bath while the temperature was regulated to 0° C. or less, and then the mixture was stirred for 30 minutes as it was. While the temperature was further regulated to 0° C. or less, a solution in which 12.8 g (17.1 mmol) of 2′,3′,4′,5′,6′-pentaiodoacetophenone was dissolved in 15 mL of toluene was added dropwise, and then the mixture was stirred for 4 hours as it was. Thereafter, the mixture was further washed with 10 mL of water, 10 mL of 10% sodium bisulfite, 10 mL of 5% sodium bicarbonate water, and 10 mL of pure water, in the order presented. 7.6 g of 2′,3′,4′,5′,6′-pentaiodo-4-isopropenylbenzene as the target substance was isolated using a silica gel column.

The following peaks were found by carrying out ¹H-NMR measurement under the above measurement conditions, and the compound was confirmed to have the chemical structure.

δ (ppm) (d6-DMSO): 2.1 (1H, —CH═), 5.1 (1H, ═CH2), 5.2 (1H, ═CH2) (MZ12)

### Example AZ9

### Synthesis of Compound MZ9 Represented by Formula (MZ9)

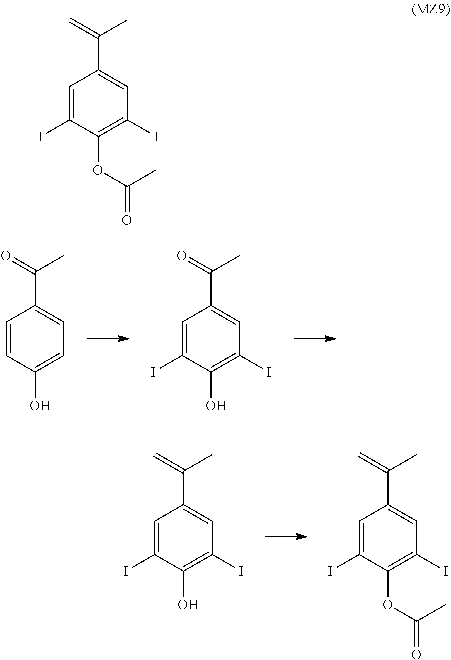

### (Step 1) Iodination Reaction

Using a 200 mL glass flask as a reaction vessel, 5.45 g (40 mmol) of 4-hydroxyacetophenone was dissolved using butanol as a solvent, a 20% by mass aqueous iodine chloride solution (81.2 g, 100 mmol) was added dropwise thereto at 50° C. over 60 minutes, and then the mixture was stirred at 50° C. for 2 hours to allow 4-hydroxybenzaldehyde to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 15.2 g of a white solid. The white solid sample was analyzed by liquid chromatography-mass spectrometry (LC-MS) and, as a result, confirmed to be 4-hydroxy-3,5-diiodoacetophenone.

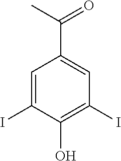

### (Step 2) Malonic Acid Addition Reaction

Using a 200 mL eggplant flask equipped with a Dienstark reflux tube, 15.2 g (39 mmol) of 4-hydroxy-3,5-diiodoacetophenone was mixed with malonic acid (6.24 g, 60 mmol), piperidine (3.4 g, 40 mmol), acetic acid (2.4 g, 40 mmol), and 40 mL of benzene, and allowed to react for 3 hours under reflux conditions. The obtained reaction solution was washed with 20 mL of a 5% by mass aqueous HCl solution, and then washed with a 5% aqueous NaHCO3 solution. The obtained organic phase was dried over magnesium sulfate, and then concentrated under reduced pressure to obtain 16.3 g of a reaction product (MZ9-CA) (a mixture of cis form and trans form) consisting of a cinnamic acid derivative.

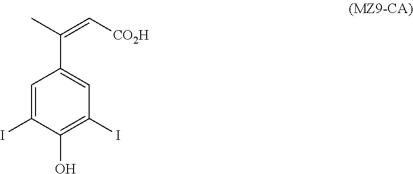

### (Step 3) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 16.3 g (38 mmol) of the cinnamic acid derivative MZ9-CA prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 14.2 g of a compound (MZ9-OH) represented by the formula (MZ9-OH).

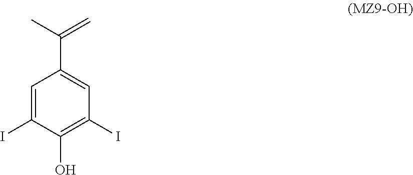

### (Step 4) Acetyl Protecting Group Introduction Reaction

Using a 1 L eggplant flask, 6.1 g (60 mmol) of acetic anhydride, 6.0 g (60 mmol) of triethylamine, 0.8 g (6 mmol) of DMAP, and 350 mL of a solvent (dichloromethane), which were cooled to 4° C. with ice water, were put thereinto, and stirred and dissolved to prepare a reaction solution. Under cooling with ice at 4° C., 14.2 g, 37 mmol of the compound MZ9-OH prepared in the previous step was dissolved in 50 mL of dichloromethane to prepare a solution of the compound MZ9-OH, which was added to the solution prepared in the 1 L eggplant flask over 30 minutes. Thereafter, the mixture was stirred at 4° C. for 2 hours to allow the reaction to proceed sufficiently and washed with 400 mL of ice water and 400 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 14.9 g of the target compound MZ9.

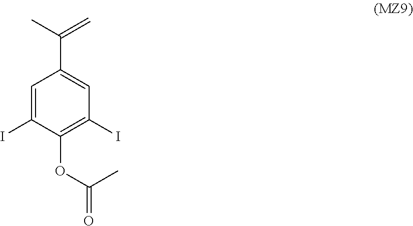

The following peaks were found by carrying out ¹H-NMR measurement under the above measurement conditions, and the compound was confirmed to have the chemical structure of the compound MZ.

δ (ppm) (d6-DMSO): 2.3 (3H, —CH3), 7.7 (2H, Ph), 2.1 (1H, —CH═), 5.0 (1H, ═CH2), 5.3 (1H, ═CH2)

### Example AZ10

### Synthesis of Compound MZ10 Represented by Formula (MZ10)

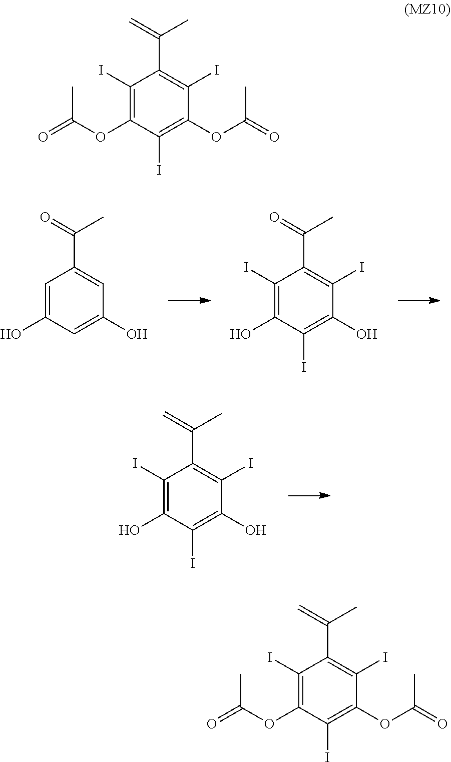 [SMILES: C=C(C)c1c(I)c(OC(C)=O)c(I)c(OC(C)=O)c1I ; CC(=O)c1cc(O)cc(O)c1 ; CC(=O)c1c(I)c(O)c(I)c(O)c1I ; C=C(C)c1c(I)c(O)c(I)c(O)c1I ; C=C(C)c1c(I)c(OC(C)=O)c(I)c(OC(C)=O)c1I]

### (Step 1) Iodination Reaction

Using a 200 mL glass flask as a reaction vessel, 6.09 g (40 mmol) of 3,5-dihydroxyacetophenone was dissolved using butanol as a solvent, a 20% by mass aqueous iodine chloride solution (121.8 g, 150 mmol) was added dropwise thereto at 50° C. over 60 minutes, and then the mixture was stirred at 50° C. for 2 hours to allow 4-hydroxybenzaldehyde to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 20.1 g of a white solid. The white solid sample was analyzed by liquid chromatography-mass spectrometry (LC-MS) and, as a result, confirmed to be 3,5-dihydroxy-2,4,6-triiodoacetophenone.

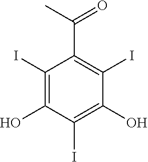

### (Step 2) Malonic Acid Addition Reaction

Using a 200 mL eggplant flask equipped with a Dienstark reflux tube, 20.1 g (38 mmol) of 3,5-dihydroxy-2,4,6-triiodoacetophenone was mixed with malonic acid (6.24 g, 60 mmol), piperidine (3.4 g, 40 mmol), acetic acid (2.4 g, 40 mmol), and 40 mL of benzene, and allowed to react for 3 hours under reflux conditions. The obtained reaction solution was washed with 20 mL of a 5% by mass aqueous HCl solution, and then washed with a 5% aqueous NaHCO3 solution. The obtained organic phase was dried over magnesium sulfate, and then concentrated under reduced pressure to obtain 21.1 g of a reaction product (MZ10-CA) (a mixture of cis form and trans form) consisting of a cinnamic acid derivative.

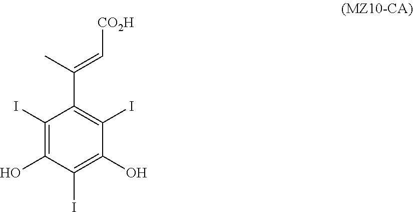 [SMILES: C/C(=C\C(=O)O)c1c(I)c(O)c(I)c(O)c1I]

### (Step 3) Decarboxylation Reaction

Using a 1 L eggplant flask, a solution in which 0.13 g (0.4 mmol) of tetrabutylammonium fluoride trihydrate was dissolved in 20 mL of dimethylsulfoxide was slowly added to a solution in which 21.1 g (37 mmol) of the cinnamic acid derivative MZ10-CA prepared above was dissolved in 40 mL of dimethylsulfoxide at 10° C. and stirred, and then the mixture was warmed to 40° C. and stirred for 12 hours. The obtained reaction solution was washed three times with 20 mL of pure water, dried over magnesium sulfate, and the filtrate obtained after filtration was concentrated under reduced pressure to obtain 19.0 g of a compound (MZ10-OH) represented by the formula (MZ10-OH).

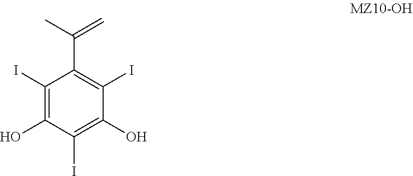

### (Step 4) Acetyl Protecting Group Introduction Reaction

Using a 1 L eggplant flask, 6.1 g (60 mmol) of acetic anhydride, 6.0 g (60 mmol) of triethylamine, 0.8 g (6 mmol) of DMAP, and 350 mL of a solvent (dichloromethane), which were cooled to 4° C. with ice water, were put thereinto, and stirred and dissolved to prepare a reaction solution. Under cooling with ice at 4° C., 19.0 g, 36 mmol of the compound MZ10-OH prepared in the previous step was dissolved in 50 mL of dichloromethane to prepare a solution of the compound MZ10-OH, which was added to the solution prepared in the 1 L eggplant flask over 30 minutes. Thereafter, the mixture was stirred at 4° C. for 2 hours to allow the reaction to proceed sufficiently and washed with 400 mL of ice water and 400 mL of brine sufficiently, the obtained organic phase was dried over magnesium sulfate, the filtrate after filtration was concentrated under reduced pressure to obtain a reaction product. The reaction product was further purified by column and the developing solvent was distilled off to collect 21.1 g of the target compound MZ10.

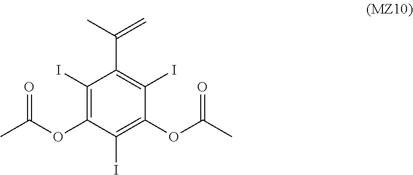 [SMILES: C=C(C)c1c(I)c(OC(C)=O)c(I)c(OC(C)=O)c1I]

The following peaks were found by carrying out ¹H-NMR measurement under the above measurement conditions, and the compound was confirmed to have the chemical structure of the compound MZ10.

δ (ppm) (d6-DMSO): 2.3 (6H, —CH3), 2.1 (1H, —CH═), 5.1 (1H, ═CH2), 5.2 (1H, ═CH2)

### Example AZ11

### Synthesis of Compound MZ11 Represented by Formula (MZ11)

The compound MZ11 represented by the formula (MZ11) was synthesized by the method described below.

(Step 1) Diiodination of 4-methoxyacetophenone

Using a 200 mL glass flask as a reaction vessel, 6.8 g (45 mmol) of 4-methoxyacetophenone was dissolved using butanol as a solvent, a 20% by mass aqueous iodine chloride solution (81.2 g, 100 mmol) was added dropwise thereto at 50° C. over 60 minutes, and then the mixture was stirred at 50° C. for 2 hours to allow 4-methoxyacetophenone to react with iodine chloride. An aqueous sodium thiosulfate solution was added to the reaction solution after the reaction, the mixture was stirred for 1 hour, and then the liquid temperature was cooled to 10° C. The precipitate precipitated by cooling was filtered off, washed, and dried to obtain 15.3 g of a white solid. The white solid sample was analyzed by liquid chromatography-mass spectrometry (LC-MS) and, as a result, confirmed to be a compound represented by the formula (MZ11-AP).

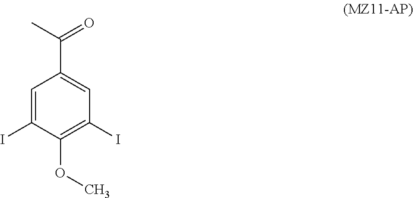

### Step 3: Styrenation Reaction

Using a 200 mL glass flask as a reaction vessel, 6.4 g (16.8 mmol) of triphenylphosphonium methyl bromide and 20 mL of toluene were put thereinto and dissolved. After a KTB solution in which 2.2 g (19.6 mmol) of potassium tert-butoxide was dissolved in 9 mL of THF was prepared, the KTB solution was added dropwise into a toluene solution which was put in an ice bath while the temperature was regulated to 0° C. or less, and then the mixture was stirred for 30 minutes as it was. While the temperature was further regulated to 0° C. or less, a solution in which 8.0 g (20 mmol) of the compound represented by the formula (MZ11-AP) was dissolved in 15 mL of toluene was added dropwise, and then the mixture was stirred for 4 hours as it was. Thereafter, the mixture was further washed with 10 mL of water, 10 mL of 10% sodium bisulfite, 10 mL of 5% sodium bicarbonate water, and 10 mL of pure water, in the order presented. 9.6 g of the compound represented by the formula (MZ11) as the target substance was isolated using a silica gel column.

The following peaks were found by carrying out ¹H-NMR measurement under the above measurement conditions, and the compound was confirmed to have the chemical structure of the compound represented by the formula (MZ11).

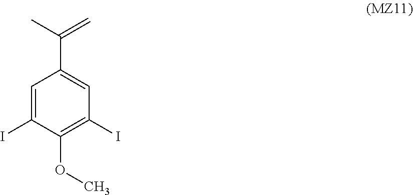

δ (ppm) (d6-DMSO): 3.9 (3H, —CH3), 7.7 (2H, Ph), 2.1 (1H, —CH═), 5.0 (1H, ═CH2), 5.3 (1H, ═CH2)

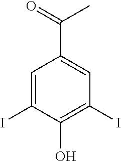

### (Raw Material: Acetophenone Derivative) Mw 387.94

### Reference Example AA1-D

MAD-1 was synthesized by conducting step 2 and subsequent steps without conducting step 1 in Example A9 (synthesis of M9).

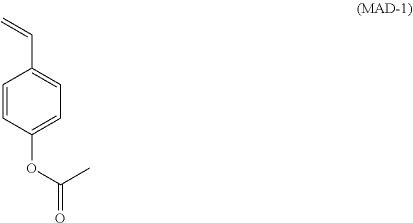

### Reference Example AA2-D

MAD-2 was synthesized by conducting step 2 and subsequent steps without conducting step 1 in Example A10 (synthesis of M10).

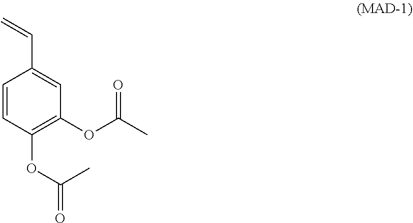 [SMILES: C=Cc1ccc(OC(C)=O)c(OC(C)=O)c1]

### Comparative Example A1

As a compound AR1, p-hydroxystyrene (a compound represented by the following formula (MR1) manufactured by TOHO Chemical Industry Co., Ltd.) was used. The inorganic element content and the organic impurity content were measured by the aforementioned method, and the results are shown in Table 1.

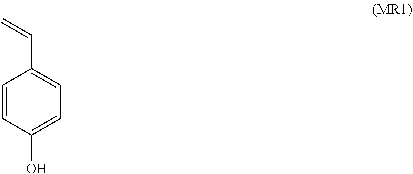

### Comparative Example A2

### Synthesis of Compound AR2 Represented by Formula (MR2)

In a 2 L flask, 400 mL of dichloromethane, 13.3 g of the compound AR1, 16.2 g of triethylamine, and 0.7 g of N-(4-pyridyl)dimethylamine (DMAP) was dissolved in a nitrogen flow. 33.6 g of di-tert-butyl dicarbonate was dissolved in 100 mL of dichloromethane, which was added dropwise into the above 2 L flask with stirring, and the mixture was stirred at room temperature for 3 hours. Thereafter, water washing was conducted three times by a separation operation using 100 mL of water, the solvent was distilled off from the obtained organic phase, original components were removed by silica gel chromatography using dichloromethane/hexane, and the solvent was further distilled off to obtain 4.1 g of a BOC group-substituted compound (a compound represented by the following formula (MR2), hereinafter, also referred to as the “compound AR2”) of the compound AR1, as the target component. The inorganic element content and the organic impurity content were measured by the aforementioned method, and the results are shown in Table 1.

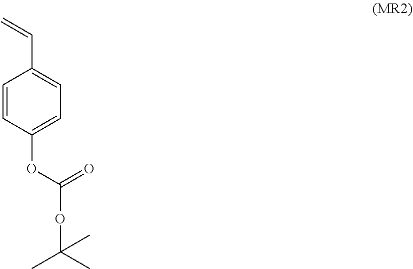 [SMILES: C=Cc1ccc(OC(=O)OC(C)(C)C)cc1]

### Comparative Example A3

### Synthesis of Compound AR3 Represented by Formula (MR3)

Using a 200 mL glass flask as a reaction vessel, MnO₂ (3.4 g, 40 mmol) was added to a methylene chloride solvent and stirred, which was then stirred for 1 hour while a 50% by mass solution in which 5.4 g (40 mmol) of 4-isopropylphenol was dissolved in methylene chloride was added dropwise and stirred at room temperature for 4 hours, and then the reaction solution was filtered off and the solvent was distilled off to obtain 5.3 g (35 mmol) of 1′-hydroxy-4-isopropylphenol.

Using a 500 mL glass flask attached with a Dienstark tube as a reaction vessel, the total amount of the obtained 1′-hydroxy-4-isopropylphenol was dissolved in a toluene solvent, and 0.6 g (6 mmol) of a concentrated sulfuric acid was added dropwise thereto with stirring, which were allowed to react under reflux conditions for 4 hours to obtain 4.9 g of the compound AR3 (αmethyl-4-hydroxystyrene (a compound represented by the formula (MR3))). The inorganic element content and the organic impurity content were measured by the aforementioned method, and the results are shown in Table 1.

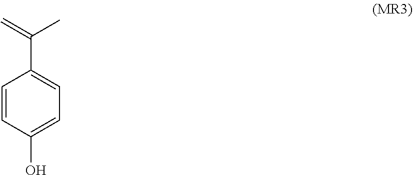

### Comparative Example A4

As the compound AR1, 3,4-dihydroxystyrene (a compound represented by the following formula (MR4) manufactured by TOHO Chemical Industry Co., Ltd.) was used. The inorganic element content and the organic impurity content were measured by the aforementioned method, and the results are shown in Table 1.

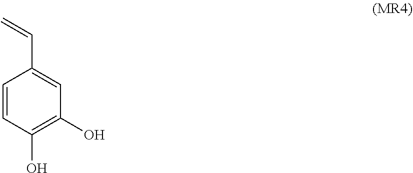

For the compounds synthesized in the above Examples and Comparative Examples, the inorganic element content and the organic impurity content were measured by the aforementioned method, and the results are shown in Table 1.

TABLE 1

| | | | Monomer physical properties | | | | |
|---|---|---|---|---|---|---|---|
| | | | Impurity content (ppm) | | | | |
| | Com- | | | | | | Phosphorus- |
| | pound | Structural | | | | Maleic | containing |
| | No. | formula | Mn | Al | K | acid | compound |
| Example A1 | A1 | M1 | 2 | 2 | 11 | 3 | 5 |
| Example A2 | A2 | M2 | 2 | 2 | 11 | 3 | 5 |
| Example A3 | A3 | M3 | 2 | 2 | 11 | 3 | 5 |
| Example A4 | A4 | M4 | 2 | 2 | 11 | 3 | 5 |
| Example A5 | A5 | M5 | 2 | 2 | 11 | 3 | 5 |
| Example A6 | A6 | M6 | 2 | 2 | 11 | 3 | 5 |
| Example A7 | A7 | M7 | 2 | 2 | 11 | 3 | 5 |
| Example A8 | A8 | M8 | 2 | 2 | 6 | 3 | 5 |
| Example A9 | A9 | M9 | 2 | 2 | 9 | 2 | 4 |
| Example A10 | A10 | M10 | 1 | 1 | 14 | 2 | 6 |
| Example A11 | A11 | M11 | 2 | 2 | 3 | 4 | 6 |
| Example Al2 | A12 | M12 | 2 | 2 | 5 | 2 | 3 |
| Example A13 | A13 | M13 | 2 | 1 | 7 | 2 | 5 |
| Example A14 | A14 | M14 | 2 | 1 | 6 | 4 | 7 |
| Example A15 | A15 | M15 | 1 | 1 | 13 | 4 | 4 |
| Example A16 | A16 | M16 | 1 | 1 | 12 | 6 | 5 |
| Example AH1 | AH1 | MH1 | 2 | 2 | 11 | DL | 34 |
| Example AH2 | AH2 | MH2 | 2 | 2 | 11 | DL | 25 |
| Example ACL1 | MCL1 | MCL1 | 2 | 2 | 8 | DL | 72 |
| Example AZ1 | AZ1 | MZ1 | 2 | 1 | 5 | DL | 31 |
| Example AZ2 | AZ2 | MZ2 | 1 | 3 | 5 | DL | 22 |
| Example AZ3 | AZ3 | MZ3 | 2 | 1 | 4 | DL | 14 |
| Example AZ4 | AZ4 | MZ4 | 2 | 1 | 4 | DL | 9 |
| Example AZ5 | AZ5 | MZ5 | 1 | 2 | 5 | DL | 23 |
| Example AZ6 | AZ6 | MZ6 | 3 | 1 | 4 | DL | 21 |
| Example AZ7 | AZ7 | MZ7 | 1 | 3 | 6 | DL | 15 |
| Example AZ8 | AZ8 | MZ8 | 2 | 1 | 5 | DL | 32 |
| Example AZ9 | AZ9 | MZ9 | 1 | 3 | 6 | DL | 26 |
| Example AZ10 | AZ10 | MZ10 | 2 | 1 | 6 | DL | 13 |
| Example AZ11 | AZ11 | MZ11 | 3 | 1 | 4 | DL | 11 |
| Comparative | AR1 | MR1 | 2 | 2 | 11 | 3 | 5 |
| Example A1 | | | | | | | |
| Comparative | AR2 | MR2 | 2 | 2 | 11 | 3 | 5 |
| Example A2 | | | | | | | |
| Comparative | AR3 | MR3 | 1 | 2 | 5 | DL | DL |
| Example A3 | | | | | | | |
| Comparative | AR4 | MR4 | 2 | 2 | 11 | 3 | 5 |
| Example A4 | | | | | | | |

DL: Below detection limit

<Aging Stability in Solution State>

As an index of stability of materials, the stability of the prepared compounds in a solution state was evaluated by the following method. That is:

A clean bottle manufactured by AICELLO CORPORATION was charged with the prepared compound A alone or a mixture of a plurality of compounds, and then a solvent and capped, the prepared solution sample was stirred by a mixing rotor for 2 hours to prepare a dissolved sample. The prepared sample in clean bottle was subjected to aging test under the predetermined temperature conditions. The prepared test sample was analyzed and evaluated by high-performance liquid chromatography, and the aging stability of the solution was evaluated from the purity value at the main peak.

As aging conditions, two conditions of a temperature of 4° C. (condition A) and a temperature of 40° C. (condition B) were selected, and the sample was evaluated based on the index value determined according to the following equation from the amount of change of the purity value at the main peak after 240 hours.

Index value=(purity at 40° C.)/(purity at 4° C.)×100

A Index value≥99.5

B 99.5>Index value≥99.0

C 99.0>Index value≥98.0

D 98.0>Index value≥95.0

E 95>Index value

### Example B1

### Synthesis of Polymer

1.5 g of the compound A1, 3.0 g of 2-methyl-2-adamantyl methacrylate, 2.0 g of γ-butyrolactone methacrylate, and 1.5 g of hydroxyadamantyl methacrylate were dissolved in 45 mL of tetrahydrofuran, and 0.20 g of azobisisobutyronitrile was added thereto. After the mixture was refluxed for 12 hours, the reaction solution was added dropwise to 2 L of n-heptane. The polymer precipitated was filtered off and dried under reduced-pressure to obtain a white powdery polymer B1 represented by the following formula (MA1). This polymer had a weight average molecular weight (Mw) of 1,2000 and a dispersity (Mw/Mn) of 1.90. As a result of the measurement of ¹³C-NMR, the composition ratio (molar ratio) in the following formula (MA1) was a:b:c:d=40:30:15:15. Although the following formula (MA1) is illustrated in a simplified form to show the ratio of respective constitutional units, the order of arrangement of respective constitutional units is random and this polymer is not a block copolymer in which respective constitutional units form an independent block. The inorganic element content and organic impurity content of the polymer were measured by the aforementioned method, and the results are shown in Table 3. The molar ratio was determined based on the integral ratio of each of the carbon on the bottom of a benzene ring for the polystyrene monomer (the compound A1) and the carbonyl carbon of an ester bond for methacrylate monomer (2-methyl-2-adamantyl methacrylate, γ-butyrolactone methacrylate, and hydroxyadamantyl methacrylate). The kind of respective monomers, the ratio thereof, and the composition ratio in the polymer obtained in Example B1 are shown in Table 2. The kind of respective monomers, the ratio thereof, and the composition ratio in the polymers obtained in Examples described below are also shown in Table 2.

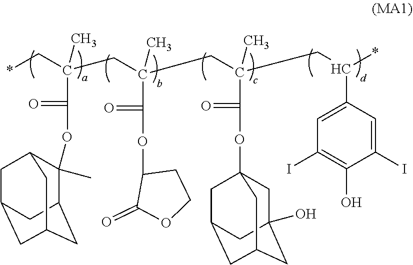

### Example B3 and B5 to B9 and Comparative Example B1 to B2

### Synthesis of Polymer

The polymers B3 and B5 to B9 and BR1 to BR2 represented by the formulas (MA2) to (MA7) and the formulas (MAR1) to (MAR2) were obtained by the method described in Example B1, except that 1.5 g of the compound A1 was changed to the kind and the amount shown in Table 2. The inorganic element content and organic impurity content of the polymer were measured by the aforementioned method, and the results are shown in Table 3.

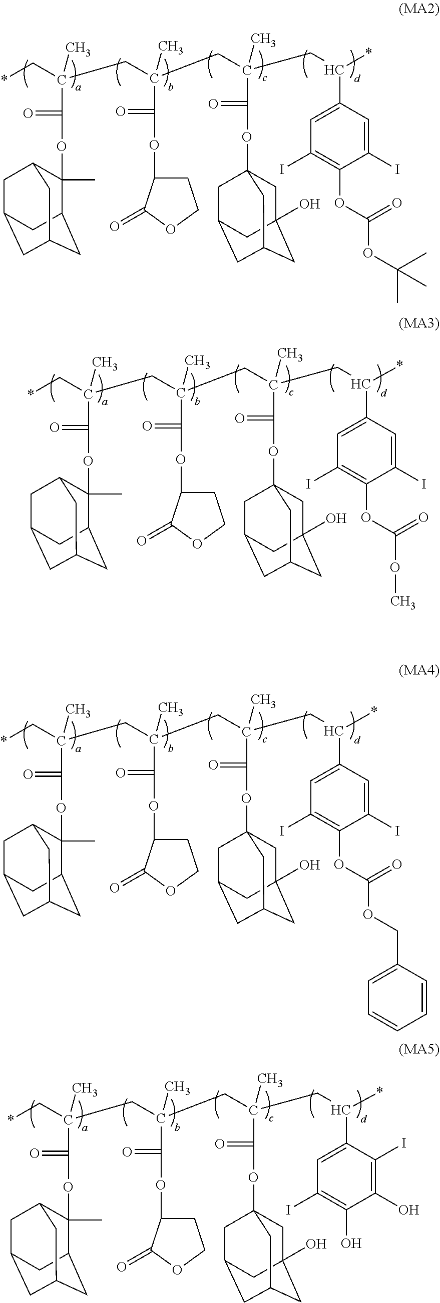

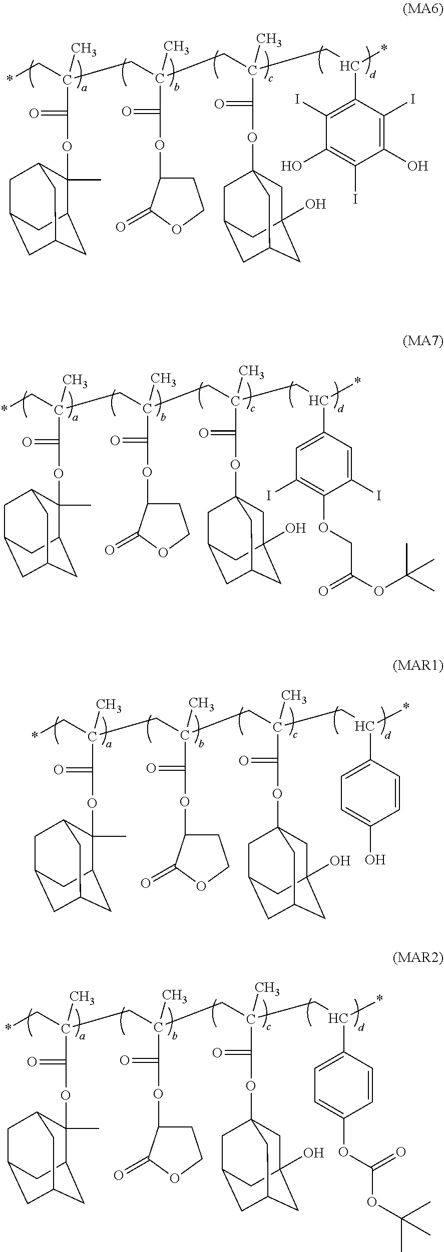

### Example B2

### Synthesis of Polymer B2

For the synthesized compound A1, purification treatments of respective raw materials were additionally conducted before synthesis of the polymer. Using ethyl acetate (PrimePure manufactured by Kanto Chemical Co., Inc.) as a solvent, a 10% by mass ethyl acetate solution of the compound A1 in which the compound A1 was dissolved was prepared. To remove metal impurities, an ion exchange resin “AMBERLYST MSPS2-lDRY” (product name, manufactured by ORGANO CORPORATION) was immersed in ethyl acetate (PrimePure manufactured by Kanto Chemical Co., Inc.), stirred for 1 hour, and then the solvent was removed. Washing of the ion exchange resin was carried out by repeating washing in this manner 10 times. In the above ethyl acetate solution of the compound A1, the same mass of the washed ion exchange resin as the resin solid content was put and stirred at room temperature for a day, and then the ion exchange resin was filtered off. Washing in which an ion exchange treatment was carried out in this manner was repeated three times to prepare an ion-exchanged ethyl acetate solution of the compound A1. Furthermore, the same treatment was carried out for other monomers, thereby preparing an ion-exchanged monomer-containing ethyl acetate solution. Synthesis was carried out according to the same scheme as the synthesis of the polymer B1 in Example B1 by using the obtained ion exchange treated monomer-containing ethyl acetate solution, using Pruimepure manufactured by Kanto Chemical Co., Inc., which is electronic grade as the solvent such as n-heptane and tetrahydrofuran, and further using instruments, all of which were immersed in nitric acid for a day and then washed with ultrapure water, as the reaction vessel such as a flask. Further in the treatment after synthesis, a purification treatment was carried out by using a 5 nm Nylon filter (manufactured by Pall) and a 15 nm PTFE filter (manufactured by Entegris) in the order presented, and the purified product was dried under reduced pressure to obtain a white powdery polymer B2 (a polymer whose chemical structure is represented by the formula (MA1)). The inorganic element content and organic impurity content of the polymer were measured by the aforementioned method, and the results are shown in Table 3.

### Example B4

### Synthesis of Polymer B4

The polymer B4 (a polymer whose chemical structure is represented by the formula (MA1)) was obtained in the same manner as Example B2 except for using the compound M2 instead of the compound M1. The inorganic element content and organic impurity content of the polymer were measured by the aforementioned method, and the results are shown in Table 3.

### Examples B10 to 20

### Synthesis of Polymers B10 to B20

The polymers B10 to B20 were obtained in the same manner as Example B2 except for using the compounds M8 to M16, MCL1, and AH2 described in Table 2 instead of the compound M1. The inorganic element content and organic impurity content of the polymer were measured by the aforementioned method, and the results are shown in Table 3.

TABLE 2

| | Monomer | | | | | Polymer | | | | | |
|---|---|---|---|---|---|---|---|---|---|---|---|
| | Compound | | MAMA | BLMA | HAMA | | Struc- | a | b | c | d |
| | | Amount | Amount | Amount | Amount | | tural | (Molar | (Molar | (Molar | (Molar |
| | No. | (g) | (g) | (g) | (g) | No. | formula | ratio) | ratio) | ratio) | ratio) |
| Example B1 | A1 | 1.5 | 3 | 2 | 1.5 | B1 | MA1 | 40 | 30 | 15 | 15 |
| Example B2 | A1 | 1.5 | 3 | 2 | 1.5 | B2 | MA1 | 40 | 20 | 15 | 15 |
| Example B3 | A2 | 1.9 | 3 | 2 | 1.5 | B3 | MA2 | 40 | 30 | 15 | 15 |
| Example B4 | A2 | 1.9 | 2 | 2 | 1.5 | B4 | MA2 | 40 | 20 | 15 | 15 |
| Example B5 | A3 | 2 | 3 | 2 | 1.5 | B5 | MA3 | 40 | 30 | 15 | 15 |
| Example B6 | A4 | 2 | 3 | 2 | 1.5 | B6 | MA4 | 40 | 30 | 15 | 15 |
| Example B7 | A5 | 1.5 | 3 | 2 | 1.5 | B7 | MA5 | 40 | 30 | 15 | 15 |
| Example B8 | A6 | 2.1 | 3 | 2 | 1.5 | B8 | MA6 | 40 | 30 | 15 | 15 |
| Example B9 | A7 | 2 | 3 | 2 | 1.5 | B9 | MA7 | 40 | 30 | 15 | 15 |
| Example B10 | A8 | 2 | 3 | 2 | 1.5 | B10 | MA8 | 40 | 30 | 15 | 15 |
| Example B11 | A9 | 2 | 3 | 2 | 1.5 | B11 | MA9 | 40 | 30 | 15 | 15 |
| Example B12 | A10 | 2 | 3 | 2 | 1.5 | B12 | MA10 | 40 | 30 | 15 | 15 |
| Example B13 | A11 | 2 | 3 | 2 | 1.5 | B13 | MA11 | 40 | 30 | 15 | 15 |
| Example B14 | A12 | 2 | 3 | 2 | 1.5 | B14 | MA12 | 40 | 30 | 15 | 15 |
| Example B15 | A13 | 2 | 3 | 2 | 1.5 | B15 | MA13 | 40 | 30 | 15 | 15 |
| Example B16 | A14 | 2 | 3 | 2 | 1.5 | B16 | MA14 | 40 | 30 | 15 | 15 |
| Example B17 | A15 | 2 | 3 | 2 | 1.5 | B17 | MA15 | 40 | 30 | 15 | 15 |
| Example B18 | A16 | 2 | 3 | 2 | 1.5 | B18 | MA16 | 40 | 30 | 15 | 15 |
| Example B19 | MCL1 | 2 | 3 | 2 | 1.5 | B19 | MA17 | 40 | 30 | 15 | 15 |
| Example B20 | AH2 | 2 | 3 | 2 | 1.5 | B20 | MA18 | 40 | 30 | 15 | 15 |
| Comparative | AR1 | 0.5 | 3 | 2 | 1.5 | BR1 | MAR1 | 40 | 30 | 15 | 15 |
| Example B1 | | | | | | | | | | | |
| Comparative | AR2 | 0.9 | 3 | 2 | 1.5 | BR2 | MAR2 | 40 | 30 | 15 | 15 |
| Example B2 | | | | | | | | | | | |

| | Monomer | | | | | | | | | | | | | |
|---|---|---|---|---|---|---|---|---|---|---|---|---|---|---|
| | Compound | | Compound | | | | | Polymer | | | | | | |
| | a1 | | a2 | | MAMA | BLMA | HAMA | | Struc- | a1 | a2 | b | c | d |
| | | Amount | | Amount | Amount | Amount | Amount | | tural | (Molar | (Molar | (Molar | (Molar | (Molar |
| | No. | (g) | No. | (g) | (g) | (g) | (g) | No. | formula | ratio) | ratio) | ratio) | ratio) | ratio) |
| Example | A1 | 1.3875 | MD1 | 0.225 | 3 | 2 | 1.5 | BD1 | PMD1 | 37 | 3 | 30 | 15 | 15 |
| BD1 | | | | | | | | | | | | | | |
| Example | A1 | 1.4813 | MD1 | 0.0375 | 3 | 2 | 1.5 | BD2 | PMD2 | 39.5 | 0.5 | 30 | 15 | 15 |
| BD2 | | | | | | | | | | | | | | |
| Example | A1 | 1.4906 | MD1 | 0.0188 | 3 | 2 | 1.5 | BD3 | PMD2 | 39.75 | 0.25 | 30 | 15 | 15 |
| BD3 | | | | | | | | | | | | | | |
| Example | A1 | 1.4985 | MD1 | 0.003 | 3 | 2 | 1.5 | BD4 | PMD2 | 39.96 | 0.04 | 30 | 15 | 15 |
| BD4 | | | | | | | | | | | | | | |
| Example | A1 | 1.4175 | MD2 | 0.165 | 3 | 2 | 1.5 | BD5 | PMD3 | 37.8 | 2.2 | 30 | 15 | 15 |
| BD5 | | | | | | | | | | | | | | |
| Example | A1 | 1.47 | MD2 | 0.06 | 3 | 2 | 1.5 | BD6 | PMD2 | 39.2 | 0.8 | 30 | 15 | 15 |
| BD6 | | | | | | | | | | | | | | |
| Example | A1 | 1.4925 | MD2 | 0.015 | 3 | 2 | 1.5 | BD7 | PMD2 | 39.8 | 0.2 | 30 | 15 | 15 |
| BD7 | | | | | | | | | | | | | | |
| Example | A1 | 1.4955 | MD2 | 0.009 | 3 | 2 | 1.5 | BD8 | PMD4 | 39.88 | 0.12 | 30 | 15 | 15 |
| BD8 | | | | | | | | | | | | | | |
| Example | A1 | 1.4025 | MD3 | 0.195 | 3 | 2 | 1.5 | BD9 | PMD5 | 37.4 | 2.6 | 30 | 15 | 15 |
| BD9 | | | | | | | | | | | | | | |
| Example | A1 | 1.4865 | MD3 | 0.027 | 3 | 2 | 1.5 | BD10 | PMD6 | 39.64 | 0.36 | 30 | 15 | 15 |
| BD10 | | | | | | | | | | | | | | |
| Example | A1 | 1.38 | MD4 | 0.24 | 3 | 2 | 1.5 | BD11 | PMD7 | 36.8 | 3.2 | 30 | 15 | 15 |
| BD11 | | | | | | | | | | | | | | |
| Example | A1 | 1.491 | MD4 | 0.018 | 3 | 2 | 1.5 | BD12 | PMD8 | 39.76 | 0.24 | 30 | 15 | 15 |
| BD12 | | | | | | | | | | | | | | |
| Example | A8 | 1.41 | MD3 | 0.18 | 3 | 2 | 1.5 | BD13 | PMD5 | 37.6 | 2.4 | 30 | 15 | 15 |
| BD13 | | | | | | | | | | | | | | |
| Example | A8 | 1.488 | MD3 | 0.024 | 3 | 2 | 1.5 | BD14 | PMD6 | 39.68 | 0.32 | 30 | 15 | 15 |
| BD14 | | | | | | | | | | | | | | |
| Example | A8 | 1.3875 | MD4 | 0.225 | 3 | 2 | 1.5 | BD15 | PMD7 | 37 | 3 | 30 | 15 | 15 |
| BD15 | | | | | | | | | | | | | | |
| Example | A8 | 1.4955 | MD4 | 0.009 | 3 | 2 | 1.5 | BD16 | PMD8 | 39.88 | 0.12 | 30 | 15 | 15 |
| BD16 | | | | | | | | | | | | | | |
| Example | A3 | 1.3875 | MAD1 | 0.225 | 3 | 2 | 1.5 | BD17 | PMD9 | 37 | 3 | 30 | 15 | 15 |
| BD17 | | | | | | | | | | | | | | |
| Example | A3 | 1.4985 | MAD1 | 0.003 | 3 | 2 | 1.5 | BD18 | PMD10 | 39.96 | 0.04 | 30 | 15 | 15 |
| BD18 | | | | | | | | | | | | | | |
| Example | A5 | 1.4175 | MAD2 | 0.165 | 3 | 2 | 1.5 | BD19 | PMD11 | 37.8 | 2.2 | 30 | 15 | 15 |
| BD19 | | | | | | | | | | | | | | |
| Example | A5 | 1.4955 | MAD2 | 0.009 | 3 | 2 | 1.5 | BD20 | PMD12 | 39.88 | 0.12 | 30 | 15 | 15 |
| BD20 | | | | | | | | | | | | | | |
| Example | A1 | 1.3875 | MR1 | 0.225 | 3 | 2 | 1.5 | BD21 | PMD13 | 37 | 3 | 30 | 15 | 15 |
| BD21 | | | | | | | | | | | | | | |
| Example | A1 | 1.4985 | MR1 | 0.003 | 3 | 2 | 1.5 | BD22 | PMD14 | 39.96 | 0.04 | 30 | 15 | 15 |
| BD22 | | | | | | | | | | | | | | |
| Example | A5 | 1.4175 | MR4 | 0.165 | 3 | 2 | 1.5 | BD23 | PMD15 | 37.8 | 2.2 | 30 | 15 | 15 |
| BD23 | | | | | | | | | | | | | | |
| Example | A5 | 1.4955 | MR4 | 0.009 | 3 | 2 | 1.5 | BD24 | PMD16 | 39.88 | 0.12 | 30 | 15 | 15 |
| BD24 | | | | | | | | | | | | | | |

| | Monomer | | | | | | | | | | | | | | | | |
|---|---|---|---|---|---|---|---|---|---|---|---|---|---|---|---|---|---|
| | Compound | | Compound | | Compound | | | | | Polymer | | | | | | | |
| | a1 | | a2 | | a3 | | MAMA | BLMA | HAMA | | Struc- | a1 | a2 | a3 | b | c | d |
| | | Amount | | Amount | | Amount | Amount | Amount | Amount | | tural | (Molar | (Molar | (Molar | (Molar | (Molar | (Molar |
| | No. | (g) | No. | (g) | No. | (g) | (g) | (g) | (g) | No. | formula | ratio) | ratio) | ratio) | ratio) | ratio) | ratio) |
| Example | A1 | 1.3875 | MD3 | 0.225 | MR1 | 0.165 | 3 | 2 | 1.5 | BD25 | PMD25 | 34.8 | 3 | 2.2 | 30 | 15 | 15 |
| BD25 | | | | | | | | | | | | | | | | | |
| Example | A1 | 1.555 | MD3 | 0.045 | MR1 | 0.03 | 3 | 2 | 1.5 | BD26 | PMD26 | 39 | 0.6 | 0.4 | 30 | 15 | 15 |
| BD26 | | | | | | | | | | | | | | | | | |
| Example | A1 | 1.5916 | MD3 | 0.003 | MR1 | 0.003 | 3 | 2 | 1.5 | BD27 | PMD27 | 39.92 | 0.04 | 0.04 | 30 | 15 | 15 |
| BD27 | | | | | | | | | | | | | | | | | |
| Example | A5 | 1.4175 | MD4 | 0.165 | MR4 | 0.225 | 3 | 2 | 1.5 | BD28 | PMD28 | 34.8 | 2.2 | 3 | 30 | 15 | 15 |
| BD28 | | | | | | | | | | | | | | | | | |
| Example | A5 | 1.5804 | MD4 | 0.135 | MR4 | 0.09 | 3 | 2 | 1.5 | BD29 | PMD29 | 38.8 | 0.72 | 0.48 | 30 | 15 | 15 |
| BD29 | | | | | | | | | | | | | | | | | |
| Example | A5 | 1.6195 | MD4 | 0.009 | MR4 | 0.009 | 3 | 2 | 1.5 | BD30 | PMD30 | 39.76 | 0.12 | 0.12 | 30 | 15 | 15 |
| BD30 | | | | | | | | | | | | | | | | | |

The meanings of the abbreviations in the Tables are as follows.
MAMA: 2-methyl-2-adamantyl methacrylate
BLMA: γ-butyrolactone methacrylate
HAMA: hydroxyadamantyl methacrylate

TABLE 3

| | | Monomer physical properties | | | | |
|---|---|---|---|---|---|---|
| | | Impurity content (ppm) | | | | |
| | | | | | | Phosphorus- |
| | | | | | Maleic | containing |
| Example | Polymer | Mn | Al | K | acid | compound |
| Example B1 | B1 | 0.01 | 0.01 | 0.01 | 0.5 | 0.1 |
| Example B2 | B2 | 0.0001 | 0.0001 | 0.0001 | 0.1 | 0.1 |
| Example B3 | B3 | 0.01 | 0.01 | 0.01 | 0.01 | 0.1 |
| Example B4 | B4 | 0.0001 | 0.0001 | 0.0001 | 0.1 | 0.1 |
| Example B5 | B5 | 0.01 | 0.01 | 0.01 | 0.01 | 0.1 |
| Example B6 | B6 | 0.01 | 0.01 | 0.01 | 0.01 | 0.1 |
| Example B7 | B7 | 0.01 | 0.01 | 0.01 | 0.01 | 0.1 |
| Example B8 | B8 | 0.01 | 0.01 | 0.01 | 0.01 | 0.1 |
| Example B9 | B9 | 0.01 | 0.01 | 0.01 | 0.01 | 0.1 |
| Example B10 | B10 | 0.01 | 0.01 | 0.01 | 0.01 | 0.1 |
| Example B11 | B11 | 0.01 | 0.01 | 0.01 | 0.01 | 0.1 |
| Example B12 | B12 | 0.01 | 0.01 | 0.01 | 0.01 | 0.1 |
| Example B13 | B13 | 0.01 | 0.01 | 0.01 | 0.01 | 0.1 |
| Example B14 | B14 | 0.01 | 0.01 | 0.01 | 0.01 | 0.1 |
| Example B15 | B15 | 0.01 | 0.01 | 0.01 | 0.01 | 0.1 |
| Example B16 | B16 | 0.01 | 0.01 | 0.01 | 0.01 | 0.1 |
| Example B17 | B17 | 0.01 | 0.01 | 0.01 | 0.01 | 0.1 |
| Example B18 | B18 | 0.01 | 0.01 | 0.01 | 0.01 | 0.1 |
| Example B19 | B19 | 0.01 | 0.01 | 0.01 | 0.01 | 0.1 |
| Example B20 | B20 | 0.01 | 0.01 | 0.01 | 0.01 | 0.1 |
| Comparative | BR1 | 0.1 | 0.1 | 0.1 | 0.5 | 0.5 |
| Example B1 | | | | | | |
| Comparative | BR2 | 0.1 | 0.1 | 0.1 | 0.5 | 0.5 |
| Example B2 | | | | | | |
| Example BD1 | BD1 | 0.0001 | 0.0001 | 0.0001 | 0.1 | 0.1 |
| Example BD2 | BD2 | 0.0001 | 0.0001 | 0.0001 | 0.1 | 0.1 |
| Example BD3 | BD3 | 0.0001 | 0.0001 | 0.0001 | 0.1 | 0.1 |
| Example BD4 | BD4 | 0.0001 | 0.0001 | 0.0001 | 0.1 | 0.1 |
| Example BD5 | BD5 | 0.0001 | 0.0001 | 0.0001 | 0.1 | 0.1 |
| Example BD6 | BD6 | 0.0001 | 0.0001 | 0.0001 | 0.1 | 0.1 |
| Example BD7 | BD7 | 0.0001 | 0.0001 | 0.0001 | 0.1 | 0.1 |
| Example BD8 | BD8 | 0.0001 | 0.0001 | 0.0001 | 0.1 | 0.1 |
| Example BD9 | BD9 | 0.0001 | 0.0001 | 0.0001 | 0.1 | 0.1 |
| Example BD10 | BD10 | 0.0001 | 0.0001 | 0.0001 | 0.1 | 0.1 |
| Example BD11 | BD11 | 0.0001 | 0.0001 | 0.0001 | 0.1 | 0.1 |
| Example BD12 | BD12 | 0.0001 | 0.0001 | 0.0001 | 0.1 | 0.1 |
| Example BD13 | BD13 | 0.0001 | 0.0001 | 0.0001 | 0.1 | 0.1 |
| Example BD14 | BD14 | 0.0001 | 0.0001 | 0.0001 | 0.1 | 0.1 |
| Example BD15 | BD15 | 0.0001 | 0.0001 | 0.0001 | 0.1 | 0.1 |
| Example BD16 | BD16 | 0.0001 | 0.0001 | 0.0001 | 0.1 | 0.1 |
| Example BD17 | BD17 | 0.0001 | 0.0001 | 0.0001 | 0.1 | 0.1 |
| Example BD18 | BD18 | 0.0001 | 0.0001 | 0.0001 | 0.1 | 0.1 |
| Example BD19 | BD19 | 0.0001 | 0.0001 | 0.0001 | 0.1 | 0.1 |
| Example BD20 | BD20 | 0.0001 | 0.0001 | 0.0001 | 0.1 | 0.1 |
| Example BD21 | BD21 | 0.0001 | 0.0001 | 0.0001 | 0.1 | 0.1 |
| Example BD22 | BD22 | 0.0001 | 0.0001 | 0.0001 | 0.1 | 0.1 |
| Example BD23 | BD23 | 0.0001 | 0.0001 | 0.0001 | 0.1 | 0.1 |
| Example BD24 | BD24 | 0.0001 | 0.0001 | 0.0001 | 0.1 | 0.1 |
| Example BD25 | BD25 | 0.0001 | 0.0001 | 0.0001 | 0.1 | 0.1 |
| Example BD26 | BD26 | 0.0001 | 0.0001 | 0.0001 | 0.1 | 0.1 |
| Example BD27 | BD27 | 0.0001 | 0.0001 | 0.0001 | 0.1 | 0.1 |
| Example BD28 | BD28 | 0.0001 | 0.0001 | 0.0001 | 0.1 | 0.1 |
| Example BD29 | BD29 | 0.0001 | 0.0001 | 0.0001 | 0.1 | 0.1 |
| Example BD30 | BD30 | 0.0001 | 0.0001 | 0.0001 | 0.1 | 0.1 |

### Examples BD1 to BD30

### Synthesis of Polymers PMD1 to PMD30

The polymers BD1 to BD30 (polymers whose chemical structures are represented by the formulas (PMD1 to PMD30)) were obtained in the same manner as in Example B2 except for using the compound a1, the compound a2, and the compound a3 described in Table 2-2 instead of the compound M1, in the described ratio. The inorganic element content and organic impurity content of the polymer were measured by the aforementioned method, and the results are shown in Table 3-2.

### [Evaluation]

Evaluations of the compounds A1 to A16, AH1, AH2, MCL1, and AR1 to AR3, and the polymers B1 to B20, BR1, and BR2 obtained in the aforementioned Examples and Comparative Examples were carried out as follows. The results are shown in Table 4, Table 5, and Table A.

### (EUV Sensitivity-Aqueous TMAH Solution Development)

A solution was prepared by blending 5 parts by mass of the compound or the polymer obtained in Examples or Comparative Examples, 1 part by mass of triphenylsulfonium nonafluoromethanesulfonate, 0.2 parts by mass of tributylamine, 80 parts by mass of PGMEA, and 12 parts by mass of PGME.

The solution was applied on a silicon wafer, which was baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure without masking by incrementing the exposure amount by 1 mJ/cm² from 1 mJ/cm² to 80 mJ/cm², baked (PEB) at 110° C. for 90 seconds, and developed with a 2.38% by mass aqueous tetramethylammonium hydroxide (TMAH) solution for 60 seconds to obtain a wafer on which 80 shots of shot exposure was carried out. For each obtained shot exposure area, the film thickness was measured by using an optical interference film thickness meter “VM3200” (product name, manufactured by SCREEN Semiconductor Solutions Co., Ltd.), profile data about the film thickness relative to the exposure amount was obtained, and the exposure amount at which the slope of film thickness variation relative to an exposure amount was the largest was calculated as a sensitivity value (mJ/cm²), which was used as an index of the EUV sensitivity of the resist.

### (Evaluation of Sensitivity Due to Aging)

The solution prepared in the aforementioned EUV sensitivity evaluation was subjected to a forced aging treatment under light-shielding conditions at 40° C./240 hours, and the solution after the aging treatment was evaluated for EUV sensitivity in the same manner. Evaluations were conducted based on the amount of change of sensitivity. As the specific evaluation method, in a film thickness-sensitivity curve after development when the abscissa was set to sensitivity and the ordinate was set to film thickness in the EUV sensitivity evaluation, the sensitivity value at which the slope value was maximum was measured as the standard sensitivity. The standard sensitivities of the solutions before and after being subjected to a forced aging treatment were determined, and sensitivity variations due to the aging treatment were evaluated from the numerical value obtained by the following calculation expression. The evaluation criteria are as follows.

[Sensitivity variation]=1−([standard sensitivity of solution after aging]÷[standard sensitivity of solution before aging])

### (Evaluation Criteria)

A: [sensitivity variation]≤0.005

B: 0.005<[sensitivity variation]≤0.02

C: 0.02<[sensitivity variation]≤0.05

D: 0.05<[sensitivity variation]

### (EB Pattern-Aqueous TMAH Solution Development)

A solution was prepared by blending 5 parts by mass of the compound or the polymer obtained in Examples or Comparative Examples, 1 part by mass of triphenylsulfonium nonafluoromethanesulfonate, 0.1 parts by mass of tributylamine, and 92 parts by mass of PGMEA.

The solution was applied on a silicon wafer, which was baked at 110 to 130° C. for 60 seconds to form a resist film having a film thickness of 100 nm.

Then, the resist film was exposed using an electron beam lithography system “ELS-7500” (product name, manufactured by ELIONIX INC., 50 keV), baked (PEB) at 115° C. for 90 seconds, and developed with a 2.38 mass % tetramethylammonium hydroxide (TMAH) aqueous solution for 60 seconds to obtain a positive type pattern. The exposure amount was regulated to have a line and space pattern with a half pitch of 50 nm.

For the obtained resist pattern, 80 pattern images were obtained with a scanning electron microscope “S-4800” (product name, manufactured by Hitachi, Ltd.) at 100000 times magnification, the number of residues on the space portion between resist patterns were counted, and evaluations were carried out from the total amount of the residues. The evaluation criteria are as follows.

### (Evaluation Criteria)

A: Number of residues≤less than 10

B: 10<number of residues≤80

C: 80<number of residues≤400

D: 400<number of residues

### (Evaluation of Etching Defect)

A solution was prepared by blending 5 parts by mass of the compound or the polymer obtained in Examples or Comparative Examples, 1 part by mass of triphenylsulfonium nonafluoromethanesulfonate, 0.2 parts by mass of tributylamine, 80 parts by mass of PGMEA, and 12 parts by mass of PGME.

The solution was applied on a 8 inch silicon wafer, on the outermost layer of which an oxide film having a film thickness of 100 nm was formed, and this was baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure with an exposure amount that is 10% less than the EUV sensitivity value obtained in the above EUV sensitivity evaluation on the entire wafer surface, further baked (PEB) at 110° C. for 90 seconds, and developed with a 2.38% by mass aqueous tetramethylammonium hydroxide (TMAH) solution for 60 seconds to obtain a wafer on the entire surface of which 80 shots of shot exposure was carried out.

The prepared exposed wafer was subjected to an etching treatment with an etching apparatus “Telius SCCM” (product name, manufactured by Tokyo Electron Ltd.), using CF₄/Ar gas until the oxide film was etched by 50 nm. Evaluation of defects were carried out for the wafer prepared by etching with a defect inspection apparatus “Surfscan SP5” (product name, manufactured by KLA), and the number of cone defects with a size of 19 nm or more was determined as an index of the etching defect.

### (Evaluation Criteria)

A: Number of cone defects≤less than 10

B: 10<number of cone defects≤80

C: 80<number of cone defects≤400

D: 400<number of cone defects

TABLE 4

| | | | Evaluation | | | | |
|---|---|---|---|---|---|---|---|
| | | | EUV | | | | |
| | | | sensitivity | | EB | | |
| | | | aqueous | | pattern | | |
| | | | TMAH | Change of | aqueous | | |
| | | | solution | sensitivity | TMAH | | |
| | | Structural | development | due to | solution | Etching | |
| | Polymer | formula | (mJ/cm²) | aging | development | defect | |
| Example B1 | B1 | MA1 | 21 | B | B | B | |
| Example B2 | B2 | MA1 | 19 | A | A | A | |
| Example B3 | B3 | MA2 | 22 | B | B | B | |
| Example B4 | B4 | MA2 | 19 | A | A | A | |
| Example B5 | B5 | MA3 | 17 | B | B | B | |
| Example B6 | B6 | MA4 | 17 | B | B | B | |
| Example B7 | B7 | MA5 | 19 | B | B | B | |
| Example B8 | B8 | MA6 | 19 | B | B | B | |
| Example B9 | B9 | MA7 | 19 | B | B | B | |
| Example B10 | B10 | MA8 | 20 | B | B | B | |
| Example B11 | B11 | MA9 | 18 | B | B | B | |
| Example B12 | B12 | MA10 | 21 | B | B | B | |
| Example B13 | B13 | MA11 | 18 | B | B | B | |
| Example B14 | B14 | MA12 | 16 | B | B | B | |
| Example B15 | B15 | MA13 | 16 | B | B | B | |
| Example B16 | B16 | MA14 | 18 | B | B | B | |
| Example B17 | B17 | MA15 | 18 | B | B | B | |
| Example B18 | B18 | MA16 | 18 | B | B | B | |
| Example B19 | B19 | MA17 | 18 | B | B | B | |
| Example B20 | B20 | MA18 | 20 | B | B | B | |
| Comparative | BR1 | MAR1 | 49 | C | C | C | |
| Example B1 | | | | | | | |
| Comparative | BR2 | MAR2 | 39 | C | C | C | |
| Example B2 | | | | | | | |
| | | | | Evaluation | | | |
| | | | | EUV | | | |
| | | | | sensitivity | | EB | |
| | | | | aqueous | | pattern | |
| | | | | TMAH | Change of | aqueous | |
| | | Polymer | | solution | sensitivity | TMAH | |
| | | Structural | Polymer | development | due to | solution | Etching |
| | Compound | formula | No. | (mJ/cm²) | aging | development | defect |
| Example BD1 | BD1 | PMD1 | BD1 | 21 | B | B | B |
| Example BD2 | BD2 | PMD2 | BD2 | 22 | A | A | B |
| Example BD3 | BD3 | PMD3 | BD3 | 22 | A | A | B |
| Example BD4 | BD4 | PMD4 | BD4 | 23 | B | A | B |
| Example BD5 | BD5 | PMD5 | BD5 | 21 | B | B | B |
| Example BD6 | BD6 | PMD6 | BD6 | 22 | A | A | B |
| Example BD7 | BD7 | PMD7 | BD7 | 22 | A | A | B |
| Example BD8 | BD8 | PMD8 | BD8 | 23 | B | A | B |
| Example BD9 | BD9 | PMD9 | BD9 | 21 | B | B | B |
| Example BD10 | BD10 | PMD10 | BD10 | 22 | B | A | B |
| Example BD11 | BD11 | PMD11 | BD11 | 22 | B | B | B |
| Example BD12 | BD12 | PMD12 | BD12 | 23 | B | A | B |
| Example BD13 | BD13 | PMD13 | BD13 | 21 | A | A | B |
| Example BD14 | BD14 | PMD14 | BD14 | 22 | B | A | B |
| Example BD15 | BD15 | PMD15 | BD15 | 21 | A | A | B |
| Example BD16 | BD16 | PMD16 | BD16 | 22 | B | A | B |
| Example BD17 | BD17 | PMD17 | BD17 | 23 | A | B | B |
| Example BD18 | BD18 | PMD18 | BD18 | 22 | B | B | B |
| Example BD19 | BD19 | PMD19 | BD19 | 23 | A | B | B |
| Example BD20 | BD20 | PMD20 | BD20 | 22 | B | B | B |
| Example BD21 | BD21 | PMD21 | BD21 | 23 | A | B | B |
| Example BD22 | BD22 | PMD22 | BD22 | 22 | B | B | B |
| Example BD23 | BD23 | PMD23 | BD23 | 23 | A | B | B |
| Example BD24 | BD24 | PMD24 | BD24 | 22 | B | B | B |
| Example BD25 | BD25 | PMD25 | BD25 | 23 | B | B | B |
| Example BD26 | BD26 | PMD26 | BD26 | 22 | A | A | B |
| Example BD27 | BD27 | PMD27 | BD27 | 23 | B | A | B |
| Example BD28 | BD28 | PMD28 | BD28 | 22 | B | B | B |
| Example BD29 | BD29 | PMD29 | BD29 | 23 | A | A | B |
| Example BD30 | BD30 | PMD30 | BD30 | 22 | B | A | B |

### (Evaluation of Aging Stability of Composition Containing Compound)

For the stability of the composition containing the compound obtained in Examples or Comparative Examples, the amount of change of the purity before and after the aging test in a solution state in which a single or a plurality of compounds are combined was evaluated as an index of the stability.

As the sample for evaluation, a solution in which the compounds of Examples or Comparative Examples and a solvent described in the table A were mixed was prepared, which was filled in an inactivation-treated 100 mL brown glass container until 90 mL and capped to prepare a sample. As aging conditions, the sample was subjected to an aging treatment in a light-shielded constant temperature tester at 45° C. for 30 days.

For the prepared sample, the purity before and after aging treatment was measured by HPLC analysis.

The amount of change of the HPLC purity before and after aging was determined as follows and used as an index of the evaluation.

The obtained results were described in Table A.

Amount of change of purity due to aging=area % of target component before aging−area % of target component after aging

### (Evaluation Criteria)

A: Amount of change of purity due to aging≤0.2%

B: 0.2%<Amount of change of purity due to aging≤0.5%

C: 0.5%<Amount of change of purity due to aging≤1.0%

D: 1.0%<Amount of change of purity due to aging≤3.0%

E: 3.0%<Amount of change of purity due to aging

TABLE A

| | Monomer solution | | | | | | | |
|---|---|---|---|---|---|---|---|---|
| | Compound a1 | | Compound a2 | | Compound a2 | | Solvent | |
| | | Amount | | Amount | | Amount | Toluene | Aging |
| | No. | (g) | No. | (g) | No. | (g) | (g) | evaluation |
| Example SL1 | A1 | 1.3875 | MAD1 | 0.1125 | — | — | 13.5 | A |
| Example SL2 | A1 | 1.4985 | MAD1 | 0.0015 | — | — | 13.5 | B |
| Example SL3 | A5 | 1.4175 | MAD2 | 0.0825 | — | — | 13.5 | A |
| Example SL4 | A5 | 1.4955 | MAD2 | 0.0045 | — | — | 13.5 | B |
| Example SL5 | A3 | 1.3875 | MR1 | 0.1125 | — | — | 13.5 | A |
| Example SL6 | A3 | 1.4985 | MR1 | 0.0015 | — | — | 13.5 | B |
| Example SL7 | A5 | 1.4175 | MR4 | 0.0825 | — | — | 13.5 | A |
| Example SL8 | A5 | 1.4955 | MR4 | 0.0045 | — | — | 13.5 | B |
| Example SL9 | A1 | 1.305 | MD3 | 0.1125 | MR1 | 0.0825 | 13.5 | A |
| Example SL10 | A1 | 1.497 | MD3 | 0.0015 | MR1 | 0.0015 | 13.5 | B |
| Example SL11 | A5 | 1.305 | MD4 | 0.0825 | MR4 | 0.1125 | 13.5 | A |
| Example SL12 | A5 | 1.491 | MD4 | 0.0045 | MR4 | 0.0045 | 13.5 | B |
| Example SL13 | A1 | 1.5 | — | — | — | — | 13.5 | C |
| Example SL14 | A3 | 1.5 | — | — | — | — | 13.5 | C |
| Example SL15 | AS | 1.5 | — | — | — | — | 13.5 | C |

The results were obtained from Table A that determines that the compound A of the present invention can improve the stability in a solution state by containing a trace amount of the compound of the formula (1C), or the compound of the formula (1D), or the compound of the formula (1E).

### (EUV Sensitivity—Organic Solvent Development)

A solution containing the compound or the polymer obtained in Examples or Comparative Examples was prepared in the same manner as the EUV sensitivity-aqueous TMAH solution development, which was applied on a silicon wafer and baked at 110° C. for 60 seconds to form a photoresist layer having a film thickness of 100 nm.

Then, using an extreme ultraviolet (EUV) exposure apparatus “EUVES-7000” (product name, manufactured by Litho Tech Japan Corporation), the photoresist layer was subjected to shot exposure without masking by incrementing the exposure amount by 1 mJ/cm² from 1 mJ/cm² to 80 mJ/cm², baked (PEB) at 110° C. for 90 seconds, and developed with butyl acetate for 30 seconds to obtain a wafer on which 80 shots of shot exposure was carried out. For each obtained shot exposure area, the film thickness was measured by using an optical interference film thickness meter “VM3200” (product name, manufactured by SCREEN Semiconductor Solutions Co., Ltd.), profile data about the film thickness relative to the exposure amount was obtained, and the exposure amount at which the slope of film thickness variation relative to an exposure amount was the largest was calculated as a sensitivity value (mJ/cm²), which was used as an index of the EUV sensitivity of the resist.

### (EB Pattern—Organic Solvent Development)

A solution containing the compound or the polymer obtained in Examples or Comparative Examples was prepared in the same manner as the EB pattern-aqueous TMAH solution development, which was applied on a silicon wafer and baked at 110 to 130° C. for 60 seconds to form a resist film having a film thickness of 100 nm.

Then, the resist film was exposed using an electron beam lithography system “ELS-7500” (product name, manufactured by ELIONIX INC., 50 keV), baked (PEB) at 115° C. for 90 seconds, and developed with butyl acetate for 30 seconds to obtain a negative type pattern. The exposure amount was regulated to have a line and space pattern with a half pitch of 50 nm.

For the obtained resist pattern, 80 pattern images were obtained with a scanning electron microscope “S-4800” (product name, manufactured by Hitachi, Ltd.) at 100000 times magnification, the number of residues on the space portion between resist patterns were counted, and evaluations were carried out from the total amount of the residues. The evaluation criteria are as follows.

### (Evaluation Criteria)

A: Number of residues≤less than 10

B: 10<number of residues≤80

C: 80<number of residues≤400

D: 400<number of residues

TABLE 6

| | | | Evaluation | |
|---|---|---|---|---|
| | | | EUV sensitivity | EB pattern |
| | | | organic solvent | organic |
| | | Structural | development | solvent |
| | Compound | formula | (mJ/cm²) | development |
| Example A1 | A1 | M1 | 26 | B |
| Example A2 | A2 | M2 | 26 | B |
| Example A3 | A3 | M3 | 22 | B |
| Example A4 | A4 | M4 | 22 | B |
| Example A5 | A5 | M5 | 24 | B |
| Example A6 | A6 | M6 | 24 | B |
| Comparative | A1 | Mr1 | 57 | D |
| Example A1 | | | | |
| Comparative | A2 | Mr2 | 46 | D |
| Example A2 | | | | |

TABLE 7

| | | | Evaluation | |
|---|---|---|---|---|
| | | | EUV sensitivity | EB pattern |
| | | | organic solvent | organic |
| | | Structural | development | solvent |
| | Polymer | formula | (mJ/cm²) | development |
| Example B1 | B1 | MA1 | 25 | B |
| Example B2 | B2 | MA1 | 24 | A |
| Example B3 | B3 | MA2 | 26 | B |
| Example B4 | B4 | MA2 | 24 | A |
| Example B5 | B5 | MA3 | 22 | B |
| Example B6 | B6 | MA4 | 22 | B |
| Example B7 | B7 | MA5 | 25 | B |
| Example B8 | B8 | MA6 | 24 | B |
| Comparative | B1 | MA51 | 55 | C |
| Example B1 | | | | |
| Comparative | B2 | MA52 | 45 | C |
| Example B2 | | | | |

### Example C1

### Synthesis of Polymer C1

The polymer C1 (a polymer whose chemical structure is represented by the following formula (P-M1-CLMAA)) was obtained in the same manner as Example B2 except that 8.3 g of the compound A1 and 1.9 g of methyl 2-chloroacrylate (see the following formula for the structure, hereinafter also referred to as “CLMAA”) were used as the monomer raw materials. This polymer had a weight average molecular weight (Mw) of 13100 and a dispersity (Mw/Mn) of 1.9. As a result of the measurement of ¹³C-NMR, the composition ratio (molar ratio) in the following formula (P-M1-CLMAA) was a:b=50:50. Although the following formula (P-M1-CLMAA) is illustrated in a simplified form to show the ratio of respective constitutional units, the polymer C1 is not a block copolymer in which respective constitutional units form an independent block. The inorganic element content and organic impurity content of the polymer were measured by the aforementioned method, and the results are shown in Table 8.

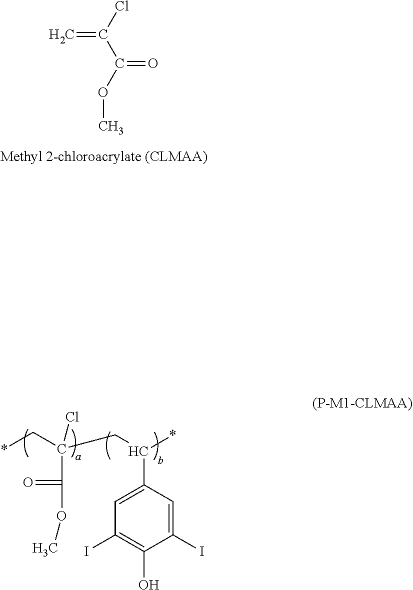

### Example C2

### Synthesis of Polymer C2

The polymer C2 (a polymer whose chemical structure is represented by the following formula (P-M2-CLMAA)) was obtained in the same manner as Example B2 except that 10.6 g of the compound A2 and 1.9 g of methyl 2-chloroacrylate were used as the monomer raw materials. This polymer had a weight average molecular weight (Mw) of 14400 and a dispersity (Mw/Mn) of 2.0. As a result of the measurement of ¹³C-NMR, the composition ratio (molar ratio) in the following formula (P-M2-CLMAA) was a:b=50:50. Although the following formula (P-M2-CLMAA) is illustrated in a simplified form to show the ratio of respective constitutional units, the polymer C2 is not a block copolymer in which respective constitutional units form an independent block. The inorganic element content and organic impurity content of the polymer were measured by the aforementioned method, and the results are shown in Table 8.

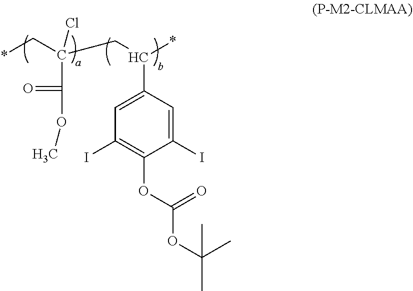

### Example C3

### Synthesis of Polymer C3

The polymer C3 (a polymer whose chemical structure is represented by the following formula (P-M5-CLMAA)) was obtained in the same manner as Example B2 except that 8.7 g of the compound A5 and 1.9 g of methyl 2-chloroacrylate were used as the monomer raw materials. This polymer had a weight average molecular weight (Mw) of 12400 and a dispersity (Mw/Mn) of 2.1. As a result of the measurement of ¹³C-NMR, the composition ratio (molar ratio) in the following formula (P-M5-CLMAA) was a:b=50:50. Although the following formula (P-M5-CLMAA) is illustrated in a simplified form to show the ratio of respective constitutional units, the polymer C3 is not a block copolymer in which respective constitutional units form an independent block. The inorganic element content and organic impurity content of the polymer were measured by the aforementioned method, and the results are shown in Table 8.

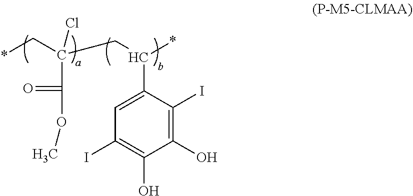

### Example C4

### Synthesis of Polymer C4

The polymer C4 (a polymer whose chemical structure is represented by the following formula (P-M6-CLMAA)) was obtained in the same manner as Example B2 except that 11.6 g of the compound A6 and 1.9 g of methyl 2-chloroacrylate were used as the monomer raw materials. This polymer had a weight average molecular weight (Mw) of 14400 and a dispersity (Mw/Mn) of 2.0. As a result of the measurement of ¹³C-NMR, the composition ratio (molar ratio) in the following formula (P-M6-CLMAA) was a:b=50:50. Although the following formula (P-M6-CLMAA) is illustrated in a simplified form to show the ratio of respective constitutional units, the polymer C4 is not a block copolymer in which respective constitutional units form an independent block. The inorganic element content and organic impurity content of the polymer were measured by the aforementioned method, and the results are shown in Table 8.

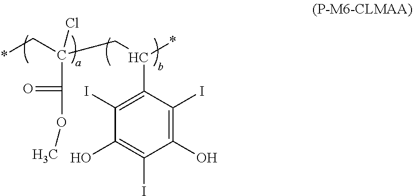

### Example C5

### Synthesis of Polymer C5

The polymer C5 (a polymer whose chemical structure is represented by the following formula (P-M1-CLMAA)) was obtained in the same manner as Example B2 except that 11.1 g of the compound AZ1 and 1.9 g of methyl 2-chloroacrylate were used as the monomer raw materials. This polymer had a weight average molecular weight (Mw) of 18100 and a dispersity (Mw/Mn) of 1.9. As a result of the measurement of ¹³C-NMR, the composition ratio (molar ratio) in the following formula (P-MZ1-CLMAA) was a:b=50:50. Although the following formula (P-MZ1-CLMAA) is illustrated in a simplified form to show the ratio of respective constitutional units, the polymer C5 is not a block copolymer in which respective constitutional units form an independent block. The inorganic element content and organic impurity content of the polymer were measured by the aforementioned method, and the results are shown in Table 8.

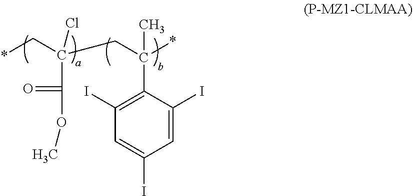

### Example C6

### Synthesis of Polymer C6

The polymer C6 (a polymer whose chemical structure is represented by the following formula (P-MZ1-ITBAA)) was obtained in the same manner as Example B2 except that 11.1 g of the compound AZ1 and 5.7 g of 2-iodoacrylic acid tert-butyl ester (hereinafter also referred to as “ITBAA”) were used as the monomer raw materials. This polymer had a weight average molecular weight (Mw) of 9300 and a dispersity (Mw/Mn) of 1.7. As a result of the measurement of ¹³C-NMR, the composition ratio (molar ratio) in the following formula (P-MZ1-ITBAA) was a:b=50:50. Although the following formula (P-MZ1-ITBAA) is illustrated in a simplified form to show the ratio of respective constitutional units, the polymer C6 is not a block copolymer in which respective constitutional units form an independent block. The inorganic element content and organic impurity content of the polymer were measured by the aforementioned method, and the results are shown in Table 8.

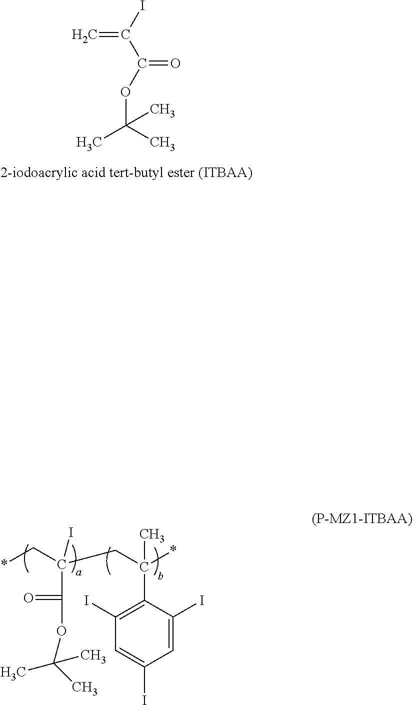

### Comparative Example CR1

### Synthesis of Polymer C51

The polymer C51 (a polymer whose chemical structure is represented by the following formula (P-AMPHS-CLMAA)) was obtained in the same manner as Example B2 except that 3.0 g of the compound AR3 and 1.9 g of methyl 2-chloroacrylate were used as the monomer raw materials. This polymer had a weight average molecular weight (Mw) of 21300 and a dispersity (Mw/Mn) of 2.1. As a result of the measurement of ¹³C-NMR, the composition ratio (molar ratio) in the following formula (P-AMPHS-CLMAA) was a:b=50:50. Although the following formula (P-AMPHS-CLMAA) is illustrated in a simplified form to show the ratio of respective constitutional units, the polymer C51 is not a block copolymer in which respective constitutional units form an independent block. The inorganic element content and organic impurity content of the polymer were measured by the aforementioned method, and the results are shown in Table 8.

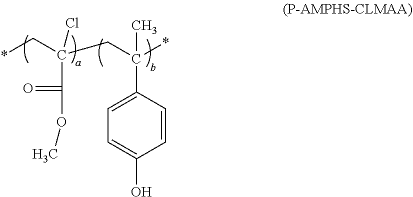

### Example C11 to Example C22

### Synthesis of Polymers C11 to C21

The polymers C11 to C22 were obtained in the same manner as Example Cl except that using the compounds AZ2 to AZ11 instead of the compound A1 as the monomer 1 to be used and using CLMAA or MCL1 as the monomer 2, in Example C1. The physical properties of the obtained polymer are also shown in Table 8. These polymers are not the block copolymer, as the polymer C1.

### [Evaluation]

The aforementioned polymers obtained in Examples C1 to C22 and Comparative Example CR1 were evaluated as follows. The results are shown in Table 9.

### (Resolution Evaluation—Resolution And Sensitivity—)

The solution of the polymer obtained in Example was applied on a silicon wafer, which was baked at 110° C. for 60 seconds to form a photoresist layer having a film thickness of 100 nm. Here, the solution of the polymer was prepared by blending the polymer: 7 parts by mass and PGMEA: 93.9 parts by mass.

Then, the photoresist layer was exposed using an electron beam lithography system “ELS-7500” (product name, manufactured by ELIONIX INC., 50 keV), baked (PEB) at 115° C. for 90 seconds, and developed for 60 seconds by using isoamyl acetate as developing solution to obtain a positive type pattern. The results of resolution and sensitivity are shown in Table 9.

### (Change of Sensitivity Due to Aging)

Using the solution of the polymer obtained in Examples, solutions of the polymer before and after aging in a light-shielded state at 40° C. for 30 days were prepared, and these solutions were the same except for whether or not the aging treatment was performed. Each of these solutions was used to form a film on a silicon wafer by spin coating, which was subjected to a development treatment using isoamyl acetate as a developing solution, the sensitivity before and after aging were determined, and the change rate was derived from the following index, thereby evaluating sensitivity due to aging. The results of the change rate are shown in Table 9.

[Change rate]=[“sensitivity of resin solution before aging”−“sensitivity of resin solution after aging”)/“sensitivity of resin solution before aging”]×100

A: Amount of change is less than 2%

B: Change rate is 2% or more and less than 5%

C: Change rate is 5% or more and less than 10%

D: Change rate is 10% or more

TABLE 8

| | | | | | | | Physical properties of monomer 1 | | | | |
|---|---|---|---|---|---|---|---|---|---|---|---|
| | | | Polymer | | | | Impurity content (ppm) | | | | |
| | Monomer | | | | a | b | | | | | Phosphorus- |
| | Monomer | Monomer | | Structural | (Molar | (Molar | | | | Maleic | containing |
| Example | 1 | 2 | No. | formula | ratio) | ratio) | Mn | Al | K | acid | compound |
| Example C1 | Compound A1 | CLMAA | C1 | (P-M1-CLMAA) | 50 | 50 | 0.003 | 0.003 | 0.001 | 0.05 | 0.03 |
| Example C2 | Compound A2 | CLMAA | C2 | (P-M2-CLMAA) | 50 | 50 | 0.01 | 0.01 | 0.001 | 0.11 | 0.12 |
| Example C3 | Compound A5 | CLMAA | C3 | (P-M5-CLMAA) | 50 | 50 | 0.01 | 0.004 | 0.0001 | 0.06 | 0.08 |
| Example C4 | Compound A6 | CLMAA | C4 | (P-M6-CLMAA) | 50 | 50 | 0.004 | 0.002 | 0.0001 | 0.11 | 0.11 |
| Example C5 | Compound AZ1 | CLMAA | C5 | (P-MZ1- CLMAA) | 50 | 50 | 0.006 | 0.005 | 0.0005 | ND | ND |
| Example C6 | Compound AZ1 | ITBAA | C5 | (P-MZ1-ITBAA) | 50 | 50 | 0.008 | 0.004 | 0.0006 | ND | ND |
| Example C11 | Compound AZ2 | CLMAA | C11 | (P-MZ2- CLMAA) | 50 | 50 | 0.003 | 0.003 | 0.001 | ND | 0.13 |
| Example C12 | Compound AZ3 | CLMAA | C12 | (P-MZ3- CLMAA) | 50 | 50 | 0.01 | 0.01 | 0.001 | ND | 0.21 |
| Example C13 | Compound AZ4 | CLMAA | C13 | (P-MZ4-CLMAA) | 50 | 50 | 0.01 | 0.004 | 0.0001 | ND | 0.14 |
| Example C14 | Compound AZ5 | CLMAA | C14 | (P-MZ5-CLMAA) | 50 | 50 | 0.004 | 0.002 | 0.0001 | ND | 0.13 |
| Example C15 | Compound AZ6 | CLMAA | C15 | (P-MZ6-CLMAA) | 50 | 50 | 0.006 | 0.005 | 0.0005 | ND | 0.11 |
| Example C16 | Compound AZ7 | CLMAA | C16 | (P-MZ7-CLMAA) | 50 | 50 | 0.003 | 0.003 | 0.001 | ND | 0.03 |
| Example C17 | Compound AZ8 | CLMAA | C17 | (P-MZ8-CLMAA) | 0 | 50 | 0.01 | 0.01 | 0.001 | ND | 0.12 |
| Example C18 | Compound AZ9 | CLMAA | C18 | (P-MZ9-CLMAA) | 50 | 50 | 0.01 | 0.004 | 0.0001 | 0.18 | 0.08 |
| Example C19 | Compound AZ10 | CLMAA | C19 | (P-MZ10- CLMAA) | 50 | 50 | 0.004 | 0.002 | 0.0001 | 0.21 | 0.11 |
| Example C20 | Compound AZ11 | CLMAA | C20 | (P-MZ11-CLMAA) | 50 | 50 | 0.002 | 0.001 | 0.001 | ND | 0.08 |
| Example C21 | Compound AZ1 | MCL1 | C21 | (P-MZ11- MCL1) | 50 | 50 | 0.006 | 0.005 | 0.0005 | ND | ND |
| Example C22 | Compound AZ8 | MCL1 | C22 | (P-MZ12-MCL1) | 50 | 50 | 0.004 | 0.002 | 0.0001 | ND | 0.15 |
| Comparative | Compound AR3 | CLMAA | C51 | (P-AMPHS-CLMAA) | 50 | 50 | 0.009 | 0.007 | 0.001 | 0.07 | 0.08 |
| Example CR1 | | | | | | | | | | | |

ND: below detection limit

TABLE 9

| | | Evaluation | | |
|---|---|---|---|---|
| | | | | Change of |
| | | Resolution | Sensitivity | sensitivity due |
| Example | Polymer | (nm L/S) | (μC/cm²) | to aging |
| Example C1 | C1 | 38 | 16 | A |
| Example C2 | C2 | 39 | 15 | A |
| Example C3 | C3 | 39 | 14 | A |
| Example C4 | C4 | 38 | 13 | A |
| Example C5 | C5 | 37 | 13 | A |
| Example C6 | C6 | 36 | 12 | A |
| Example C11 | C11 | 38 | 16 | A |
| Example C12 | C12 | 39 | 15 | A |
| Example C13 | C13 | 39 | 14 | A |
| Example C14 | C14 | 38 | 13 | A |
| Example C15 | C15 | 37 | 13 | A |
| Example C16 | C16 | 38 | 16 | A |
| Example C17 | C17 | 38 | 16 | A |
| Example C18 | C18 | 39 | 15 | A |
| Example C19 | C19 | 39 | 14 | A |
| Example C20 | C20 | 37 | 15 | A |
| Example C21 | C21 | 38 | 13 | A |
| Example C22 | C22 | 37 | 13 | A |
| Comparative | C51 | 38 | 45 | A |
| Example CR1 | | | | |

The results of Table 9 show that a resin composition that can achieve high sensitivity in EUV exposure and has good pattern formability can be obtained by using the compound according to the present invention.

The results of Examples and Comparative Examples above show that the compound (A) and the polymer (A) according to the present embodiment can provide a composition for film formation excellent in the sensitivity to an exposure light source.

### [Measurement Method]

### [Nuclear Magnetic Resonance (NMR)]

The structure of the compound was verified by carrying out NMR measurement under the following conditions using a nucleus magnetic resonance apparatus “Advance 600 II spectrometer” (product name, manufactured by Bruker).

### [1H-NMR Measurement]

Frequency: 400 MHz

Solvent: CDC13, or d6-DMSO

Internal standard: TMS

Measurement temperature: 23° C.

### [13C-NMR Measurement]

Frequency: 500 MHz

Solvent: CDC13, or d6-DMSO

Internal standard: TMS

Measurement temperature: 23° C.

### Example 1

### Synthesis of 4′-hydroxy-3′,5′-diiodoacetophenone

A reactor was charged with 61.27 g of 4′-hydroxyacetophenone, 91.38 g of iodine, 1,620 mL of methanol, and 180 mL of pure water, the reactor was immersed in an ice bath, and stirring was initiated. Subsequently, 44.06 g of an aqueous iodic acid solution with a mass percent concentration of 71.9 was added dropwise thereto for 30 minutes. Subsequently, the reactor was immersed in a water bath at 35° C., and stirring was continued over 3.5 hours. Subsequently, 13.37 g of an aqueous sodium hydrogen sulfite solution with a mass percent concentration of 35 was added to quench the reaction. Subsequently, the content of the reactor was gradually added to 3,600 mL of pure water with vigorous stirring and mixed. Subsequently, the precipitate was filtered off with a suction filter, compressed, and washed with 540 mL of an aqueous methanol solution with a volume percent concentration of 33.3. Subsequently, the precipitate was dried in vacuo at 40° C. to obtain 169.54 g of 4′-hydroxy-3′,5′-diiodoacetophenone. The yield was 97.1%.

As a result of the analysis by liquid chromatography-mass spectrometry (LC-MS), the molecular weight was found to be 388, and the substance was confirmed to be 4′-hydroxy-3′,5′-diiodoacetophenone.

The following peaks were found by carrying out ¹H-NMR measurement under the above measurement conditions, and the compound was confirmed to have the chemical structure.

δ (ppm) (d6-DMSO): 10.5 (1H, OH), 8.3 (2H, Ph), 2.5 (3H, —CH3)

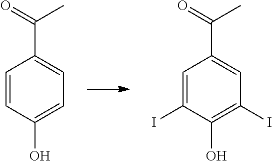

### Example 2

### Synthesis of 1-(4-hydroxyphenyl)ethanol

The reactor was charged with 8.77 g of sodium borohydride and 180 mL of tetrahydrofuran, the reactor was immersed in an ice bath, and stirring was initiated. Subsequently, a mixed solution consisting of 21.00 g of 4′-hydroxyacetophenone, 9.32 g of isopropanol, and 180 mL of tetrahydrofuran were added dropwise thereto over 3 hours. Subsequently, stirring was continued over 8 hours while the reactor was immersed in the ice bath. Subsequently, 59.47 g of methanol was added to quench the reaction. Subsequently, the pressure in the reactor was reduced to 50 hPa, immersed in a water bath at 20° C. to concentrate the reaction solution. Subsequently, the reactor was immersed in an ice bath, and 120 mL of cold methanol was added to dilute the reaction solution. Subsequently, the pressure in the reactor was reduced to 50 hPa, immersed in a water bath at 20° C. to concentrate the reaction solution. Subsequently, the reactor was immersed in an ice bath, and 600 mL of cold methanol was added to dilute the reaction solution. Subsequently, the reaction solution was gradually added to 1,200 g of dilute sulfuric acid with a mass percent concentration of 1 with vigorous stirring and mixed. Subsequently, the precipitate was filtered off with a suction filter, compressed, and washed with 300 mL of an aqueous methanol solution with a volume percent concentration of 33.3. Subsequently, the precipitate was dried in vacuo at 40° C. to obtain 20.3 g of 1-(4-hydroxyphenyl)ethanol. The yield was 95.2%.

The following peaks were found by carrying out ¹H-NMR measurement under the above measurement conditions, and the compound was confirmed to have the chemical structure.

δ (ppm) (d6-DMSO): 9.4 (1H, —OH), 7.7 (4H, Ph), 5.2 (1H, —CH—OH), 4.6 (1H, —CH—OH), 1.3 (3H, —CH3)

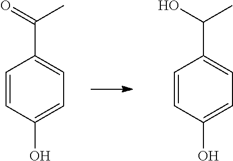

### Example 3

### Synthesis of 1-(4-hydroxy-3,5-diiodophenyl)ethanol

A reactor was charged with 1.2000 g of 1-(4-hydroxyphenyl)ethanol, 1.7630 g of iodine, and 17.37 mL of methanol, the reactor was immersed in an ice bath, and stirring was initiated. Subsequently, 0.8736 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.174 mL of an aqueous sodium hydrogen sulfite solution with a mass percent concentration of 35 was added to quench the reaction. Subsequently, the reaction solution was gradually added to 34.74 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 3.0969 g of a mixture of 1-(4-hydroxy-3,5-diiodophenyl)ethanol and 2,6-diiodo-4-(1-methoxyethyl)phenol. As a result of the HPLC analysis using a UV detector at a measurement wavelength of 254 nm, the ratio between 1-(4-hydroxy-3,5-diiodophenyl)ethanol and 2,6-diiodo-4-(1-methoxyethyl)phenol was 50.88:47.15.

As a result of the analysis by liquid chromatography-mass spectrometry (LC-MS), the molecular weights were found to be 390 and 404, and the substance was confirmed to be a mixture of 1-(4-hydroxy-3,5-diiodophenyl)ethanol and 2,6-diiodo-4-(1-methoxyethyl)phenol.

The following peaks were found by carrying out ¹H-NMR measurement under the above measurement conditions, and the compound was confirmed to have the chemical structure.

δ (ppm) (d6-DMSO): 9.4 (1H, —OH), 7.7 (2H, Ph), 5.2 (0.5H, —CH—OH), 4.6-4.3 (1H, —CH—OH), 3.0 (1.5H, —O—CH3), 1.3 (3H, —CH3)

### Example 4

### Synthesis of 1-(4-hydroxy-3,5-diiodophenyl)ethanol

A reactor was charged with 1.1881 g of 1-(4-hydroxyphenyl)ethanol, 1.7472 g of iodine, 15.48 mL of methanol, and 1.72 mL of pure water, the reactor was immersed in an ice bath, and stirring was initiated. Subsequently, 0.8687 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.172 mL of an aqueous sodium hydrogen sulfite solution with a mass percent concentration of 35 was added to quench the reaction. Subsequently, the reaction solution was gradually added to 34.40 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 3.1023 g of a mixture of 1-(4-hydroxy-3,5-diiodophenyl)ethanol and 2,6-diiodo-4-(1-methoxyethyl)phenol. As a result of the HPLC analysis using a UV detector at a measurement wavelength of 254 nm, the ratio between 1-(4-hydroxy-3,5-diiodophenyl)ethanol and 2,6-diiodo-4-(1-methoxyethyl)phenol was 83.16:16.03.

As a result of the analysis by liquid chromatography-mass spectrometry (LC-MS), the molecular weights were found to be 390 and 404, and the substance was confirmed to be a mixture of 1-(4-hydroxy-3,5-diiodophenyl)ethanol and 2,6-diiodo-4-(1-methoxyethyl)phenol.

### Example 5

### Synthesis of 1-(4-hydroxy-3,5-diiodophenyl)ethanol

A reactor was charged with 1.2086 g of 1-(4-hydroxyphenyl)ethanol, 1.7787 g of iodine, 14.00 mL of methanol, and 3.50 mL of pure water, the reactor was immersed in an ice bath, and stirring was initiated. Subsequently, 0.8795 g of an aqueous iodic acid solution with a mass percent concentration of 70 was added dropwise thereto for 30 minutes. Subsequently, the reactor was immersed in a water bath at 25° C., and stirring was continued over 3.5 hours. Subsequently, 0.175 mL of an aqueous sodium hydrogen sulfite solution with a mass percent concentration of 35 was added to quench the reaction. Subsequently, the reaction solution was gradually added to 35.00 mL of pure water with vigorous stirring and mixed. Subsequently, the precipitate was filtered off with a suction filter, compressed, and washed with an aqueous methanol solution with a volume percent concentration of 33.3. Subsequently, the precipitate was dried in vacuo at 40° C. to obtain 3.1655 g of a mixture of 1-(4-hydroxy-3,5-diiodophenyl)ethanol and 2,6-diiodo-4-(1-methoxyethyl)phenol. As a result of the HPLC analysis using a UV detector at a measurement wavelength of 254 nm, the ratio between 1-(4-hydroxy-3,5-diiodophenyl)ethanol and 2,6-diiodo-4-(1-methoxyethyl)phenol was 73.88:25.39.

As a result of the analysis by liquid chromatography-mass spectrometry (LC-MS), the molecular weights were found to be 390 and 404, and the substance was confirmed to be a mixture of 1-(4-hydroxy-3,5-diiodophenyl)ethanol and 2,6-diiodo-4-(1-methoxyethyl)phenol.

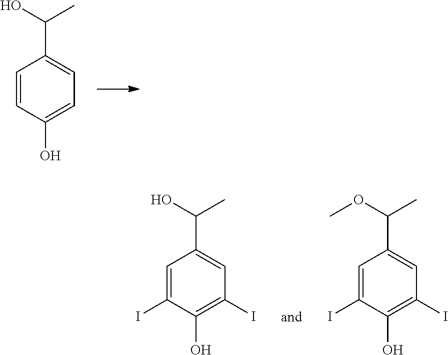

### Example 6

### Synthesis of 1-(4-hydroxy-3,5-diiodophenyl)ethanol

The reactor was charged with 8.77 g of sodium borohydride and 180 mL of tetrahydrofuran, the reactor was immersed in an ice bath, and stirring was initiated. Subsequently, a mixed solution consisting of 60.00 g of 4′-hydroxy-3′,5′-diiodoacetophenone, 9.31 g of isopropanol, and 180 mL of tetrahydrofuran were added dropwise thereto over 3 hours. Subsequently, stirring was continued over 9 hours while the reactor was immersed in the ice bath. Subsequently, 59.47 g of methanol was added to quench the reaction. Subsequently, the pressure in the reactor was reduced to 50 hPa, immersed in a water bath at 20° C. to concentrate the reaction solution. Subsequently, the reactor was immersed in an ice bath, and 120 mL of cold methanol was added to dilute the reaction solution. Subsequently, the pressure in the reactor was reduced to 50 hPa, immersed in a water bath at 20° C. to concentrate the reaction solution. Subsequently, the reactor was immersed in an ice bath, and 600 mL of cold methanol was added to dilute the reaction solution. Subsequently, the reaction solution was gradually added to 1,200 g of dilute sulfuric acid with a mass percent concentration of 1 with vigorous stirring and mixed. Subsequently, the precipitate was filtered off with a suction filter, compressed, and washed with 300 mL of an aqueous methanol solution with a volume percent concentration of 33.3. Subsequently, the precipitate was dried in vacuo at 40° C. to obtain 58.64 g of 1-(4-hydroxy-3,5-diiodophenyl)ethanol. The yield was 97.2%. As a result of the analysis by liquid chromatography-mass spectrometry (LC-MS), the molecular weight was found to be 390, and the substance was confirmed to be 1-(4-hydroxy-3,5-diiodophenyl)ethanol.

The following peaks were found by carrying out ¹H-NMR measurement under the above measurement conditions, and the compound was confirmed to have the chemical structure.

δ (ppm) (d6-DMSO): 9.4 (1H, —OH), 7.7 (2H, Ph), 5.2 (1H, —CH—OH), 4.6 (1H, —CH—OH), 1.3 (3H, —CH3)

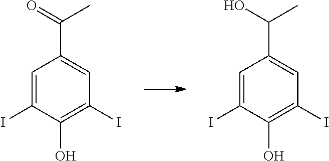

### Example 7

### Synthesis of 4-hydroxy-3,5-diiodostyrene

The reactor was charged with 120.00 g of 1-(4-hydroxy-3,5-diiodophenyl)ethanol, 7.94 g of concentrated sulfuric acid, 0.30 g of 4-hydroxy-2,2,6,6-tetramethylpiperidine1-oxyl free radical, and 1,500 mL of dimethylsulfoxide, and stirring was initiated. Subsequently, the pressure in the reactor was reduced to 30 hPa, and blowing of air into the reaction solution at a flow rate of 9 mL/minute was initiated. Subsequently, the reactor was immersed in a water bath at 90° C., and stirring was continued over 5 hours. Subsequently, the reactor was immersed in a water bath at 25° C. to cool the reaction solution. Subsequently, the reaction solution was gradually added to 3,000 g of an aqueous sodium hydrogen sulfite solution with a mass percent concentration of 0.1 with vigorous stirring and mixed. Subsequently, the precipitate was filtered off with a suction filter, compressed, and washed with 1,500 mL of an aqueous methanol solution with a volume percent concentration of 33.3. Subsequently, the precipitate was dried in vacuo at 40° C. to obtain 109.69 g of 4-hydroxy-3,5-diiodostyrene. The yield was 95.8%.

As a result of the analysis by liquid chromatography-mass spectrometry (LC-MS), the molecular weight was found to be 372, and the substance was confirmed to be 4-hydroxy-3,5-diiodostyrene.

The following peaks were found by carrying out ¹H-NMR measurement under the above measurement conditions, and the compound was confirmed to have the chemical structure.

δ (ppm) (d6-DMSO): 9.6 (1H, OH), 7.9 (2H, Ph), 6.6 (1H, —CH2-), 5.7 (1H, ═CH2), 5.1 (1H, ═CH2)

### Example 8

### Synthesis of 4-hydroxy-3,5-diiodostyrene

The reactor was charged with 2.0045 g of a mixture in which the ratio between 1-(4-hydroxy-3,5-diiodophenyl)ethanol and 2,6-diiodo-4-(1-methoxyethyl)phenol is 74.40:24.18, 0.2895 mL of concentrated sulfuric acid, 0.0020 g of 4-hydroxy-2,2,6,6-tetramethylpiperidine1-oxyl free radical, and 20 mL of dimethylsulfoxide, and stirring was initiated. Subsequently, the pressure in the reactor was reduced to 30 hPa, the reactor was immersed in a water bath at 90° C., and stirring was continued over 3 hours. Subsequently, the reactor was immersed in a water bath at 25° C. to cool the reaction solution. As a result of the HPLC analysis using a UV detector at a measurement wavelength of 254 nm, the ratio among 1-(4-hydroxy-3,5-diiodophenyl)ethanol, 2,6-diiodo-4-(1-methoxyethyl)phenol, and 4-hydroxy-3,5-diiodostyrene in the reaction solution was 0.08:0.01:98.12.

As a result of the analysis by liquid chromatography-mass spectrometry (LC-MS), the molecular weight was found to be 372, and the substance was confirmed to be 4-hydroxy-3,5-diiodostyrene.

The compound was confirmed to have a similar chemical structure by carrying out ¹H-NMR measurement under the above measurement conditions.

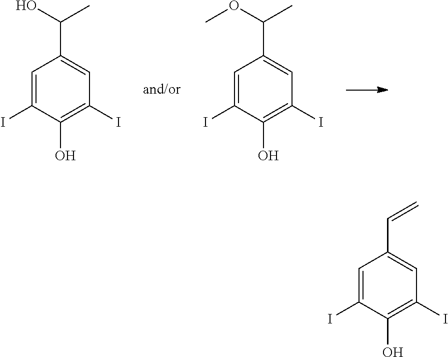

### Example 9

### Synthesis of 4-acetoxy-3,5-diiodostyrene

Using a 100 mL glass flask as a reaction vessel, 16.7 g (45 mmol) of 4-hydroxy-3,5-diiodostyrene was dissolved by using dimethylsulfoxide a solvent, 2 eq. of acetic anhydride and 1 eq. of sulfuric acid were added thereto, and then the mixture was warmed to 80° C. and stirred for 3 hours. Thereafter, the stirred solution was cooled, and the precipitate was filtered off, washed, and dried to obtain 9.0 g of a white solid. The white solid sample was analyzed by liquid chromatography-mass spectrometry (LC-MS) and, as a result, the molecular weight was found to be 414 and the white solid was confirmed to be 4-acetoxy-3,5-diiodostyrene.

The following peaks were found by carrying out ¹H-NMR measurement under the above measurement conditions, and the compound was confirmed to have the chemical structure.

δ (ppm) (d6-DMSO): 7.9 (2H, Ph), 6.6 (1H, —CH2-), 5.7 (1H, ═CH2), 5.1 (1H, ═CH2), 2.3 (3H, —CH3)

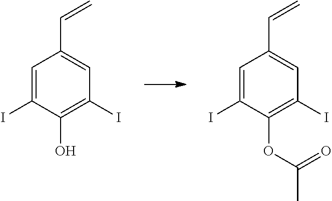

### INDUSTRIAL APPLICABILITY

The present invention can provide a compound, a polymer, a composition, a composition for film formation, a pattern formation method, and an insulating film formation method, by which a film having excellent sensitivity to an exposure light source can be obtained, and they can be utilized for a photoresist to be used in photolithography in the production of semiconductor elements and liquid crystal display elements.

1. A compound having one or more halogens and an unsaturated double bond.

2. The compound according to claim 1 having one or more hydrophilic groups or one decomposable group.

3. The compound according to claim 1 represented by the following formula (1): 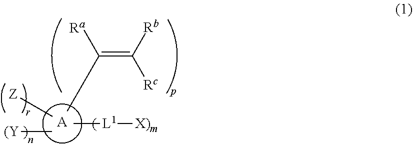
wherein
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
each of R^{a}, R^{b}, and R^{c} is independently H, I, F, Cl, Br, or an organic group having 1 to 60 carbon atoms and optionally having a substituent;
A is an organic group having 1 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
p is an integer of 1 or more, m is an integer of 1 or more, n is an integer of 0 or more, and r is an integer of 0 or more.

4. The compound according to claim 3 represented by the following formula (1a): 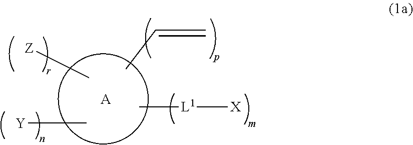
wherein
X, L¹, Y, A, Z, p, m, n, and r are as defined in the formula (1).

5. The compound according to claim 3 represented by the following formula (1b): 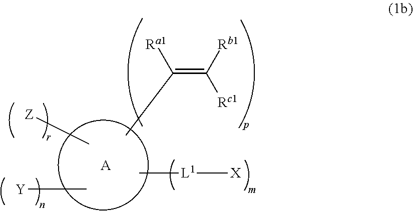
wherein
X, L¹, Y, A, Z, p, m, n, and r are as defined in the formula (1);
each of R^{a1}, R^{b1}, and R^{c1} is independently H, I, F, Cl, Br, or an organic group having 1 to 60 carbon atoms and optionally having a substituent; and
at least one of R^{a1}, R^{b1}, and R^{c1} is I, F, Cl, Br, or an organic group having 1 to 60 carbon atoms and optionally having a substituent.

6. The compound according to claim 3 wherein n+r is an integer of 1 or more.

7. The compound according to claim 3 wherein each Y is independently a group represented by the following formula (Y-1):
-L²-R² (Y-1)
wherein
L² is a group which is cleaved by an action of an acid or a base; and
R² is a linear, branched, or cyclic aliphatic group having 1 to 30 carbon atoms, an aromatic group having 1 to 30 carbon atoms, a linear, branched, or cyclic aliphatic group containing a heteroatom and having 1 to 30 carbon atoms, or an aromatic group containing a heteroatom and having 1 to 30 carbon atoms, and the aliphatic group, the aromatic group, the aliphatic group containing a heteroatom, and the aromatic group containing a heteroatom of R² optionally further have a substituent.

8. The compound according to claim 3 wherein A is an aromatic ring.

9. The compound according to claim 3 wherein A is an alicyclic structure.

10. The compound according to claim 3 wherein A is a heterocyclic structure.

11. The compound according to claim 3 wherein n is 2 or more.

12. The compound according to claim 1, comprising a functional group for improving solubility in an alkaline developing solution by the action of an acid or a base.

13. The compound according to claim 3 wherein X is I, and L¹ is a single bond.

14. The compound according to claim 3 wherein X is an aromatic group into which one or more F, Cl, Br, or I are introduced.

15. The compound according to claim 3 wherein X is an alicyclic group into which one or more F, Cl, Br, or I are introduced.

16. A composition comprising a compound represented by the formula (1C) in an amount of 1 ppm by mass or more and 10% by mass or less, based on the total compound according to claim 1: 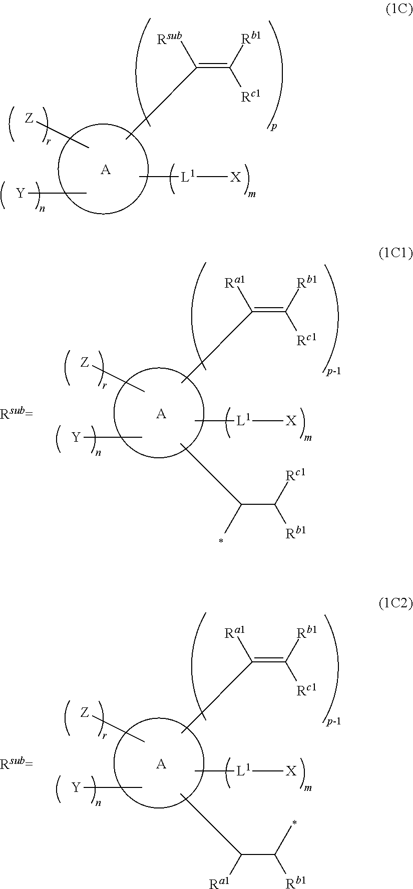
wherein
X, L¹, Y, A, Z, p, m, n, and r are as defined in the formula (1);
R^{sub} represents the formula (1C1) or the formula (1C2);
each of R^{a1}, R^{b1}, and R^{c1} is independently H, I, F, Cl, Br, or an organic group having 1 to 60 carbon atoms and optionally haying a substituent;
at least one of R^{a1}, R^{b1}, and R^{c1} is I, F, Cl, Br, or an organic group haying 1 to 60 carbon atoms and optionally having a substituent;
p-1 is an integer of 0 or more; and
* is a site for binding with an adjacent constitutional unit.

17. A composition comprising the compound according to claim 1 and a compound represented by the formula (1D) in an amount of 1 ppm by mass or more and 10% by mass or less, based on the compound according to claim 1: 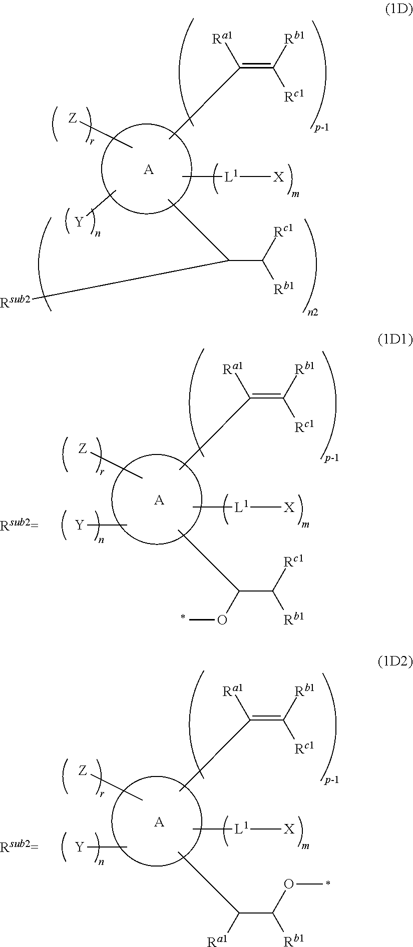
wherein
X, L¹, Y, A, Z, p, m, n, and r are as defined in the formula (1);
R^{sub2} represents the formula (1D1) or the formula (1D2);
each of R^{a1}, R^{b1}, and R^{c1} is independently H, I, F, Cl, Br, or an organic group having 1 to 60 carbon atoms and optionally haying a substituent;
at least one of R^{a1}, R^{b1}, and R^{c1} is I, F, Cl, Br, or an organic group haying 1 to 60 carbon atoms and optionally having a substituent;
n2 represents an integer of 0 or more and 4 or less;
p-1 is an integer of 0 or more; and
* is a site for binding with an adjacent constitutional unit.

18. A composition comprising a compound represented by the formula (1E) in an amount of 1 ppm by mass or more and 10% by mass or less, based on the compound according to claim 3: 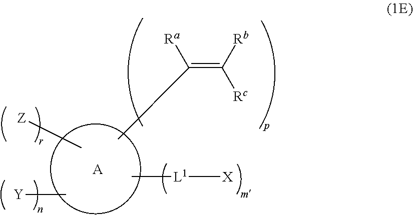
wherein
each X is independently F, Cl, Br, or an organic group haying 1 to 30 carbon atoms and having 1 or more and 5 or less substituents selected from the group consisting of F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;
each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
each of R^{a}, R^{b}, and R^{c} is independently H, F, Cl, Br, or an organic group having 1 to 60 carbon atoms and optionally having a substituent;
A is an organic group having 1 to 30 carbon atoms;
each Z is independently an alkoxy group, an ester group, an acetal group, or a carbonate ester group;
provided that none of X, L¹, Y, R^{a}, R^{b}, R^{c}, A, and Z contains I; and
p is an integer of 1 or more, m′ is an integer of 0 or more, n is an integer of 0 or more, and r is an integer of 0 or more.

19. A composition comprising the compound according to claim 1,
wherein impurities containing K are 1 ppm by mass or less, in terms of element, based on the compound.

20. The composition according to claim 19, wherein peroxide is 10 ppm by mass or less, based on the compound.

21. The composition according to claim 19, wherein impurities containing one or more elements selected from the group consisting of Mn, Al, Si, and Li are 1 ppm by mass or less, in terms of element, based on the compound.

22. The composition according to claim 19, wherein a phosphorus-containing compound is 10 ppm by mass or less, based on the compound.

23. The composition according to claim 19, wherein maleic acid is 10 ppm by mass or less, based on the compound.

24. A polymer comprising a constitutional unit derived from the compound according to claim 1.

25. The polymer according to claim 24, further comprising a constitutional unit represented by the following formula (C6): 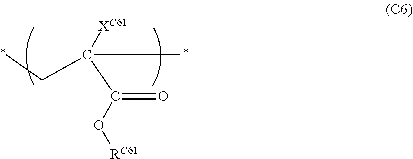
wherein
X^{C61} is a hydroxyl group or a halogen group;
each R^{C61} is independently an alkyl group having 1 to 20 carbon atoms; and
* is a site for binding with an adjacent constitutional unit.

26. A composition for film formation comprising the polymer according to claim 24.

27. The composition for film formation according to claim 26, further comprising an acid generating agent, a base generating agent, or a base compound.

28. A resist pattern formation method comprising:
a step of forming a resist film on a substrate by using a composition for film formation containing the polymer according to claim 24;
a step of exposing a pattern on the resist film; and
a step of subjecting the resist film to a development treatment after exposure.

29. An insulating film formation method comprising the method according to claim 28.

30. A method for producing a compound represented by the following formula (0), comprising a double bond introduction step of introducing an unsaturated double bond into a substituent Q of a compound represented by the following formula (S1): 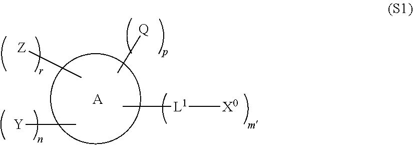
wherein
X⁰ is an organic group having 1 to 30 carbon atoms;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group;
each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
A is an organic group having 1 to 30 carbon atoms;
each Z is independently an alkoxy group, an ester group, an acetal group, or a carbonate ester group;
Q is an organic group having 1 to 30 carbon atoms and having a hydroxyl group, an aldehyde group, a carboxyl group, or a ketone group; and
p is an integer of 1 or more, m′ is an integer of 0 or more, n is an integer of 0 or more, and r is an integer of 0 or more; 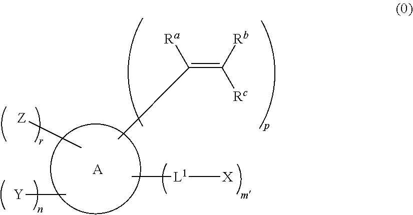
wherein
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group;
each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
each of R^{a}, R^{b}, and R^{c} is independently H, I, F, Cl, Br, or an organic group having 1 to 60 carbon atoms and optionally having a substituent;
A is an organic group having 1 to 30 carbon atoms;
each Z is independently an alkoxy group, an ester group, an acetal group, or a carbonate ester group; and
p is an integer of 1 or more, m′ is an integer of 0 or more, n is an integer of 0 or more, and r is an integer of 0 or more.

31. The method for producing a compound according to claim 30, wherein the compound represented by the formula (S1) is a compound represented by the following formula (SA1), and
the method comprises a step designated by the following A1 and a step designated by the following A2:
A1) a step of obtaining a compound represented by the following formula (SA2) by using the compound represented by the formula (SA1), and a compound represented by the following formula (RM1) or malononitrile; and
A2) a step of obtaining a compound represented by the formula (0) by using a compound represented by the formula (SA2) and a fluoride source, 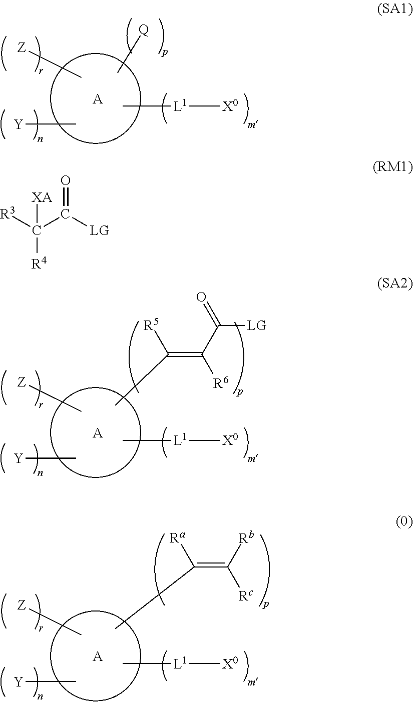
wherein
X⁰, L¹, Y, A, Z, p, m′, n, and r are as defined in the formulas (S1) and (0);
Q¹ is aldehyde or ketone;
LG is a group selected from a hydroxy groups, an alkoxy group, a carbonate ester group, an acetal group, and a carboxy group, and the alkoxy group, the carbonate ester group, the acetal group, and the carboxy group contain an aliphatic group or an aromatic group optionally having a substituent having 1 to 60 carbon atoms;
R³ is a hydrogen group, or a carboxy group or ester group optionally having a substituent having 1 to 60 carbon atoms;
R⁴ is a hydrogen group;
each of R⁵ and R⁶ is independently H, F, Cl, Br, or an organic group having 1 to 60 carbon atoms and optionally having a substituent; and
XA is a group selected from a hydrogen group and a halogen group.

32. The method for producing a compound according to claim 31, wherein in the step designated by A2, the compound represented by the formula (SA2) is subjected to a decarboxylation reaction at 100° C. or less by using the fluoride source.

33. The method for producing a compound according to claim 31, wherein in the step designated by A1, the compound represented by the formula (SA2) is obtained by further using a reducing agent.

34. The method for producing a compound according to claim 30, wherein in the above formula (S1), A is benzene, toluene, or a heteroaromatic ring.

35. A method for producing the compound represented by the following formula (1), comprising: a step of forming a compound represented by the following formula (SB1) by at least one of compounds represented by the following formula (SB2A) and the following formula (SB3A) obtained through a step designated by the following B1A and at least one of steps designated by the following B2A and B3A; and a double bond introduction step of introducing an unsaturated double bond into a substituent Qb of the compound represented by the formula (SB1):
B1A) a step of providing the following substrate SB1A comprising one or more amino groups, and a core B having an aldehyde group or a ketone group;
B2A) a step of obtaining the compound represented by the following formula (SB2A) in which iodine is introduced into the core B; and
B3A) a step of obtaining the compound represented by the formula (SB3A) in which an amino group is substituted with a halogen group by a Sandmeyer reaction, 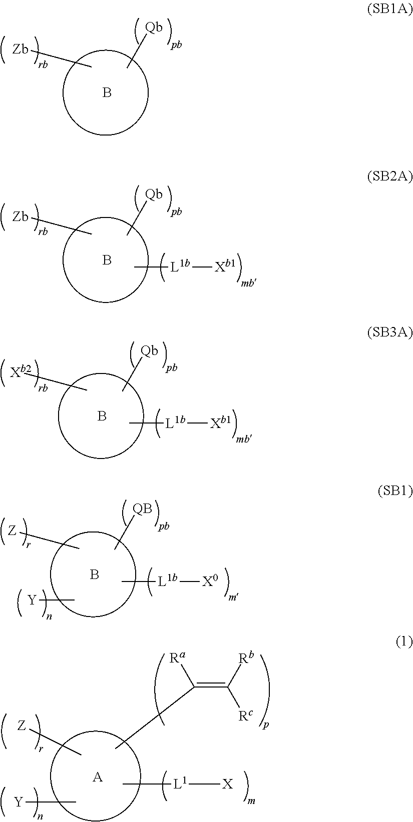
wherein, in the formula (1),
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group, and the ether group, the ester group, the thioether group, the amino group, the thioester group, the acetal group, the phosphine group, the phosphone group, the urethane group, the urea group, the amide group, the imide group, or the phosphate group of L¹ optionally has a substituent;
each Y is independently a hydroxyl group, an alkoxy group, an ester group, an acetal group, a carboxyalkoxy group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
each of R^{a}, R^{b}, and R^{c} is independently H, I, F, Cl, Br, or an organic group having 1 to 60 carbon atoms and optionally having a substituent;
A is an organic group having 1 to 30 carbon atoms;
each Z is independently an alkoxy group, an ester group, an acetal group, a carboxyalkoxy group, or a carbonate ester group, and the alkoxy group, the ester group, the acetal group, the carboxyalkoxy group, or the carbonate ester group of Z optionally has a substituent; and
p is an integer of 1 or more, m is an integer of 1 or more, n is an integer of 0 or more, and r is an integer of 0 or more;
wherein, in the formulas (SB1A), (SB2A), (SB3A), and (SB1),
Zb represents an amino group optionally having a substituent consisting of a hydrogen group or a hydrocarbon group having 1 to 30 carbon atoms and optionally having a substituent, rb represents an integer of 1 or more, L^{1b}, X^{b1}, B, pb, and mb′ have the same meanings as L, X, A, p, and m in the formula (1), respectively; X^{b2} represents I, F, Cl, Br, or an organic group having 1 to 30 carbon atoms and having 1 or more and 5 or less substituents selected from the group consisting of I, F, Cl, and Br; and Qb is an organic group having 1 to 30 carbon atoms and having a hydroxyl group, an aldehyde group, a carboxyl group, or a ketone group.

36. The production method according to claim 35, wherein the step of introducing a double bond comprises using an organic phosphorus compound and a base.

37. The method for producing a compound according to claim 30, comprising a halogen introduction step of introducing a halogen atom into the compound represented by the above formula (S1) by reaction with a halogenating agent.

38. The method for producing a compound according to claim 30, wherein the compound represented by the formula (SA1) is at least one of the compounds represented by the following formula (SB2A) and the following formula (SB3A) obtained through a step designated by the following B1A and at least one of steps designated by the following B2A and B3A:
B1A) a step of providing the following substrate SB1A comprising one or more amino groups, and a core B having an aldehyde group or a ketone group;
B2A) a step of obtaining the compound represented by the following formula (SB2A) in which iodine is introduced into the core B; and
B3A) a step of obtaining the compound represented by the formula (SB3A) in which an amino group is substituted with a halogen group by a Sandmeyer reaction, 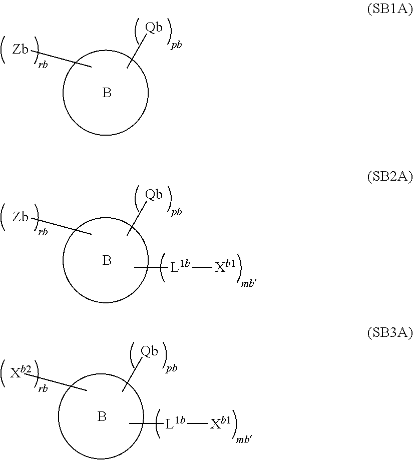
wherein
Zb represents an amino group optionally having a substituent consisting of a hydrogen group or a hydrocarbon group having 1 to 30 carbon atoms and optionally having a substituent, rb represents an integer of 1 or more, L^{1b}, X^{b1}, B, pb, and mb′ have the same meanings as L, X, A, p, and m in the formula (1), respectively; X^{b2} represents I, F, Cl, Br, or an organic group having 1 to 30 carbon atoms and having 1 or more and 5 or less substituents selected from the group consisting of I, F, Cl, and Br; and Qb has the same meanings as Q in the formula (S1).

39. The method for producing a compound according to claim 36, wherein in the step designated by B2A, iodine is introduced into the core B by at least using an iodine source and an oxidizing agent.

40. The method for producing a compound according to claim 30, wherein the compound represented by the formula (SA1) is a compound produced by a step designated by the following B1B, and at least either one of steps designated by the following B2B and B3B:
B1B) a step of providing the following substrate SB1B comprising one or more amino groups, and a core B having an aldehyde group or a ketone group;
B2B) a step of obtaining a compound represented by the formula (SB2B) in which iodine is introduced into the core B; and
B3B) a step of obtaining a compound represented by the formula (SB3B) in which an amino group is substituted with a halogen group, 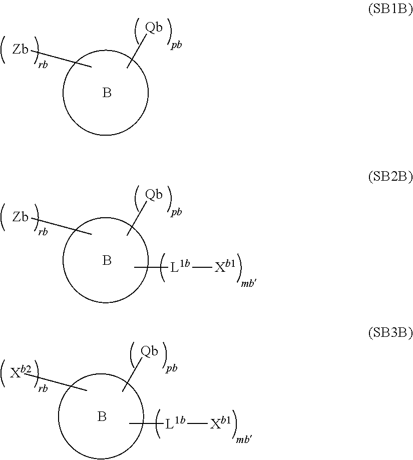
wherein
Zb represents an amino group optionally having a substituent consisting of a hydrogen group or a hydrocarbon group having 1 to 30 carbon atoms and optionally having a substituent, rb represents an integer of 1 or more, L^{1b}, X^{b1}, B, pb, and mb′ have the same meanings as L, X, A, p, and m in the formula (1), respectively; X^{b2} represents I, F, Cl, Br, or an organic group having 1 to 30 carbon atoms and having 1 or more and 5 or less substituents selected from the group consisting of I, F, Cl, and Br; and Qb has the same meanings as Q in the formula (S1).

41. The method for producing a compound according to claim 40, further comprising a step designated by the following B4a:
B4a) Wittig step.

42. The method for producing a compound according to claim, wherein in the step designated by B2B, iodine is introduced into the core B by at least using an iodine source and an oxidizing agent.

43. The method for producing a compound according to claim 40, wherein the core B has an aromatic ring structure optionally having a heteroatom.

44. A method for producing a compound represented by the following formula (1), comprising:
a halogen introduction step of introducing a halogen atom into a compound represented by the following formula (S1) by reaction with a halogenating agent; and
a double bond introduction step of introducing an unsaturated double bond into a substituent Q;
wherein the double bond introduction step comprises using an organic phosphorus compound and a base: 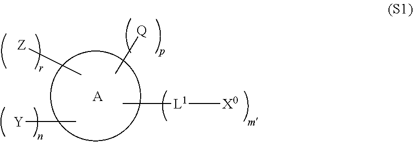
wherein
X⁰ is an organic group having 1 to 30 carbon atoms;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group;
each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
A is an organic group having 1 to 30 carbon atoms;
each Z is independently an alkoxy group, an ester group, an acetal group, or a carbonate ester group;
Q is an organic group having 1 to 30 carbon atoms and having a hydroxyl group, an aldehyde group, a carboxyl group, or a ketone group; and
p is an integer of 1 or more, m′ is an integer of 0 or more, n is an integer of 0 or more, and r is an integer of 0 or more, 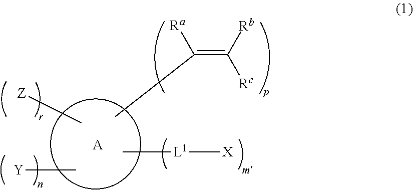
wherein
each X is independently I, F, Cl, Br, or an organic group having 1 to 30 carbon atoms and having 1 or more and 5 or less substituents selected from the group consisting of I, F, Cl, and Br;
each L¹ is independently a single bond, an ether group, an ester group, a thioether group, an amino group, a thioester group, an acetal group, a phosphine group, a phosphone group, a urethane group, a urea group, an amide group, an imide group, or a phosphate group;
each Y is independently a hydroxyl group, an alkoxy group, an ester group, an acetal group, a carbonate ester group, a nitro group, an amino group, a carboxyl group, a thiol group, an ether group, a thioether group, a phosphine group, a phosphone group, a urethane group, a urea group, an amide group, an imide group, or a phosphate group, and the alkoxy group, the ester group, the carbonate ester group, the amino group, the ether group, the thioether group, the phosphine group, the phosphone group, the urethane group, the urea group, the amide group, the imide group, and the phosphate group of Y optionally have a substituent;
each of R^{a}, R^{b}, and R^{c} is independently H, I, F, Cl, Br, or an organic group having 1 to 60 carbon atoms and optionally having a substituent;
A is an organic group having 1 to 30 carbon atoms;
each Z is independently an alkoxy group, an ester group, an acetal group, or a carbonate ester group; and
p is an integer of 1 or more, m is an integer of 1 or more, n is an integer of 0 or more, and r is an integer of 0 or more.

45. A method for producing an iodine-containing vinyl monomer comprising:
a) a step of providing an iodine-containing alcohol substrate having a general structure represented by the formula (1-1): 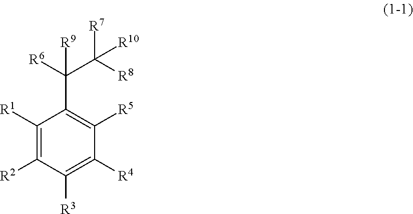
wherein
each of R¹ to R⁵ is independently H, OH, OCH₃, a halogen, or a linear or branched alkyl,
each of R⁶ to R¹⁰ is independently H, OH, OCH₃, a halogen, or a cyano group,
provided that at least one of R¹ to R⁵ is OH, at least one of R¹ to R⁵ is iodine, and at least one of R⁶ to R¹⁰ is OH or OCH₃; and
b) a step of dehydrating the iodine-containing alcohol substrate to obtain the iodine-containing vinyl monomer having a general structure represented by the formula (1): 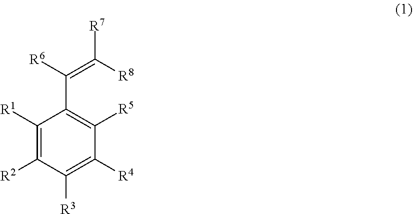
wherein
each of R¹ to R⁵ is independently H, OH, OCH₃, a halogen, or a linear or branched alkyl,
each of R⁶ to R⁸ is independently H, OH, OCH₃, a halogen, or a cyano group,
provided that at least one of R¹ to R⁵ is OH, and at least one of R¹ to R⁵ is iodine.

46. The method for producing an iodine-containing vinyl monomer according to claim 45, wherein the step of providing an iodine-containing alcohol substrate having a general structure represented by the formula (1-1) comprises:
c) a step of providing an iodine-containing ketone substrate having a general structure represented by the formula (1-2): 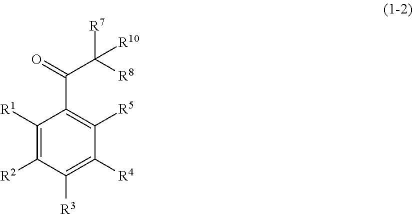
wherein
each of R¹ to R⁵ is independently H, OH, OCH₃, a halogen, or a linear or branched alkyl,
each of R⁷, R⁸, and R¹⁰ is independently H, OH, OCH₃, a halogen, or a cyano group,
provided that at least one of R¹ to R⁵ is OH and at least one of R¹ to R⁵ is iodine; and
d) a step of reducing the iodine-containing ketone substrate to obtain the iodine-containing alcohol substrate having the general structure represented by the formula (1-1).

47. The method for producing an iodine-containing vinyl monomer according to claim 45, wherein the step of providing an iodine-containing alcohol substrate having a general structure represented by the formula (1-1) comprises:
e) a step of providing an alcohol substrate having a general structure represented by the formula (1-3): 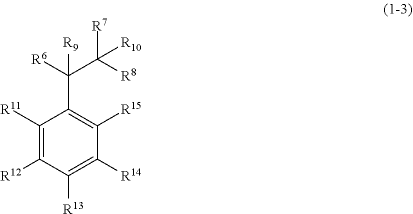
wherein
each of R¹¹ to R¹⁵ is independently H, OH, OCH₃, or a linear or branched alkyl,
each of R⁶ to R¹⁰ is independently H, OH, OCH₃, a halogen, or a cyano group,
provided that at least one of R¹¹ to R¹⁵ is OH and at least one of R⁶ to R¹⁰ is OH or OCH₃; and
f) a step of introducing iodine into the alcohol substrate to obtain the iodine-containing alcohol substrate having the general structure represented by the formula (1-1).

48. The method for producing an iodine-containing vinyl monomer according to claim 46, wherein the step of providing an iodine-containing ketone substrate having a general structure represented by the formula (1-2) comprises:
g) a step of providing a ketone substrate having a general structure represented by the formula (1-4): 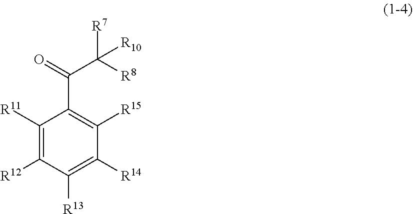
wherein
each of R¹¹ to R¹⁵ is independently H, OH, OCH₃, or a linear or branched alkyl,
each of R⁷, R⁸, and R¹⁰ is independently H, OH, OCH₃, a halogen, or a cyano group,
provided that at least one of R¹¹ to R¹⁵ is OH; and
h) a step of introducing iodine into the ketone substrate to obtain the iodine-containing ketone substrate having the general structure represented by the formula (1-2).

49. The method for producing an iodine-containing vinyl monomer according to claim 47, wherein the step of providing an alcohol substrate having a general structure represented by the formula (1-3) comprises:
i) a step of providing a ketone substrate having a general structure represented by the formula (1-4): 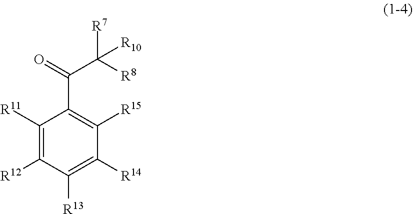
wherein
each of R¹¹ to R¹⁵ is independently H, OH, OCH₃, or a linear or branched alkyl,
each of R⁷, R⁸, and R¹⁰ is independently H, OH, OCH₃, a halogen, or a cyano group, provided that at least one of R¹¹ to R¹⁵ is OH; and
j) a step of reducing the ketone substrate to obtain the alcohol substrate having the general structure represented by the formula (1-3).

50. A method for producing an iodine-containing acetylated vinyl monomer comprising:
k) a step of providing an iodine-containing vinyl monomer having a general structure represented by the formula (1): 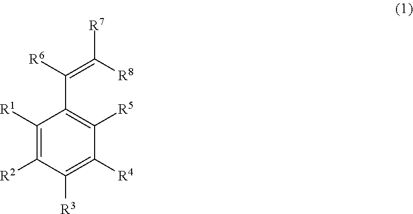
wherein
each of R¹ to R⁵ is independently H, OH, OCH₃, a halogen, or a linear or branched alkyl,
each of R⁶ to R⁸ is independently H, OH, OCH₃, a halogen, or a cyano group,
provided that at least one of R¹ to R⁵ is OH and at least one of R¹ to R⁵ is iodine; and
l) a step of acetylating the iodine-containing vinyl monomer to obtain the iodine-containing acetylated vinyl monomer having a general structure represented by the formula (2): 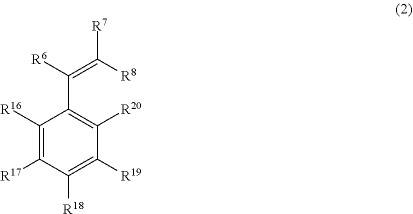
wherein
each of R¹⁶ to R²⁰ is independently H, OH, OCH₃, OAc, a halogen, or a linear or branched alkyl,
each of R⁶ to R⁸ is independently H, OH, OCH₃, a halogen, or a cyano group,
provided that at least one of R¹⁶ to R²⁰ is OAc and at least one of R¹⁶ to R²⁰ is iodine.

51. A method for producing an iodine-containing alcohol substrate comprising:
c) a step of providing an iodine-containing ketone substrate having a general structure represented by the formula (1-2): 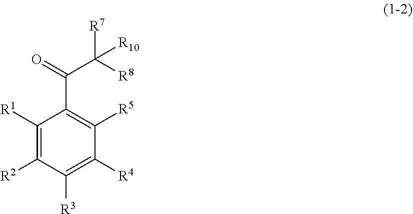
wherein
each of R¹ to R⁵ is independently H, OH, OCH₃, a halogen, or a linear or branched alkyl,
each of R⁷, R⁸, and R¹⁰ is independently H, OH, OCH₃, a halogen, or a cyano group,
provided that at least one of R¹ to R⁵ is OH and at least one of R¹ to R⁵ is iodine; and
d) a step of reducing the iodine-containing ketone substrate to obtain the iodine-containing alcohol substrate having a general structure represented by the formula (1-1): 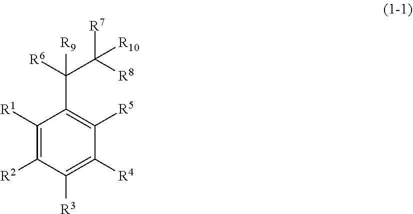
wherein
each of R¹ to R⁵ is independently H, OH, OCH₃, a halogen, or a linear or branched alkyl,
each of R⁶ to R¹⁰ is independently H, OH, OCH₃, a halogen, or a cyano group,
provided that at least one of R¹ to R⁵ is OH, at least one of R¹ to R⁵ is iodine, and at least one of R⁶ to R¹⁰ is OH or OCH₃.

52. A method for producing an iodine-containing alcohol substrate comprising:
e) a step of providing an alcohol substrate having a general structure represented by the formula (1-3): 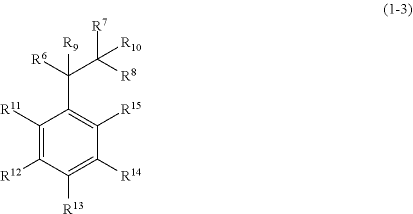
wherein
each of R¹¹ to R¹⁵ is independently H, OH, OCH₃, or a linear or branched alkyl,
each of R⁶ to R¹⁰ is independently H, OH, OCH₃, a halogen, or a cyano group,
provided that at least one of R¹¹ to R¹⁵ is OH and at least one of R⁶ to R¹⁰ is OH or OCH₃; and
f) a step of introducing iodine into the alcohol substrate to obtain the iodine-containing alcohol substrate having a general structure represented by the formula (1-1): 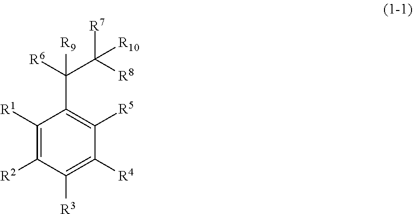
wherein
each of R¹ to R⁵ is independently H, OH, OCH₃, a halogen, or a linear or branched alkyl,
each of R⁶ to R¹⁰ is independently H, OH, OCH₃, a halogen, or a cyano group,
provided that at least one of R¹ to R⁵ is OH, at least one of R¹ to R⁵ is iodine, and at least one of R⁶ to R¹⁰ is OH or OCH₃.

53. A method for producing an iodine-containing ketone substrate comprising:
g) a step of providing a ketone substrate having a general structure represented by the formula (1-4): 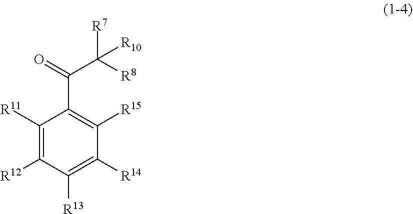
wherein
each of R¹¹ to R¹⁵ is independently H, OH, OCH₃, or a linear or branched alkyl,
each of R⁷, R⁸, and R¹⁰ is independently H, OH, OCH₃, a halogen, or a cyano group,
provided that at least one of R¹¹ to R¹⁵ is OH; and
h) a step of introducing iodine into the ketone substrate to obtain the iodine-containing ketone substrate having a general structure represented by the formula (1-2): 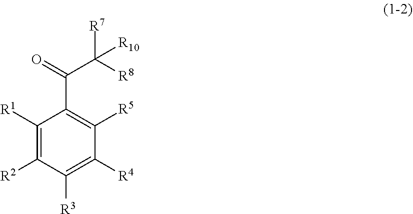
wherein
each of R¹ to R⁵ is independently H, OH, OCH₃, a halogen, or a linear or branched alkyl,
each of R⁷, R⁸, and R¹⁰ is independently H, OH, OCH₃, a halogen, or a cyano group,
provided that at least one of R¹ to R⁵ is OH, and at least one of R¹ to R⁵ is iodine.

54. A method for producing an alcohol substrate comprising:
i) a step of providing a ketone substrate having a general structure represented by the formula (1-4): 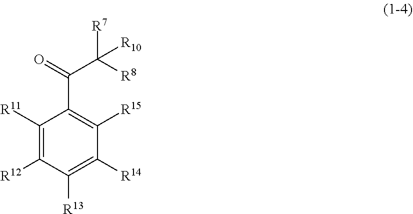
wherein
each of R¹¹ to R¹⁵ is independently H, OH, OCH₃, or a linear or branched alkyl,
each of R⁷, R⁸, and R¹⁰ is independently H, OH, OCH₃, a halogen, or a cyano group,
provided that at least one of R¹¹ to R¹⁵ is OH; and
j) a step of reducing the ketone substrate to obtain the alcohol substrate having a general structure represented by the formula (1-3): 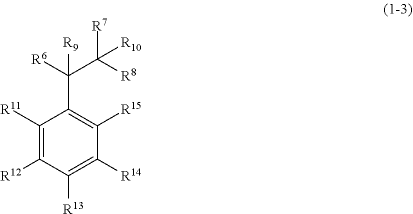
wherein
each of R¹¹ to R¹⁵ is independently H, OH, OCH₃, or a linear or branched alkyl,
each of R⁶ to R¹⁰ is independently H, OH, OCH₃, a halogen, or a cyano group,
provided that at least one of R¹¹ to R¹⁵ is OH and at least one of R⁶ to R¹⁰ is OH or OCH₃.
